(19) United States
(12) Patent Application Publication
(10) Pub. No.: US 2012/0040975 A1
(43) Pub. Date: Feb. 16, 2012

(54) BRIDGED BICYCLIC HETEROCYCLE DERIVATIVES AND METHODS OF USE THEREOF

(76) Inventors: Santhosh Francis Neelamkavil, Edison, NJ (US); Bernard R. Neustadt, West Orange, NJ (US); Andrew Stamford, Chatham Township, NJ (US); Yan Xia, Edison, NJ (US); Joel M. Harris, Blaine, MN (US); Craig D. Boyle, Branchburg, NJ (US); Samuel Chackalamannil, Califon, NJ (US); Dipshikha Biswas, Woodbridge, NJ (US); Hong Liu, Hillsborough, NJ (US); Jinsong Hao, Belle Mead, NJ (US); Claire M. Lankin, High Bridge, NJ (US); Unmesh Shah, Green Brook, NJ (US)

(21) Appl. No.: 13/260,950

(22) PCT Filed: Apr. 1, 2010
(86) PCT No.: PCT/US2010/029555

§ 371 Date: Sep. 29, 2011

Related U.S. Application Data
(60) Provisional application No. 61/166,422, filed on Apr. 3, 2009.

(51) Int. Cl. A61K 31/537; C07D 401/12; C07D 491/08; A61P 3/00; A61K 31/506; C07D 487/08; A61P 3/10; A61P 3/04; C07D 451/06; C07D 498/08

(52) U.S. Cl. 514/230.5; 544/319; 544/105; 514/269; 544/230

(57) ABSTRACT

The present invention relates to Bridged Bicyclic Heterocycle Derivatives, compositions comprising a Bridged Bicyclic Heterocycle Derivative, and methods of using the Bridged Bicyclic Heterocycle Derivatives for treating or preventing obesity, diabetes, a metabolic disorder, a cardiovascular disease or a disorder related to the activity of a GPCR in a patient.

### FIELD OF THE INVENTION

The present invention relates to Bridged Bicyclic Heterocycle Derivatives, compositions comprising a Bridged Bicyclic Heterocycle Derivative, and methods of using the Bridged Bicyclic Heterocycle Derivatives for treating or preventing obesity, diabetes, a diabetic complication, a metabolic disorder, a cardiovascular disease or a disorder related to the activity of a G-Protein Coupled Receptor (“GPCR”) in a patient.

### BACKGROUND OF THE INVENTION

Although a number of receptor classes exist in humans, by far the most abundant and therapeutically relevant is represented by the GPCR class. It is estimated that there are some 100,000 genes within the human genome, and of these, approximately 2% or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products, as evidenced by the fact that pharmaceutical products have been developed from approximately 20 of the 100 known GPCRs. This distinction is not merely semantic, particularly in the case of GPCRs.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., Life Sciences 43, 1095 (1988). Although other G proteins exist, currently, Gq, Gs, Gi, and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces a biological response. A receptor can be stabilized in an active state by an endogenous ligand or a compound such as a drug.

Modulation of G-protein coupled receptors has been well-studied for controlling various metabolic disorders. Small molecule modulators of the receptor GPR119, a G-protein coupled-receptor described in, for example, GenBank (see, e.g., accession numbers XM.sub.—066873 and AY288416), have been shown to be useful for treating or preventing certain metabolic disorders. GPR119 is a G protein-coupled receptor that is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo. See, e.g., International Publication Nos. WO 04/065380, WO 04/076413, and EP 1338651, the disclosure of each of which is herein incorporated by reference in its entirety.

U.S. Pat. No. 7,136,426 discloses pyrazolo[3,4-d]pyrimidine ethers and related compounds as modulators of the GPR119 receptor that are useful for the treatment of various metabolic-related disorders such as type I diabetes, type II diabetes, inadequate glucose tolerance, insulin resistance, hyperglycemia, hyperlipidemia, hypertriglyceridemia, hypercholesterolemia, dyslipidemia or syndrome X. The compounds are also reported as being useful for controlling weight gain, controlling food intake, and inducing satiety in mammals. The promising nature of these GPCR modulators indicates a need in the art for additional small molecule GPCR modulators with improved efficacy and safety profiles. This invention addresses that need.

### SUMMARY OF THE INVENTION

In one aspect, the present invention provides compounds of Formula (I):

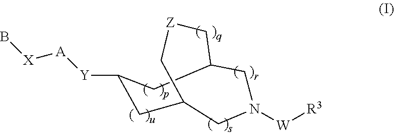

and pharmaceutically acceptable salts, solvates, esters, prodrugs and stereoisomers thereof, wherein:

A is pyridyl or pyrimidinyl, each of which can be optionally substituted with up to 2 groups, which can be the same or different, and are selected from alkyl, cycloalkyl, halo and —O-alkyl;

B is phenyl or 6-membered heteroaryl, any of which can be optionally substituted with up to 3 groups, which can be the same or different, and are selected from alkyl, heterocycloalkyl, heteroaryl, halo, —CN, —S(O)₂alkyl and —S(O)₂cycloalkyl, wherein a heterocycloalkyl or heteroaryl group can be unsubstituted or optionally substituted with alkyl, and wherein a ring —CH₂— group on a heterocycloalkyl group can be optionally replaced with a —C(O)— group;

W is a bond, —C(O)—, —C(O)NH—, —C(O)—O—, —C(O)—S— or —S(O)₂—;

X is —O-(alkylene)ₜ- or —NH—;

Y is —O— or —NH—;

Z is a bond, —C(O)—, —C(R¹)₂—, —O—, —S(O)₂— or —N(R⁴)—

each occurrence of R¹ is independently H or —OH; wherein two R¹ groups, together with the carbon atom(s) to which they are attached, can join to form a 3- to 6-membered cycloalkyl group or a 3- to 6-membered heterocycloalkyl group;

R³ is alkyl, -alkylene-aryl, -(alkylene)ₜ-cycloalkyl, haloalkyl, heteroaryl, -alkylene-O-alkyl,

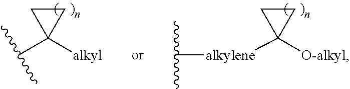

wherein a haloalkyl group can be optionally substituted with an —OH group, wherein a heteroaryl group can be optionally substituted with a group selected from alkyl, halo, —O-alkyl and cycloalkyl;

R⁴ is H, haloalkyl, aryl, cycloalkyl, cycloalkenyl, heterocycloalkyl, heterocycloalkenyl or heteroaryl;

n is an integer ranging from 1 to 4.

p is 0 or 1;

q is 0 or 1;

r is 0 or 1;

s is 0 or 1;

t is 0 or 1; and

u is 0 or 1.

The compounds of formula (I) and pharmaceutically acceptable salts, solvates, esters or prodrugs thereof (referred to collectively herein as the “Bridged Bicyclic Heterocycle Derivatives”) can be useful for treating or preventing obesity, diabetes, a diabetic complication, metabolic syndrome, a cardiovascular disease or a disorder related to the activity of a GPCR (each being a “Condition”) in a patient.

Also provided by the invention are methods for treating or preventing a Condition in a patient, comprising administering to the patient an effective amount of one or more Bridged Bicyclic Heterocycle Derivatives.

The present invention further provides compositions comprising an effective amount of one or more Bridged Bicyclic Heterocycle Derivatives or a pharmaceutically acceptable salt, solvate, ester, prodrug or stereoisomer thereof, and a pharmaceutically acceptable carrier. The compositions can be useful for treating or preventing a Condition in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

In an embodiment, the present invention provides Bridged Bicyclic Heterocycle Derivatives of formula (I), compositions comprising one or more Bridged Bicyclic Heterocycle Derivatives, and methods of using the Bridged Bicyclic Heterocycle Derivatives for treating or preventing a Condition in a patient.

### DEFINITIONS AND ABBREVIATIONS

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “obesity-related disorder” as used herein refers to: (i) disorders which result from a patient having a BMI of 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more succeptible to cardiovascular disease and/or type 2 diabetes. A patient is said to have metabolic syndrome if the patient simultaneously has three or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “effective amount” as used herein, refers to an amount of Bridged Bicyclic Heterocycle Derivative and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 5 to about 7 ring atoms. The term “cycloalkyl” also encompasses a cycloalkyl group, as defined above, that is fused to an aryl (e.g., benzene) or heteroaryl ring. A cycloalkyl group can be joined via a ring carbon or ring nitrogen atom. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

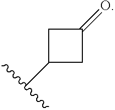

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted. In another embodiment, a cycloalkenyl group is a 5-membered cycloalkenyl. In another embodiment, a cycloalkenyl group is a 6-membered cycloalkenyl.

The term “heteroalkylene,” as used herein, refers to group having the formula -alkylene-X-alkylene- wherein X is —O—, —S— or —NH—. Non-limiting examples of heteroalkylene groups include —CH₂OCH₂—, —CH₂SCH₂—, —CH₂N(H)CH₂—, —CH₂OCH₂CH₂—, —CH₂SCH₂CH₂— and —CH₂N(H)CH₂CH₂—. In one embodiment, a heteroalkylene group has from 2 to about 6 carbon atoms. In another embodiment, a heteroalkylene group has from 2 to about 3 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, that is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is unsubstituted. In another embodiment, a heteroaryl group is a 5-membered heteroaryl. In another embodiment, a heteroaryl group is a 6-membered heteroaryl.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. A heterocycloalkyl group can be joined via a ring carbon or ring nitrogen atom. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, that is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone and the like, and all isomers thereof. A ring carbon atom of a heterocycloalkyl group may be functionalized as a carbonyl group. An illustrative example of such a heterocycloalkyl group is pyrrolidonyl:

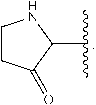

In one embodiment, a heterocycloalkyl group is unsubstituted. In another embodiment, a heterocycloalkyl group is a 5-membered heterocycloalkyl. In another embodiment, a heterocycloalkyl group is a 6-membered heterocycloalkyl.

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 5 to 10 ring atoms. In another embodiment, a heterocycloalkenyl group is monocyclic and has 5 or 6 ring atoms. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group. In one embodiment, a heterocycloalkenyl group is unsubstituted. In another embodiment, a heterocycloalkenyl group is a 5-membered heterocycloalkenyl. In another embodiment, a heterocycloalkenyl group is a 6-membered heterocycloalkenyl.

It should also be noted that tautomeric forms such as, for example, the moieties:

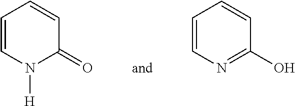

are considered equivalent in certain embodiments of this invention.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkyl-aryl, -aryl-alkyl, -alkylene-heteroaryl, -alkenylene-heteroaryl, -alkynylene-heteroaryl, hydroxy, hydroxyalkyl, haloalkyl, —O-alkyl, —O-haloalkyl, -alkylene-O-alkyl, —O-aryl, aralkoxy, acyl, aroyl, halo, nitro, cyano, carboxy, —C(O)O-alkyl, —C(O)O-aryl, —C(O)O-alkelene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NS(O)₂— and —S(O)₂NY₁Y₂, wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

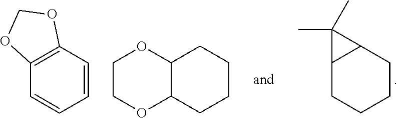

“Halo” means —F, —Cl, —Br or —I. In one embodiment, halo refers to —F, —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Bridged Bicyclic Heterocycle Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Bridged Bicyclic Heterocycle Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Bridged Bicyclic Heterocycle Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a Bridged Bicyclic Heterocycle Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy (C₁-C₆)alkyl, amino(C₁-C₄)alkyl or mono-N- or di-N,N-(C₁-C₆)alkylaminoalkyl, —C(Y⁴)Y⁵ wherein Y⁴ is H or methyl and Y⁵ is mono-N- or di-N,N-(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Bridged Bicyclic Heterocycle Derivatives can form salts which are also within the scope of this invention. Reference to a Bridged Bicyclic Heterocycle Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Bridged Bicyclic Heterocycle Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of the Formula (I) may be formed, for example, by reacting a Bridged Bicyclic Heterocycle Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or —O—C₁₋₄alkyl or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Bridged Bicyclic Heterocycle Derivatives may be atropisomers (e.g., substituted biaryls) and are considered as part of this invention. Enantiomers can also be separated by use of chiral HPLC column.

It is also possible that the Bridged Bicyclic Heterocycle Derivatives may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Bridged Bicyclic Heterocycle Derivative incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Pyrimidine Derivatives (e.g., those labeled with ³H and ¹⁴C) are useful in compound and/or substrate tissue distribution assays. In one embodiment, ritiated (i.e., ³H) and carbon-14 (i.e., ¹⁴C) isotopes are employed for their ease of preparation and detectability. In another embodiment, substitution with heavier isotopes such as deuterium (i.e., ²H) may afford certain therapeutic advantages resulting from greater metabolic stability (e.g., increased in vivo half-life or reduced dosage requirements). Isotopically labelled compounds of Formula (I) can generally be prepared by following procedures analogous to those disclosed in the Schemes and/or in the Examples herein below, by substituting an appropriate isotopically labelled reagent for a non-isotopically labelled reagent.

Polymorphic forms of the Bridged Bicyclic Heterocycle Derivatives, and of the salts, solvates, hydrates, esters and prodrugs of the Bridged Bicyclic Heterocycle Derivatives, are intended to be included in the present invention.

The following abbreviations are used below and have the following meanings:

AcOH is acetic acid, 9-BBN is 9-borabicyclo[3.3.1]nonane, BINAP is [1,1′-binaphthalene]-2,2′-diylbis(diphenylphosphine), Boc or BOC is t-butyloxycarbonyl, (Boc)₂O is t-butyloxycarbonyl anhyride, BSA is bovine serum albumin, t-butyl is tertiary butyl, t-BuOK is potassium tert-butoxide, dba is dibenzylidene acetone, DCM is dichloromethane, DIBAL-H is diisobutylaluminum hydride, DIEA or DIPEA is diisopropylethylamine, DMEM is Dulbecco's modified eagle medium, DMF is N,N-dimethylformamide, DMSO is dimethylsulfoxide, DSC is N,N′-disuccinimidyl carbonate, EDC is 1-(dimethylaminopropyl)-3-ethylcarbodiimide, Et is ethyl, EtOAc is ethyl acetate, EtOH is ethanol, Et₃N is triethylamine, HATU is O-(7-azabenzotriazol-1-yl)-N,N,N′,N′-tetramethyluronium hexafluorophosphate, HEPES is 2-[4-(2-hydroxyethyl)-1-piperazinyl] ethanesulfonic acid, LCMS is liquid chromatography mass spectrometry, MeCN is acetonitrile, MeI is methyl iodide, MeOH is methanol, NaBH(OAc)₃ is sodium triacetoxy borohydride, NaOEt is sodium ethoxide, NaOMe is sodium methoxide, NaOtBu is sodium t-butoxide, NMA is 3a,4,7,7a-tetrahydro-4-methyl 4,7-methanoisobenzofuran-1,3-dione, NMR is nuclear magnetic resonance, Pd/C is palladium on carbon, Pd(OAc)₂ is palladium(II)acetate, Ph is phenyl, PLC is preparative thin-layer chromatography, TBAI is tetrabutyl ammonium iodide, THF is tetrahydrofuran, TLC is thin-layer chromatography and X-Phos is 2-dicyclohexylphosphino-2′,4′,6′-triisopropylbiphenyl.

### The Bridged Bicyclic Heterocycle Derivatives of Formula (I)

The present invention provides Bridged Bicyclic Heterocycle Derivatives of Formula (I):

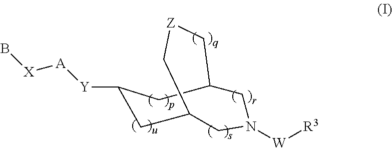

and pharmaceutically acceptable salts, solvates, esters, prodrugs and stereoisomers thereof, wherein A, B, W, X, Y, Z, R³, p, q, r, s and u are defined above for the compounds of formula (I).

In one embodiment, A is pyridyl or pyrimidinyl, wherein said pyridyl group can be optionally substituted with a 5-membered heteroaryl group, and wherein said pyrimidinyl group can be optionally substituted with alkyl, halo, cycloalkyl or —O-alkyl.

In another embodiment, A is:

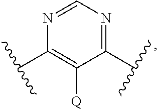

wherein is H, alkyl, halo, cycloalkyl or —O-alkyl.

In another embodiment, A is:

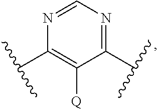

wherein is H, methyl, ethyl, cyclopropyl, Cl, F, methoxy or ethoxy.

In still another embodiment, A is:

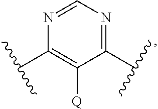

wherein is F or methoxy.

In one embodiment, A is:

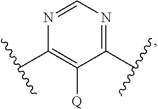

wherein is alkyl, halo, cycloalkyl or —O-alkyl.

In another embodiment, A is:

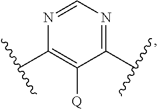

wherein is methyl, ethyl, F, methoxy or ethoxy.

In another embodiment, A is:

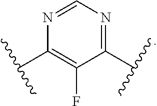

In one embodiment, A is pyridyl.

In another embodiment, A is:

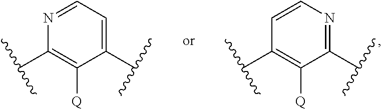

wherein Q is H, alkyl, halo, cycloalkyl or —O-alkyl.

In another embodiment, A is:

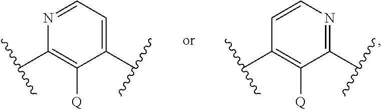

wherein Q is H, methyl, ethyl, cyclopropyl, Cl, F, methoxy or ethoxy.

In still another embodiment, A is:

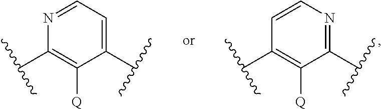

wherein Q is F or methoxy.

In one embodiment, B is phenyl or pyridyl.

In another embodiment, B is phenyl, which is unsubstituted or optionally substituted with up to 3 groups, which can be the same or different, and are selected from alkyl, heterocycloalkyl, heteroaryl, halo, —CN, —S(O)₂alkyl and —S(O)₂cycloalkyl.

In still another embodiment, B is phenyl, which is unsubstituted or optionally substituted with up to 3 groups, each independently selected from methyl, oxadiazolyl, triazolyl, imidazolidinone, —CN, —Cl, —F, —C(O)NH₂, —S(O)₂CH₃ and —S(O)₂-cyclopropyl.

In still another embodiment, B is pyridyl, which is unsubstituted or optionally substituted with up to 3 groups, each independently selected from methyl, oxadiazolyl, triazolyl, imidazolidinone, —CN, —Cl, —F, —S(O)₂CH₃ and —S(O)₂-cyclopropyl.

In one embodiment, B is:

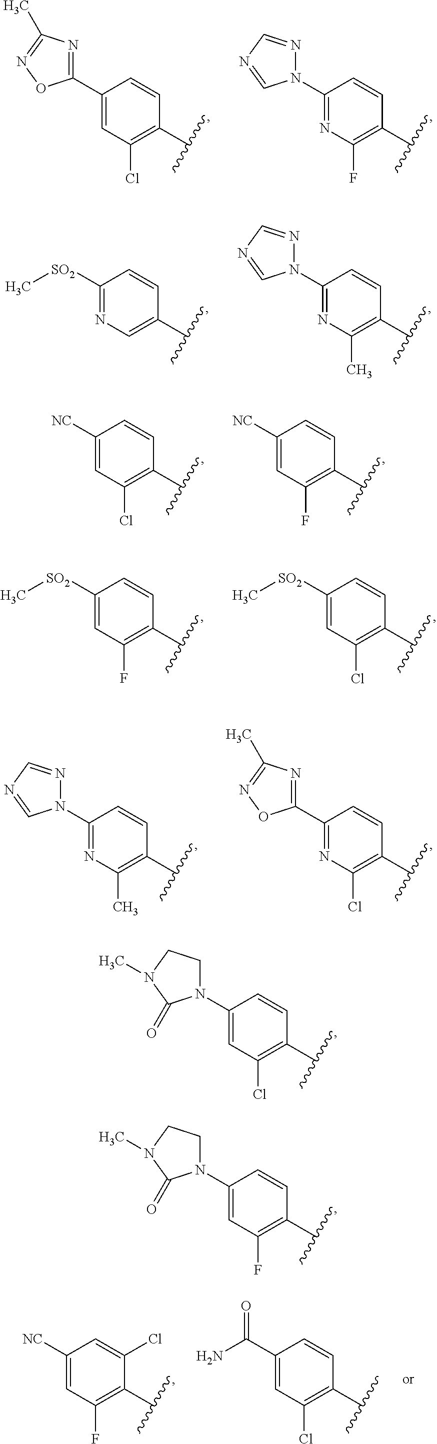

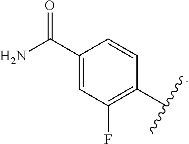

In another embodiment, B is selected from:

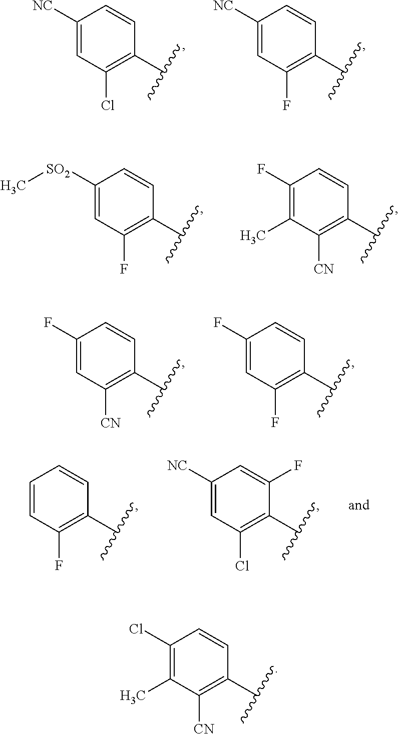

In another embodiment, B is selected from:

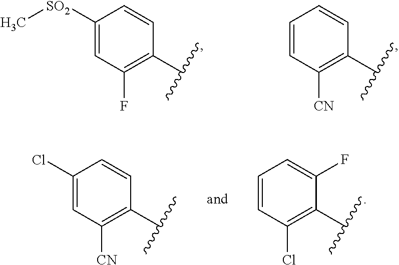

In one embodiment, W is —C(O)O— or —S(O)₂—.

In another embodiment, W is a —C(O)O— or a bond.

In another embodiment, W is a bond.

In another embodiment, W is —C(O)O—.

In still another embodiment, W is —S(O)₂—.

In another embodiment, W is —C(O)—.

In yet another embodiment, W is —C(O)S—.

In one embodiment, X is —NH—.

In another embodiment, X is —O—.

In another embodiment, X is —O-alkylene-.

In still another embodiment, X is —O—CH₂—.

In one embodiment, Y is —NH—.

In another embodiment, Y is —O—.

In one embodiment, Y is —O— and X is —O— or —NH—.

In another embodiment, X is —NH— and Y is —NH—.

In another embodiment, X is —O— and Y is —O—.

In another embodiment, X is —O— and Y is —NH—.

In another embodiment, X and Y are each —O—.

In one embodiment, Z is a bond.

In another embodiment, Z is —O—.

In another embodiment, Z is —C(R¹)₂—.

In still another embodiment, Z is —C(O)—.

In another embodiment, Z is —N(R⁴)—.

In another embodiment, Z is —S(O)₂—.

In yet another embodiment, Z is —CH₂—.

In a further embodiment, Z is:

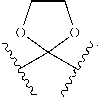

In one embodiment, p is 0.

In another embodiment, p is 1.

In one embodiment, q is 0.

In another embodiment, q is 1.

In one embodiment, r is 0.

In another embodiment, r is 1.

In one embodiment, s is 0.

In another embodiment, s is 1.

In one embodiment, u is 0.

In another embodiment, u is 1.

In one embodiment, p and u are each 0 and r and s are each 1.

In another embodiment, p and u are each 1 and r and s are each 0.

In one embodiment, q is 1 and Z is —O—.

In another embodiment, q is 0 and Z is —O—.

In another embodiment, q is 1 and Z is a bond.

In still another embodiment, q is 1 and Z is —C(R¹)₂.

In another embodiment, q is 1 and Z is —C(O)—.

In another embodiment, q is 1 and Z is —N(R⁴)—.

In yet another embodiment, q is 1 and Z is —N(Boc)-.

In another embodiment, q is 1 and Z is —CH(OH)—.

In a further embodiment, q is 1 and Z is:

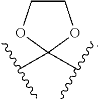

In one embodiment, R³ is alkyl.

In another embodiment, R³ is -alkylene-aryl.

In another embodiment, R³ is cycloalkyl, which is optionally substituted with an alkyl group.

In still another embodiment, R³ is -alkylene-cycloalkyl, wherein the cycloalkyl moiety is optionally substituted with alkyl or -alkylene-O-alkyl.

In another embodiment, R³ is haloalkyl.

In yet another embodiment, R³ is heteroaryl.

In another embodiment, R³ is -alkylene-O-alkyl.

In a further embodiment, R³ is

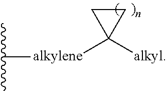

In another embodiment, R³ is

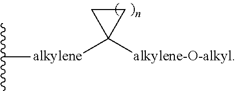

In one embodiment, R³ is cycloalkyl or alkyl, wherein a cycloalkyl group is unsubstituted or optionally substituted with an alkyl group.

In another embodiment, R³ is

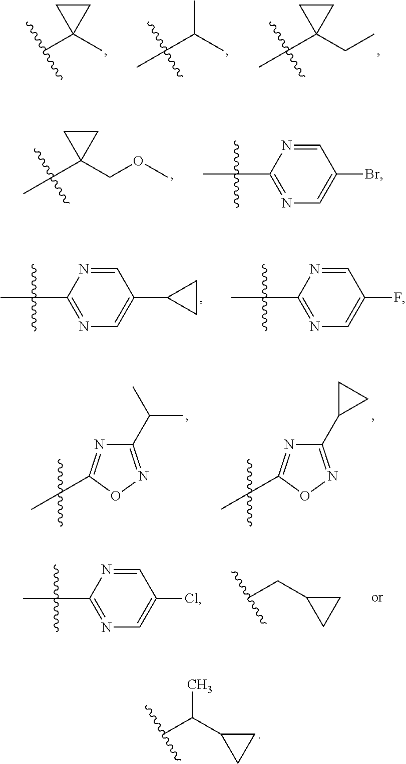

In another embodiment, R³ is:

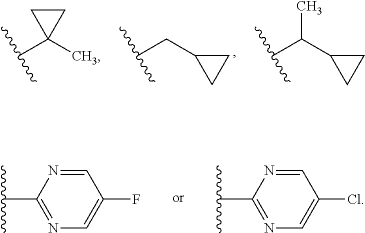

In another embodiment, R³ is:

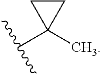

In one embodiment, W is —C(O)O— and R³ is alkyl.

In another embodiment, W is —C(O)O— and R³ is cycloalkyl, which is optionally substituted with an alkyl group.

In another embodiment, W is —C(O)O— and R³ is -alkylene-cycloalkyl, wherein the cycloalkyl moiety is optionally substituted with alkyl or -alkylene-O-alkyl.

In another embodiment, W is a bond and R³ is heteroaryl.

In another embodiment, W is a bond and R³ is pyrimidinyl, which is substituted with a halo group.

In still another embodiment, W is a bond and R³ is phenyl.

In another embodiment, W is a bond and R³ is benzyl.

In yet another embodiment, W is —C(O)O— or a bond, and R³ is alkyl, 5- or 6-membered heteroaryl or -(alkylene)ₜ-cycloalkyl, wherein said 5- or 6-membered heteroaryl can be optionally substituted with alkyl, cycloalkyl or halo, and wherein the cycloalkyl moiety of said -(alkylene)ₜ-cycloalkyl group can be optionally substituted with alkyl or -alkylene-O-alkyl.

In another embodiment, W is —C(O)O— or a bond, and R³ is selected from:

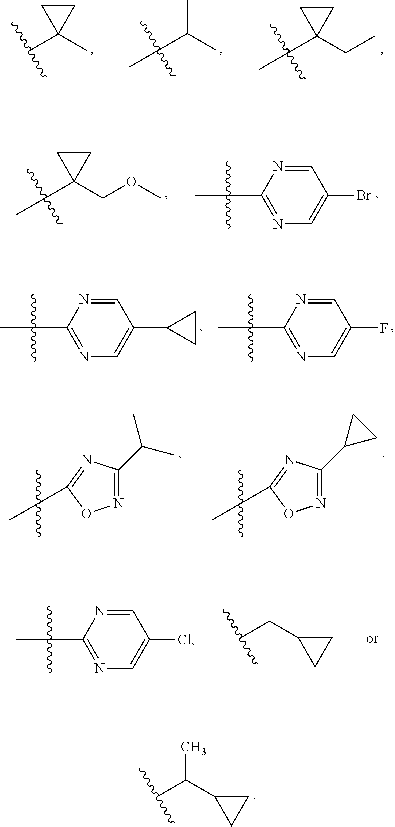

In another embodiment, W is —C(O)O— or —C(O)S— and R³ is:

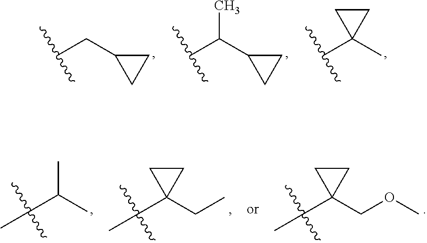

In another embodiment, W is a bond and R³ is:

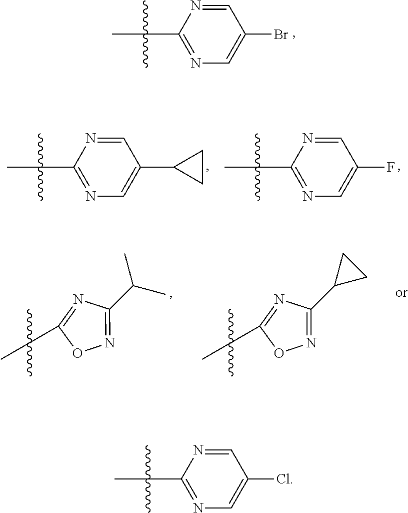

In one embodiment, the group —W—R³ is —S(O)₂-cyclopropyl, —S(O)₂-cyclobutyl, —S(O)₂CF₃, —S(O)₂CH₂CH₂OCH₃, —C(O)O-cyclopropyl, —C(O)O-cyclobutyl, —C(O)O-(1-methylcyclopropyl), —C(O)O-(1-methylcyclobutyl), —(O)O-(1-methylcyclopropyl), —C(O)O-isopropyl, —C(O)O-(1-ethylcyclopropyl), —C(O)O-(1-methoxymethylcyclopropyl), 5-bromopyrimidinyl, 5-fluoropyrimidinyl, 5 cyclopropyl-pyrimidinyl, 3-cyclopropyl-1,2,4-oxadiazolyl, 3-isopropyl-1,2,4-oxadiazolyl or benzyl.

In another embodiment, the group W—R³ is:

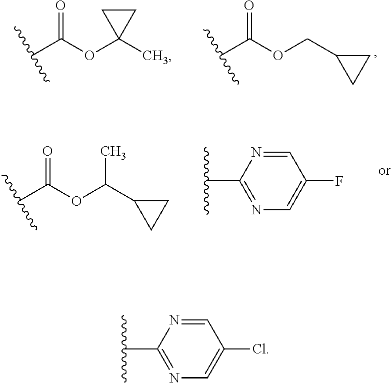

In another embodiment, the group W—R³ is:

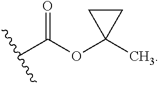

In one embodiment, A is pyridyl or pyrimidinyl, wherein said pyridyl group can be optionally substituted with a 5-membered heteroaryl group, and wherein said pyrimidinyl group can be optionally substituted with alkyl, halo, cycloalkyl or —O-alkyl, and B is: (i) phenyl, which is optionally substituted with up to 3 groups, which can be the same or different, and are selected from alkyl, —S(O)₂alkyl, halo and —CN, or (ii) pyridyl, which is optionally substituted with up to 2 groups, which can be the same or different, and are selected from 5-membered heteroaryl and —S(O)₂alkyl.

In another embodiment, A is pyridyl or pyrimidinyl, wherein said pyridyl group can be optionally substituted with a 5-membered heteroaryl group, and wherein said pyrimidinyl group can be optionally substituted with alkyl, halo, cycloalkyl or —O-alkyl; B is: (i) phenyl, which is optionally substituted with up to 3 groups, which can be the same or different, and are selected from alkyl, —S(O)₂alkyl, halo and —CN, or (ii) pyridyl, which is optionally substituted with up to 2 groups, which can be the same or different, and are selected from 5-membered heteroaryl and —S(O)₂alkyl; X is —NH— or —O—; and Y is —O—.

In one embodiment, A is:

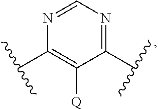

wherein Q is H, alkyl, halo, cycloalkyl or —O-alkyl; and B is phenyl or pyridyl.

In another embodiment, A is:

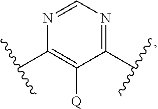

wherein Q is H, alkyl, halo, cycloalkyl or —O-alkyl; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

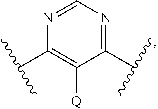

wherein Q is alkyl, halo or —O-alkyl; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In still another embodiment, A is:

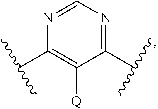

wherein Q is F, methyl, ethyl, methoxy or ethoxy; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

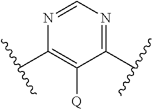

wherein Q is H, alkyl, halo, cycloalkyl or —O-alkyl; and B is pyridyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In yet another embodiment, A is:

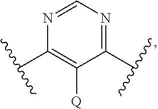

wherein Q is H, methyl, Cl, F, cyclopropyl or methoxy; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

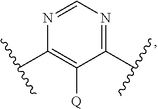

wherein Q is H, methyl, Cl, F, cyclopropyl or methoxy; and B is pyridyl, which is unsubstituted or optionally substituted with up to 3 groups, each independently selected from alkyl, —ON, —S(O)₂-alkyl, —S(O)₂-cycloalkyl, heteroaryl, heterocycloalkyl and halo.

In a further embodiment, A is:

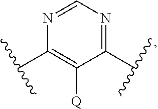

wherein Q is F or methoxy; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In one embodiment, A is:

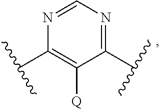

wherein Q is F or methoxy; and B is pyridyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

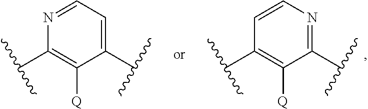

wherein Q is H, alkyl, halo, cycloalkyl or —O-alkyl; and B is phenyl or pyridyl.

In another embodiment, A is:

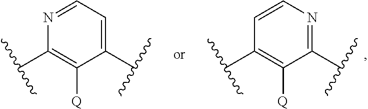

wherein Q is H, alkyl, halo, cycloalkyl or —O-alkyl; and B is phenyl, which is unsubstituted or optionally substituted with up to 3 groups, each independently selected from alkyl, —ON, —S(O)₂-alkyl, —S(O)₂-cycloalkyl, heteroaryl, heterocycloalkyl and halo.

In still another embodiment, A is:

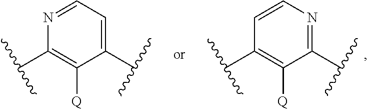

wherein Q is H, alkyl, halo, cycloalkyl or —O-alkyl; and B is pyridyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

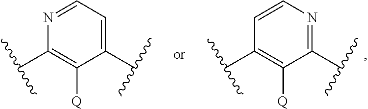

wherein Q is H, methyl, Cl, F, cyclopropyl or methoxy; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

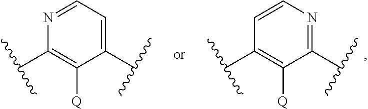

wherein Q is H, methyl, Cl, F, cyclopropyl or methoxy; and B is pyridyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In a further embodiment, A is:

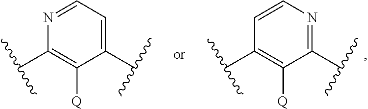

wherein Q is F or methoxy; and B is phenyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In another embodiment, A is:

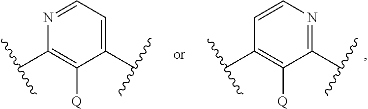

wherein Q is F or methoxy; and B is pyridyl, which is unsubstituted or optionally substituted with up to 3 groups, each independently selected from alkyl, —CN, —S(O)₂-alkyl, —S(O)₂-cycloalkyl, heteroaryl, heterocycloalkyl and halo.

In a further embodiment, A is:

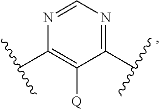

wherein Q is alkyl, halo or —O-alkyl; and B is selected from:

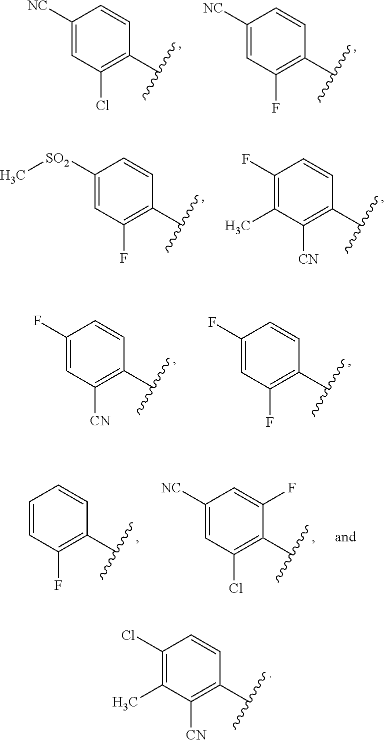

In one embodiment, the group —B—X-A-Y— is:

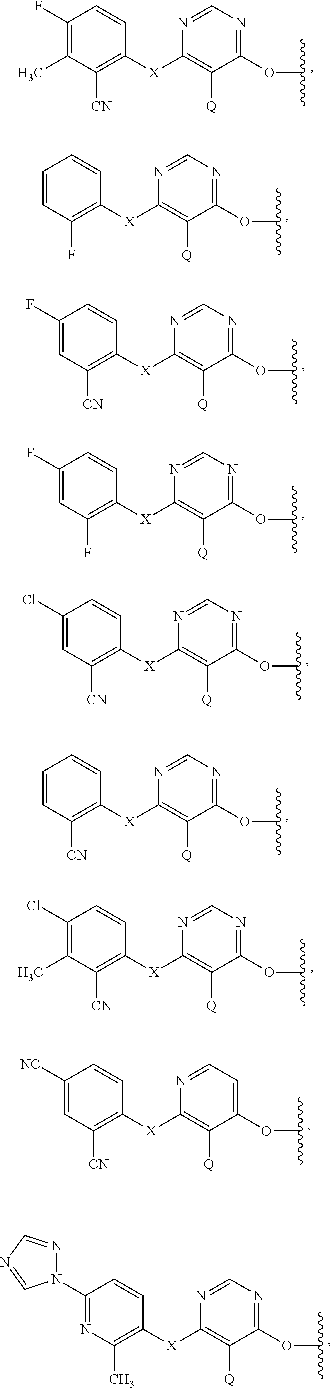

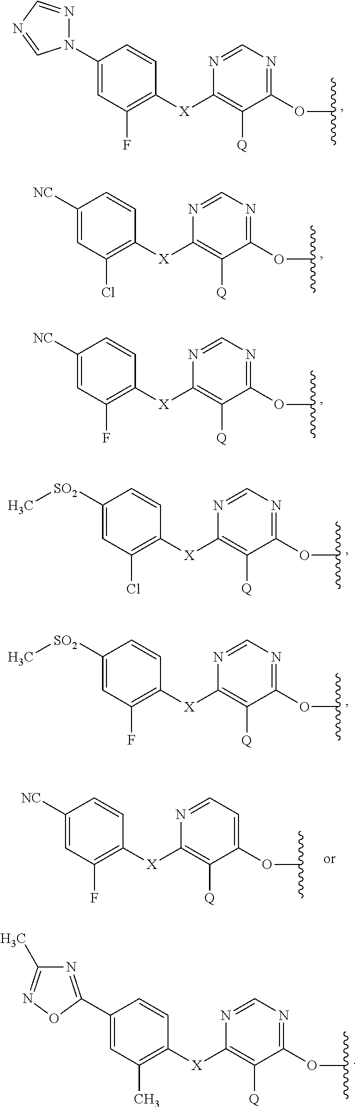

wherein X is —O— or —NH— and Q is H, halo, alkyl, cycloalkyl or —O-alkyl.

In another embodiment, the group —B—X-A-Y— is:

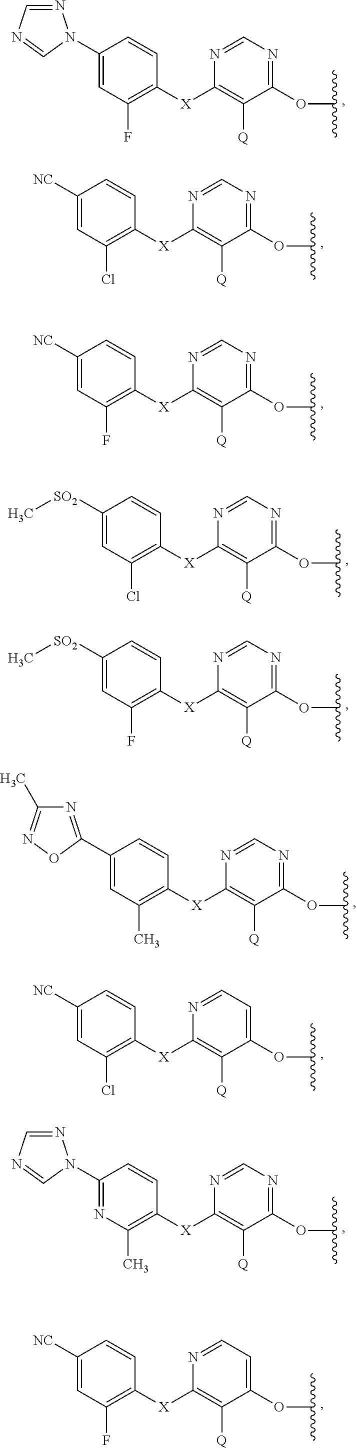

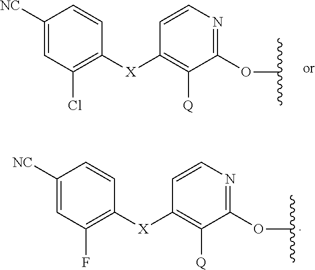

wherein X is —O— or —NH— and Q is H, halo, alkyl, cycloalkyl or —O-alkyl.

In another embodiment, the group B—X-A-Y— is:

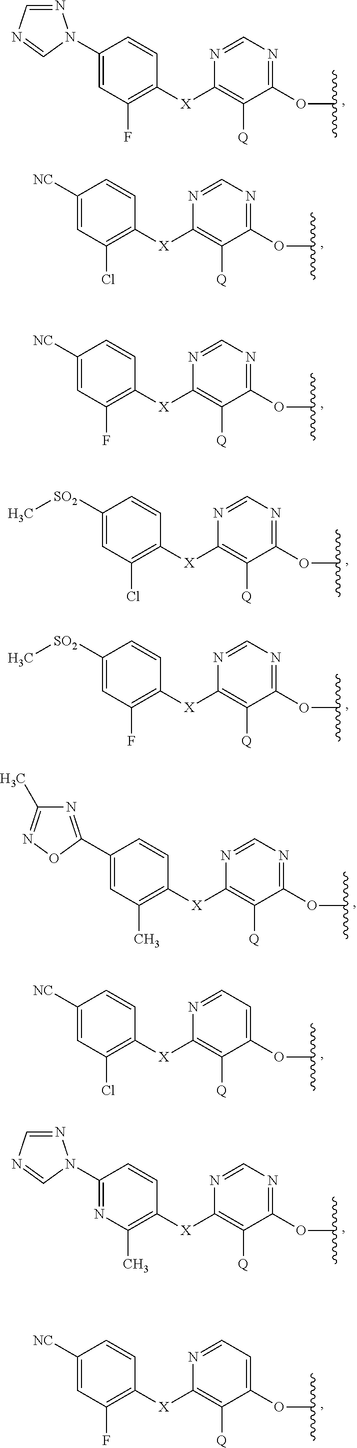

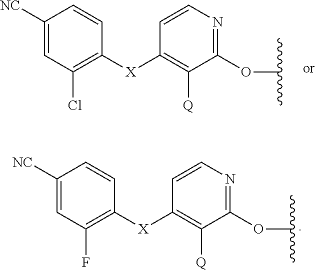

wherein X is —O— or —NH— and Q is H, F, Cl, methyl, cyclopropyl or methoxy.

In another embodiment, the group B—X-A-Y— is:

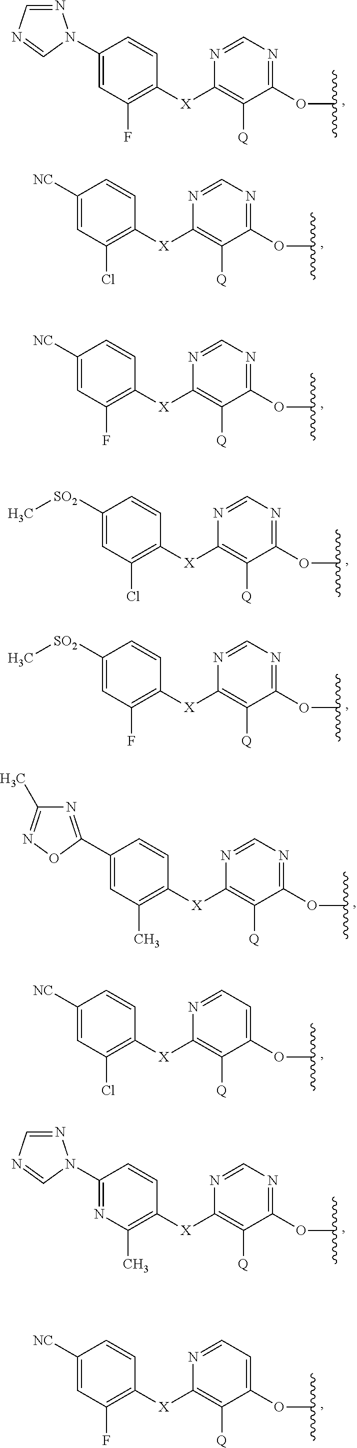

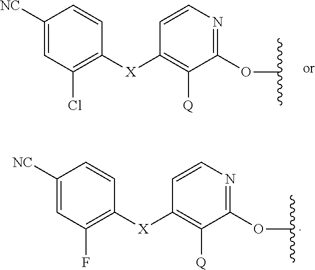

wherein X is —O— or —NH— and Q is F or methoxy.

In one embodiment, the group B—X-A-Y— is:

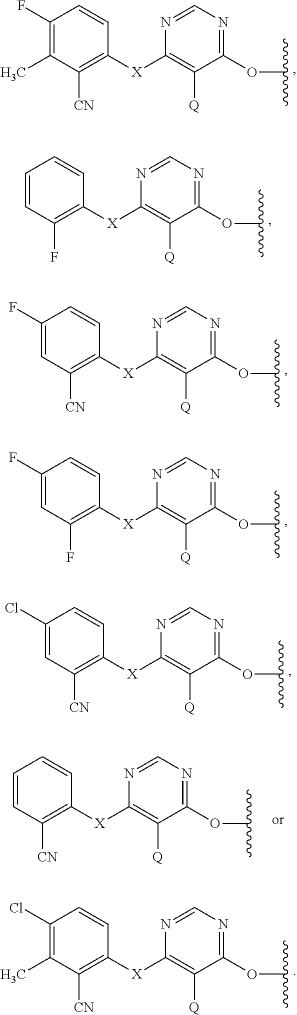

wherein X is —O— or —NH— and Q is F, methyl, ethyl, methoxy or ethoxy.

In one embodiment, the group B—X-A-Y— is:

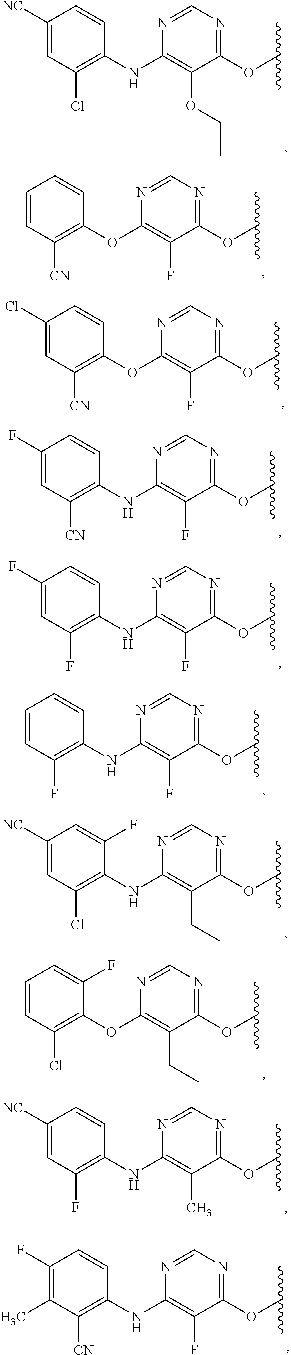

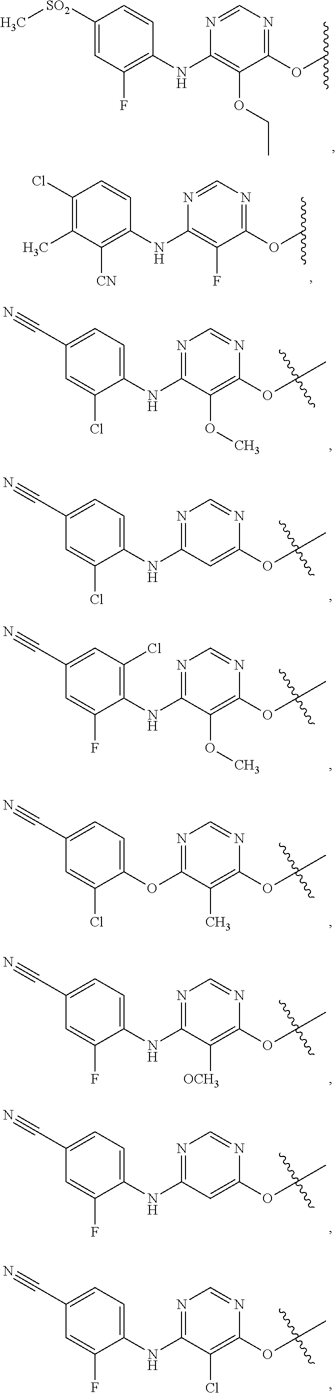

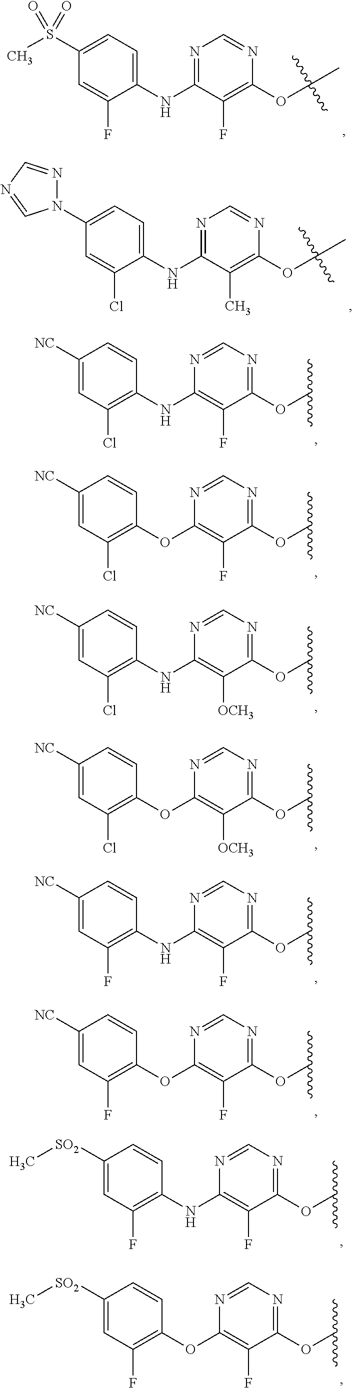

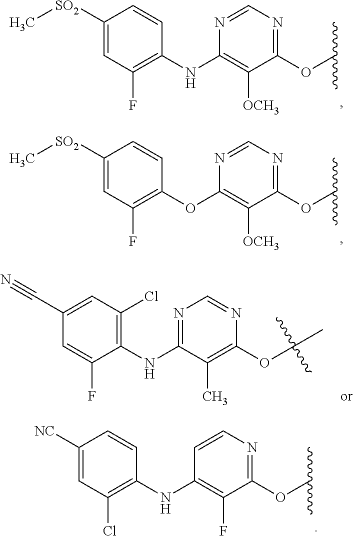

In another embodiment, the group B—X-A-Y— is:

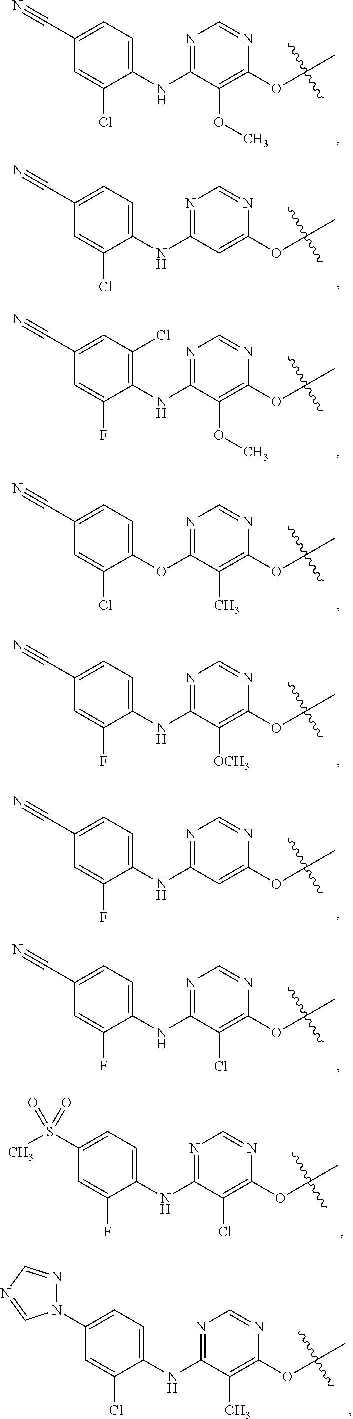

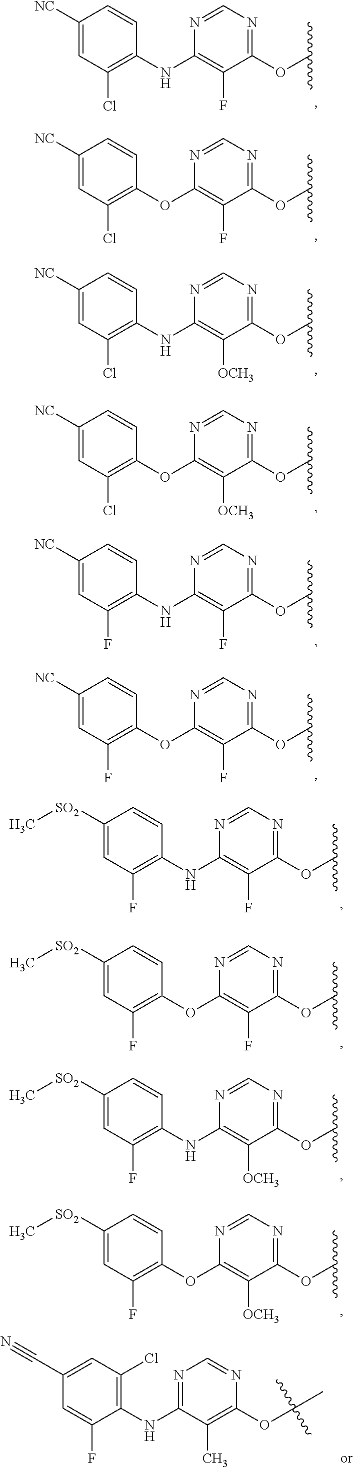

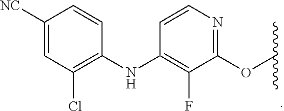

In another embodiment, the group B—X-A-Y— is:

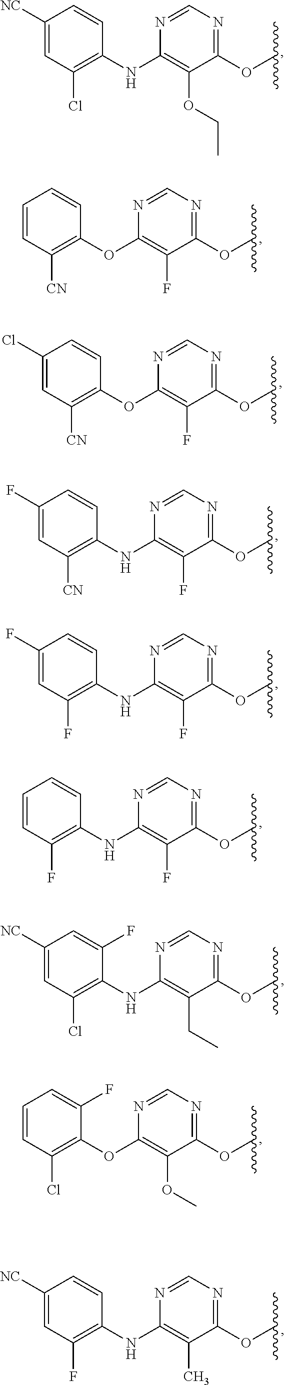

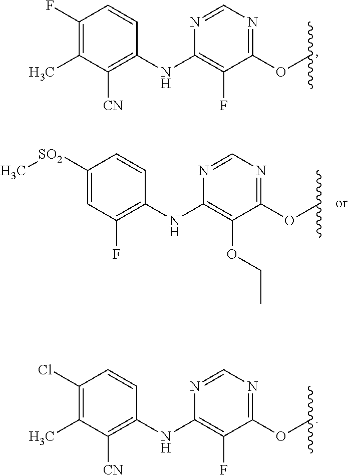

In one embodiment, the group:

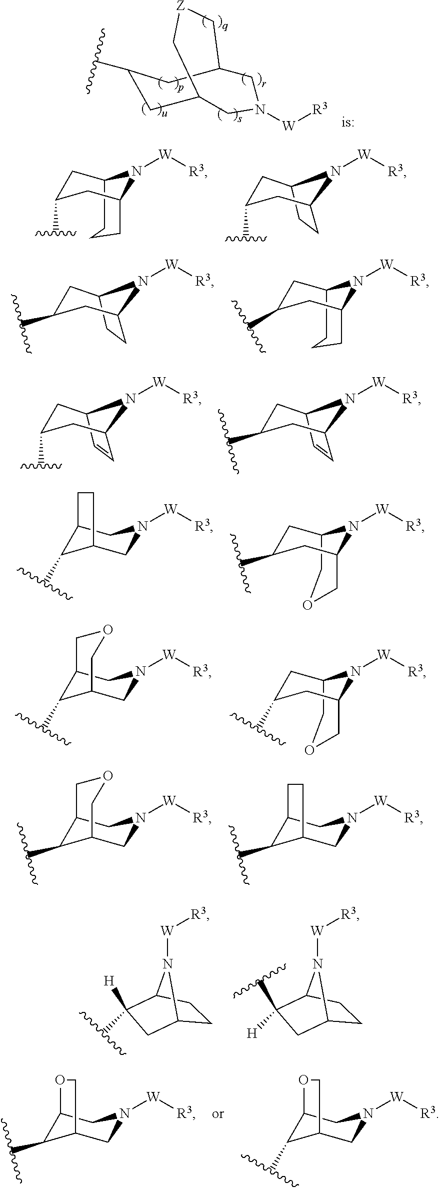

In another embodiment, the group:

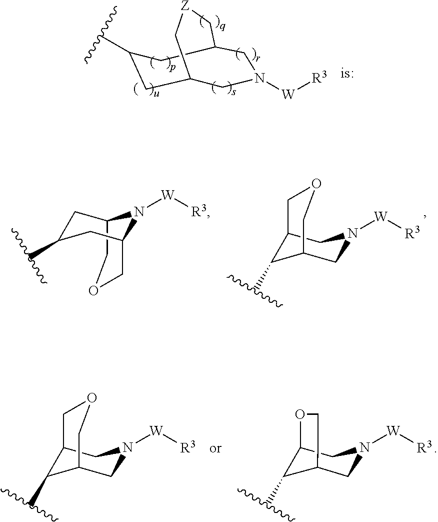

In another embodiment, the group:

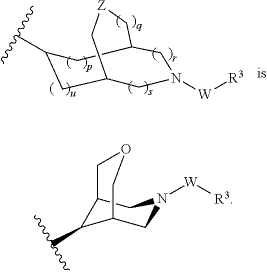

In one embodiment, the group:

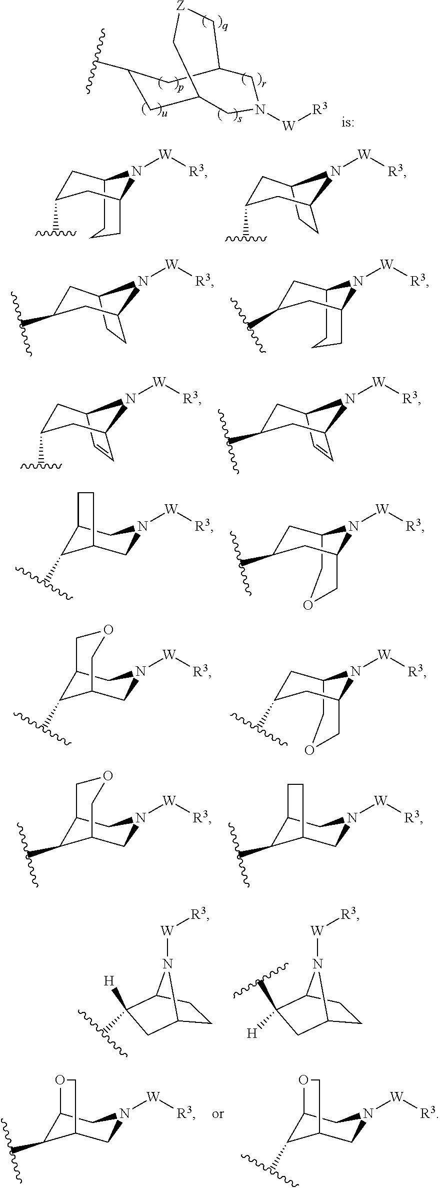

wherein W is —C(O)O— or a bond.
In another embodiment, the group:

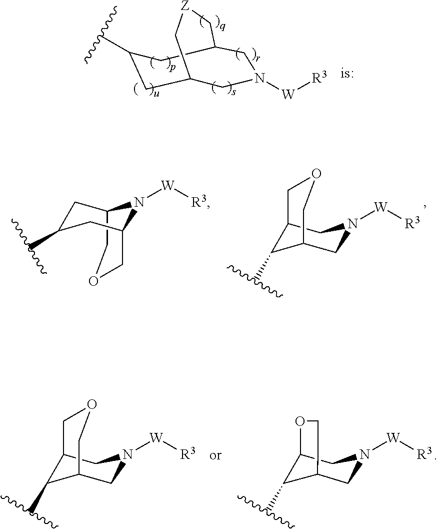

wherein W is —C(O)O— or a bond.
In another embodiment, the group:

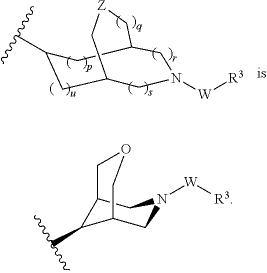

wherein W is —C(O)O— or a bond.

In one embodiment, the group:

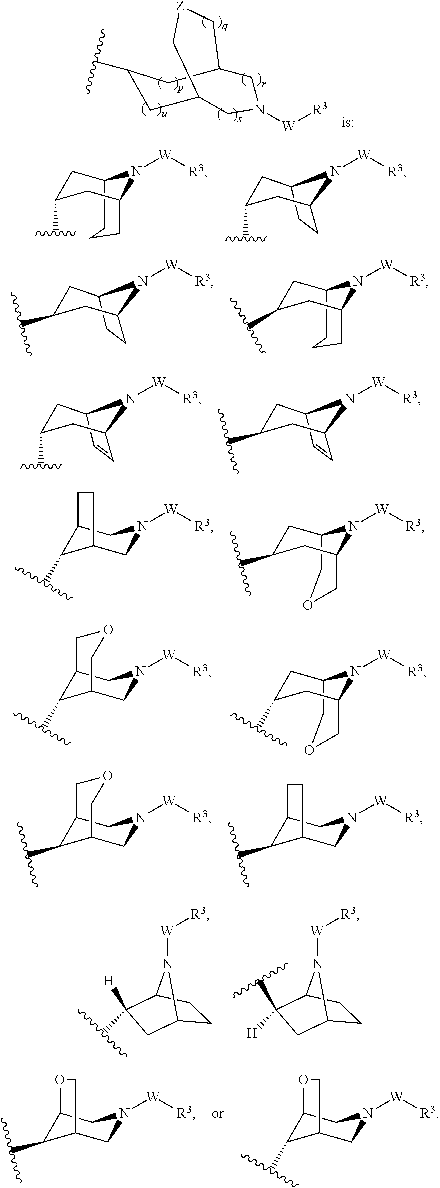

wherein W is —C(O)O— or a bond, and R³ is:

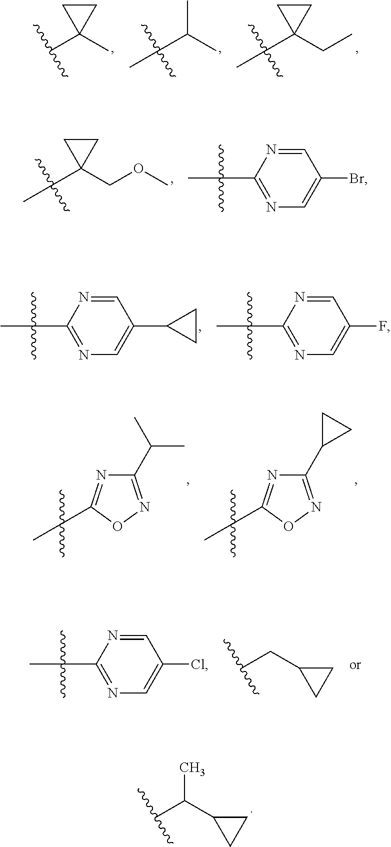

In another embodiment, the group:

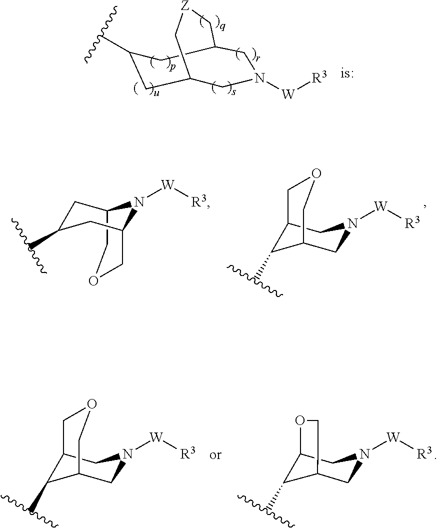

wherein W is —C(O)O— or a bond, and R³ is:

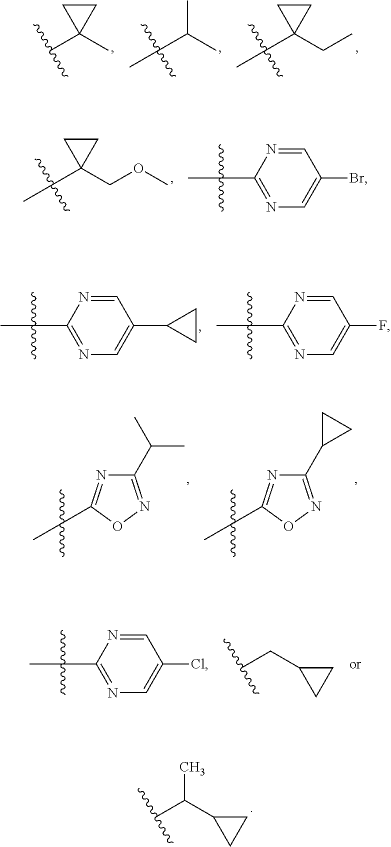

In another embodiment, the group:

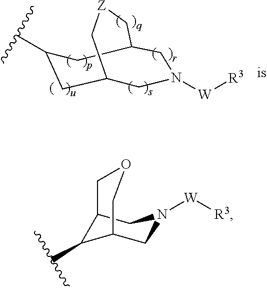

wherein W is —C(O)O— or a bond, and R³ is:

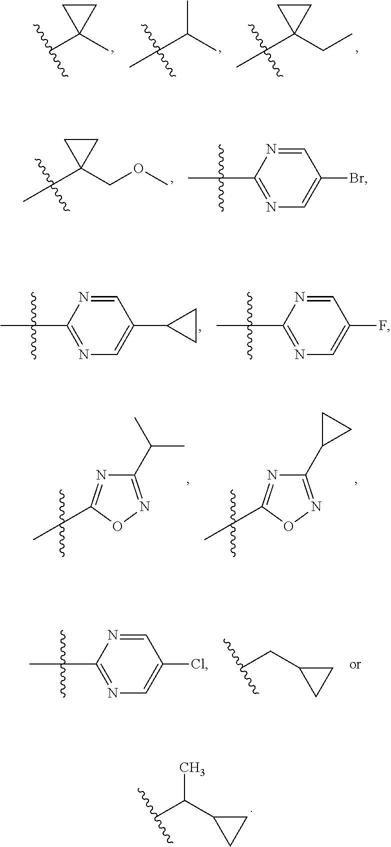

In another embodiment, the group:

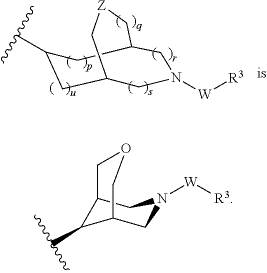

wherein the group W—R³ is:

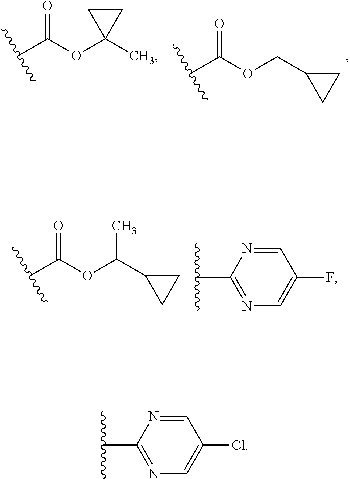

In another embodiment, the group:

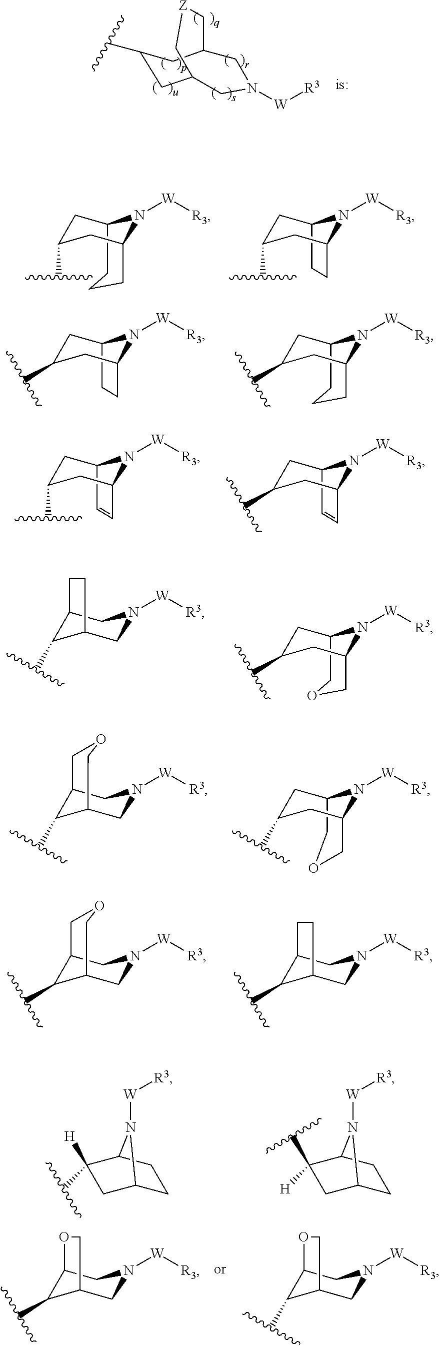

and the group —B—X-A-Y— is:

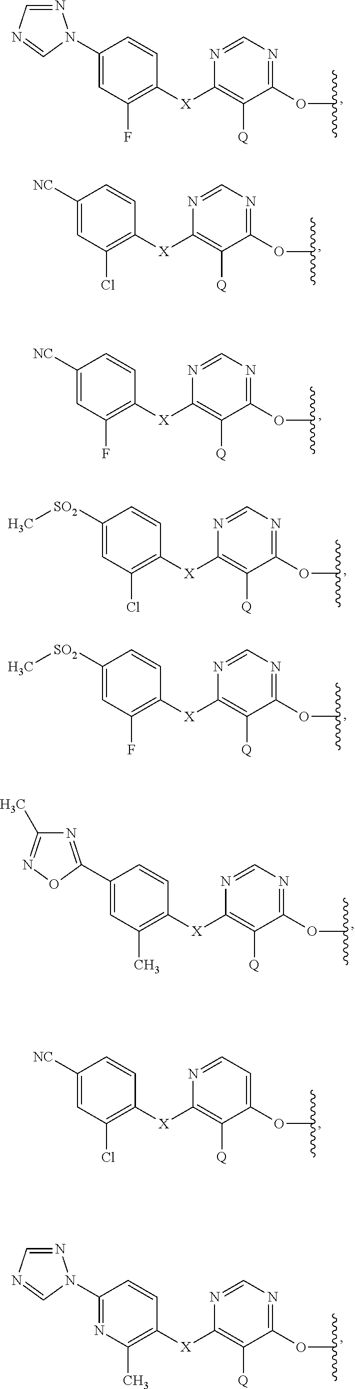

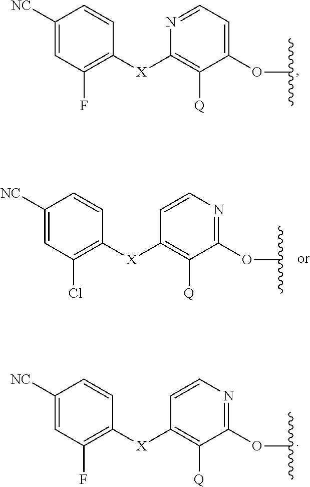

wherein X is —O— or —NH— and Q is H, halo, alkyl, cycloalkyl or —O-alkyl.

In another embodiment, the group:

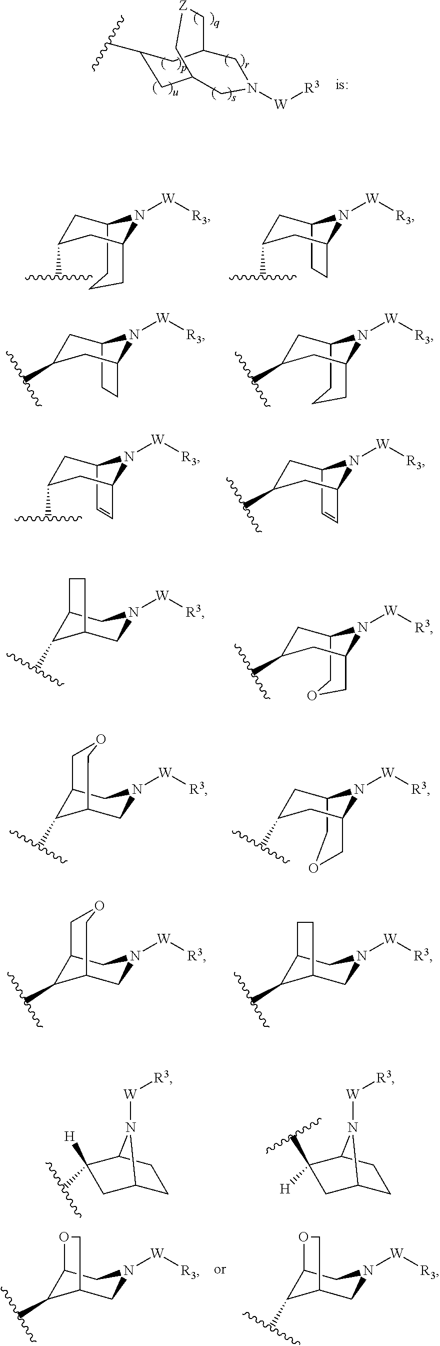

and the group —B—X-A-Y— is:

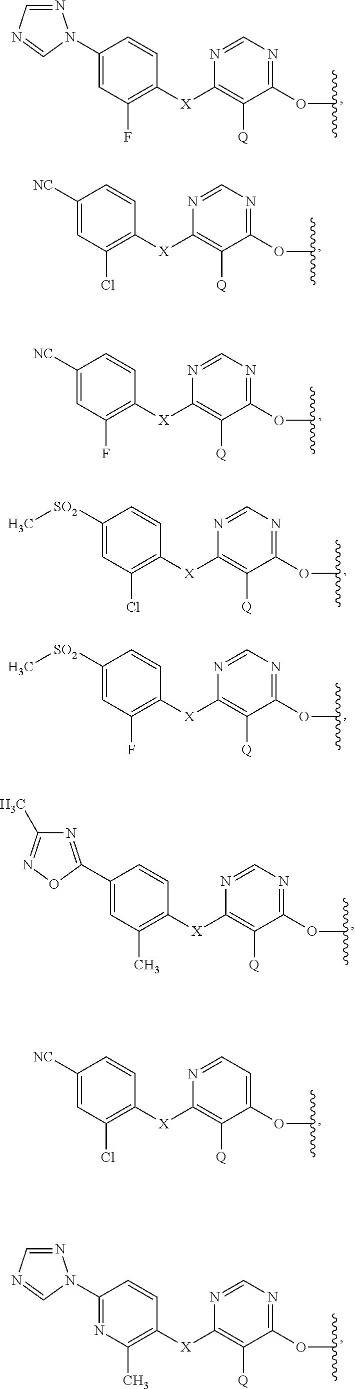

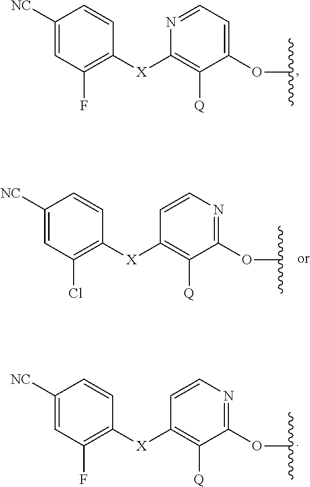

wherein X is —O— or —NH— and Q is H, methyl, Cl, F, cyclopropyl or methoxy.

In another embodiment, the group

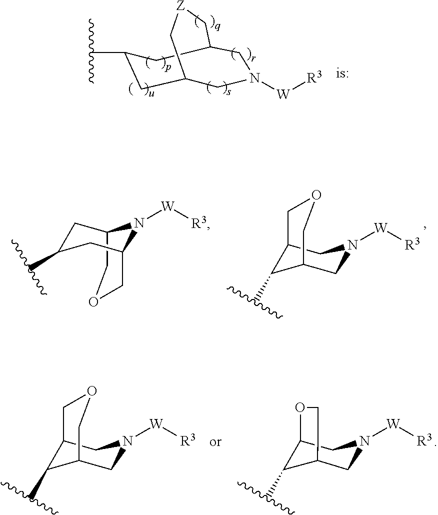

and the group —B—X-A-Y— is:

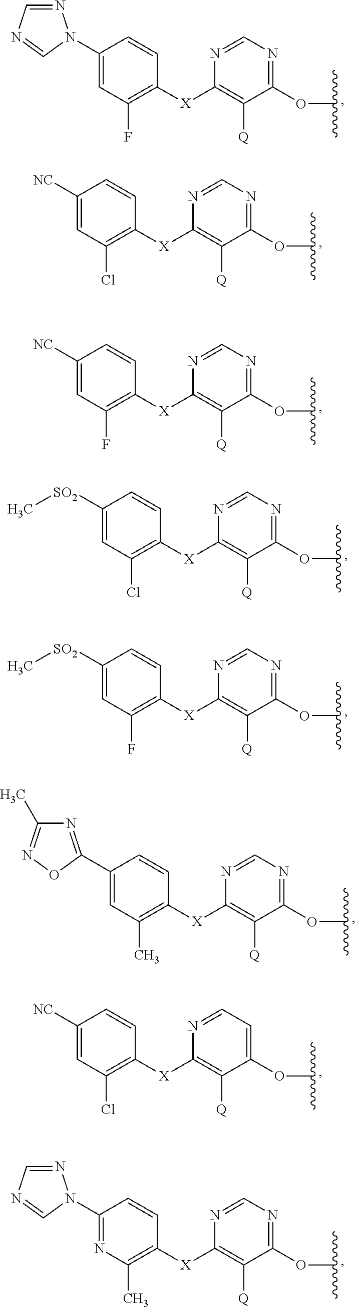

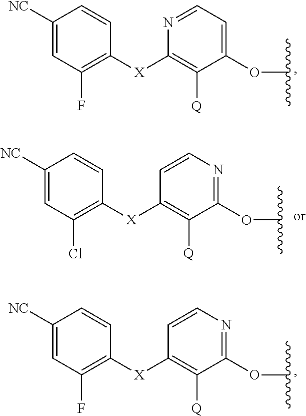

wherein X is —O— or —NH— and Q is H, halo, alkyl, cycloalkyl or —O-alkyl.

In another embodiment, the group

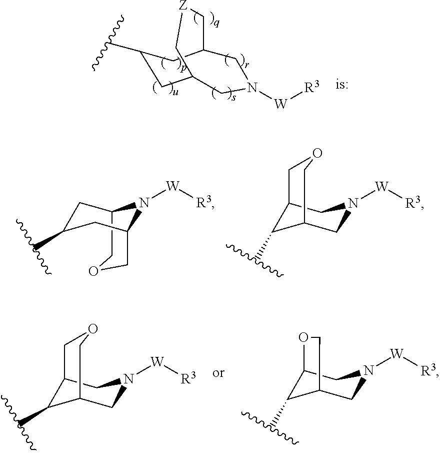

and the group —B—X-A-Y— is:

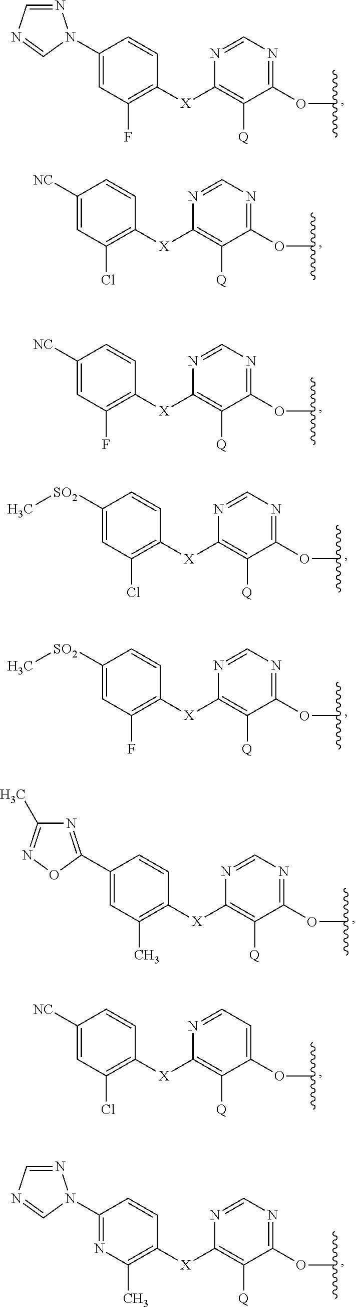

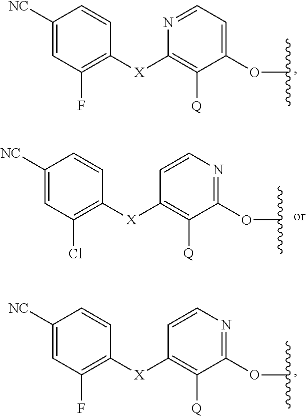

wherein X is —O— or —NH— and Q is H, methyl, Cl, F, cyclopropyl or methoxy.

In one embodiment, the group:

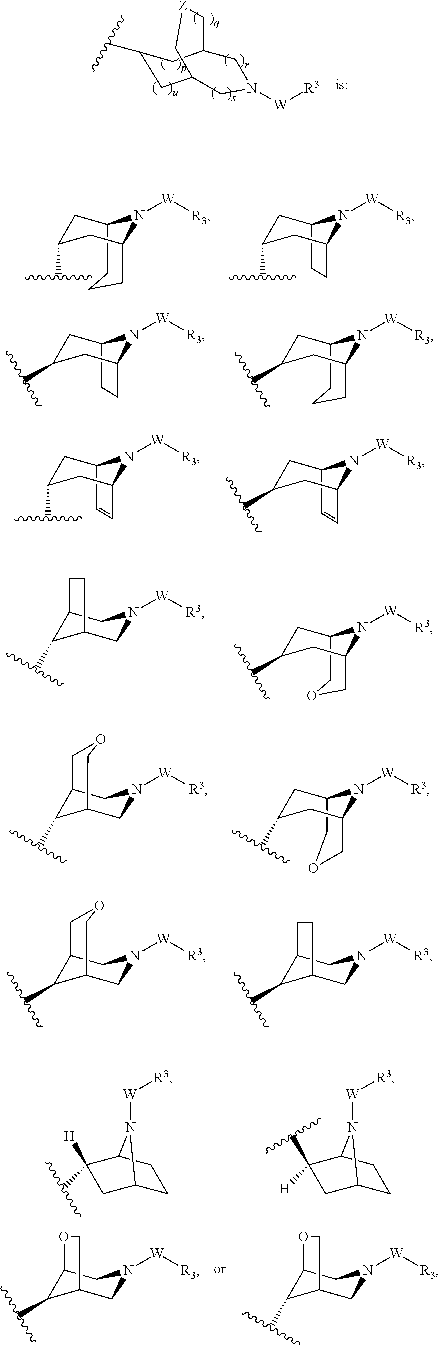

and the group B—X-A-Y— is:

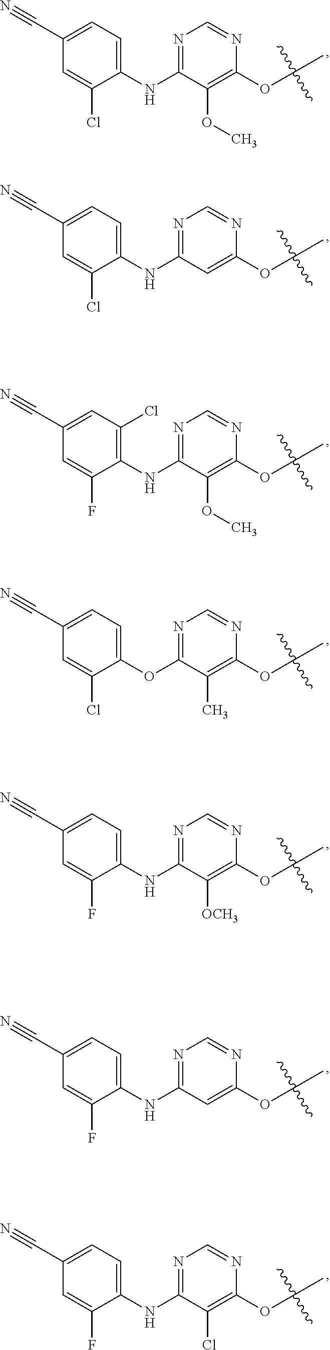

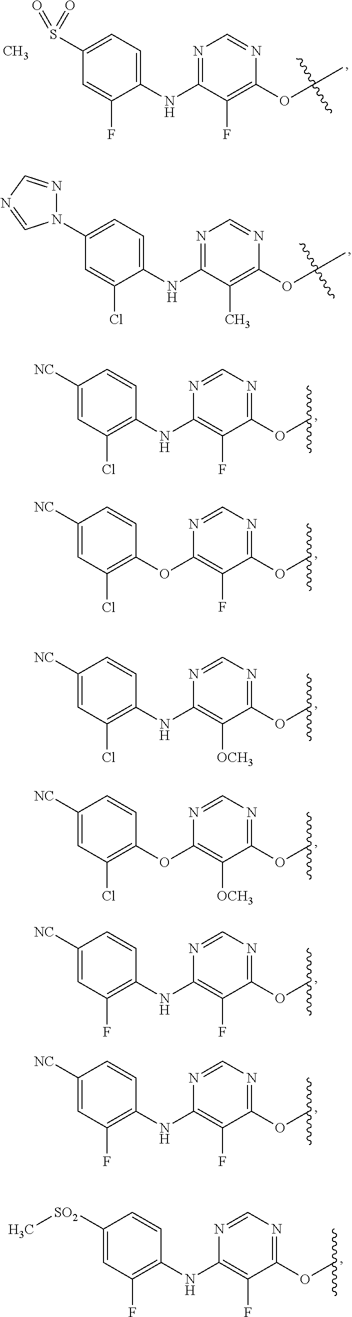

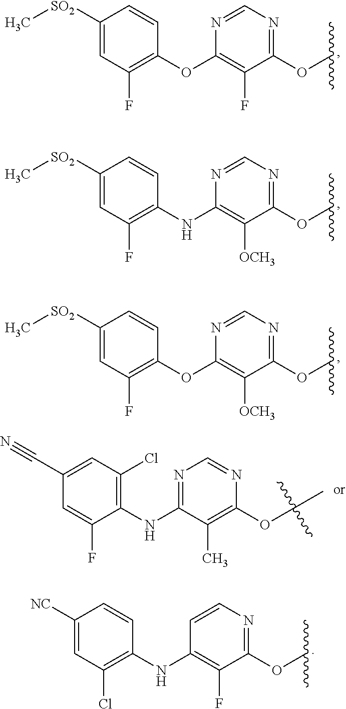

In another embodiment, the group

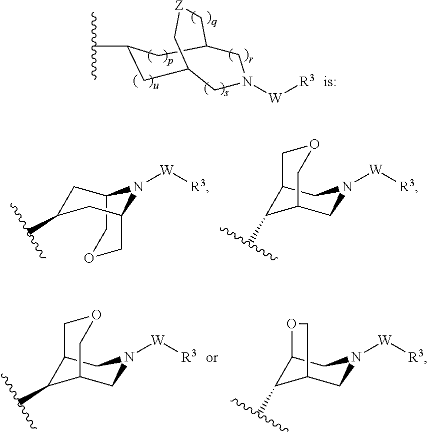

and the group —B—X-A-Y— is:

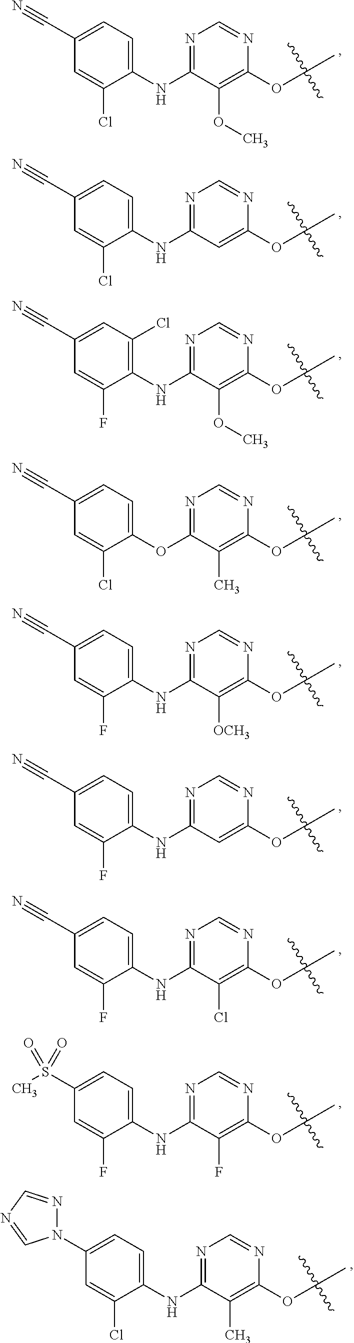

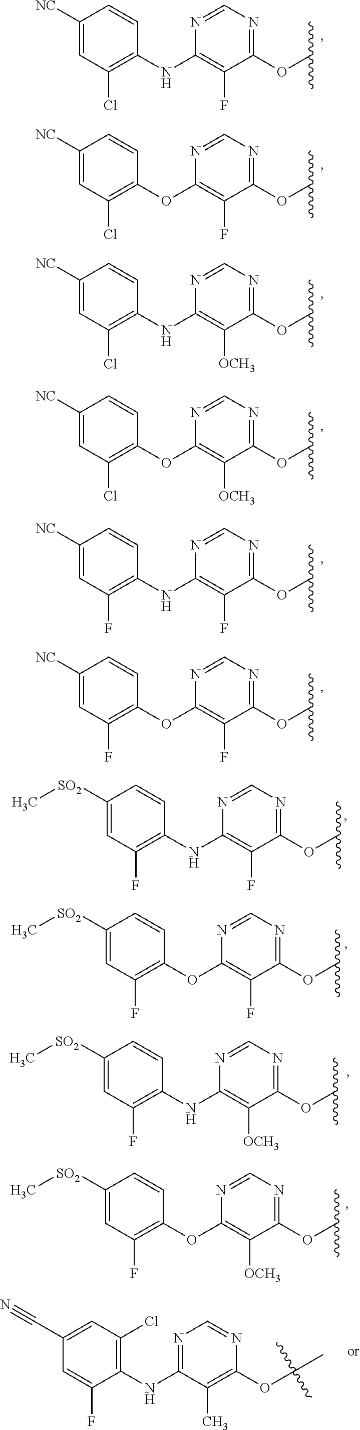

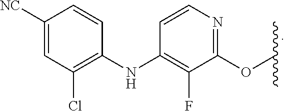

In one embodiment, the group B—X-A-Y— is:

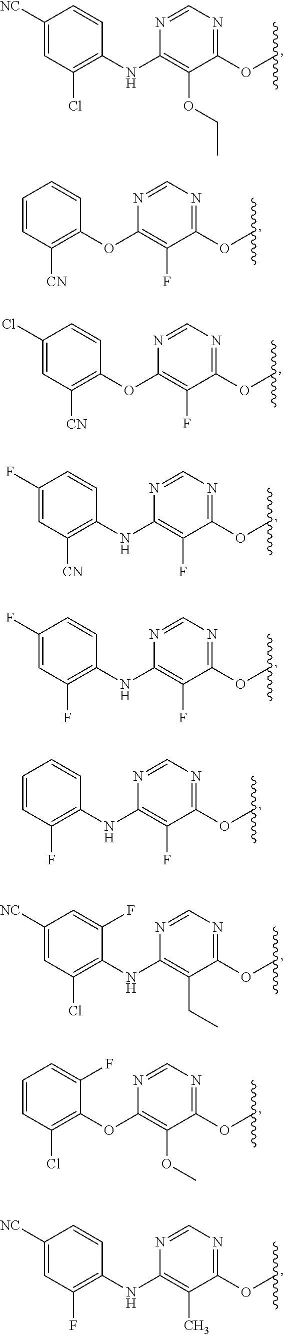

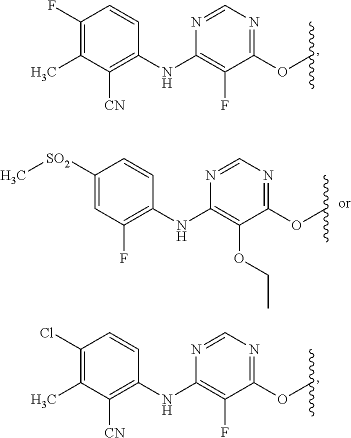

and the group

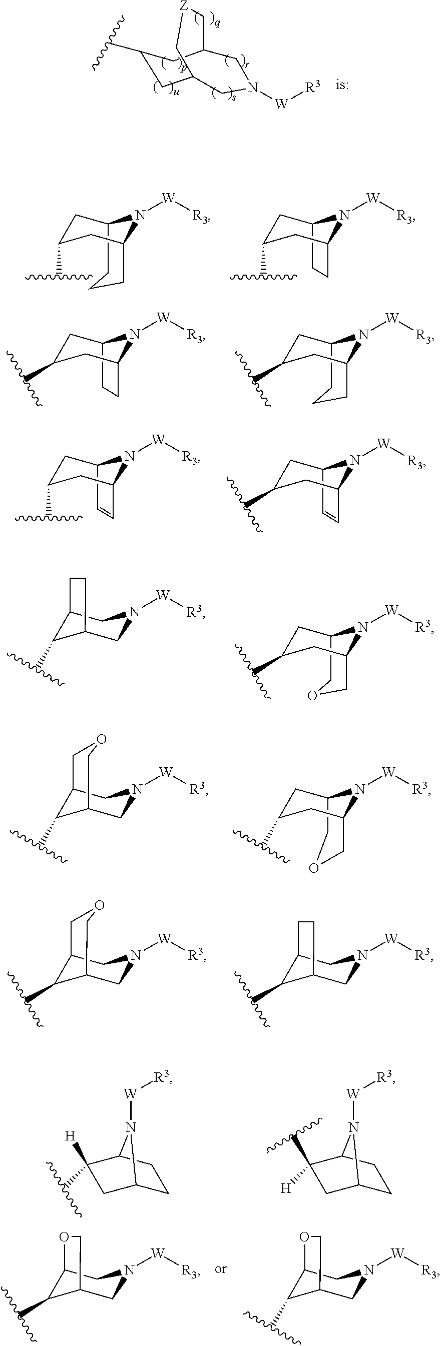

In another embodiment, the group B—X-A-Y— is:

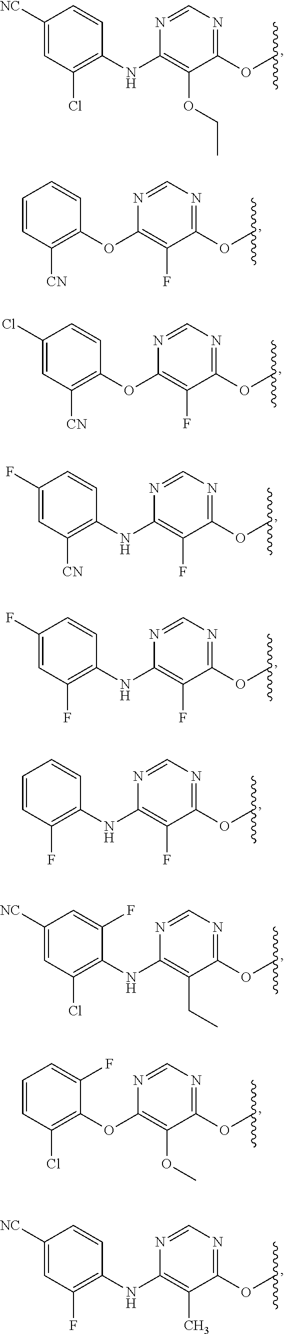

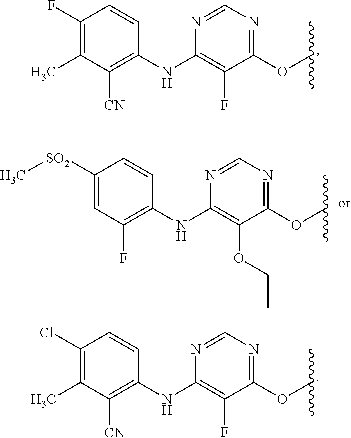

and the group

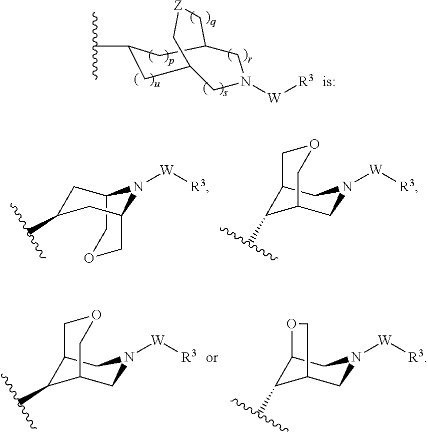

In another embodiment, the group B—X-A-Y— is:

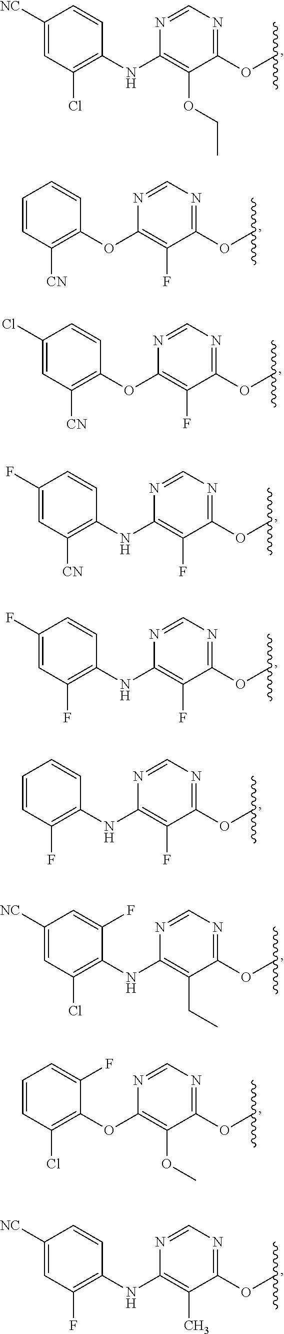

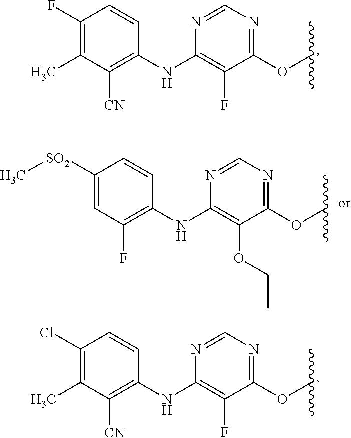

and the group

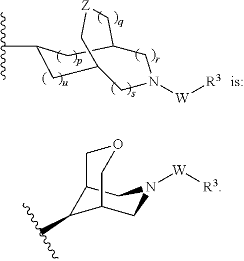

In another embodiment, the group

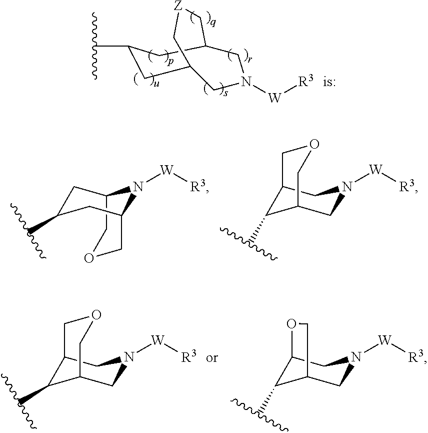

W is —C(O)O— or a bond;

R³ is:

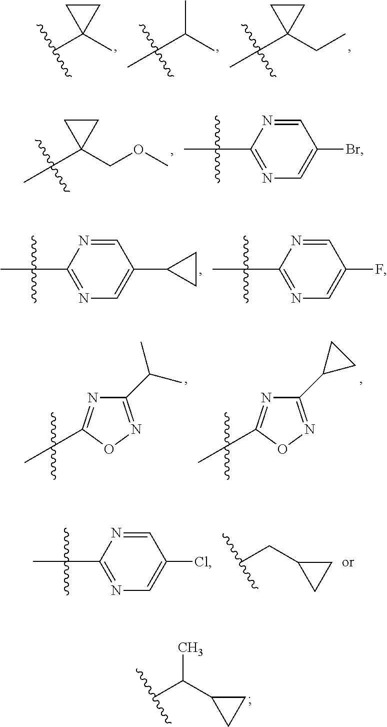

and the group —B—X-A-Y— is:

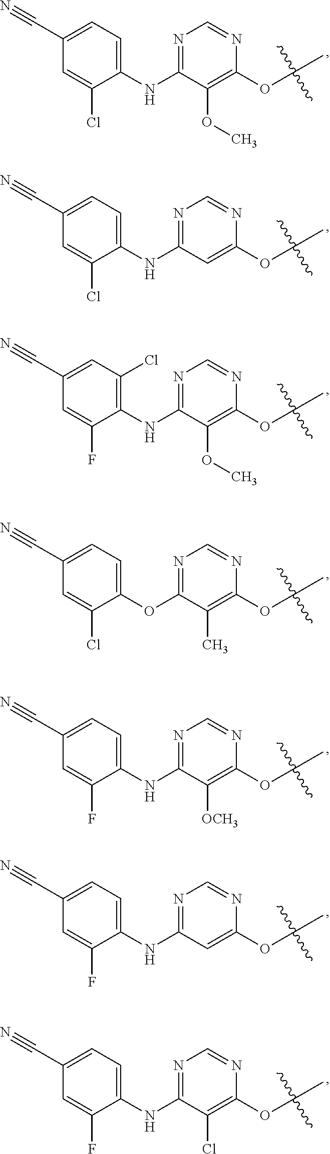

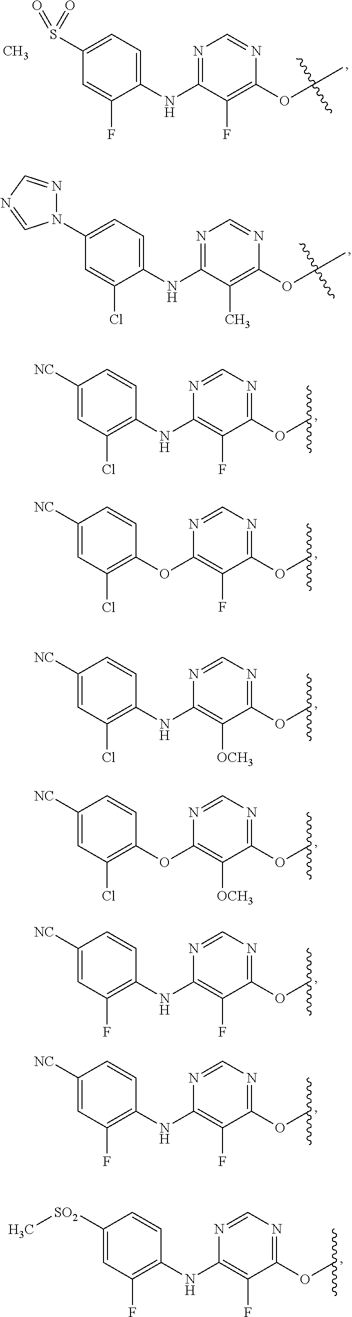

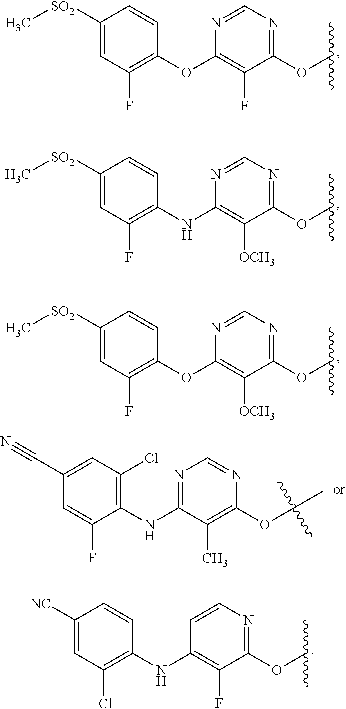

In one embodiment,

W is —C(O)O— or a bond;

R³ is selected from:

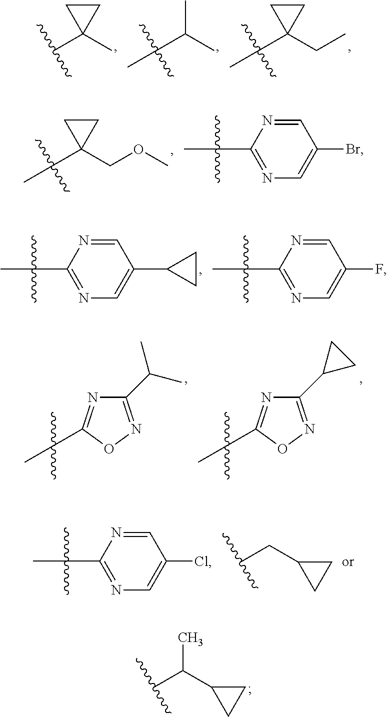

the group B—X-A-Y— is:

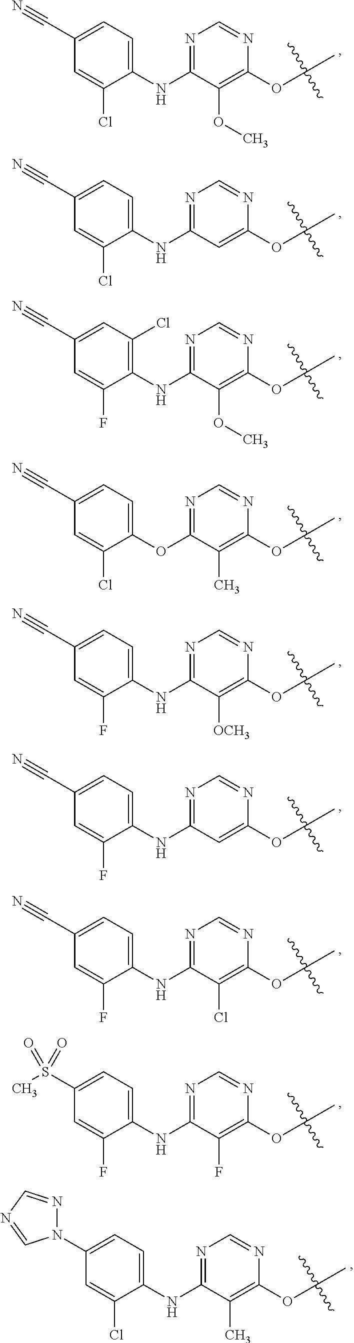

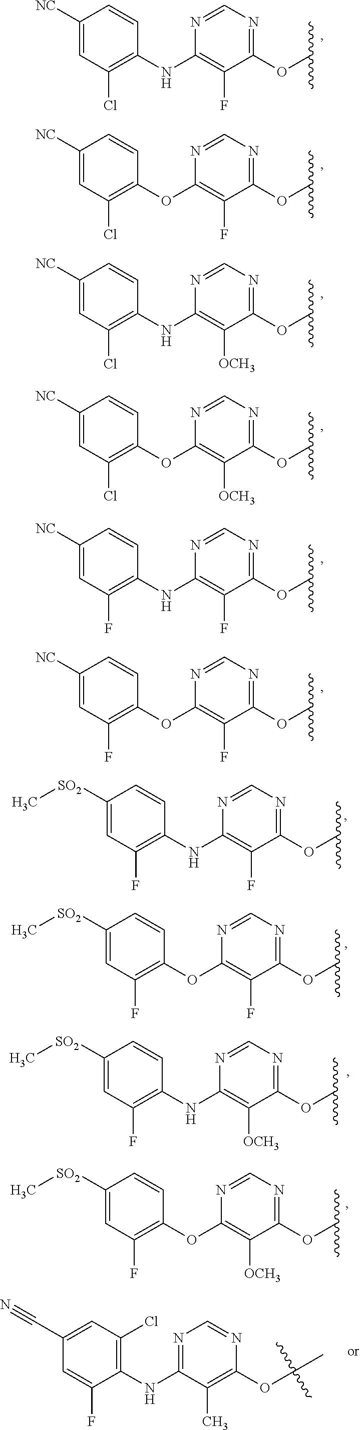

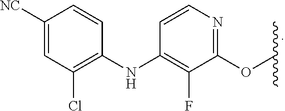

and the group

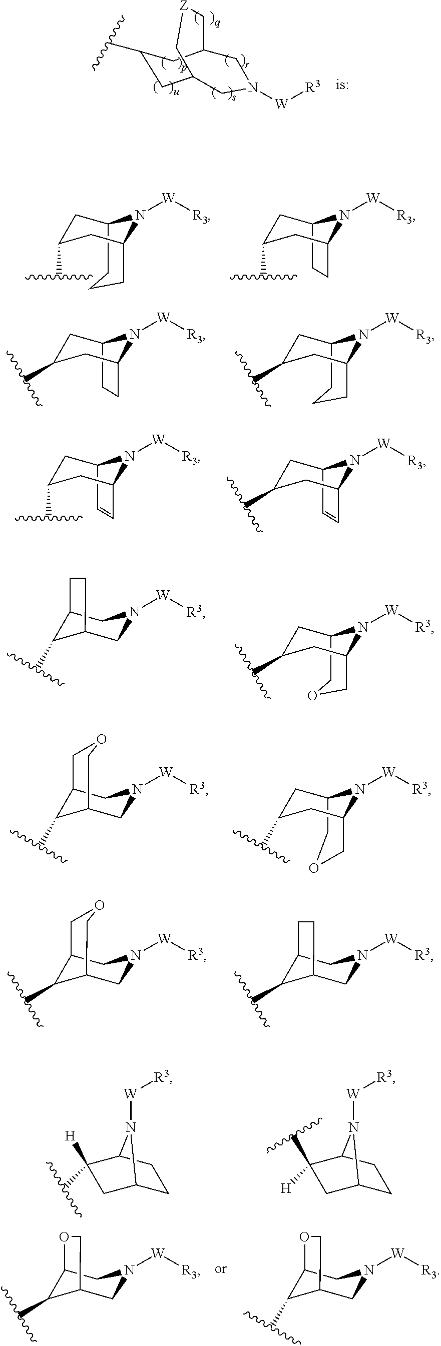

In another embodiment,

A is:

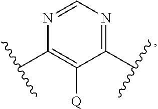

wherein Q is F, methyl, ethyl, ethoxy or methoxy;

B is selected from:

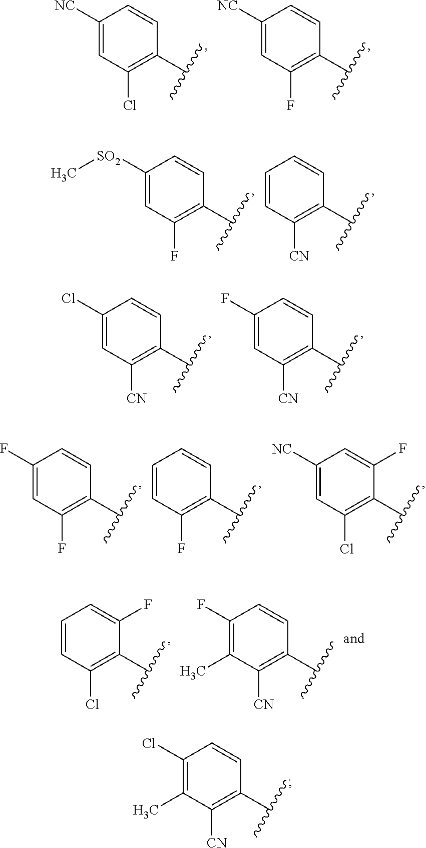

X is —O— or —N

Y is —O—;

the group W—R³ is:

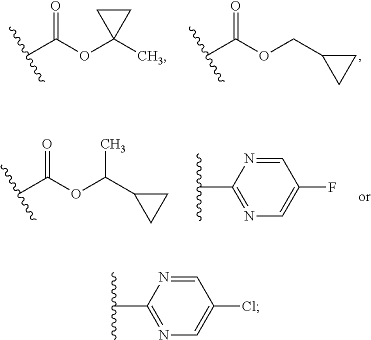

and the group

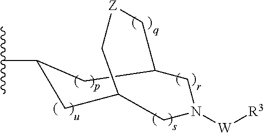

is:

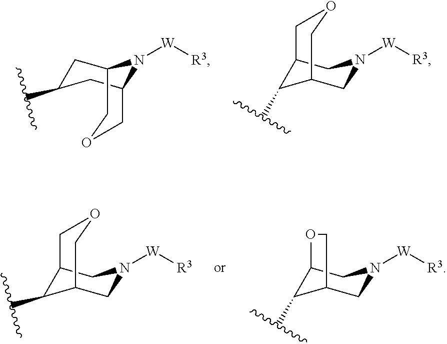

In one embodiment, the Compounds of Formula (I) have the formula:

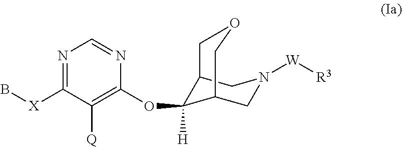

and pharmaceutically acceptable salts thereof, wherein:

B is phenyl, which is substituted with R^{a};

Q is alkyl, —O-alkyl or F;

W is a bond or —C(O)O—;

X is —O— or —NH; and

R^{a} represents from 1 to 3 ring subsituents, which can be the same or different, and are selected from alkyl, —S(O)₂alkyl, halo and —CN; and

R³ is heteroaryl or -(alkylene)ₜ-cycloalkyl, wherein said heteroaryl can be optionally substituted with a halo group and wherein the cycloalkyl moiety of said -(alkylene)-cycloalkyl group can be optionally substituted with an alkyl group.

In one embodiment, for the Compounds of Formula (Ia):

Q is —CH₃, —CH₂CH₃, —OCH₃, —OCH₂CH₃ or F;

R^{a} represents 1 to 3 ring subsituents, which can be the same or different, and are selected from —S(O)₂CH₃, F, Cl, —CN and —CH₃; and

R³ is

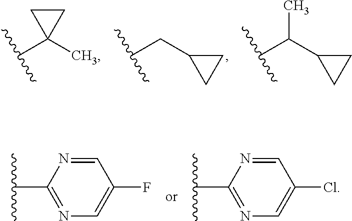

In another embodiment, for the Compounds of Formula (Ia):

Q is —CH₃, —CH₂CH₃, —OCH₃, —OCH₂CH₃ or F;

B is selected from:

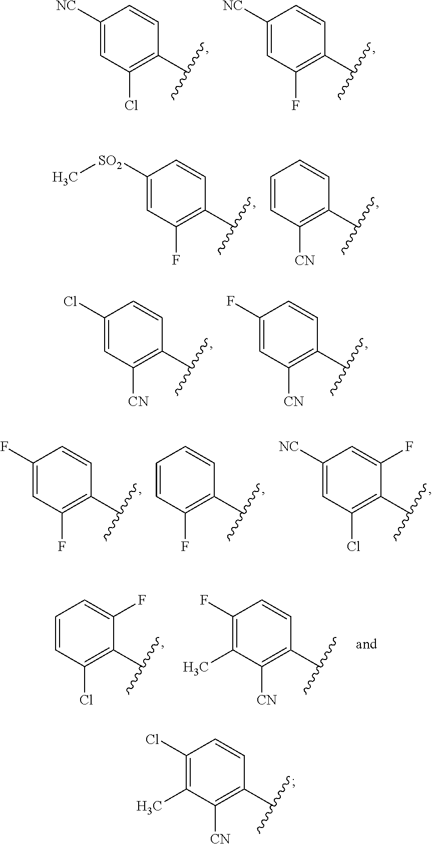

and

the group W—R³ is:

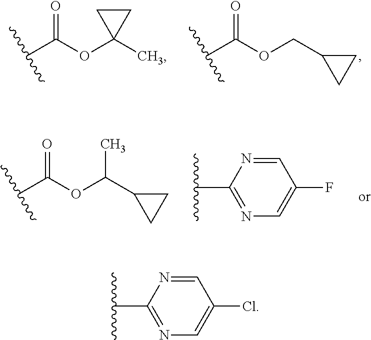

In another embodiment, for the Compounds of Formula (Ia):

X is —NH—;

B is selected from:

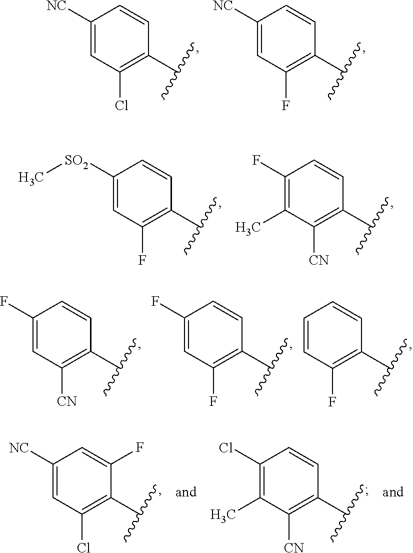

the group W—R³ is:

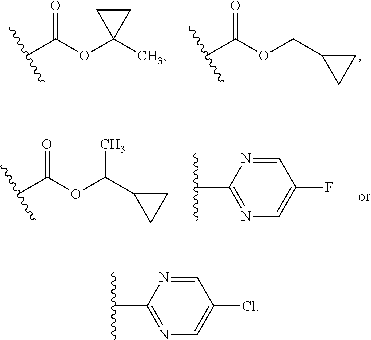

In another embodiment, for the Compounds of Formula (Ia):

X is —O—

B is selected from:

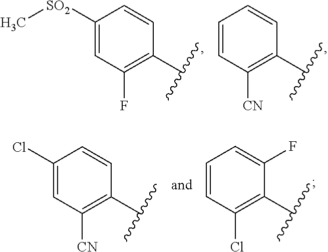

and
the group W—R³ is:

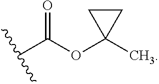

In one embodiment, the present invention provides compounds of Formula (I), wherein A, B, W, X, Y, Z, R³, p, q, r, s and u are selected independently of each other.

In another embodiment, a compound of formula (I) is in purified form.

Non-limiting examples of the Bridged Bicyclic Heterocycle Derivatives of the present invention include, but are not limited to compounds 1-64, depicted below:

| Compound | |
|---|---|
| No. | STRUCTURE |
| 1 | 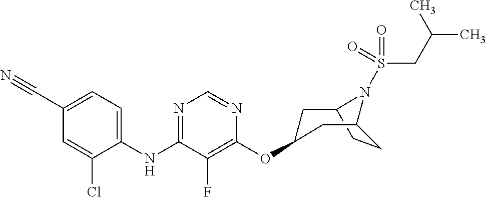 |
| 2 | 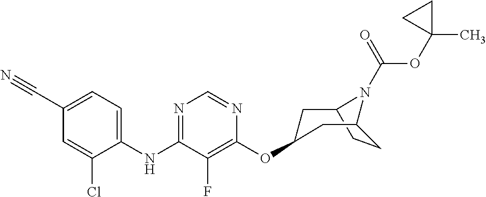 |
| 3 | 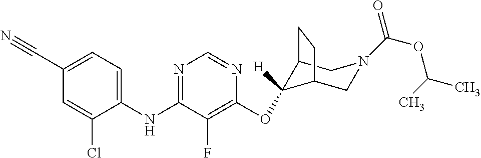 |
| 4 | 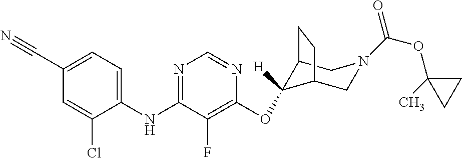 |
| 5 | 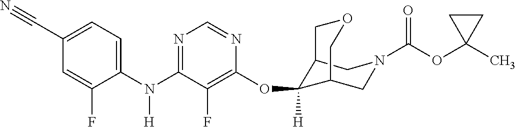 |
| 6 | 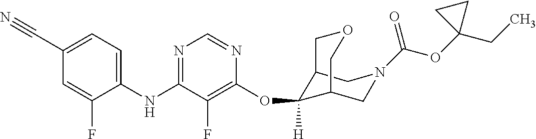 |
| 7 | 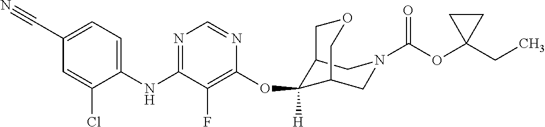 |
| 8 | 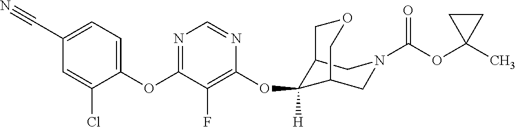 |
| 9 | 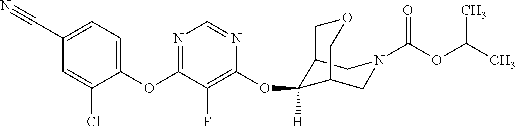 |
| 10 | 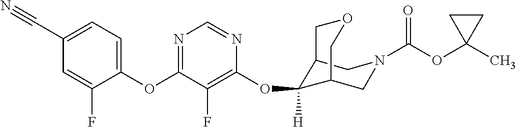 |
| 11 | 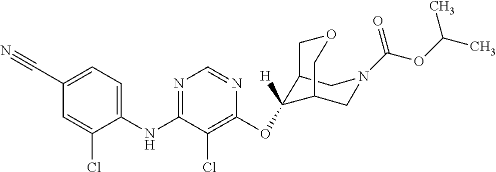 |
| 12 | 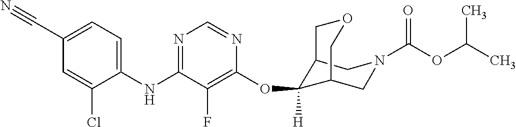 |
| 13 | 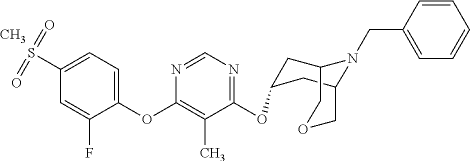 |
| 14 | 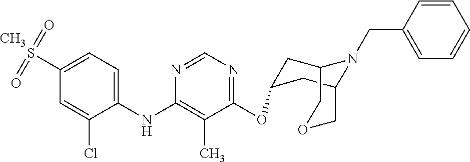 |
| 15 | 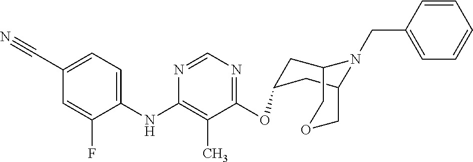 |
| 16 | 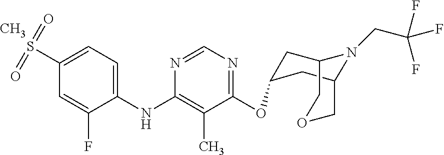 |
| 17 | 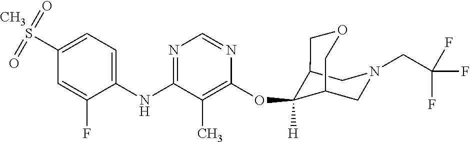 |
| 18 | 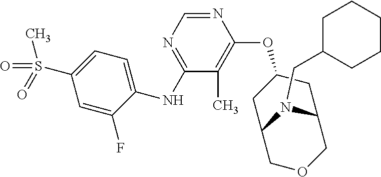 |
| 19 | 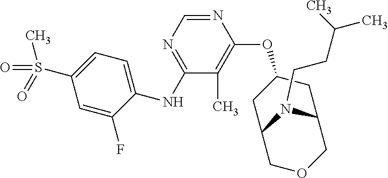 |
| 20 | 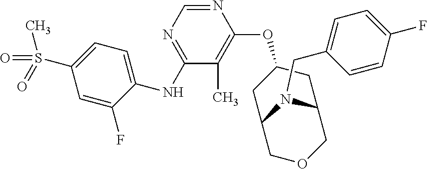 |
| 21 | 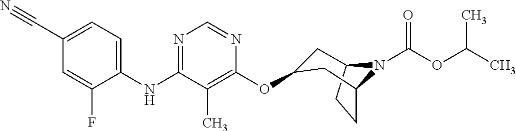 |
| 22 | 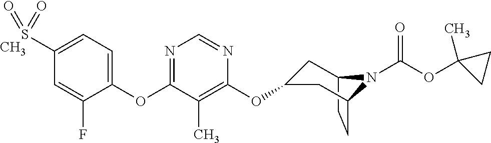 |
| 23 | 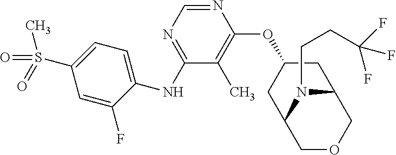 |
| 24 | 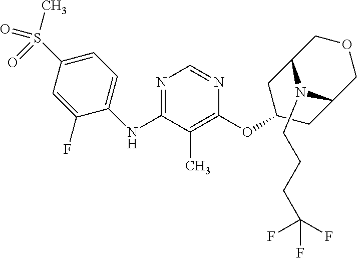 |
| 25 | 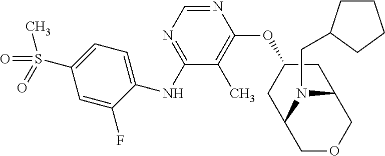 |
| 26 | 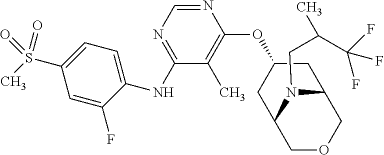 |
| 27 | 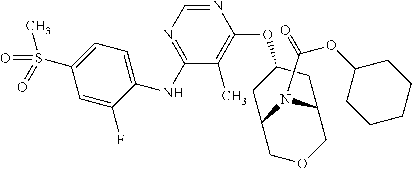 |
| 28 | 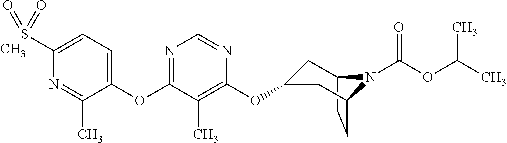 |
| 29 | 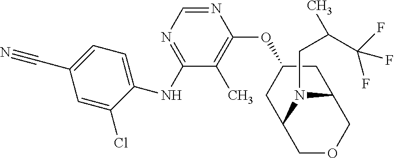 |
| 30 | 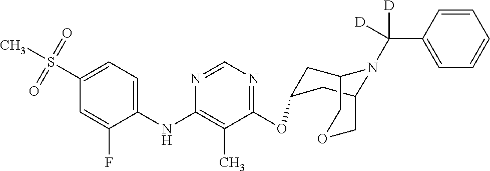 |
| 31 | 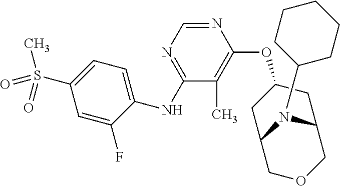 |
| 32 | 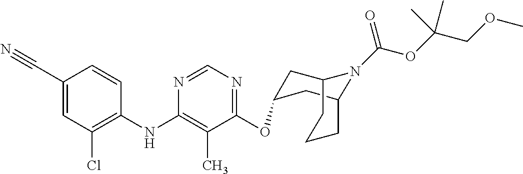 |
| 33 | 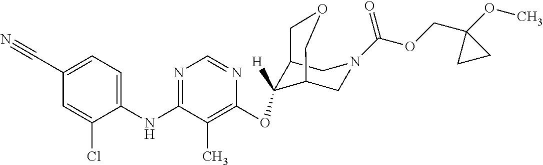 |
| 34 | 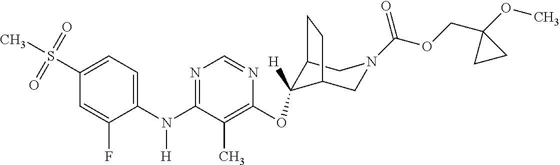 |
| 35 | 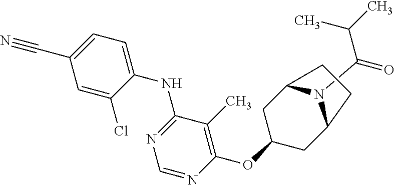 |
| 36 | 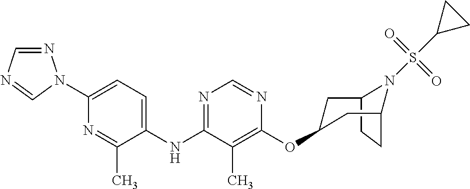 |
| 37 | 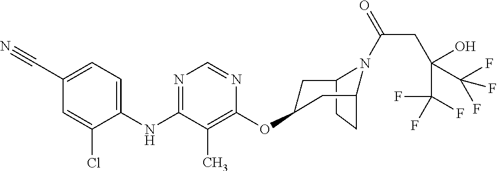 |
| 38 | 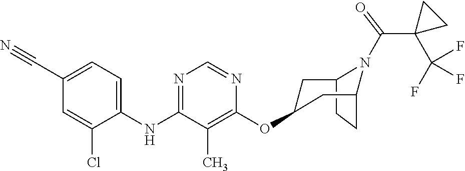 |
| 39 | 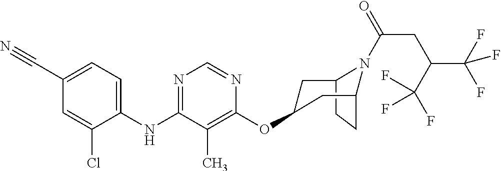 |
| 40 | 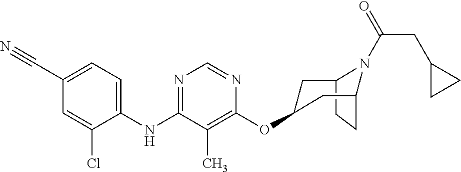 |
| 41 | 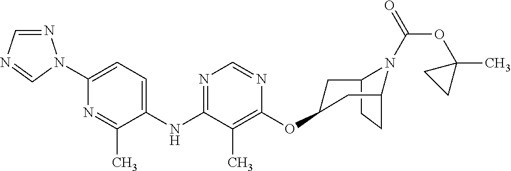 |
| 42 | 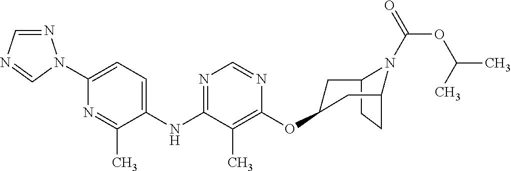 |
| 43 | 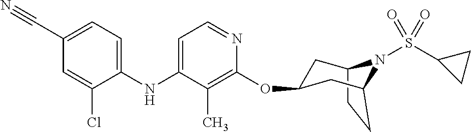 |
| 44 | 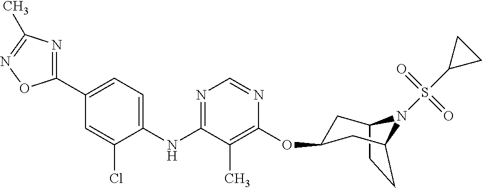 |
| 45 | 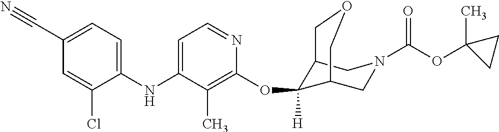 |
| 46 | 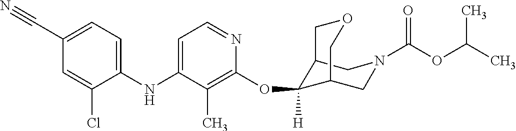 |
| 47 | 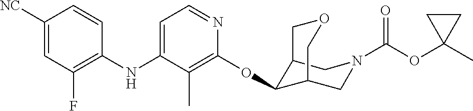 |
| 48 | 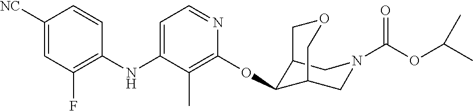 |
| 49 | 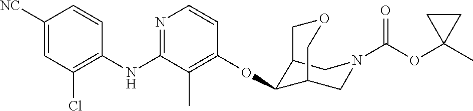 |
| 50 | 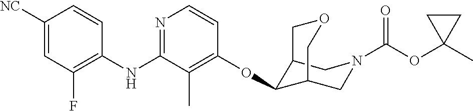 |
| 51 | 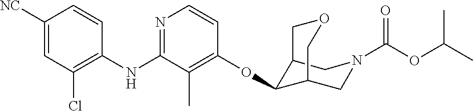 |
| 52 | 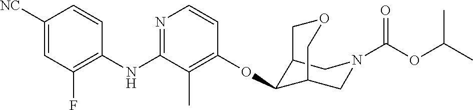 |
| 53 | 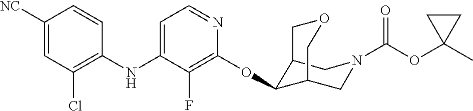 |
| 54 | 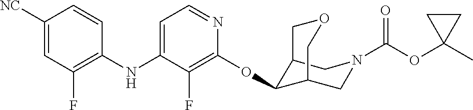 |
| 55 | 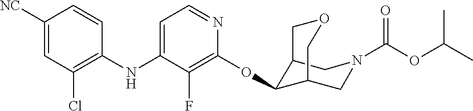 |
| 56 | 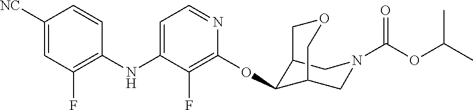 |
| 57 | 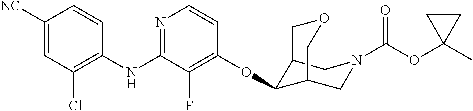 |
| 58 | 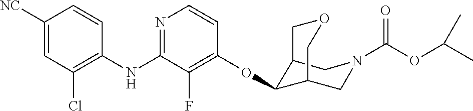 |
| 59 | 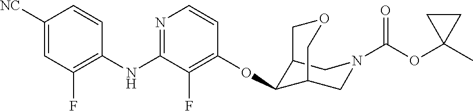 |
| 60 | 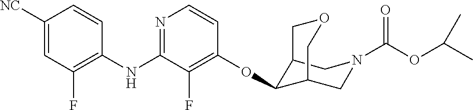 |
| 61 | 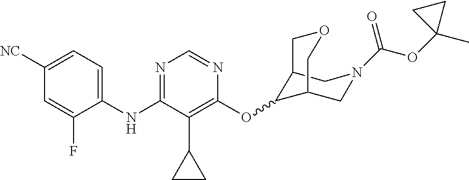 |
| 62 | 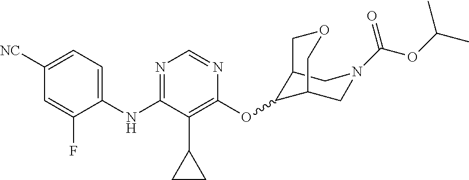 |
| 63 | 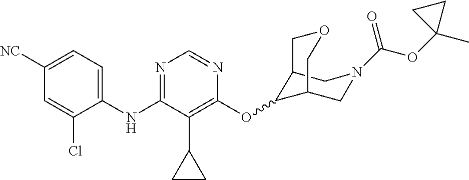 |
| 64 | 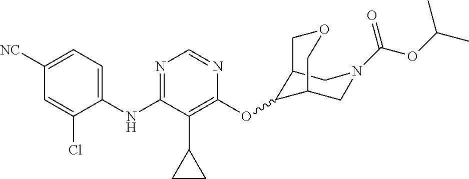 |

and pharmaceutically acceptable salts, solvates, esters, prodrugs and stereoisomers thereof.

Additional illustrative compounds of the present invention include compounds 65-204, 206-213 and 215-607 as depicted in Examples section below, and pharmaceutically acceptable salts, solvates, esters, prodrugs and stereoisomers thereof.

In one embodiment, illustrative compounds of the present invention are compounds 240, 252, 276, 301, 302, 353, 362, 363, 365, 384, 396, 406, 413, 437, 508, 550, 555, 556 and 568.

### Methods for Making the Bridged Bicyclic Heterocycle Derivatives

Methods useful for making the Bridged Bicyclic Heterocycle Derivatives are set forth in the Examples below and generalized in Schemes 1-2. Alternative synthetic pathways and analogous structures will be apparent to those skilled in the art of organic synthesis.

Scheme 1 illustrates a method useful for making the compounds of formula iii, which are useful intermediates for making the Bridged Bicyclic Heterocycle Derivatives.

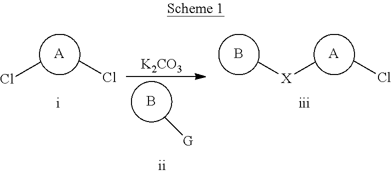

wherein A and B are defined above for the compounds of formulas (I), (II), (III) and (IV); G is —OH, —SH, —NHR¹⁰ or a carbon nucleophile; and X is —S—, —O—, —C(R¹)₂— or —NR¹⁰.

A dichloro aryl or heteroaryl compound of formula i can be reacted with a compound of formula ii in the presence of a non-nucleophilic base, such as potassium carbonate to provide the intermediate compounds of formula iii.

Scheme 2 illustrates a general method useful for making the compounds of formula (I).

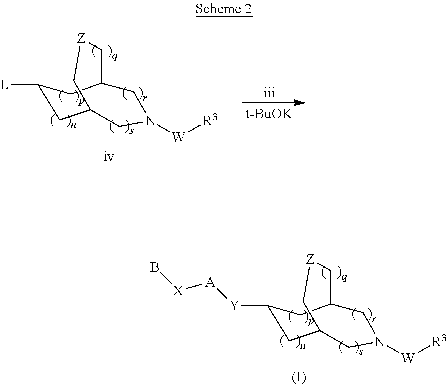

wherein L is -(alkylene)ₜ-OH, -(alkylene)ₜ-N(R¹⁰)H or —SH; and, R³, W, X, Y, Z, A, B, p, q, r, s, t and u are defined above for the compounds of formula (I).

A compound of formula iv can be coupled with a compound of formula iii in the presence of potassium tert-butoxide using the method described in International Publication No. WO 07/035,355 to Jones et al., to provide the compounds of formula (I).

The compounds of formula iv can be commercially available or can be prepared using methods well-known to one skilled in the art of organic chemistry, including, but not limited to, the methods described in the Examples section below.

The starting materials and reagents depicted in Schemes 1-2 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art will recognize that the synthesis of Bridged Bicyclic Heterocycle Derivatives may require the need for the protection of certain functional groups (i.e., derivatization for the purpose of chemical compatibility with a particular reaction condition). Suitable protecting groups for the various functional groups of the Bridged Bicyclic Heterocycle Derivatives and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

Solvents, reagents, and intermediates that are commercially available were used as received. Reagents and intermediates that are not commercially available were prepared in the manner described below. ¹H NMR spectra were obtained on a Gemini AS-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The observed parent ions are given.

### Example 1

### Preparation of Compound 1

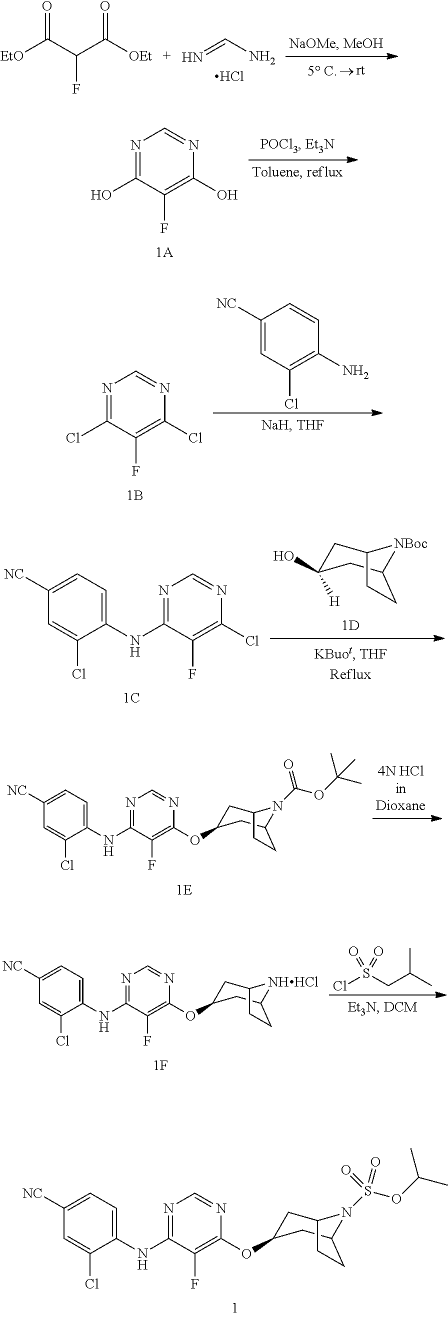

### Step A—Synthesis of Compound 1A:

To a cold suspension of sodium methoxide (30% solution in methanol) (1.46 g, 80.83 mmol) in methanol (˜36 mL) at 5° C. was added formamidine hydrochloride (1.36, 16.84 mmol) and the resulting reaction stirred for 10 minutes. Diethyl fluoromalonate (3.0 g, 16.84 mmol) was then added and the resulting reaction mixture was stirred for about 15 hours at room temperature. The reaction mixture was concentrated in vacuo and the residue obtained was dissolved in ice cold water (100 mL) and acidified to pH=7. The white precipitate obtained was filtered, washed with water and dried to provide compound 1A (1.78, 81.28%).

### Step B—Synthesis of Compound 1B:

Compound 1A (1.78 g, 13.07 mmol) was dissolved in toluene (25 mL), then triethylamine was added and the mixture was heated to near reflux. POCl₃ compound (in toluene (4 mL)) was added to the mixture slowly and the resulting reaction was refluxed for about 15 hours at 110° C. The reaction mixture was cooked to room temperature, poured over crushed ice, extracted 2 times with toluene and the combined organic layers were separated and washed with saturated NaHCO₃ solution and then with brine. The organic layer was dried over anhydrous MgSO₄, filtered and concentrated in vacuo to provide compound 1B (0.74 g, 32.5%).

### Step C—Synthesis of Compound 1C:

To a stirred solution of NaH (0.44 g, 11.08 mmol) in tetrahydrofuran (10 mL) was added a solution of 4-amino-3-chloro benzonitrile (0.32 g, 2.08 mmol) in tetrahydrofuran (15 mL) and the resulting reaction was stirred for 30 minutes. The reaction mixture was then cooled to 0° C. and a solution of compound 1B (0.37 g, 2.22 mmol) in tetrahydrofuran (15 mL) was added. The resulting reaction was stirred at 0° C. for 30 minutes and then for about 15 hours at room temperature. The reaction was quenched with water, extracted 2 times with ethyl acetate and the combined organic layers were dried over anhydrous Na₂SO₄, filtered and concentrated in vacuo. The residue obtained was purified using silica gel column chromatography using 1% (7N NH₃ in MeOH)—99% CH₂Cl₂ as eluent to provide compound 1C (0.3 g, 48.39%).

### Step D—Synthesis of 1E:

Compound 1D (0.11 g, 0.5 mmol; made according to the method described in International Publication No. WO 98/18788 to Blythin, et al.) was dissolved in tetrahydrofuran (3 mL) and KBuO^{t} (1 mL, 1 mmol) was added to it followed by a solution of compound 1C (0.14 g, 0.5 mmol) in tetrahydrofuran (5 mL). The resulting reaction was refluxed at 84° C. for about 15 hours then cooled to room temperature quenched with water and extracted 2 times with ethyl acetate. The combined organic layers were dried over anhydrous Na₂SO₄, filtered, concentrated in vacuo and purified using preparative TLC (100% CH₂Cl₂) to provide compound 1E (0.075 g, 32.05%).

### Step E—Synthesis of Compound 1F:

Compound 1E (0.065 g, 0.14 mmol) was added to 4N HCl in dioxane (1 mL) and stirred for an hour at room temperature. The mixture was concentrated in vacuo to remove excess acid to provide the amine hydrochloride salt of compound 1F (0.05 g, 96%).

### Step F—Synthesis of Compound 1:

Compound 1F (0.018 g, 0.05 mmol) was dissolved in CH₂Cl₂ (2 mL) and triethylamine (0.02 mL, 0.13 mmol) was added to it and stirred for 10 minutes. This was followed by the addition of 2-methylpropane-1-sulfonyl chloride (0.01 mL, 0.07 mmol) and the resulting reaction was stirred for 1 hour at room temperature. The reaction was quenched with saturated ammonium chloride solution and extracted with CH₂Cl₂ (2×). The combined organic layers were dried over anhydrous Na₂SO₄, filtered, concentrated in vacuo. The resulting residue was purified using preparative TLC using 100% CH₂Cl₂ (containing 15 drops of 7N NH₃ in CH₃OH per 100 ml of CH₂Cl₂) as mobile phase to provide compound 1 (0.012 g, 56%). LCMS (M+H)=494.3

### Example 2

### Preparation of Compound 2

### Step A—Synthesis of Compound 2A:

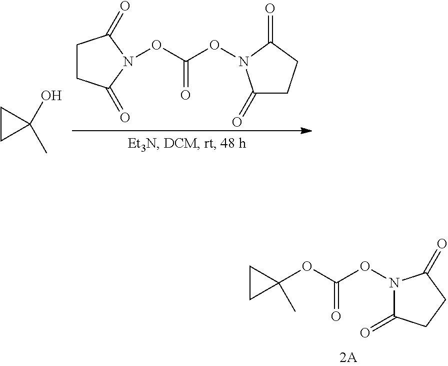

To a solution of 1-methyl cyclopropanol (1.9 g, 26.34 mmol) and disuccinimidyl carbonate (10.0 g, 39.1 mmol) in acetonitrile (40.00 mL), was added triethylamine (10.9 mL) and the resulting reaction was stirred for 48 hours. The reaction mixture was extracted with EtOAc and washed with satd. NaHCO₃, brine and water. Rotary evaporation and drying in vacuum resulted in a creamy white solid 2A (4.5 g, 80% yield).

### Step B—Synthesis of Compound 2:

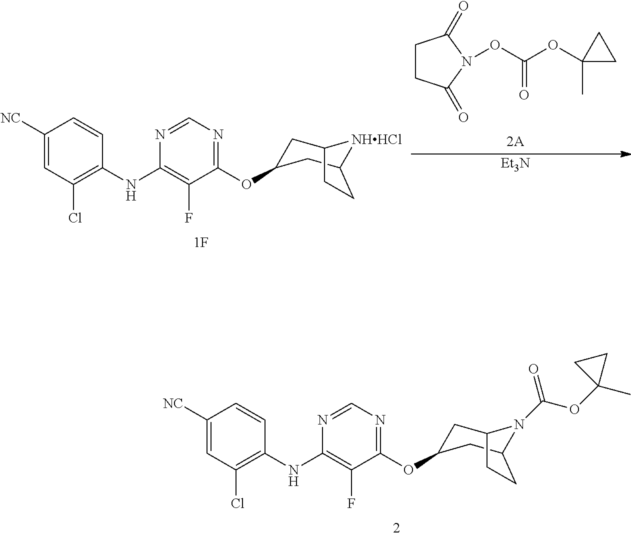

Compound 1F (0.018 g, 0.04 mmol) was dissolved in CH₂Cl₂ (2 mL) and triethylamine (0.02 mL, 0.13 mmol) was added to it and stirred for 10 minutes. This was followed by the addition of compound 2A (0.02 g, 0.09 mmol) and the resulting mixture was stirred for 1 hour at room temperature. The reaction was quenched with saturated ammonium chloride solution and extracted 2 times with CH₂Cl₂. Combined organic layers were dried over anhydrous Na₂SO₄, filtered, concentrated in vacuo, purified using preparative TLC using 20% acetone-80% hexane as mobile phase and the product, 2 (0.01 g, 48%) was isolated. LCMS (M+H)=472.3

### Example 3

### Preparation of Compound 3

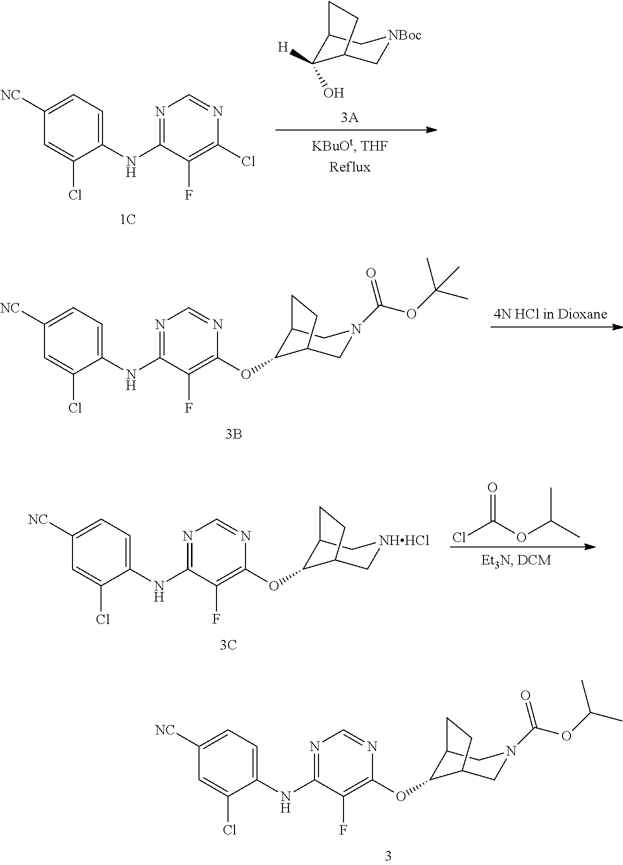

### Step A—Synthesis of Alcohol 3A:

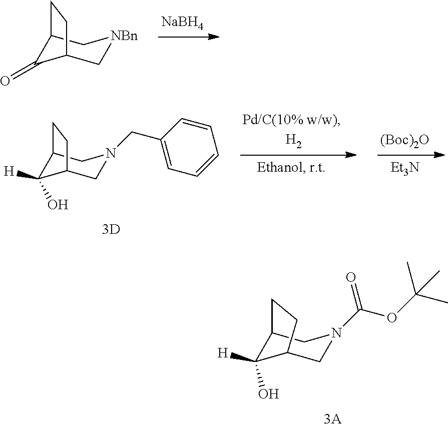

To a solution of ketone (1.0 g, 4.6 mmol, commercially available) in methanol (16 mL) at 0° C., was added NaBH₄ (0.44 g, 11.6 mmol) and the resulting reaction was stirred at room temperature for 1 hour. The reaction was carefully quenched with water and extracted with dichloromethane (2×). The combined organic layers were dried over Na₂SO₄ and concentrated in vacuo to provide compound 3D (0.6 g, 60%). Compound 3D (0.6 g, 2.8 mmol) was then dissolved in ethanol (10 mL) and hydrogenated at room temperature, 1 atmosphere in presence of Pd/C (0.06 g, 10% w/w) for 2 days. Triethylamine (1.2 mL, 8.3 mmol), di-tert-butyl dicarbonate (0.65 g, 2.98 mmol) were added to the mixture and stirred at room temperature for about 15 hours. The reaction mixture was filtered over celite and concentrated in vacuo to remove ethanol. The residue was dissolved in DCM and washed with water. The organic layer was dried over anhydrous Na₂SO₄, filtered, concentrated in vacuo to provide compound 3A (0.65 g, 95%)

### Step B—Synthesis of Compound 3B:

The endo-alcohol 3A (0.08 g, 0.35 mmol) was dissolved in tetrahydrofuran (4 mL) and KBuO^{t} (0.7 mL, 0.7 mmol) was added to it followed by a solution of compound 1C (0.10 g, 0.35 mmol) in tetrahydrofuran (5 mL). The resulting reaction was refluxed at 84° C. for about 15 hours then cooled to room temperature. KBuO^{t} (0.7 mL, 0.7 mmol) was then added to the mixture and the resulting reaction was refluxed at 84° C. for 2 hours. The reaction was cooled to room temperature, quenched with water and extracted 2 times with ethyl acetate. The combined organic layers were dried over anhydrous Na₂SO₄, filtered, and concentrated in vacuo. The resulting residue was purified using preparative TLC using 100% CH₂Cl₂ (containing 13 drops of 7N NH₃ in CH₃OH/100 ml of DCM) as mobile phase to provide compound 3B (0.06 g, 40.5%).

### Step C—Synthesis of Compound 3C:

Compound 3B (0.053 g, 0.11 mmol) was added to 4N HCl in dioxane (2 mL) and the solution was stirred for 1 hour at room temperature. The reaction mixture was then concentrated in vacuo. Provide compound 3C (0.045 g, 97%).

### Step D—Synthesis of Compound 3:

Compound 3C (0.022 g, 0.05 mmol) was dissolved in CH₂Cl₂ (3 mL) and triethylamine (0.02 mL, 0.16 mmol) was added to the solution. The mixture was stirred for 10 minutes, then isopropyl chloroformate (0.08 mL, 0.08 mmol) was added and the resulting reaction was stirred for 1 hour at room temperature. The reaction mixture was then quenched with saturated ammonium chloride solution and extracted with CH₂Cl₂ (2×). The combined organic layers were dried over anhydrous Na₂SO₄, filtered and concentrated in vacuo. The residue obtained was purified using preparative TLC using 30% acetone-70% hexane as mobile phase to provide compound 3 (0.012 g, 50%). LCMS (M+H)=460.3

### Example 4

### Preparation of Compound 4

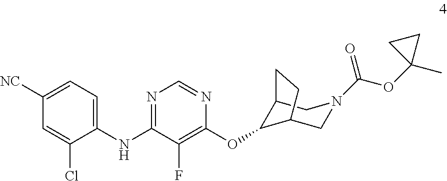

Compound 3C (0.022 g, 0.05 mmol) was dissolved in CH₂Cl₂ (2 mL) and the resulting solution was added to triethylamine (0.02 mL, 0.16 mmol). The reaction was stirred for 10 minutes, then compound 2A (0.023 g, 0.11 mmol) was added and the resulting mixture was stirred for 1 hour at room temperature. The reaction was quenched with saturated ammonium chloride solution and extracted 2 times with CH₂Cl₂. The combined organic layers were dried over anhydrous Na₂SO₄, filtered, concentrated in vacuo and purified using preparative TLC using 30% acetone-70% hexane as mobile phase to provide compound 4 (0.008 g, 32%). LCMS (M+H)=472.3

### Example 5

### Preparation of Compound 5

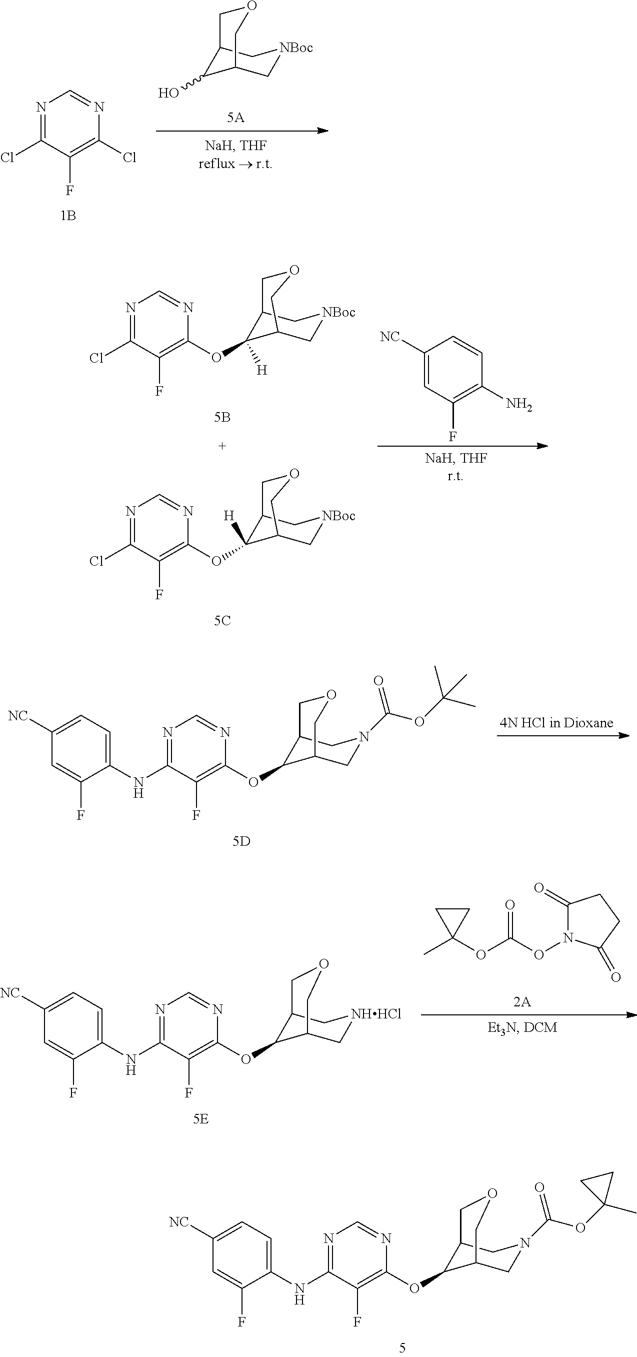

### Step A—Synthesis of Compound 5A:

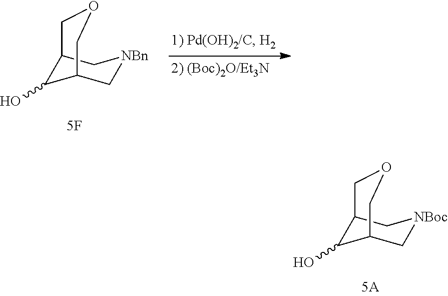

A solution of compound 5F (0.97 g, 4.16 mmol, prepared from the corresponding ketone as described in Huttenloch et al., H. Chem. Eur. J. 2002, 8, 4767-4780), 20% Pd(OH)₂/C (873 mg, 1.25 mmol) in methanol (30 mL) was reacted under 1 atm H₂ for 24 hours. Then filtered through Celite and concentrated in vacuo. The residue was dissolved in 20 mL DCM and cooled to 0° C. Followed by adding Boc₂O (0.95 mL, 4.11 mmol) and Et₃N (0.82 mL, 5.86 mmol). The reaction was warmed to room temperature for about 15 hours. The reaction was quenched with NaHCO₃, extracted with dichloromethane (3×30 mL). The combined organic layer was dried over Na₂SO₄ and concentrated in vacuo. The residue was purified on a silica gel column (ISCO) with MeOH(NH₃) in dichloromethane (0→5%) to provide compound 5A (715 mg).

### Step B—Synthesis of Compounds 5B and 5C:

To a stirred solution of NaH (0.36 g, 8.9 mmol) in THF (4 mL) was added a solution of alcohol 5A (0.42 g, 1.7 mmol) in THF (5 mL) in a sealed tube and the resulting mixture was refluxed at 84° C. for 1 hour. The reaction mixture was cooled to room temperature and a solution of starting material 1B (0.3 g, 1.8 mmol) in THF (5 mL) was added to the reaction mixture and the resulting mixture was stirred at room temperature for 30 minutes. The reaction was quenched with water and extracted 2 times with ethyl acetate. The combined organic layer was dried over anhydrous Na₂SO₄, filtered, concentrated in vacuo, and purified using silica gel column chromatography using 40% ethyl acetate-60% hexane as mobile phase and the products 5B (0.1 g, 15%) and 5C (0.12 g, 18%) were isolated.

### Step C—Synthesis of Compound 5D:

To a stirred solution of NaH (0.02 g, 0.5 mmol) in THF (3 mL) was added a solution of 4-cyano-2-fluoro aniline (0.013 g, 0.09 mmol) in THF (4 mL) and stirred at room temperature for 30 minutes. The reaction was then cooled to 0° C. and a solution of starting material 5B (0.035 g, 0.09 mmol) in THF (4 mL) and the resulting mixture was refluxed for about 15 hours at 84° C. The reaction was quenched with water and extracted 2 times with ethyl acetate. Combined organic layers were dried over anhydrous Na₂SO₄, filtered, concentrated in vacuo, and purified using preparative TLC using 40% ethyl acetate-60% hexane as mobile phase to provide compound 5D (0.02 g, 46%)

### Step D—Synthesis of Compound 5E:

5D (0.019 g, 0.04 mmol) was added to 4N HCl in dioxane (1 mL) and stirred for an hour at room temperature. The mixture was concentrated in vacuo to remove excess acid to provide the amine hydrochloride salt, 5E (0.015 g, 92%).

### Step E—Synthesis of Compound 5:

Compound 5E (0.015 g, 0.037 mmol) was dissolved in CH₂Cl₂ (2 mL) and triethylamine (0.02 mL, 0.11 mmol) was added to it and stirred for 10 minutes. This was followed by the addition of compound 2A (0.012 g, 0.05 mmol) and the resulting mixture was stirred for 1 hour at room temperature. The reaction was quenched with saturated ammonium chloride solution and extracted 2 times with CH₂Cl₂. Combined organic layers were dried over anhydrous Na₂SO₄, filtered, concentrated in vacuo, purified using preparative TLC using 30% ethyl acetate-70% hexane as mobile phase to provide compound 5 (0.015 g, 87.2%). LCMS (M+H)=472.3

### Example 6

### Preparation of Compound 6

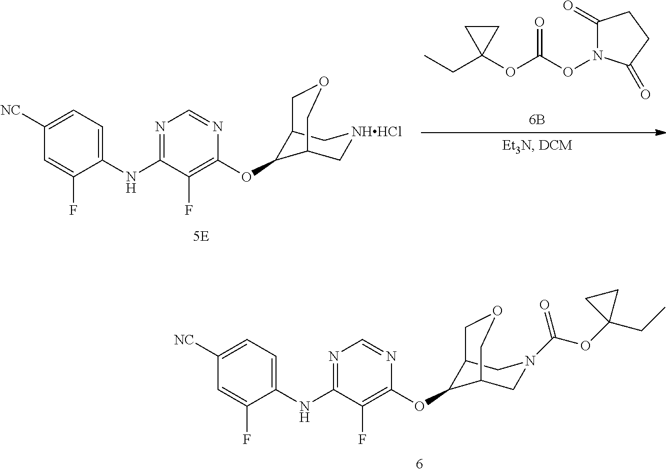

### Step A—Synthesis of Compound 6B

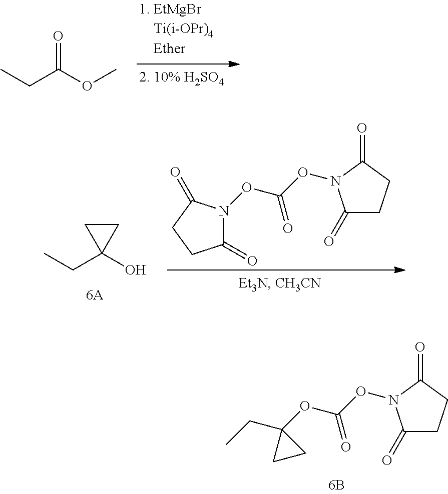

To a solution of methyl propionate (2.18 ml, 22.7 mmol) and titanium isopropoxide (0.33 ml, 1.14 mmol) in ether (80 mL) was added a solution of ethyl magnesium bromide (16.04 ml, 48.12 mmol) in ether (60 mL) over a period of 1 hour at room temperature. Stirring continued for 15 minutes at room temperature. The mixture was poured into cooled 10% aqueous sulfuric acid (250 mL) and the product was extracted 3 times with ether. The combined organic layers were washed with water, dried over anhydrous Mg₂SO₄, filtered and concentrated in vacuo to provide compound 6A (1.8 g, 92%).

Compound 6A (1.0 g, 11.61 mmol) was dissolved in acetonitrile (75 mL), then N,N′-Disuccinimidyl carbonate (5.9 g, 23.22 mmol) was added to the mixture and stirred at room temperature for 10 minutes. Triethylamine (4.9 ml, 34.83 mmol) was added to the mixture slowly and resulting mixture was stirred at room temperature for about 15 hours. The reaction was quenched with saturated NaHCO₃ solution and extracted 2 times with ethyl acetate. The combined organic layers were dried over anhydrous Na₂SO₄, filtered and concentrated in vacuo to provide compound 6B (1.2 g, 46.2%).

### Step B—Synthesis of Compound 6

Compound 5E (0.015 g, 0.04 mmol) was dissolved in CH₂Cl₂ (2 mL) and triethylamine (0.02 mL, 0.11 mmol) was added to it and stirred for 10 minutes. This was followed by the addition of compound 6B (0.010 g, 0.04 mmol) and the resulting mixture was stirred for 1 hour at room temperature. The reaction was quenched with saturated ammonium chloride solution and extracted 2 times with CH₂Cl₂. The combined organic layers were dried over anhydrous Na₂SO₄, filtered, concentrated in vacuo, purified using preparative TLC using 40% ethyl acetate-60% hexane as mobile phase. The product was repurified using preparative TLC using 2% methanol-98% DCM as mobile phase to provide compound 6 (0.011 g, 63.3%). LCMS (M+H)=486.3

### Example 7

### Preparation of Compound 7

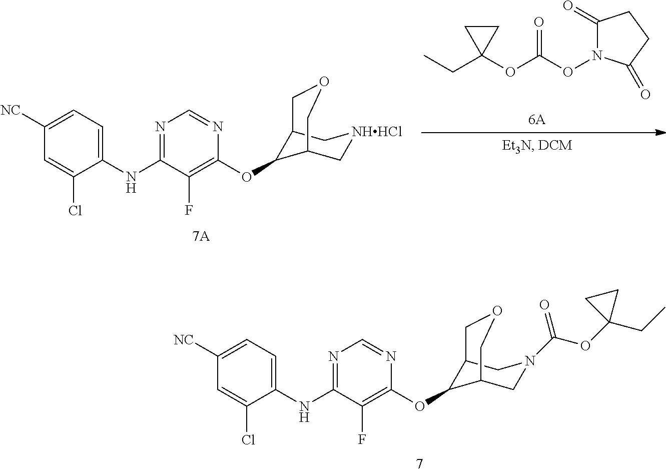

Compound 7A (0.011 g, 0.03 mmol) was dissolved in CH₂Cl₂ (2 mL) and triethylamine (0.01 mL, 0.08 mmol) was added to it and stirred for 10 minutes. This was followed by the addition of compound 6A (0.009 g, 0.04 mmol) and the resulting mixture was stirred for 1 hour at room temperature. The reaction was quenched with saturated ammonium chloride solution and extracted 2 times with CH₂Cl₂. The combined organic layers were dried over anhydrous Na₂SO₄, filtered, concentrated in vacuo and purified using preparative TLC using 50% ethyl acetate and 50% hexane as mobile phase. The product was repurified using preparative TLC using 2% methanol and 98% DCM as mobile phase and to provide compound 7 (0.07 g, 53.4%). LCMS (M+H)=502.3

The Amine hydrochloride of compound 7A can be synthesized using the same procedure used for the synthesis of compound 5E and replacing 4-cyano-2-fluoro aniline with 2-chloro-4-cyano aniline.

### Example 8

### Preparation of Compound 8

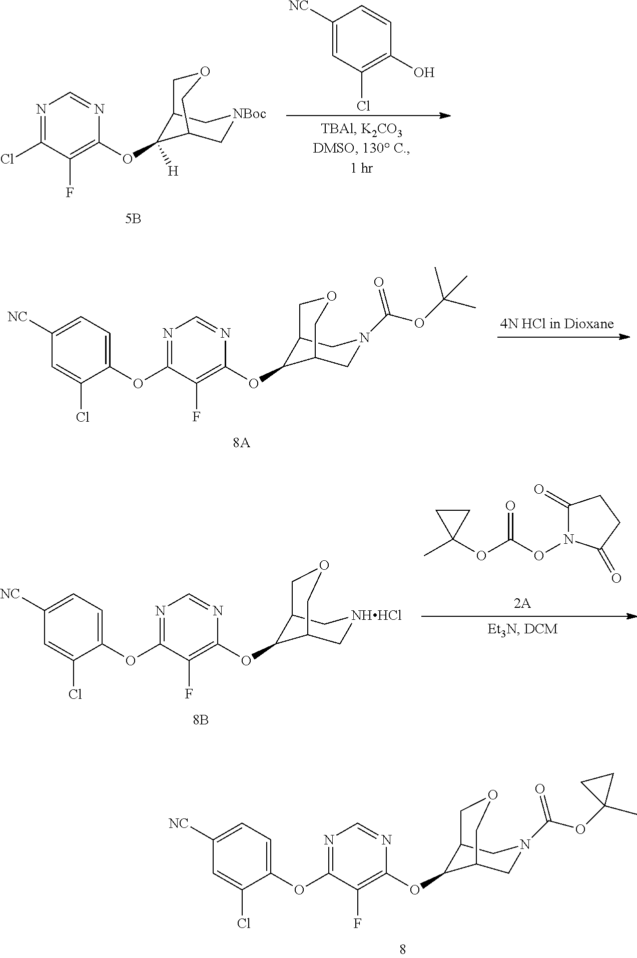

### Step A—Synthesis of Compound 8A

Compound 5B (0.037 g, 0.10 mmol), 2-chloro-4-cyano phenol (0.015 g, 0.10 mmol), tetrabutylammonium iodide (0.024 g, 0.06 mmol), K₂CO₃ (0.016 g, 0.12 mmol) were taken up in DMSO (1 mL) in a sealed tube at heated for 1 hour at 130° C. The reaction was quenched with water and extracted 3 times with ethyl acetate. The combined organic layers were dried over anhydrous Na₂SO₄, filtered, concentrated in vacuo and purified using preparative TLC using 40% ethyl acetate-60% hexane as mobile phase to provide compound 8A (0.032 g, 67%).

### Step B—Synthesis of Compound 8B

Compound 8A (0.032 g, 0.07 mmol) was added to 4N HCl in dioxane (1.5 mL) and the reaction was stirred for an hour at room temperature. The mixture was then concentrated in vacuo to provide compound 8B (0.025 g, 89%).

### Step C—Synthesis of Compound 8

Compound 8B (0.025 g, 0.06 mmol) was dissolved in CH₂Cl₂ (2 mL) and triethylamine (0.03 mL, 0.18 mmol) was added to it and stirred for 10 minutes. This was followed by the addition of compound 2A (0.015 g, 0.07 mmol) and the resulting mixture was stirred for 1 hour at room temperature. The reaction was quenched with saturated ammonium chloride solution and extracted 2 times with CH₂Cl₂. The combined organic layers were dried over anhydrous Na₂SO₄, filtered, concentrated in vacuo, purified using preparative TLC using 55% ethyl acetate-45% hexane as mobile phase. The product was repurified using preparative TLC using 20% acetone-80% hexane as mobile phase to provide compound 8 (0.016 g, 58%). LCMS (M+H)=489.3

### Example 9

### Preparation of Compound 9

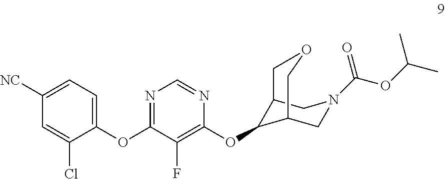

Compound 8B (0.01 g, 0.02 mmol) was dissolved in CH₂Cl₂ (1.5 mL) and triethylamine (0.006 mL, 0.07 mmol) was added to it and stirred for 10 minutes. This was followed by the addition of isopropyl chloroformate (0.03 ml, 0.03 mmol) and the resulting mixture was stirred for 1 hour at room temperature. The reaction was quenched with saturated ammonium chloride solution and extracted 2 times with CH₂Cl₂. The combined organic layers were dried over anhydrous Na₂SO₄, filtered, concentrated in vacuo, purified using preparative TLC using 45% ethyl acetate-55% hexane as mobile phase to provide compound 9 (0.006 g, 62%). LCMS (M+H)=477.3

### Example 10

### Preparation of Compound 10

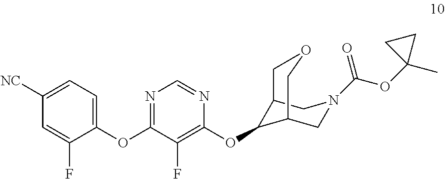

Compound 10 was made using the method described in Example 8 and replacing 3-chloro-4-hydroxybenzonitrile with 3-fluoro-4-hydroxybenzonitrile. LCMS (M+H)=473.3

### Example 11

### Preparation of Compound 11

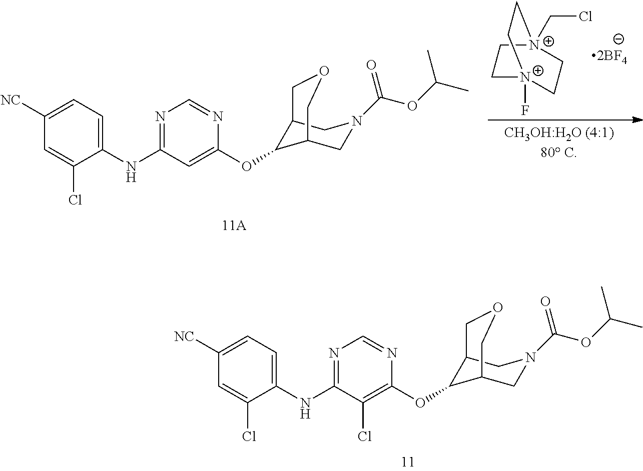

Compound 11A (0.04 g, 0.08 mmol) was dissolved in CH₃OH:H₂O (4:1, 1.25 mL) in a sealed tube. Selectfluor (0.046 g, 0.13 mmol) was added and the mixture and heated at 80° C. for about 15 hours. The reaction was concentrated in vacuo and purified using preparative TLC using 55% ethyl acetate-45% hexane as mobile phase to provide compound 11 (0.01 g, 25%). LCMS (M+H)=492.3.

Compound 11A can be synthesized by adopting the methods in Example 5E and 3 by replacing the middle core with 4,6-dichloropyrimidine.

### Example 12

### Preparation of Compound 12

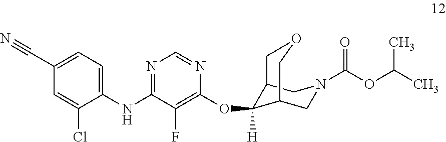

Compound 12 can be prepared from compound 7A using the method described in Example 9. LCMS (M+H)=476.3

### Example 13

### Preparation of Compound 13

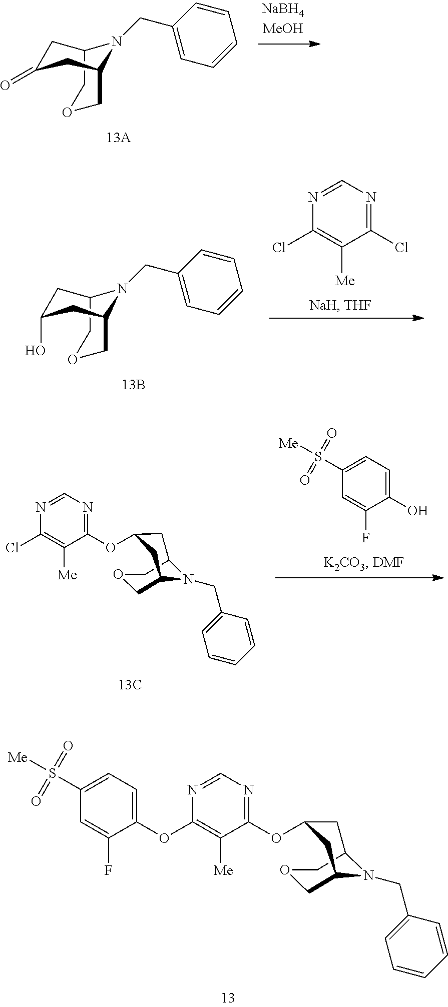

### Step A—Synthesis of Compound 13A:

To a solution of 1,4-anhdyroerythritol (5.0 g, 48 mmoL) in H₂O (60 mL) was added NalO₄ (5.1 g, 24 mmol). The solution was allowed to stir for about 15 hours at room temperature. To the solution was added MeCN (80 mL) and the solution was stirred for 30 minutes. The white precipitate formed was removed by filtration and the filtrate was concentrated in vacuo. To the remaining aqueous, solution was added 1,3-acetonedicarboxylic acid (7.0 g, 48 mmol) benzylamine (6.1 mL, 52 mmol), and concentrated HCl (2.5 mL) and the solution was stirred at room temperature for 1 hour, then at 50° C. for 2 hours. The reaction mixture was then cooled to 0° C., then basified to PH ˜10 using 1M NaOH. The basic solution was extracted with EtOAc and DCM and the combined organic layers were dried (MgSO₄) and concentrated in vacuo. The residue obtained was purified using a silica gel cartridge (eluting with EtOAc in Hexanes 30%-100%) to provide compound 13A (3.2 g, 29%).

### Step B—Synthesis of Compound 13B:

To a solution of compound 13A (1.5 g, 6.5 mmol) in MeOH (20 mL) was added NaBH₄ (320 mg, 8.4 mmol) and the solution was stirred at room temperature for 10 hours. H₂O (100 mL) was added and the mixture was extracted with EtOAc, and the organic layer was dried over MgSO₄ and concentrated in vacuo to provide compound 13B (1.4 g, 98%).

### Step C—Synthesis of Compound 13C:

To a solution of compound 13B (1.48 g, 6.5 mmol) in THF (33 mL) was added NaH (60% dispersion in oil) (390 mg, 9.8 mmol), and the solution was heated at 60° C. for 1.5 hours then allowed to cool to room temperature. 4,6-dichloro-5-methylpyrimidine was added and the reaction was stirred for about 15 hours at room temperature. The reaction was quenched with H₂O and extracted with EtOAc. The organic layer was dried (MgSO₄) and concentrated in vacuo to provide a crude residue which was chromatographed on a silica gel cartridge (0-20% EtOAc in Hexanes) to provide compound 13C (1.5 g, 65%).

### Step D—Synthesis of Compound 13:

To a solution of compound 13C (75 mg, 0.21 mmol) and 2-fluoro-4-methylsulfonylphenol (48 mg, 0.25 mmol) in DMF (2 mL) was added K₂CO₃ (44 mg, 0.32 mmol) and the solution was heated to 120° C. and allowed to stir at this temperature. The reaction mixture was then cooled to room temperature, concentrated in vacuo, and purified using preparative thin layer chromatography using (50% EtOAc-Hexanes) to provide compound 13 (25 mg, 23%). LCMS: 514.3 (M+H)⁺.

### Example 14

### Preparation of Compound 14

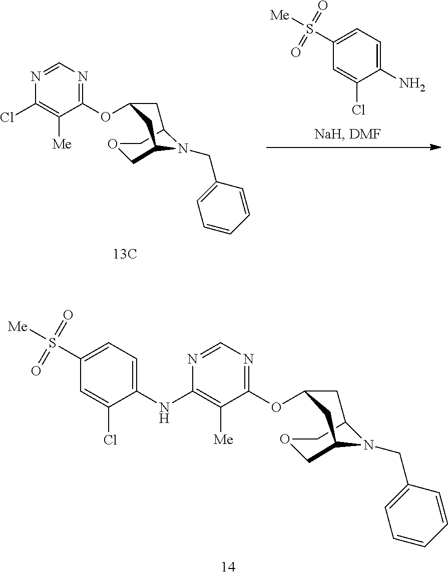

To a solution of compound 13C (75 mg, 0.21 mmol) and 2-chloro-4-methylsulfonylaniline (51 mg, 0.25 mmol) in DMF (2 mL) was added NaH (60% by wt. in hexanes, 13 mg, 0.32 mmol) and the solution was stirred and heated to 70° C. and allowed to stir at this temperature for 4 hours. The reaction mixture was then cooled to room temperature, concentrated in vacuo, and purified using preparative thin layer chromatography using (5% methanol-methylene chloride) to provide compound 14 (66 mg, 60%). LCMS: 529.3 (M+H)⁺.

### Example 15

### Preparation of Compound 15

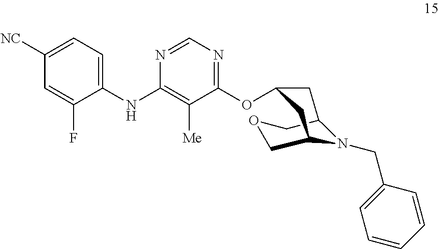

Using the method described in Example 16, and substituting 4-cyano-2-fluoroaniline for 2-chloro-4-methylsulfonylaniline, compound 15 was prepared. LCMS: 460.3 (M+H)⁺.

### Example 16

### Preparation of Compound 16

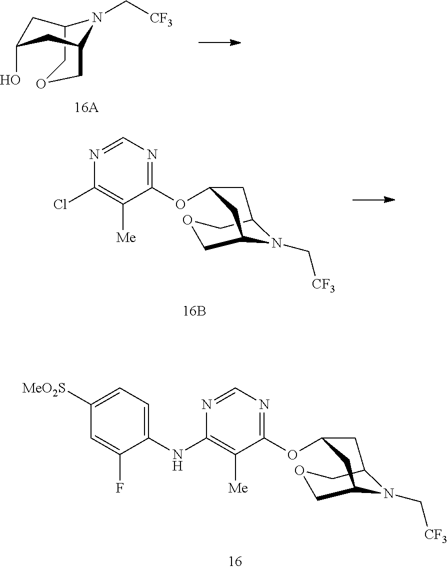

Using the methods described in Example 13, and substituting trifluoroethylamine for benzylamine, compound 16A and compound 16B were prepared. Using the method described in Example 14, and substituting compound 16B for compound 13C, compound 16 was prepared. LCMS: 505.3 (M+H)⁺.

### Example 17

### Preparation of Compound 17

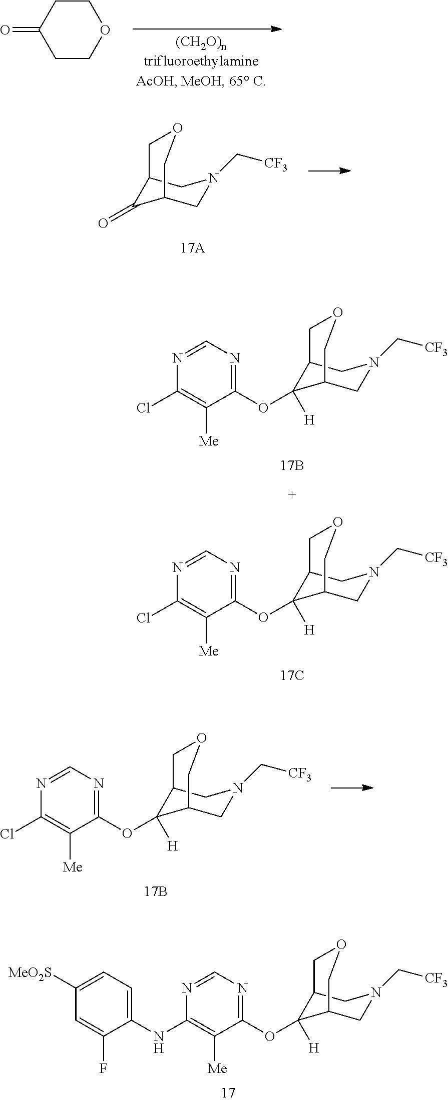

A solution of tetrahydro-4H-pyran-4-one (3.7 mL, 20 mmol), trifluoroethylamine (3.4 mL, 42 mmol), and acetic acid (2.3 mL, 40 mmol) in methanol (80 mL) was added to a solution of coarse grained paraformaldehyde (2.7 g, 88 mmol) in methanol (80 mL) over 1 h and the mixture was stirred at 65° C. for 3 hours. The solution was cooled to room temperature, added H₂O (200 mL), added 1N NaOH (50 mL), extracted the mixture with Et₂O (3×, 300 mL), dried organic layer (MgSO₄), filtered, and concentrated in vacuo. Crude residue was purified using column chromatography using (50% EtOAc-hexanes) to yield compound 17A (2.6 g, 60%).

Using the method described in Example 15, compounds 17B and 17C were prepared. Using the method described in Example 14, and substituting compound 17B for compound 13C, compound 17 was prepared. LCMS: 505.3 (M+H)⁺.

### Example 18

### Preparation of Compound 21

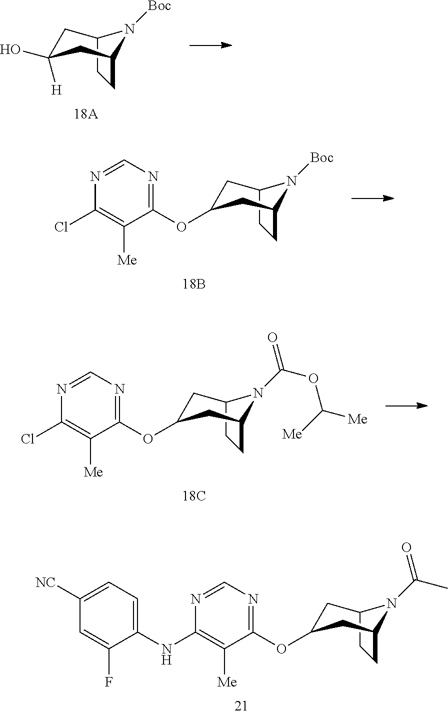

### Step A—Synthesis of Compound 18B

Using the method described in Example 13, substituting compound 18A (made according to the method described in International Publication No. WO 98/18788) for compound 13B, compound 18B was prepared.

### Step B—Synthesis of Compound 18C

To a solution of 18B (1.0 g, 2.83 mmol) in DCM (12 mL) was added TFA (2.2 mL) and the solution was stirred for 2 hours. To the resulting reaction was added 7N NH₃ in MeOH (5 mL) and the solution was concentrated in vacuo. To the crude residue obtained was added DMF (9 mL), TEA (1.2 mL, 8.5 mmol), and isopropyl chloroformate (1M in toluene, 3.4 mL, 3.4 mmol) and the solution was stirred for about 15 hours. The solution was then concentrated in vacuo and purified using column chromatography using (5% EtOAc in DCM) to provide compound 18C (0.67 g, 70%).

### Step C—Synthesis of Compound 21

Using the method described in Example 14, substituting compound 18C for compound 13C and substituting 4-cyano-2-fluoroaniline for 2-fluoro-4-methylsulfonylaniline, compound 21 was prepared. LCMS: 440.2 (M+H)⁺.

### Example 19

### Preparation of Compound 22

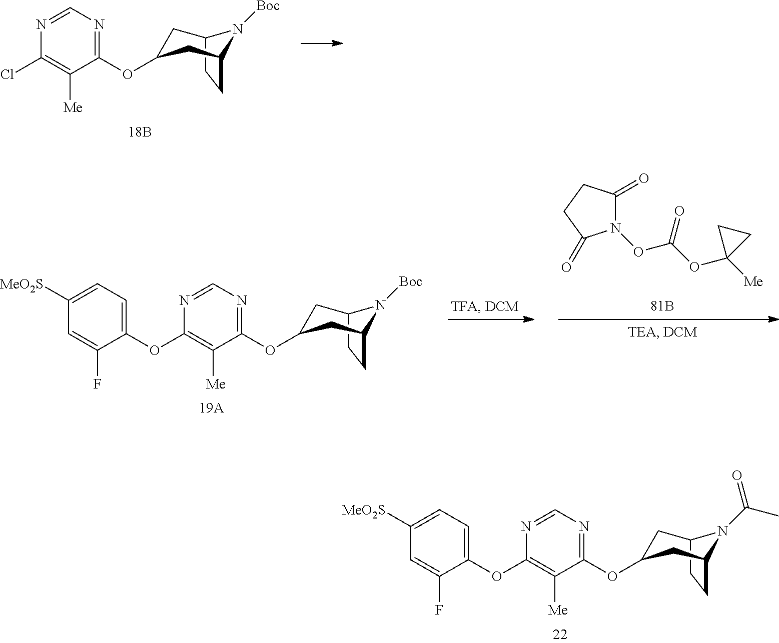

Using the method described in Example 13, and substituting compound 18B for compound 13C, compound 19A was prepared. Using the methods described in Example 18, and substituting compound 19A for compound 18B, followed by the method of Example 8, Step C, compound 22 was prepared. LCMS: 506.3 (M+H)⁺.

### Example 20

### Preparation of Compound 28

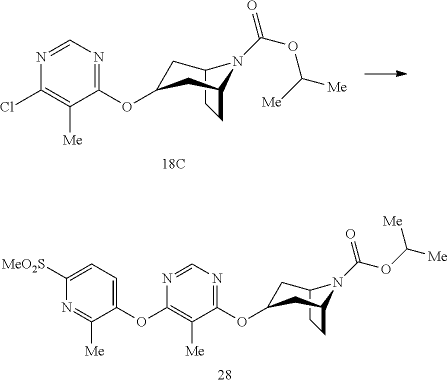

Using the method described in Example 13, and substituting compound 18C for compound 13C and substituting 2-methyl-6-(methylsulfonyl)pyridin-3-ol for 2-fluoro-4-methylsulfonylphenol, compound 28 was prepared. LCMS: 491.3 (M+H)⁺.

### Example 21

### Preparation of Compound 23

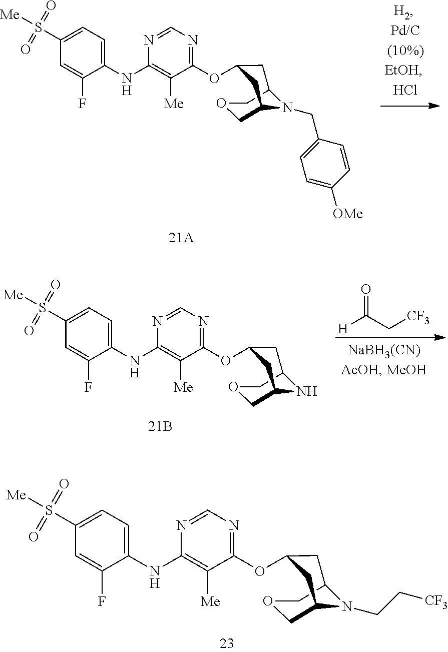

### Step A—Synthesis of Compound 21A

Using the methods described in Examples 13 and 14 substituting 4-methoxy-benzylamine for benzylamine, compound 21A was prepared.

### Step B—Synthesis of Compound 21B

To compound 21A (2.4 g, 4.5 mmol) was added EtOH (24 mL), 1N HCl (4 mL), and 10% Pd/C (480 mg). The solution was evacuated and recharged with H₂ via balloon (3×). The solution was stirred for about 15 hours under H₂ atmosphere, then filtered through celite and concentrated in vacuo to provide compound 21B (1Ag, 74%).

### Step C—Synthesis of Compound 23

To compound 21B (50 mg, 0.22 mmol) in MeOH (0.6 mL), was added acetic acid (5 □L), 3,3,3-trifluoroporpionaldehyde (22 μL, 0.24 mmol), and NaBH₃CN (15 mg, 0.24 mmol) and the reaction was stirred for about 15 hours. The reaction mixture was then concentrated in vacuo, diluted with H₂O and extracted with EtOAc. The combined organic layers were dried (MgSO₄), filtered, and concentrated in vacuo to provide a residue which was purified using preparative TLC using (40% EtOAc in DCM) to provide compound 23 (50 mg, 44%). LCMS: 519.3 (M+H)⁺.

The following compounds of the invention were similarly prepared by substituting the appropriate aldehyde or ketone for 3,3,3-trifluoroporpionaldehyde:

| Compound No. | Structure | LCMS (M + H)⁺ |
|---|---|---|
| 18 | 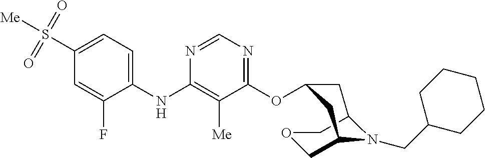 | 519.3 |
| 19 | 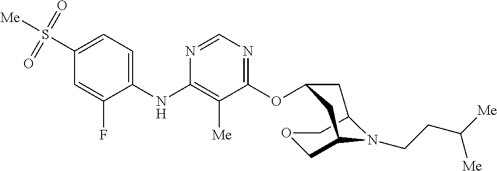 | 493.3 |
| 20 | 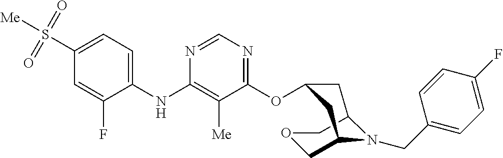 | 531.3 |
| 24 | 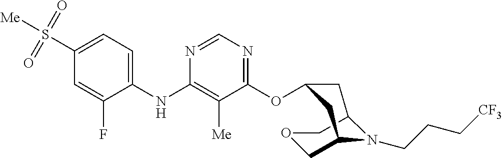 | 533.3 |
| 25 | 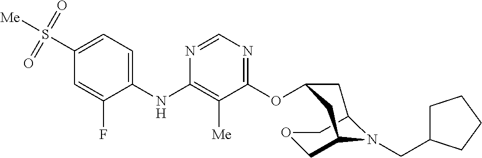 | 505.3 |
| 26 | 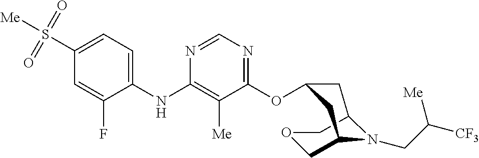 | 533.3 |
| 31 | 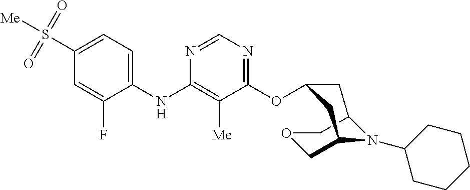 | 505.3 |

### Example 22

### Preparation of Compound 27

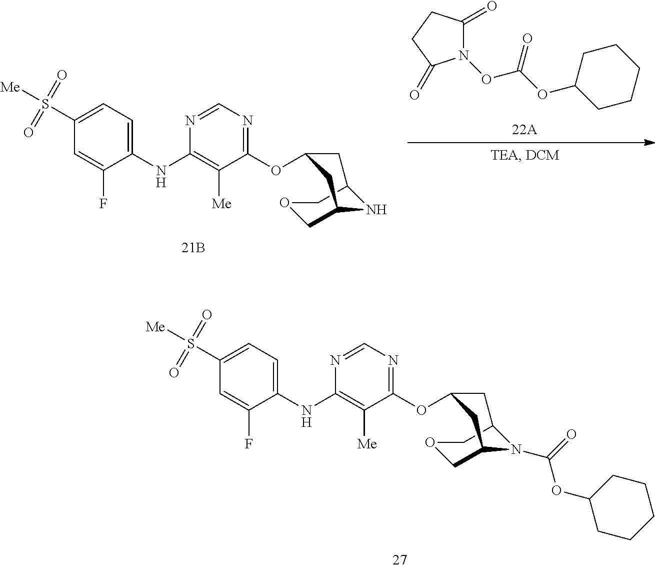

### Step A—Preparation of Compound 22A

To a solution of cyclohexanol (0.5 mL, 4.8 mmol) and TEA (2 mL, 14.5 mmol) in acetonitrile (16 mL) was added N,N′-disuccinimidyl carbonate (1.5 g, 5.8 mmol) and the resulting solution was allowed to stir at room temperature for about 15 hours. The solution was concentrated in vacuo, and the residue obtained was diluted with sat. aq. NaHCO3 and extracted with EtOAc. The organic layer was dried (MgSO4), filtered, and concentrated to provide compound 22A (0.9 g, 80%).

### Step B—Preparation of Compound 27

To a solution of compound 22A (27 mg, 0.11 mmol) and TEA (0.03 mL, 0.21 mmol) in DCM (0.5 mL) was added compound 21B (40 mg, 0.09 mmol) and the resulting reaction was allowed to stir for about 15 hours. The reaction mixture was then concentrated in vacuo and purified using preparative TLC using (20% EtOAc in DCM) to provide compound 27 (37 mg, 75%). LCMS: 549.3 (M+H)⁺.

### Example 23

### Preparation of Compound 29

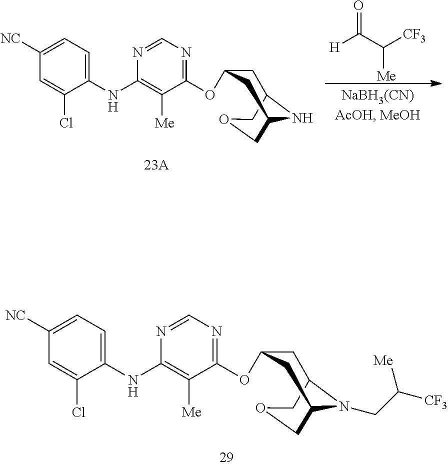

Using the methods described in Example 14, and substituting 2-chloro-4-cyanoaniline for 2-fluoro-4-methylsulfonylaniline, followed by Step A of Example 21, compound 23A was prepared. Using the method of Example 21, and substituting compound 23A for compound 21B and substituting 2-(trifluoromethyl)propionaldehyde for 3,3,3-trifluoroporpionaldehyde, compound 29 was prepared. LCMS: 496.3 (M+H)⁺.

### Example 24

### Preparation of Compound 30

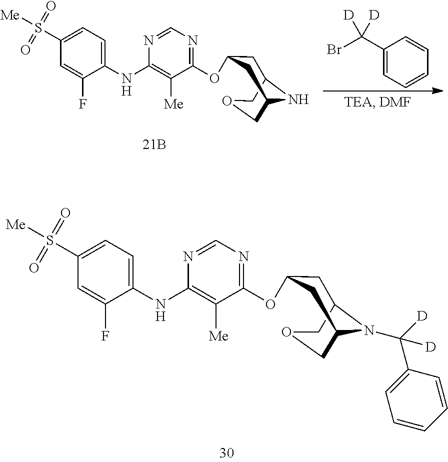

To compound 21B (75 mg, 0.18 mmol) was added DMF (1 mL), TEA (0.05 mL, 0.36 mmol), and benzyl-α,α-d₂-bromide and the solution was heated to 80° C. and allowed to stir at this temperature for about 15 hours. The reaction mixture was then cooled to room temperature, concentrated in vacuo and purified using preparative TLC using (35% EtOAc in DCM) to provide compound 30 (37 mg, 40%). LCMS: 515.3 (M+H)⁺.

### Example 25

### Preparation of Compound 32

### Step A—Preparation of Compound 25B

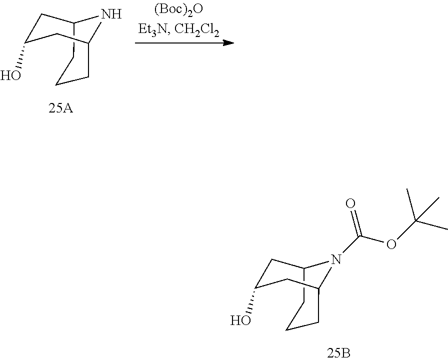

To a mixture of 4-azabicyclo[3.3.1]nonyl-3-endo-ol (25A, 120 mg, 0.85 mmol) in dichloromethane (8 mL) was added triethylamine (0.13 mL, 0.93 mmol) and the solution was put under nitrogen atmosphere. The reaction mixture was cooled to 0° C., then (Boc)₂O (203 mg, 0.93 mmol) was added. The reaction was warmed to room temperature, stirred for 18 hours, then quenched with water and extracted with dichloromethane. The organic layer was dried over MgSO₄, filtered and concentrated in vacuo to provide compound 25B (130 mg, 76%) which was used in the next reaction without further purification.

### Step B—Preparation of Compound 25C

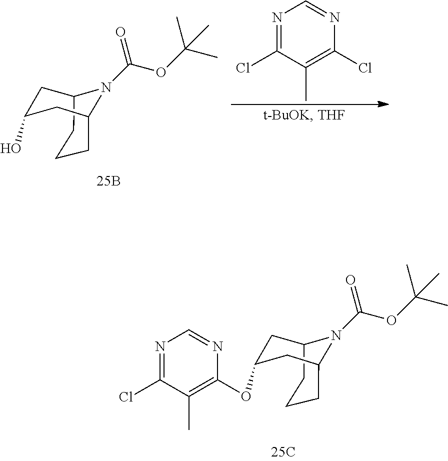

A solution of potassium t-butoxide (1.0 M in THF, 13.3 mL) was added dropwise to a solution of 4,6-dichloro-5-methylpyrimidine (2.16 g, 13.3 mmol) and compound 25B (3.20 g, 13.3 mmol) in THF (40 mL) at 0° C. under nitrogen atmosphere. The reaction was warmed to room temperature, stirred for 5 hours, then quenched with water and extracted with dichloromethane. The organic layer was dried over MgSO₄, filtered and concentrated in vacuo. The residue obtained was purified using silica gel flash chromatography (0-20% EtOAc/hexanes) to provide compound 25C (4.3 g, 88%).

### Step C—Preparation of Compound 25D

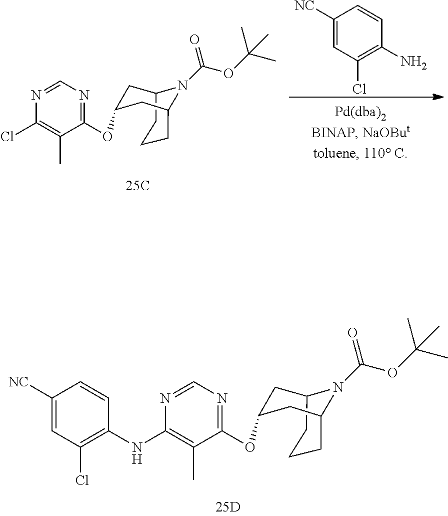

A mixture of compound 25C (163 mg, 0.44 mmol), 4-amino-3-chlorobenzonitrile (50 mg, 0.32 mmol), Pd(dba)₂ (10.0 mg), NaO-tBu (56 mg, 0.58 mmol), and BINAP (30 mg, 0.05 mmol) in toluene (1.5 mL) was placed in a sealed tube and heated to 110° C. After stirring at 110° C. for 17 hours, the reaction was cooled to room temperature and concentrated in vacuo. The resulting residue was purified using PTLC (50% acetone/hexanes) to provide compound 25D (49 mg, 23%). M+H=484

### Step D—Preparation of Compound 25E

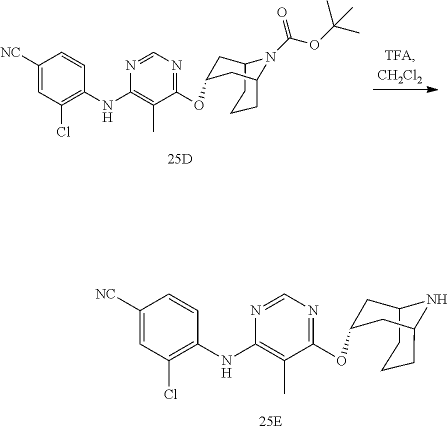

Trifluoroacetic acid (0.4 mL) was added dropwise to a solution of compound 25D (244 mg, 0.51 mmol) in dichloromethane (15 mL) at 0° C. under nitrogen. After stirring for 18 hours at 0° C., the reaction was diluted with dichloromethane and washed with saturated aqueous NaHCO₃. The organic layer was dried over MgSO₄, filtered and concentrated in vacuo to provide compound 25E (195 mg, 100%) which was used in next reaction without further purification.

### Step E—Preparation of Compound 25G

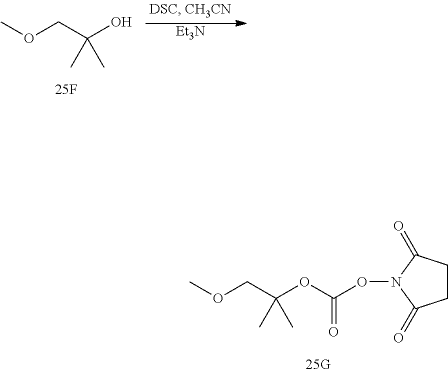

A solution of 1-methoxy-2-methylpropan-2-ol (25F, 267 mg, 2.6 mmol) in MeCN (2.5 mL) was treated with disuccinimidyl carbonate (740 mg, 2.9 mmol). The reaction was stirred for 5 minutes then Et₃N (1.0 mL, 7.2 mmol) was added. After stirring for an additional 24 hours, the reaction mixture was partitioned with EtOAc and saturated NaHCO₃. The organic layer was dried over MgSO₄, filtered and concentrated in vacuo to provide compound 25G (55 mg, 0.22 mmol) which was used in the next step without further purification.

### Step F—Preparation of Compound 32

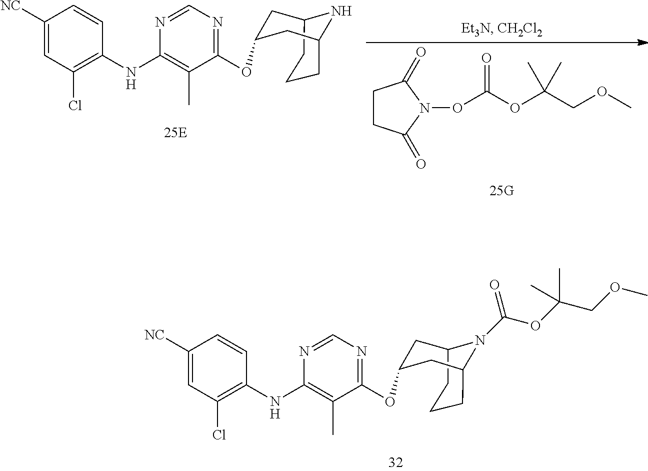

To a solution of compound 25E (36 mg, 0.09 mmol) in dichloromethane (2.0 mL) was added triethylamine (0.04 mL, 0.28 mmol), followed by a solution of compound 25G (50 mg, 0.20 mmol) in dichloromethane (2.5 mL). The resulting reaction was stirred at room temperature under nitrogen for 20 hours, then diluted with dichloromethane and washed with water. The organic layer was dried over MgSO₄, filtered and concentrated in vacuo to provide a residue which was purified using preparative thin layer chromatography (50%) EtOAc/hexanes) to provide compound 32(17 mg, 33%). M+H=514

### Example 26

### Preparation of Compound 33

### Step A—Preparation of Compound 26C

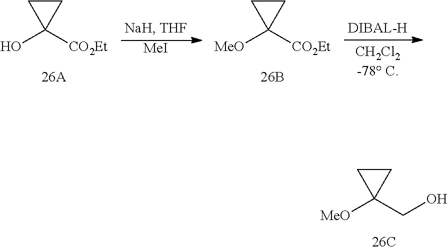

A solution of 1-hydroxy cyclopropanecarboxylic acid ethyl ester (26A, 1.0 g, 7.7 mmol) in THF (12 mL) was cooled to 0° C. (ice bath) under nitrogen, then sodium hydride (60% in oil, 410 mg, 10.2 mmol) was added portionwise, followed by methyl iodide (0.8 mL, 11.5 mmol). The reaction was warmed to room temperature and stirred for an additional 18 hours. The reaction mixture was quenched with saturated NH₄Cl and extracted with EtOAc. The combined organic layers were dried over dried over MgSO₄, filtered and concentrated in vacuo to provide compound 26B (660 mg, 61%) which was used in next reaction without further purification.

DIBAL-H (1.0 M in toluene, 10 mL) was added dropwise to a solution of compound 26B (480 mg, 3.4 mmol) in dichloromethane (15 mL) at −78° C. (dry ice/acetone). After the addition was complete, the reaction was warmed to room temperature and stirred for an additional 18 hours. The reaction was quenched with methanol and stirred for an additional 30 minutes. The resulting mixture filtered through celite and washed with methanol, followed by dichloromethane. The filtrate was dried over MgSO₄, filtered and concentrated in vacuo to provide compound 26C (186 mg, 55%) which was used in next reaction without further purification.

### Step B—Preparation of Compound 26D

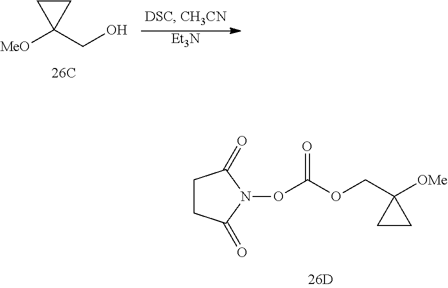

Compound 26C (186 mg, 1.8 mmol) was reacted according to the method described in example 25, step E to provide compound 26D (280 mg, 65%) which was used in the next reaction without further purification.

### Step E—Preparation of Compound 33

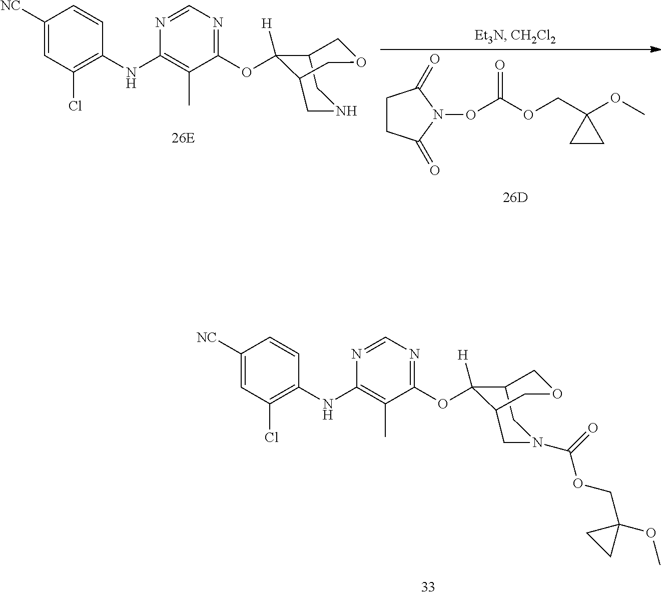

Compound 26E (27 mg, 0.07 mmol, prepared using methods described in Example 5) was reacted with compound 26D (29 mg, 0.12 mmol) using the method described in 25, step F to provide compound 33(23 mg, 65%). M+H=514.

### Example 27

### Preparation of Compound 34

### Step A—Synthesis of Compound 27A

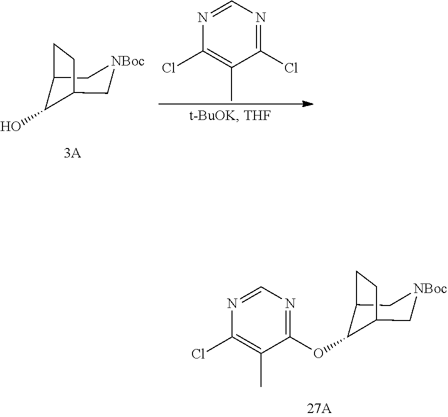

A solution of potassium t-butoxide (1.0 M in THF, 5.3 mL) was added dropwise to a solution of 4,6-dichloro-5-methylpyrimidine (860 mg, 5.3 mmol) and the compound 3A (12 g, 5.3 mmol) in THF (40 mL) at 0° C. under nitrogen. The reaction was warmed to room temperature and stirred for an additional 5 hours. The reaction mixture was then quenched with water and extracted with dichloromethane. The organic layer was dried over MgSO₄, filtered and concentrated in vacuo, and the residue obtained was purified using silica gel flash chromatography (0-20% EtOAc/hexanes) to provide compound 27A (0.97 mg, 52%).

### Step B—Preparation of Compound 27B

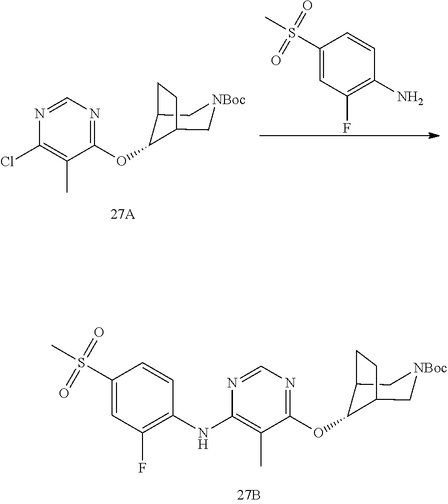

Compound 27A (250 mg, 0.71 mmol), 2-fluoro-4-(methylsulfonyl)aniline (160 mg, 0.85 mmol), Pd(OAc)₂, (32 mg, 0.14 mmol), NaO-tBu (103 mg, 1.06 mmol), and X-phos (152 mg, 0.32 mmol) were taken up in dioxane (12 mL). The mixture was heated in a sealed tube to 110 ⁹C and allowed to stir at this temperature for 18 hours, then filtered through celite and washed with ether. The filtrate was concentrated in vacuo and the resulting residue was purified using PTLC to provide compound 27B (297 mg, 84%).

### Step C—Preparation of Compound 27C

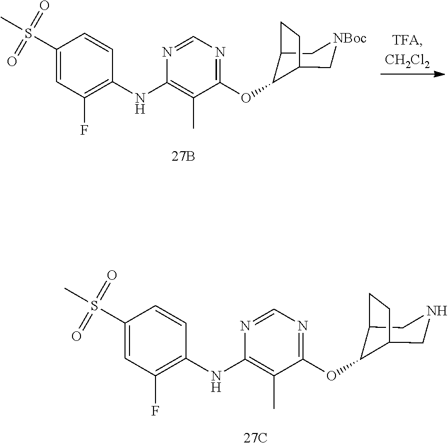

Compound 27B (290 mg, 0.57 mmol) was reacted according to the method described in example 25, step D to provide compound 27C (233 mg, 100%) which was used in the next reaction without further purification.

### Step D—Preparation of Compound 34

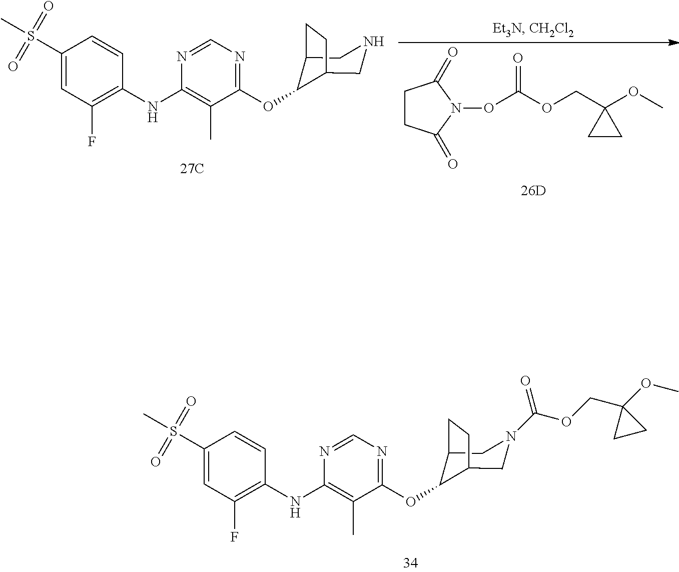

Compound 27C (64 mg, 0.16 mmol) was reacted with compound 26D (72 mg, 0.30 mmol) according to the method described in example 25, step F to provide compound 34 (35 mg, 41%). M+H=535

### Example 28

### Preparation of Compound 40

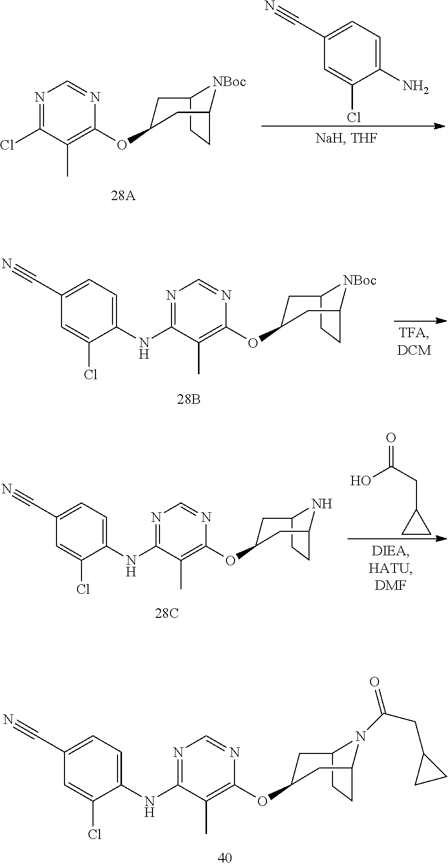

### Step A—Synthesis of Compound 28A

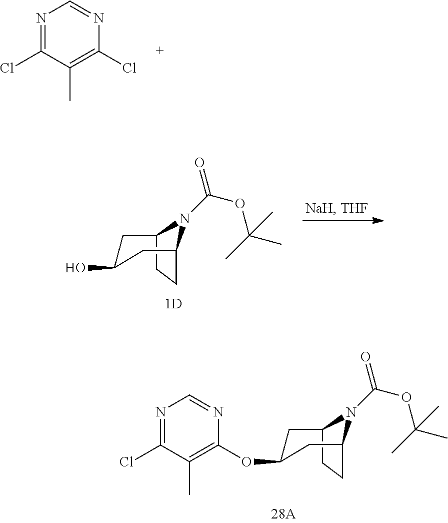

To a 0° C. solution of compound 1D (8.5 grams, 37.4 mmol, made according to the method described in International Publication No. WO 98/18788 to Blythin, et al.) in THF (200 mL) was added sodium hydride in 60% oil (6 grams, 150 mmol) and the reaction was allowed to stir for 30 minutes at 0° C. The reaction mixture was warmed to room temperature, 4,6-dichloro-5-methylpyrimidine (6.8 grams, 41.1 mmol) was added, and the reaction was permitted to stir for an additional seven hours. The reaction mixture was quenched with water and extracted with DCM. The organic phase was dried (Na₂SO₄) and concentrated in vacuo, and the resulting residue was purified using a silica gel cartridge with hexanes/ethyl acetate (50/50) to provide compound 28A as a light brown solid (12.3 grams, 93%). LCMS: 354.2 (MH⁺).

### Step B—Synthesis of Compound 28B

A solution of Sodium hydride (11 mmol, 60% in oil)) in THF (100 mL) was cooled to 0° C. 4-amino-3-chloro-benzonitrile (ca 5.6 mmol) was added and the mixture was stirred for 30 minutes at 0° C. Compound 28A (ca 2.8 mmol) was added and the reaction mixture was heated to 85° C. and allowed to stir at this temperature for four hours. The reaction mixture was quenched with water and extracted with DCM. The organic phase was dried (Na₂SO₄), concentrated in vacuo and the residue obtained was purified using a silica gel cartridge with DCM/ethyl acetate (90/10) to provide a crude product. The crude product was dissolved in 10 ml of DCM and poured into 1000 ml of hexanes. The solid precipitates were filtered, washed with hexanes, and dried to provide compound 28B (ca 60%).

### Step C—Synthesis of Compound 28C

Compound 28B (ca 35 mmol) was dissolved in THF (200 mL) and the solution was cooled to 0° C. Trifluoroacetic acid (100 mL) was added and the reaction was allowed to warm to room temperature and was allowed to stirred for six hours. The reaction mixture was concentrated in vacuo, redissolved in DCM, and neutralized with a saturated sodium bicarbonate solution. The organic phase was dried (Na₂SO₄) and concentrated in vacuo to provide compound 28C which was used without further purification.

### Step D—Synthesis of Compound 40

Compound 28C (100 mg, 0.27 mmol), 2-cyclopropylacetic acid (46 mg, 0.46 mmol), DIEA (0.15 mL, 0.92 mmol), and HATU (175 mg, 0.46 mmol) were combined in DMF (5 mL) and stirred at room temperature for 3 hours. The mixture was concentrated in vacuo, and the residue obtained was dissolved in DCM and washed with saturated NH₄Cl. The organic phase was dried (Na₂SO₄), concentrated in vacuo and the residue obtained was purified on a silica gel cartridge (eluting with ethyl acetate in hexanes (0→65%) to provide compound 40 as an off white solid (56 mg, 46%). LCMS: 452.3 (MH⁺).

The following compounds of the invention were prepared using the method described above and substituting the appropriate carboxylic acid for 2-cyclopropylacetic acid:

| Cpd. No. | Structure | LCMS (MH⁺) |
|---|---|---|
| 35 | 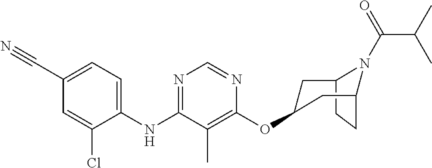 | 440.2 |
| 37 | 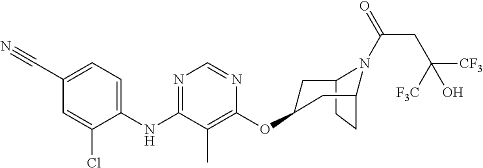 | 578.3 |
| 38 | 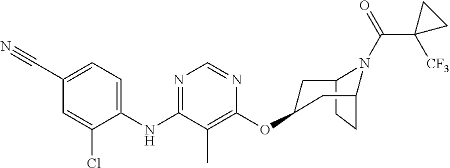 | 506.3 |
| 39 | 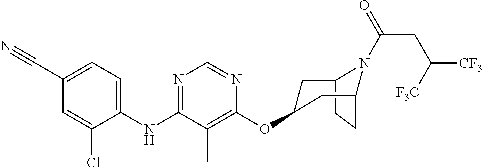 | 562.3 |

### Example 29

### Preparation of Compound 45

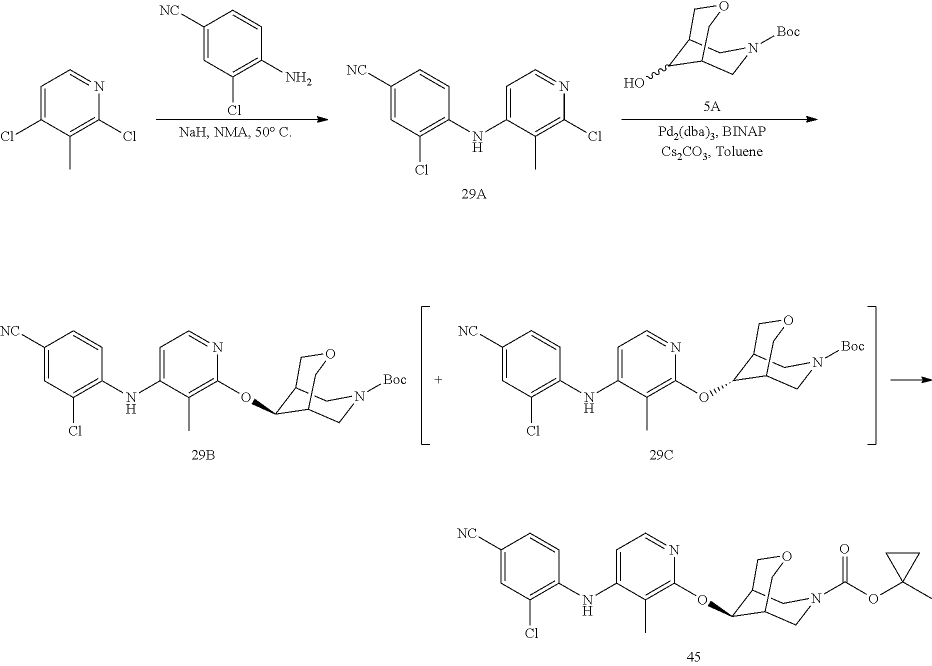

### Step A—Synthesis of Compound 29A

To a solution of 4-amino-3-chlorobenzonitrile (176 g, 11.5 mmol) in dimethylacetamide (20 mL) at room temperature was added NaH (0.67 g, 17 mmol) in portions. The reaction was stirred at room temperature for 1 hour, then a solution 2,4-dichloro-3-methylpyridine (1.50 g, 9.26 mmol) in dimethylacetamide (20 mL) was added and the reaction was stirred at 50° C. for 20 hours. The mixture was then poured into saturated NH₄Cl and the resulting solids were filtered, washed with water, dried, and separated on a silica gel cartridge (eluting with ethyl acetate in DCM (0→20%) to give compound 29A as a brown purple solid (1.00 g, 39%).

### Step B—Synthesis of Compound 29B

Compound 5A (207 mg, 0.881 mmol), compound 29A (213 mg, 0.766 mmol), cesium carbonate (0.43 g, 1.3 mmol), Pd₂(dba)₃ (67 mg, 0.073 mmol), and racemic BINAP (0.10 g, 0.21 mmol) were combined in toluene (8 mL). The reaction was purged with nitrogen and stirred at 130° C. for 5 hours. The reaction mixture was cooled to room temperature, filtered through Celite, washed with EtOAc, and concentrated in vacuo. The residue obtained was purified on a silica gel cartridge (eluting with acetone in DCM (0→15%) to provide compound 29B (64 mg, 17% yield) and compound 29C (51 mg, 14% yield).

### Step C—Synthesis of Compound 45

Compound 29B was converted to compound 45 using the methods described in example 5, Steps D and E. LCMS: 483.3 (MH⁺).

### Example 30

### Preparation of Compound 46

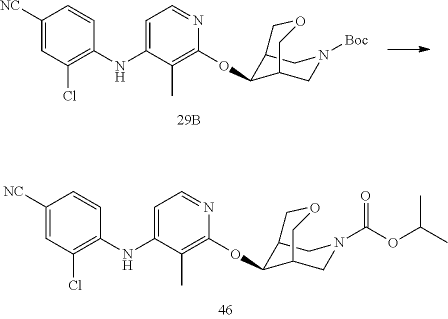

Compound 29B was converted to compound 46 using the method described in Example 9. LCMS: 471.3 (MH⁺).

Compounds 47-52 of the present invention were prepared using the method described above and substituting the appropriate starting material in place of compound 29B.

### Example 31

### Preparation of Compounds 53-60

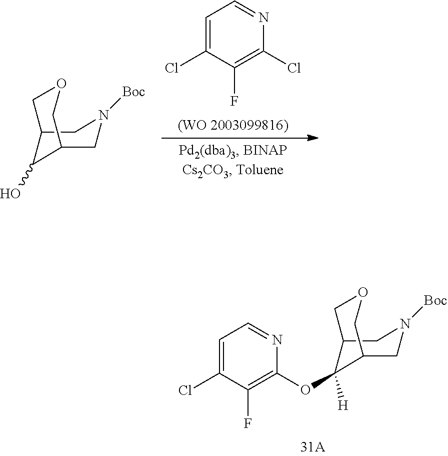

Compound 5A (90 mg, 0.36 mmol), 2,4-dichloro-3-fluoropyridine (50 mg, 0.3 mmol, prepared as described in WO 2003099816 to Goodacre et al), cesium carbonate (150 mg, 0.45 mmol), Pd₂(dba)₃ (5 mg), and BINAP (10 mg) were combined in toluene (3 mL). The mixture was purged with nitrogen and stirred at 130° C. for 5 hours. The mixture was filtered through Celite, washed with DCM and concentrated in vacuo. The residue obtained was purified using preparative TLC (60/40 Hex/EtOAc) to provide intermediate compound 31A (20 mg, 18% yield). LCMS: 373.3 (MH⁺).

Intermediate compound 31B was prepared as follows using the method described above:

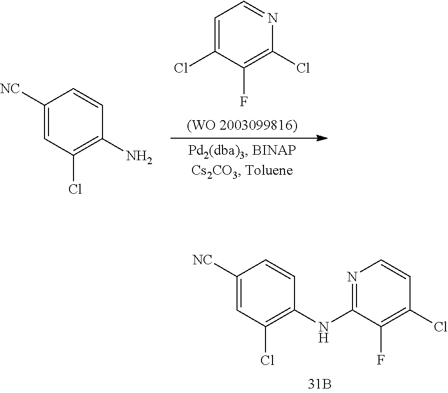

Intermediate compound 31C was prepared as follows using the method described above:

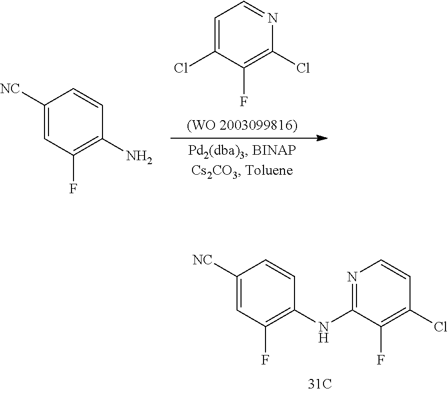

Compounds 53-56 of the present invention were prepared from compound 31A using methods described above herein.

Compounds 57-58 of the present invention were prepared from compound 31B using methods described above herein.

Compounds 59-60 of the present invention were prepared from compound 31C using methods described above herein.

### Example 32

### Preparation of Compounds 61-64

Compounds 61-64 of the present invention can be made by using the methods described in Example 5 and replacing compound 1B with 4,6-dichloro-5-cyclopropylpyrimidine (described in International Publication No. WO 05/007647).

### Example 33

### Preparation of Compound 65

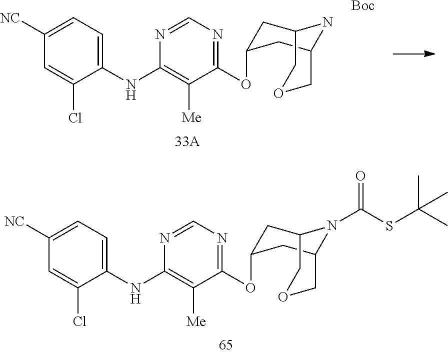

Compound 65 was prepared as a white solid from Compound 33A and t-butyl thiolchloroformate using the methods described in Examples 8 and 9. LC/MS: 502+504 (MH⁺).

The following compounds of the present invention were prepared using the method described above and substituting the appropriate reactants:

| Cpd. | | |
|---|---|---|
| No. | Structure | LC/MS |
| 66 | 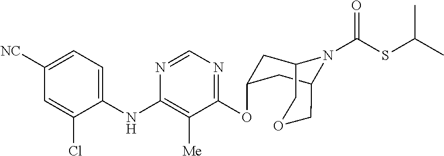 | 488 |
| 67 | 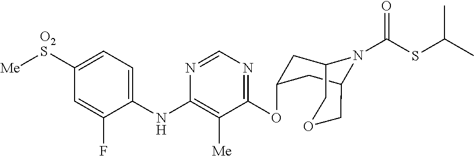 | 525 |
| 68 | 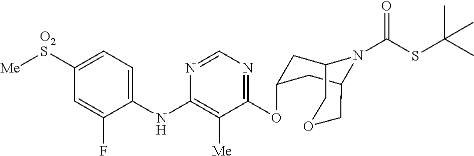 | 539 |
| 69 | 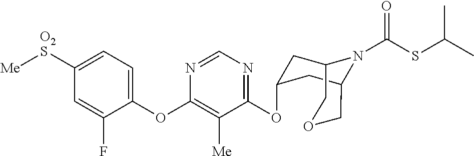 | 526 |

### Example 34

### Preparation of Compounds 70 and 71

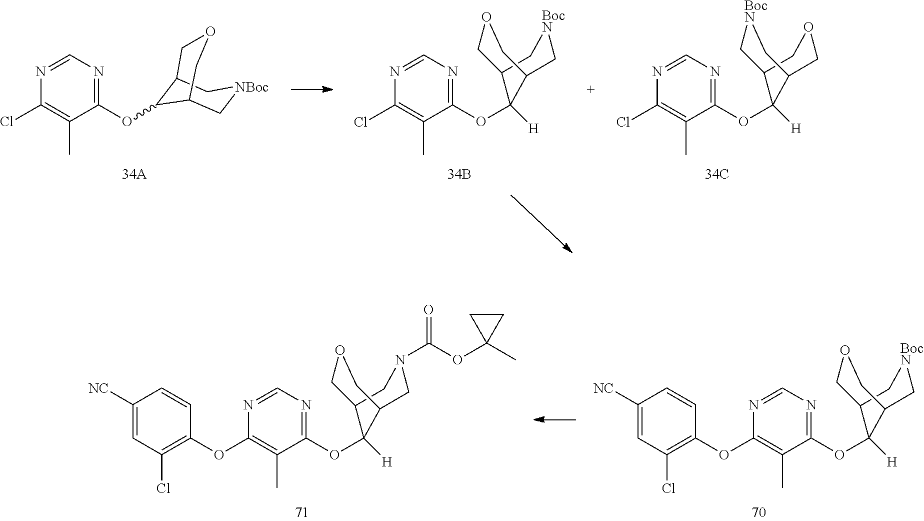

Compound 34A was separated into syn-isomer 34B and anti-isomer 34C by chromatography on silica with 20% EtOAc/hexane. Compound 34B was reacted with 2-chloro-4-cyanophenol according to the procedure of Example 8 (heating in NMP 72 h at 135° C.) to give compound 70 as a white solid, LC/MS 487.3 (MH⁺). Following the procedures of Example 8, this was converted to compound 71 as a white solid, LC/MS 471.3 (MH⁺).

The following compounds of the present invention were prepared using the method described above and substituting the appropriate reactants:

| Cpd. | | LC/ |
|---|---|---|
| No. | Structure | MS |
| 72 | 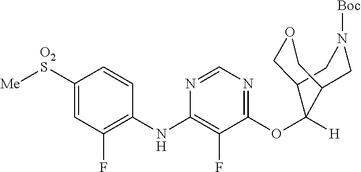 | 527 |
| 73 | 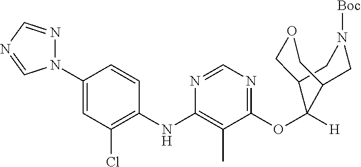 | 528, 530 |
| 74 | 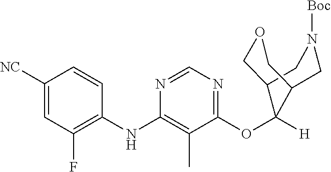 | 470 |

### Example 35

The following compounds can be made using the methods described in Example 8 and 9 and substituting the appropriate reactants:

| Cpd. | | |
|---|---|---|
| No. | Structure | LC/MS |
| 75 | 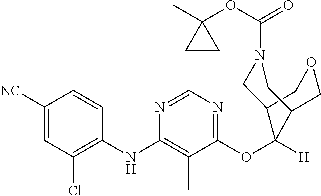 | 470, 472 |
| 76 | 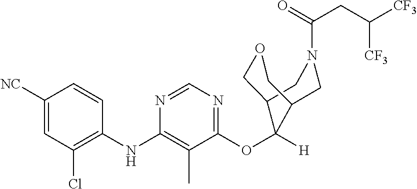 | 578, 580 |
| 77 | 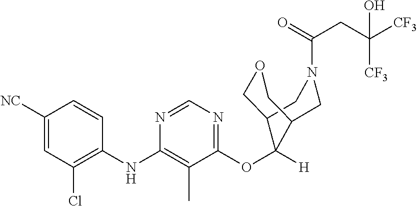 | 594, 596 |
| 78 | 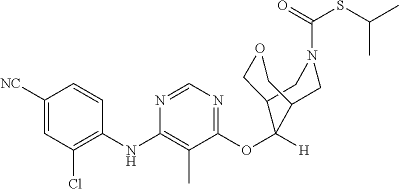 | 488 |
| 79 | 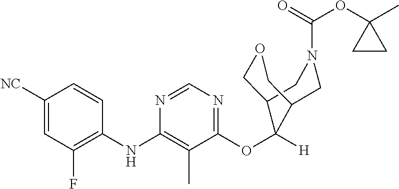 | 468 |
| 80 | 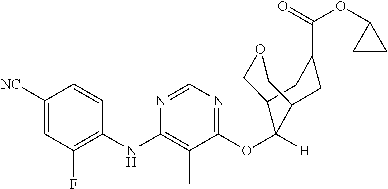 | 454 |
| 81 | 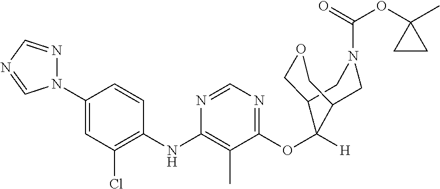 | 526, 528 |
| 82 | 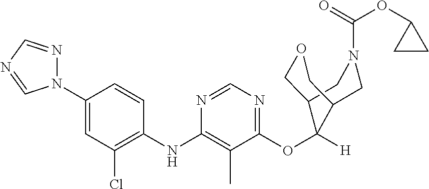 | 512, 514 |
| 83 | 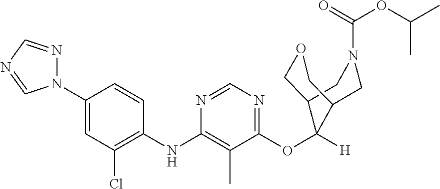 | 514, 516 |
| 84 | 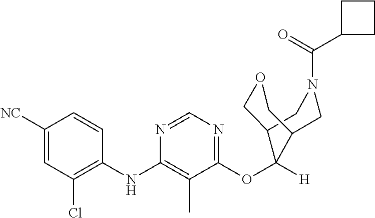 | 468, 470 |
| 85 | 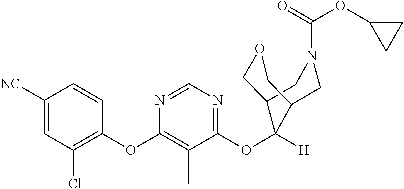 | 471, 473 |
| 86 | 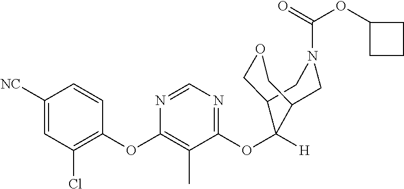 | 485, 487 |
| 87 | 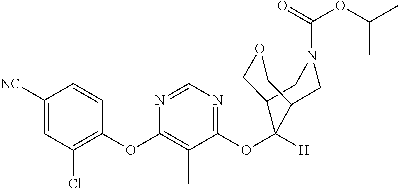 | 473, 475 |
| 88 | 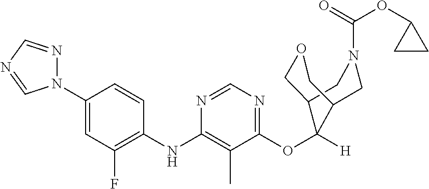 | 496 |
| 89 | 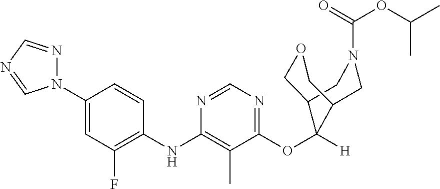 | 498 |
| 90 | 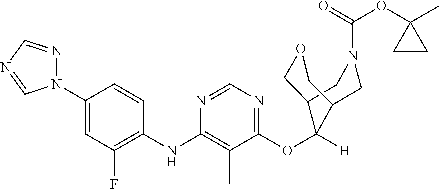 | 510 |

### Example 36

### Preparation of Compound 91

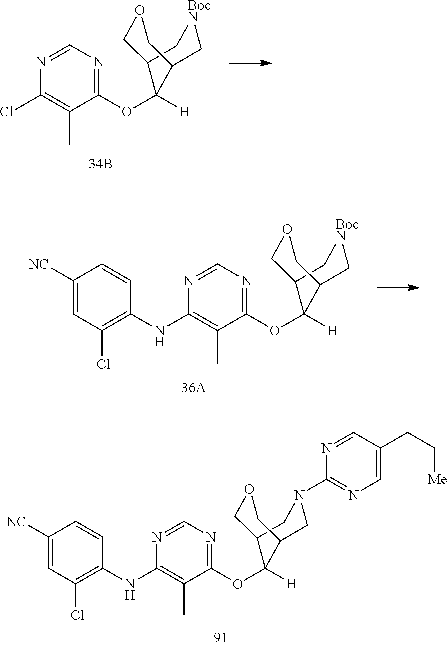

### Step A—Synthesis of Compound 36A

Compound 34B was converted to Compound 36A according to the methods of Example 5.

### Step B—Synthesis of Compound 91

Compound 36A (0.090 g, 0.19 mmol) was dissolved 1:1 MeCN—CH₂Cl₂ (3 mL) and treated with 4.0N HCl/dioxane (2.0 mL). The mixture was heated to 40° C. 0.5 h, concentrated, and taken up in DMF (3.0 mL). DIPEA (0.143 g, 1.1 mmol) was added, followed by AgF (0.071 g, 0.56 mmol) and 2-chloro-5-(n-propyl)pyrimidine (0.087 g, 0.55 mmol). The mixture was heated in a microwave apparatus at 160° C. for 1 hour, allowed to cool, concentrated, and purified using PLC to provide compound 91 as a yellow solid, LC/MS 506+508 (MH⁺).

The following compounds of the present invention were prepared using the method described above and substituting the appropriate reactants:

| Cpd. | | |
|---|---|---|
| No. | Structure | LC/MS |
| 92 | 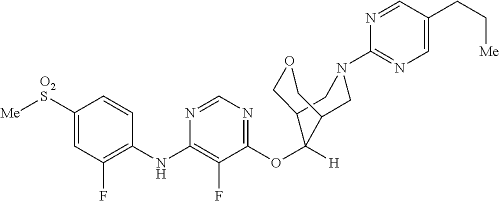 | 547 |
| 93 | 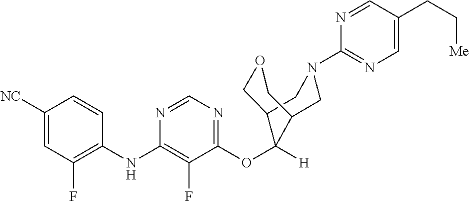 | 494 |
| 94 | 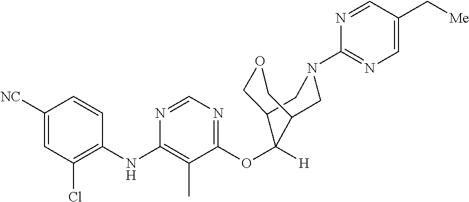 | 492, 494 |
| 95 | 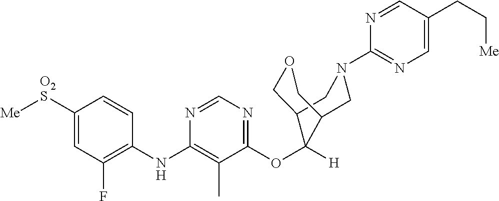 | 543 |
| 96 | 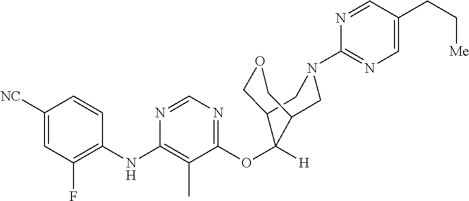 | 490 |
| 97 | 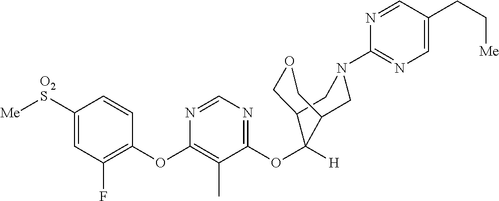 | 544 |
| 98 | 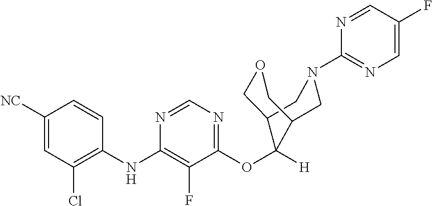 | 486, 488 |
| 99 | 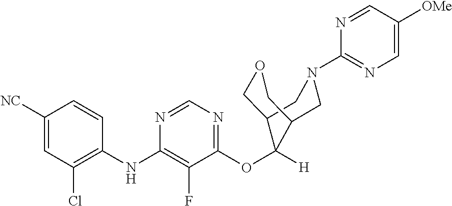 | 498, 500 |
| 100 | 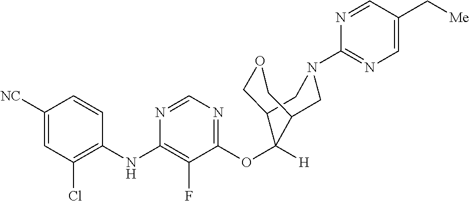 | 496, 498 |
| 101 | 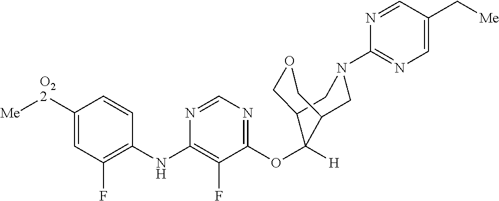 | 533 |
| 102 | 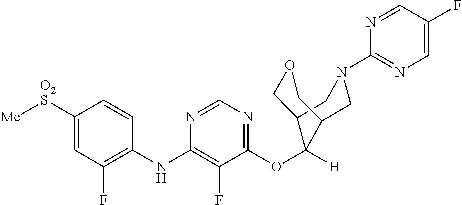 | 523 |
| 103 | 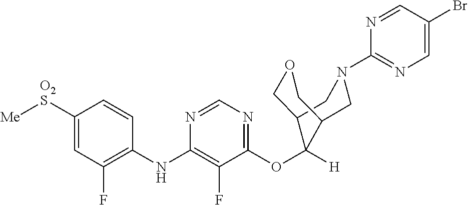 | 583, 585 |
| 104 | 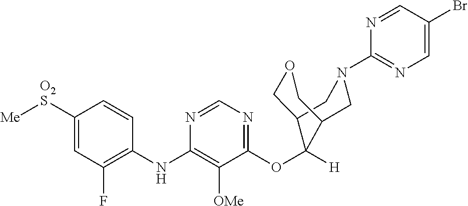 | 595, 597 |
| 105 | 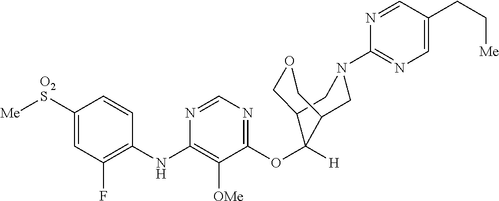 | 559 |
| 106 | 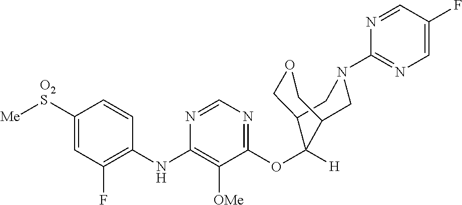 | 535 |
| 107 | 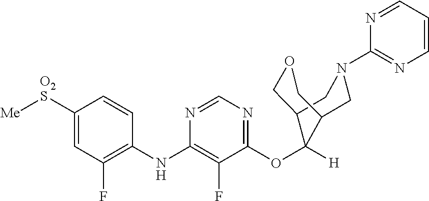 | 506 |
| 108 | 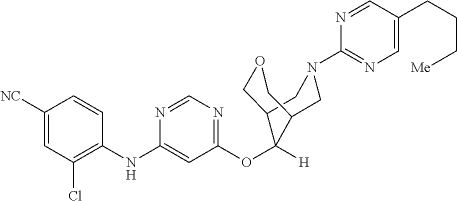 | 492, 494 |
| 109 | 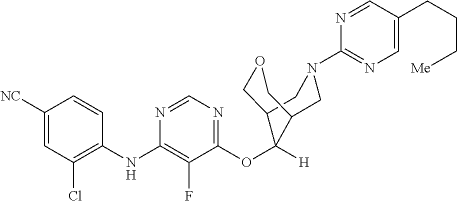 | 510, 512 |
| 110 | 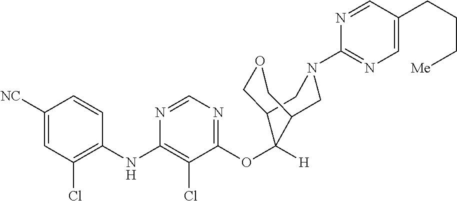 | 526, 558, 530 |
| 111 | 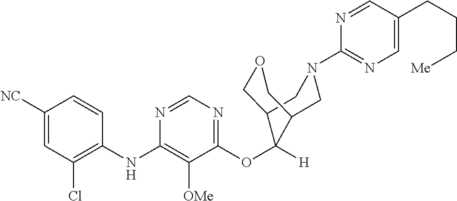 | 522, 524 |
| 112 | 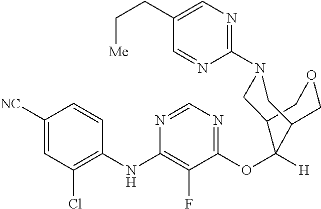 | 510, 512 |

### Example 37

### Preparation of Compound 113

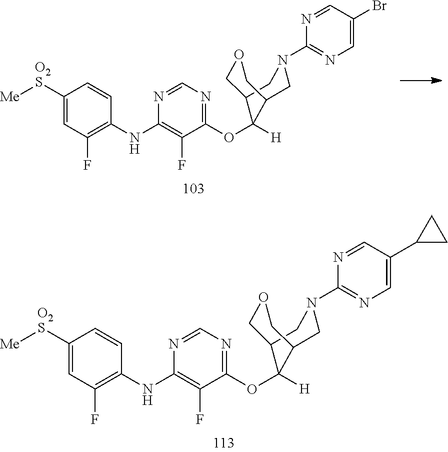

Compound 103 (0.015 g, 0.026 mmol), cyclopropaneboronic acid (0.009 g, 0.10 mmol), dppf (0.004 g, 0.005 mmol) and K₂CO₃ (0.022 g, 0.16 mmol) were combined in THF (1.0 mL) with one drop of water. The mixture was heated in a microwave apparatus at 100° C. for 0.5 hours, then cooled and concentrated in vacuo. Purification of the resulting residue by PLC gave compound 113 as a white solid, LC/MS 545 (MH⁺).

The following compound of the present invention was prepared using the method described above and substituting the appropriate reactants:

| Cpd. | | |
|---|---|---|
| No. | Structure | LC/MS |
| 114 | 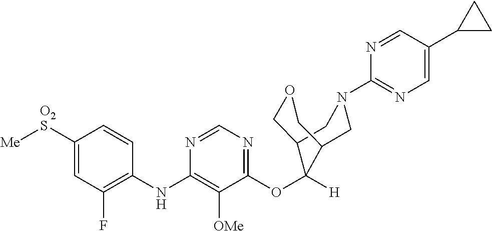 | 557 |

### Example 38

### Preparation of Compound 115

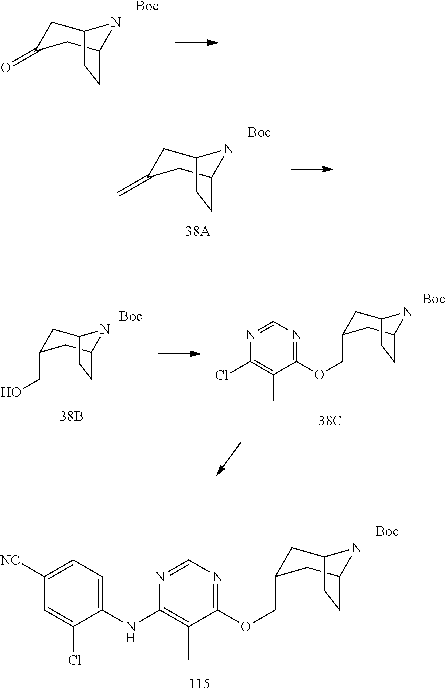

### Step A—Synthesis of Compound 388

Compound 38A (prepared from the pictured ketone with Tebbe reagent according to J. Org. Chem. 2000, 7122, 0.40 g, 1.79 mmol) in THF (2 mL) was added slowly to 9-BBN (0.5M in THF, 7.2 mL, 3.6 mmol) cooled to −30° C. The solution was allowed to warm and stirred 1 h, then cooled in ice and treated with 3M NaOH (3.0 mL), then 50% H₂O₂ (3.0 mL). The reaction was allowed to warm over 1.5 h and partitioned with EtOAc and brine. The EtOAc was dried (MgSO₄), concentrated and chromatographed on silica to provide compound 38A as a colorless oil.

### Step B—Synthesis of Compound 38B

Using the method described in Example 5, compound 38A was converted to compound 38B, a yellow oil.

### Step C—Synthesis of Compound 115

Using the method described in Example 5, compound 38B was converted to compound 70, a white solid, LC/MS 484, 486 (MH⁺).

The following compound of the present invention was prepared using the method described above and substituting the appropriate reactants:

| Cpd. | | LC/ |
|---|---|---|
| No. | Structure | MS |
| 116 | 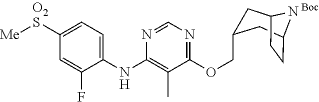 | 521 |

### Example 39

### Preparation of Compound 117

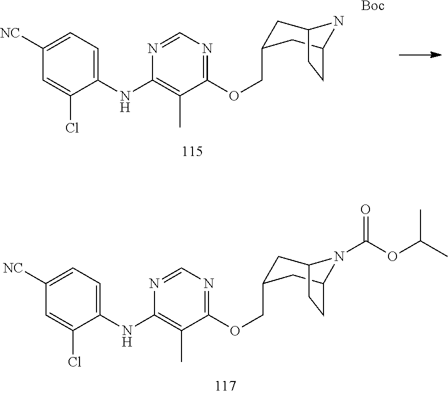

Using the method described in Example 9, Compound 115 was converted into Compound 117, a white solid, LC/MS 470, 472 (MH⁺).

The following compounds of the present invention were prepared using the method described above and the method described in Example 5 and substituting the appropriate reactants:

| Cpd. | | |
|---|---|---|
| No. | Structure | LC/MS |
| 118 | 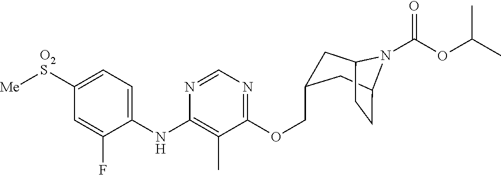 | 507 |
| 119 | 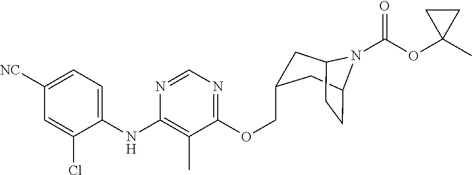 | 481, 483 |
| 120 | 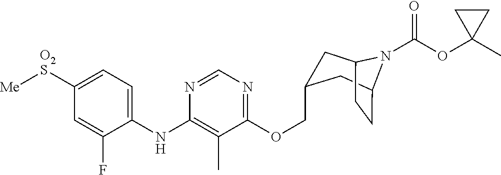 | 519 |

### Example 40

### Preparation of Compound 121

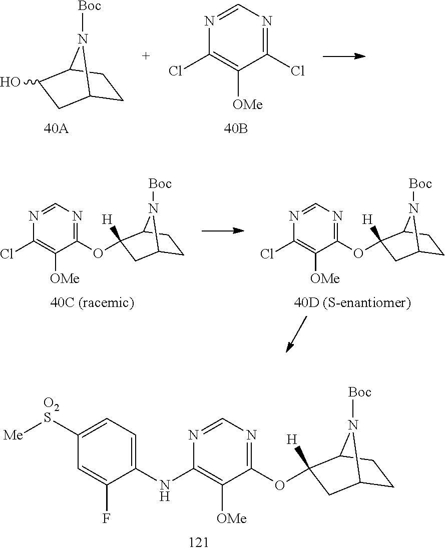

Using the method described in Example 5, Compound 40C was prepared. Separation by Chiralcel OD chiral chromatography and reaction of the faster running enantiomer according to Example 5 furnished the S-enantiomer Compound 121 as a yellow solid, LC/MS: 509 (MH⁺).

The following compounds of the present invention were prepared using the methods described above and substituting the appropriate reactants:

| Cpd. | | LC/ |
|---|---|---|
| No. | Structure | MS |
| 122 | 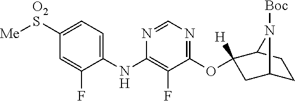 | 497 |
| 123 | 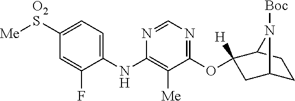 | 493 |
| 124 | 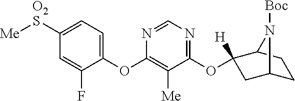 | 494 |
| 125 | 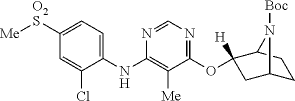 | 509, 511 |
| 126 | 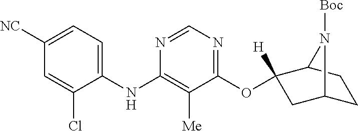 | 456, 458 |

### Example 41

### Preparation of Compound 127

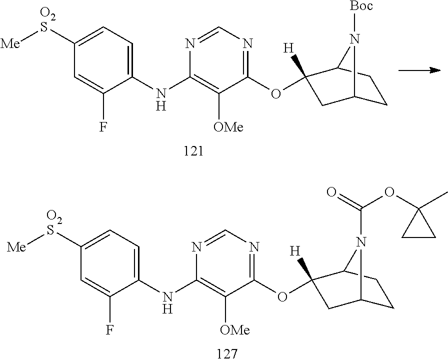

Using the method described in Example 5, Compound 121 was converted to Compound 127, a white solid, LC/MS 507 (MH⁺).

Using this method to acylate the compounds listed in Example 40, and relevant acylation reagents, the following compounds of the present invention were prepared:

| Cpd. | | |
|---|---|---|
| No. | Structure | LC/MS |
| 128 | 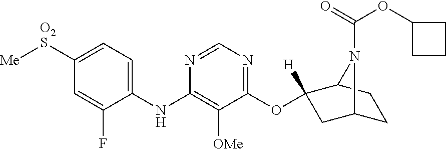 | 507 |
| 129 | 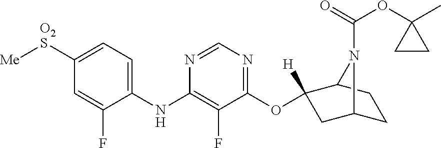 | 495 |
| 130 | 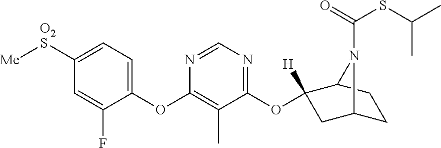 | 496 |
| 131 | 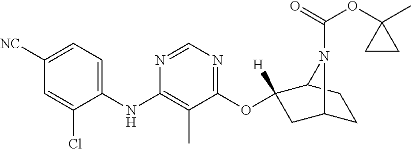 | 454, 456 |
| 132 | 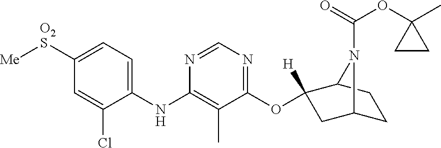 | 507, 509 |
| 133 | 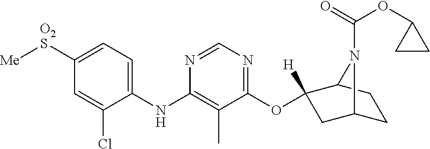 | 493, 495 |
| 134 | 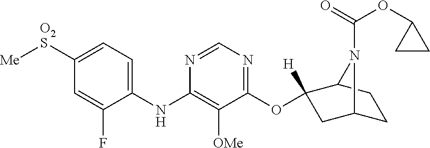 | 493 |
| 135 | 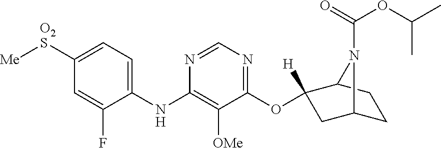 | 495 |
| 136 | 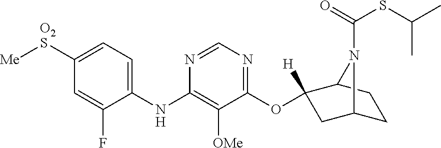 | 511 |

### Example 42

### Preparation of Compound 137

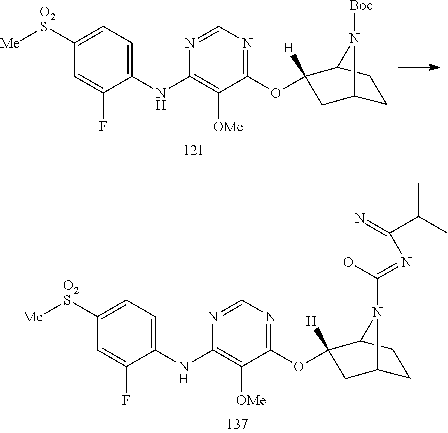

Using the methods described in International Publication No. WO 05/007647, compound 121 was converted to compound 137, a white solid, LC/MS 503 (MH⁺).

Using this method, or alternatively, the method described in Example 36, the compounds listed in Example 40 were converted to the following compounds of the present invention:

| Cpd. | | |
|---|---|---|
| No. | Structure | LC/MS |
| 138 | 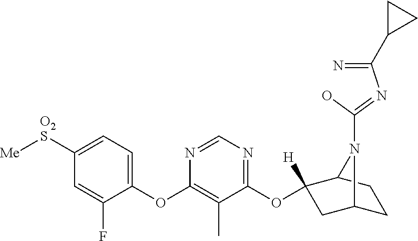 | 502 |
| 139 | 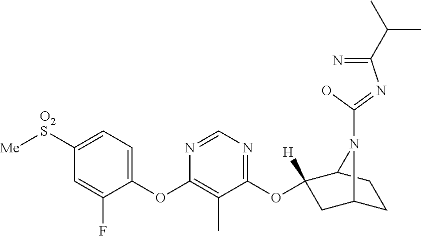 | 504 |
| 140 | 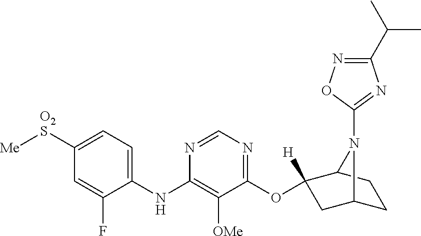 | 519 |
| 141 | 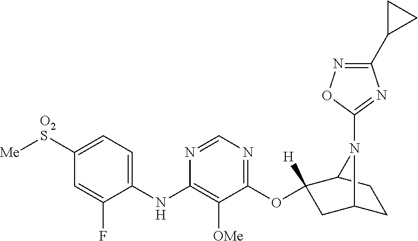 | 517 |
| 142 | 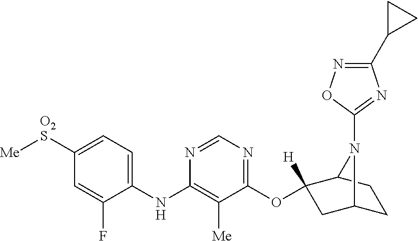 | 501 |
| 143 | 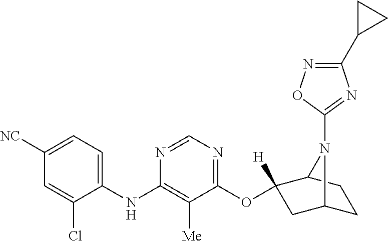 | 464, 466 |
| 144 | 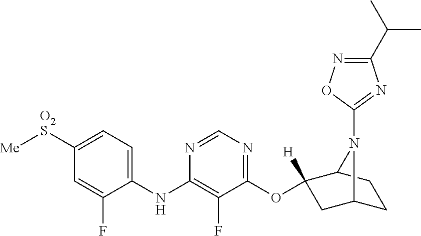 | 507 |
| 145 | 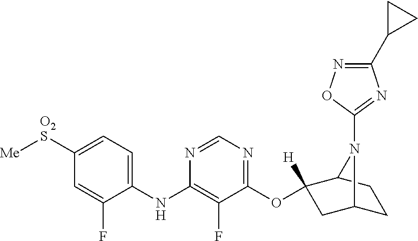 | 505 |
| 146 | 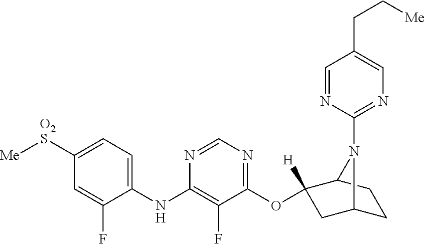 | 517 |
| 147 | 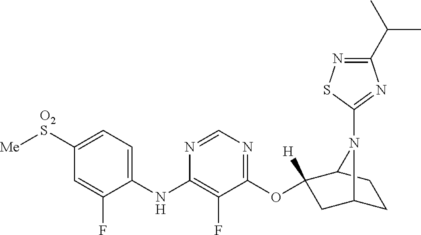 | 520 |
| 148 | 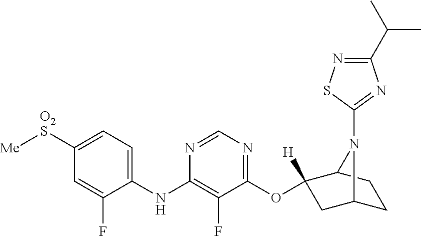 | 519 |
| 149 | 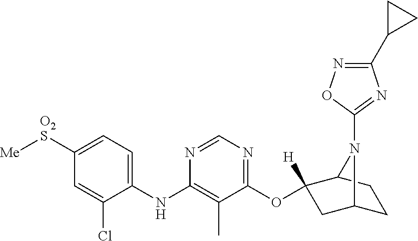 | 517, 519 |
| 150 | 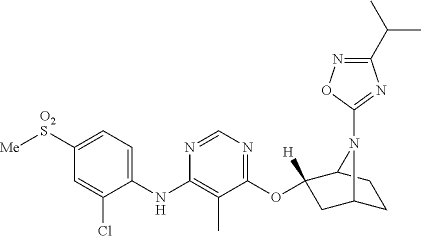 | 519, 521 |

### Example 43

### Preparation of Compound 151

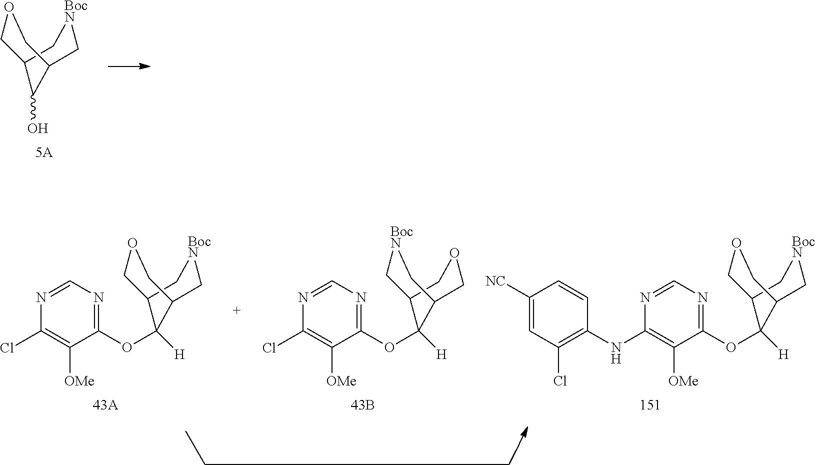

Using the method described in Example 5, Compound 5A was converted to Compound 151, a white solid, LC/MS: 502+504 (MH⁺).

In similar fashion, starting with Compound 43A or 43B, together with the appropriate aniline or phenol, the following compounds of the present invention were prepared:

| Cpd. | | |
|---|---|---|
| No. | Structure | LC/MS |
| 152 | 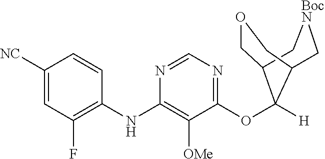 | 486 |
| 153 | 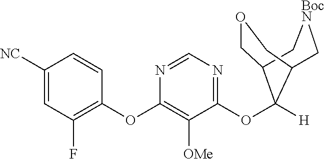 | 487 |
| 154 | 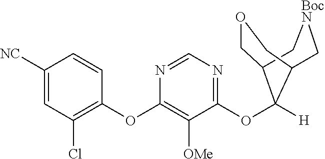 | 503, 505 |
| 155 | 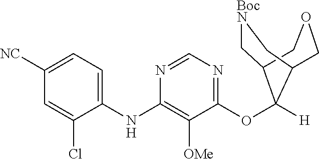 | 502, 504 |
| 156 | 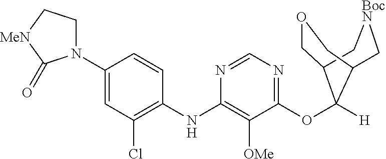 | 575, 577 |
| 157 | 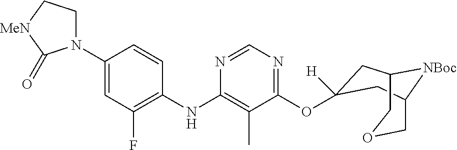 | 543 |
| 158 | 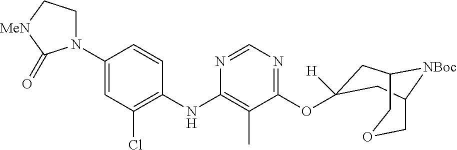 | 559, 561 |

### Example 44

### Preparation of Compound 159

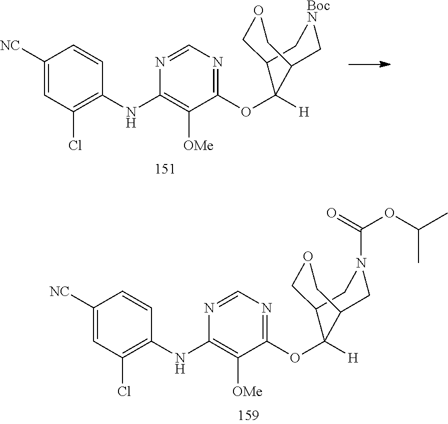

Using the method described in Example 9, Compound 151 was converted to Compound 159, a white solid, LC/MS 488+490 (MH⁺).

Utilizing compounds of Example 43 and relevant acylation methods, the following compounds of the present invention were prepared:

| Cpd. | | |
|---|---|---|
| No. | Structure | LC/MS |
| 160 | 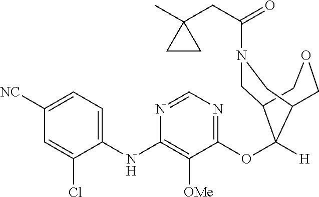 | 500, 502 |
| 161 | 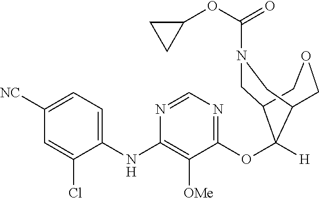 | 486, 488 |
| 162 | 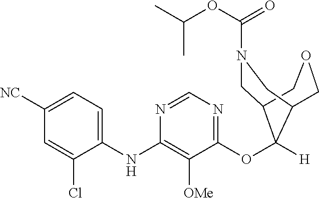 | 488, 490 |
| 163 | 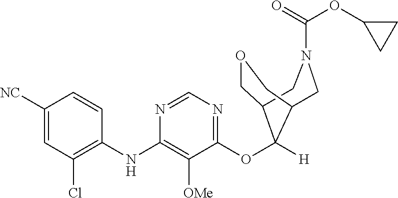 | 486, 488 |
| 164 | 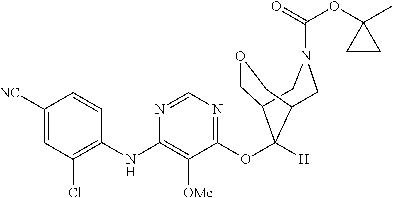 | 500, 502 |
| 165 | 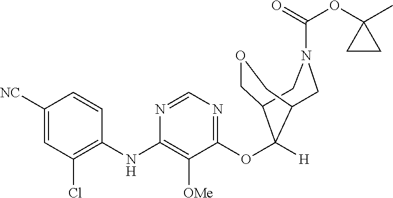 | 484 |
| 166 | 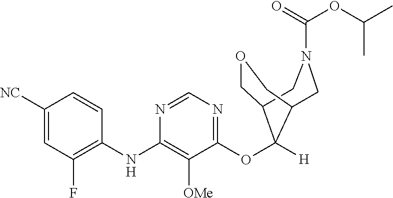 | 472 |
| 167 | 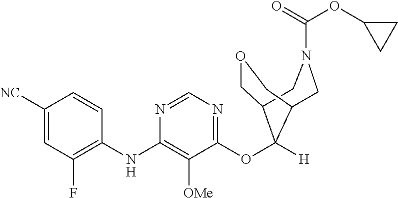 | 470 |
| 168 | 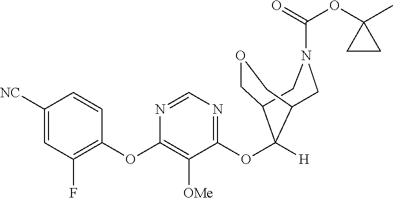 | 485 |
| 169 | 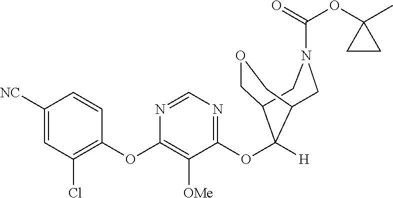 | 501, 503 |
| 170 | 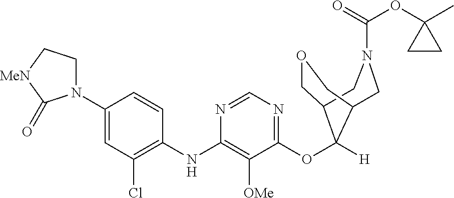 | 573, 575 |
| 171 | 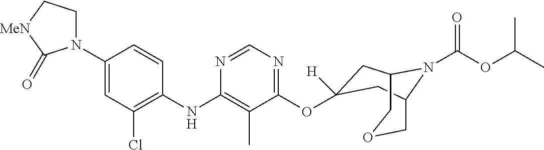 | 545, 547 |
| 172 | 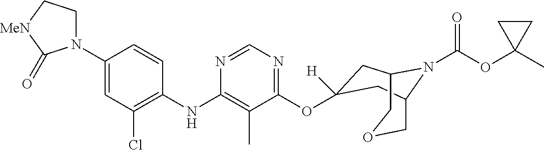 | 557, 559 |
| 173 | 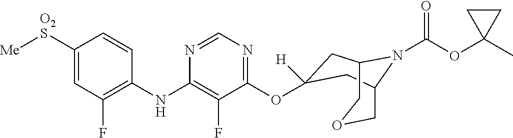 | 525 |
| 174 | 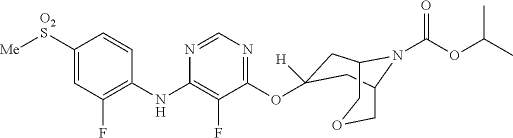 | 513 |
| 175 | 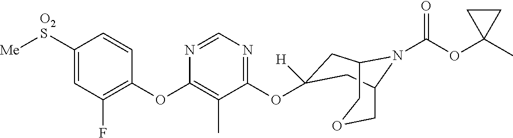 | 522 |
| 176 | 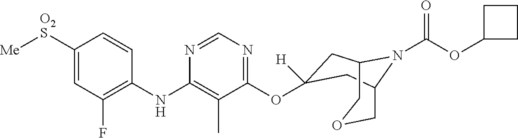 | 522 |
| 177 | 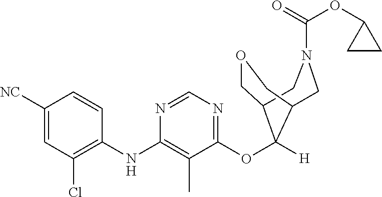 | 470, 472 |
| 178 | 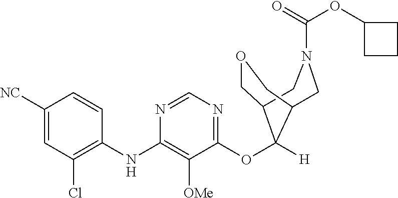 | 500, 502 |
| 179 | 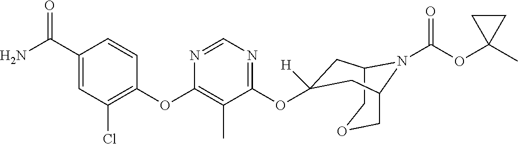 | 503, 505 |
| 180 | 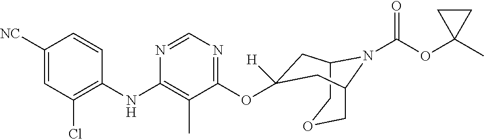 | 484, 486 |
| 181 | 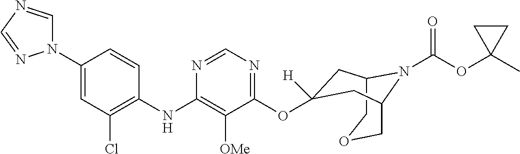 | 542, 544 |
| 182 | 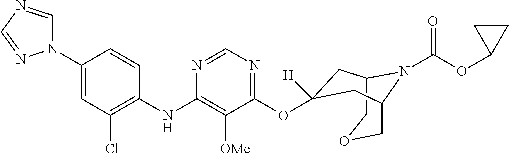 | 528, 530 |
| 183 | 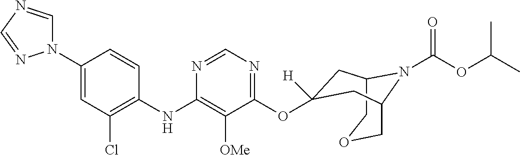 | 530, 532 |
| 184 | 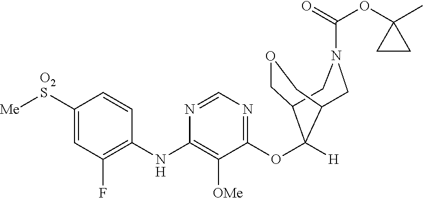 | 537 |

### Example 45

### Preparation of Compound 185

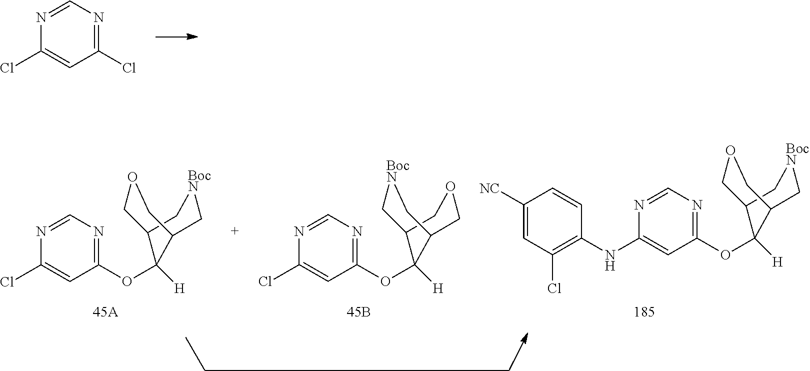

Using the method described in Example 43, 4,6-dichloropyrimidine was converted to Compound 185, a yellow solid, LC/MS: 472+474 (MH⁺).

Using this method and the appropriate reactants, the following compounds of the present invention were prepared:

| Cpd. | | |
|---|---|---|
| No. | Structure | LC/MS |
| 186 | 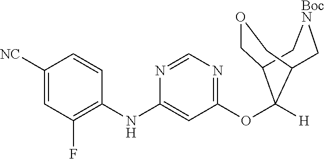 | 484 |
| 187 | 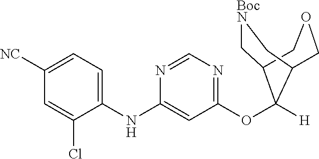 | 472, 474 |
| 188 | 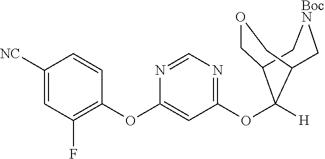 | 457 |
| 189 | 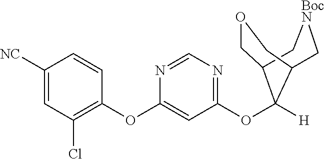 | 473, 475 |

### Example 46

### Preparation of Compound 190

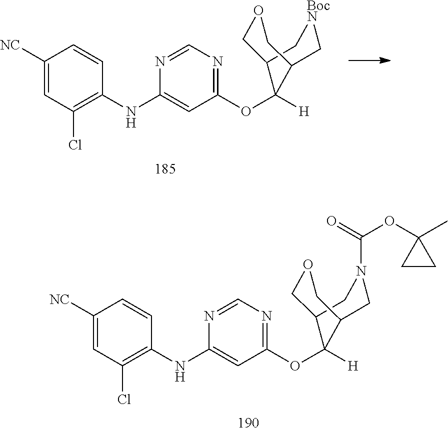

Using the method described in Example 8, Compound 185 was converted into Compound 190, a white solid, LC/MS: 470+472 (MH⁺).

Using this method and the appropriate reactants, the following compounds of the present invention were made:

| Cpd. | | LC/ |
|---|---|---|
| No. | Structure | MS |
| 191 | 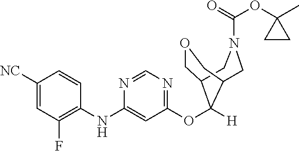 | 454 |
| 192 | 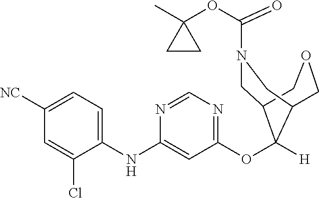 | 470, 472 |
| 193 | 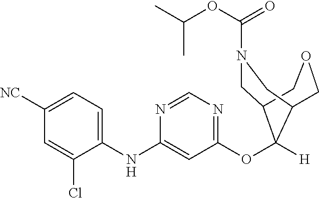 | 458, 460 |
| 194 | 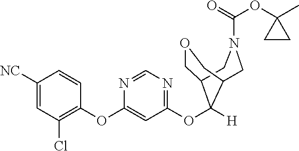 | 471, 473 |
| 195 | 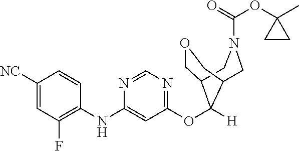 | 455 |

### Example 47

### Preparation of Compound 196

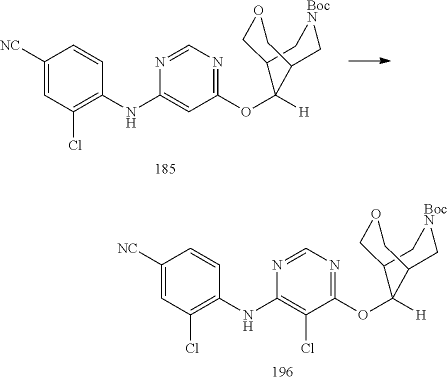

Compound 185 (0.090 g, 0.19 mmol) and N-chlorosuccinimide (0.030 g, 0.22 mmol) were combined in DMF (2.0 mL) and stirred for 18 hours at room temperature. The reaction mixture was concentrated in vacuo and purified using PLC to provide compound 196 as a yellow solid, LC/MS: 506+508+510 (MH⁺).

Using this method and the appropriate reactants, the following compounds of the present invention were prepared:

| Cpd. | | |
|---|---|---|
| No. | Structure | LC/MS |
| 197 | 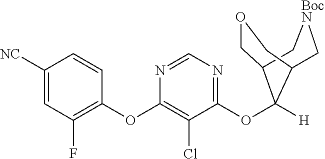 | 491, 493 |
| 198 | 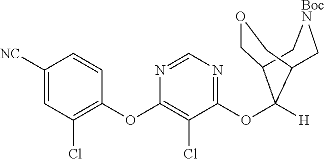 | 507, 509, 511 |

### Example 48

### Preparation of Compound 199

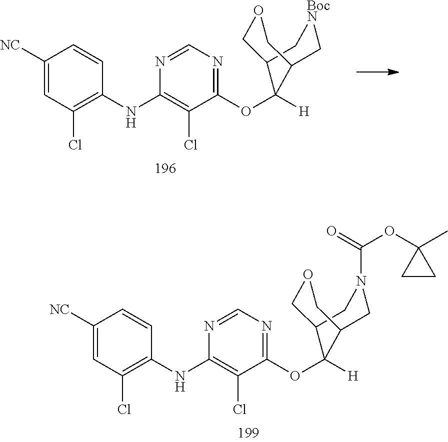

Using the method described in Example 8, Compound 196 was converted to Compound 199, a white solid, LC/MS: 504+506+508 (MH⁺).

Utilizing the appropriate starting materials and relevant acylation methods, the following compounds of the present invention were prepared:

| Cpd. | | LC/ |
|---|---|---|
| No. | Structure | MS |
| 200 | 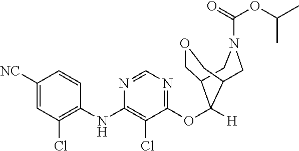 | 492, 494, 496 |
| 201 | 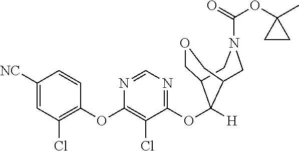 | 505, 507, 509 |
| 202 | 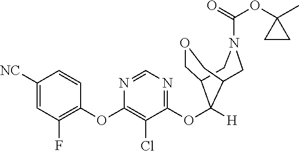 | 489, 491 |
| 203 | 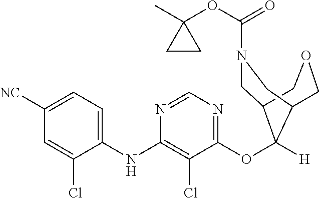 | 504, 506, 508 |
| 204 | 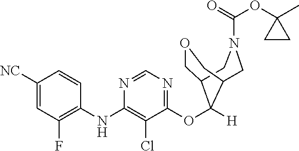 | 488, 490 |

### Example 49

### Preparation of Intermediate Compound 205

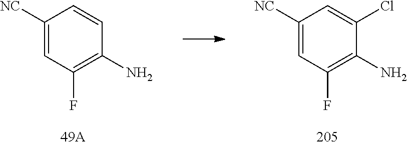

Using the method described in Example 47, and substituting acetic acid as the solvent, compound 49A was converted to compound 205, an off-white solid.

### Example 50

### Preparation of Compound 206

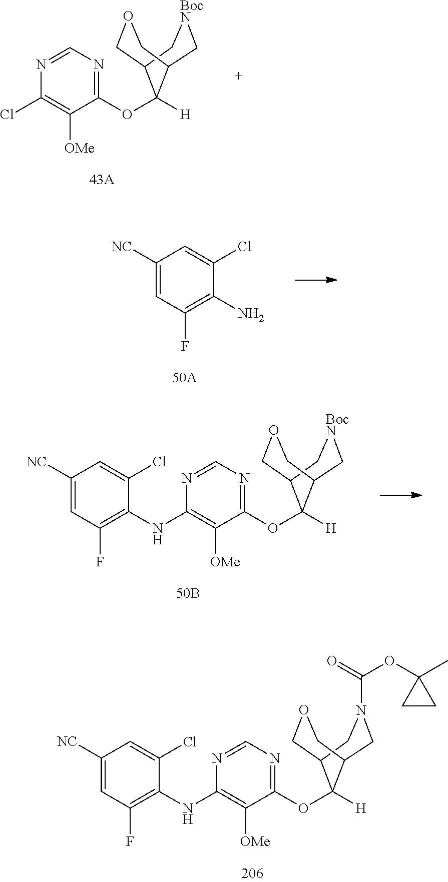

Using the methods described in Examples 43 and 45, compound 43A was converted to compound 50B. Compound 50B was subsequently converted to compound 206, a colorless gum, using the method described in Example 47. LC/MS: 518+520 (MH⁺).

Using this method and the appropriate reactants, the following compounds of the present invention were prepared:

| Cpd. | | LC/ |
|---|---|---|
| No. | Structure | MS |
| 207 | 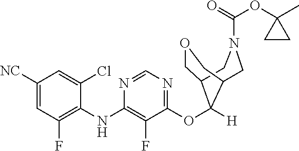 | 506, 508 |
| 208 | 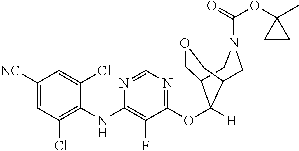 | 522, 524, 526 |
| 209 | 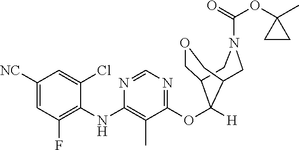 | 502, 504 |
| 210 | 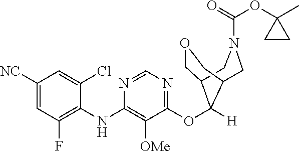 | 518, 520 |
| 211 | 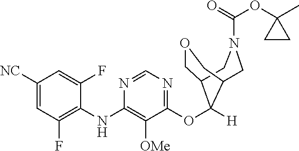 | 502 |
| 212 | 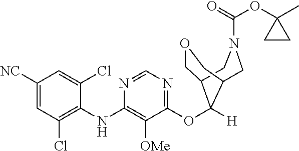 | 534, 536, 538 |
| 213 | 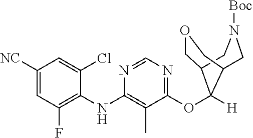 | 504, 506 |

### Example 51

### Preparation of Intermediate Compound 214

2-Chloro 4-iodoaniline was converted to Compound 214 using the methods described in International Publication No. WO 94/22855.

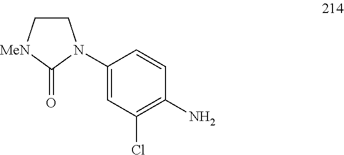

### Example 52

### Preparation of Compounds 215 and 216

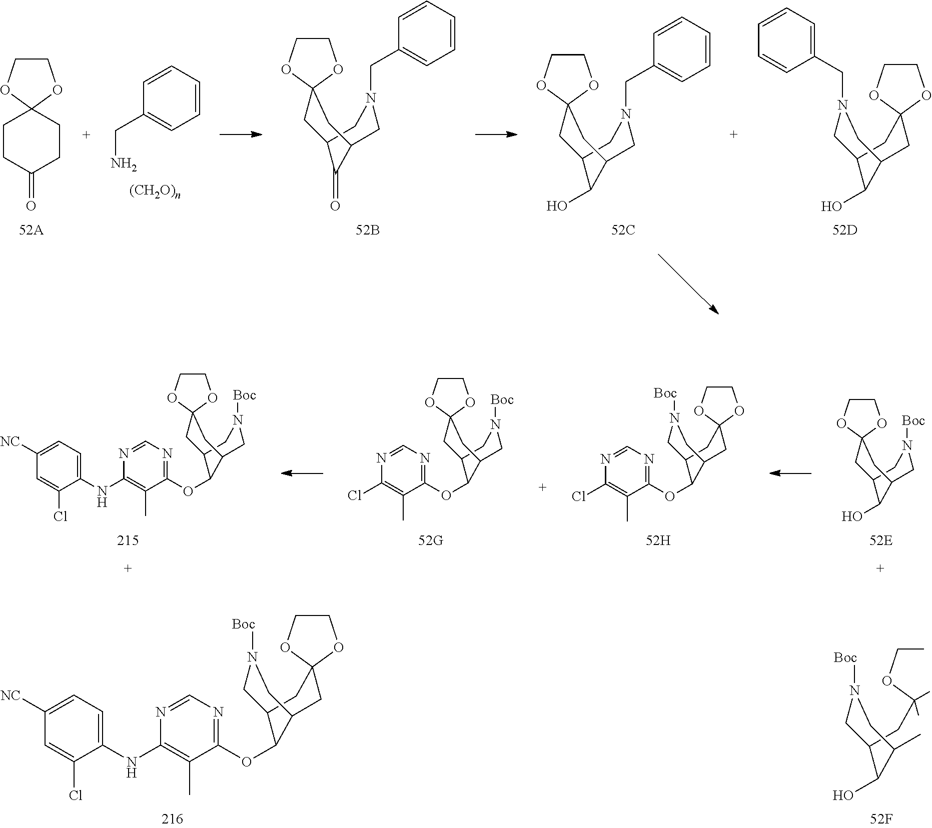

### Step A—Synthesis of Compound 52B

A solution of paraformaldehyde (2.64 g, 88 mmol formaldehyde) in MeOH was heated to 65° C. A mixture of compound 52A (6.25 g, 40.1 mmol), benzylamine (4.81 mL, 44.1 mmol), and acetic acid (2.64 g, 44.0 mmml) in MeOH (100 mL) was then added dropwise. The reaction was allowed to stir for 1 hour at 65° C., then additional paraformaldehyde (2.64 g) was added. After stirring for an additional 1 hour at 65° C., the reaction mixture was allowed to cool, concentrated in vacuo, and partitioned between ether (200 mL) and 5% NaOH (100 mL). The ether was dried (MgSO₄), filtered, concentrated in vacuo, and purified using flash column chromatography on silica (0-30% EtOAc/hexane) to provide compound 52B (1.6 g) as a yellow oil.

### Step B—Synthesis of Compounds 52C and 52D

To Compound 52B (1.60 g, 5.6 mmol) in MeOH (5 mL) was added NaBH₄ (0.212 g, 5.6 mmol). The mixture was stirred for 2 hours at room temperature, then partitioned between diethyl ether and water. The ether was dried (MgSO₄), filtered, and concentrated in vacuo to provide a mixture of compounds 52C and 52D as a yellow oil.

### Step C—Synthesis of Compounds 52E and 52F

To the above mixture of compounds 52C and 52D (1.42 g, 4.9 mmol) in EtOH (20 mL) was added 10% Pd/C (0.50 g) and Boc₂O (1.82 g, 8.3 mmol). The mixture was hydrogenated at 50 psi for 18 hours, then filtered through celite to remove catalyst, and the filtrate was concentrated in vacuo. The resulting residue was purified using flash column chromatography on silica gel (0-70% EtOAc/hexane) to provide a mixture of compounds 52E and 52F as a yellow oil.

### Step D—Synthesis of Compounds 52G and 52H

The above mixture of compounds 52E and 52F (1.42 g, 4.9 mmol) in THF (20 mL) was treated with 4,6-dichloro-5-methylpyrimidine using the method described in Example 5 (reaction for 18 hours at room temperature). Purification by PLC (30% EtOAc/hexane) provided compound 52G as the less polar component and compound 52H as the more polar component.

### Step E—Synthesis of Compounds 215 and 216

Compound 84G was treated with 4-amino-3-chlorobenzonitrile according to the method described in Example 5. Separation by PLC (3% MeOH/CH₂Cl₂) provided faster running compound 215 and slower running compound 216, each as a white solid, MS: 542+544 (MH⁺).

### Example 53

### Preparation of Compound 217

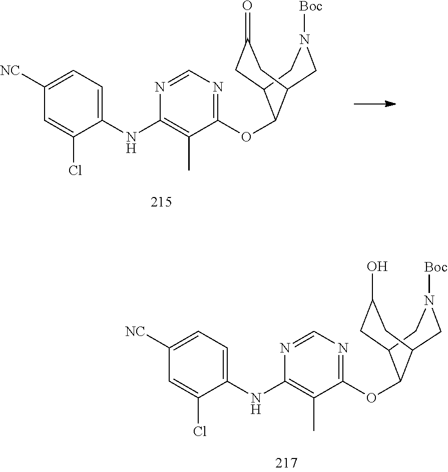

Compound 215 was reduced using NaBH₄ under standard conditions for 72 hours. Typical workup, followed by purification using PLC provided compound 217 as a white solid, MS: 500+502 (MH⁺).

### Example 54

### Preparation of Compound 218

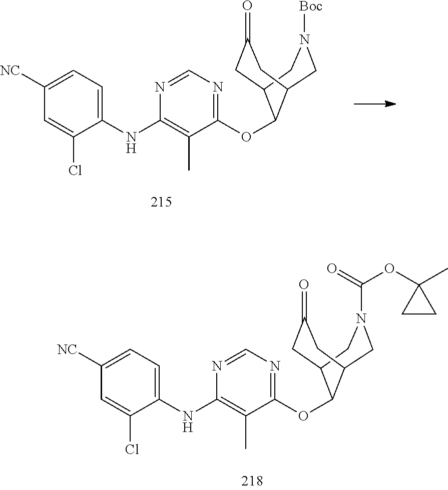

Compound 215 was modified according to the method described in Example 8 to provide compound 218 as a white solid, MS: 496+498 (MH⁺).

The following compounds of the present invention were prepared using the method described above and substituting the appropriate reactants:

| Cpd. | | LC/ |
|---|---|---|
| No. | Structure | MS |
| 219 | 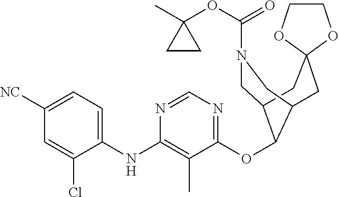 | 540, 542 |
| 220 | 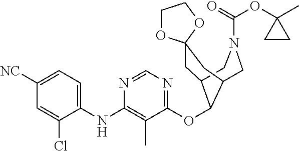 | 540, 542 |
| 221 | 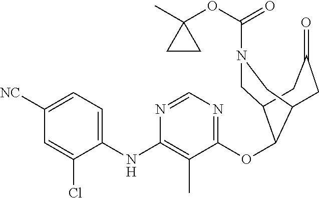 | 496, 498 |

### Example 55

### Preparation of Compound 222

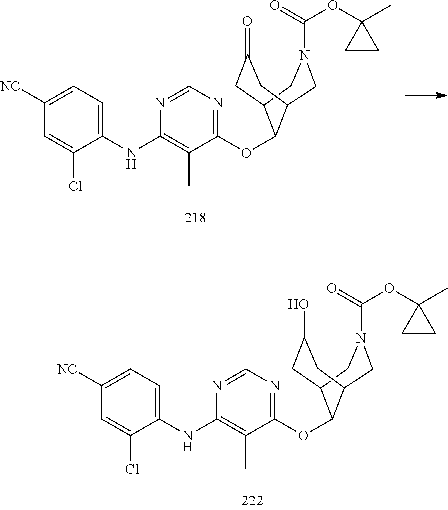

Compound 218 was reduced using NaBH₄ under standard conditions. Typical workup, followed by purification using PLC provided compound 222 as a yellow solid, MS: 498+500 (MH⁺).

### Example 56

### Preparation of Compound 223

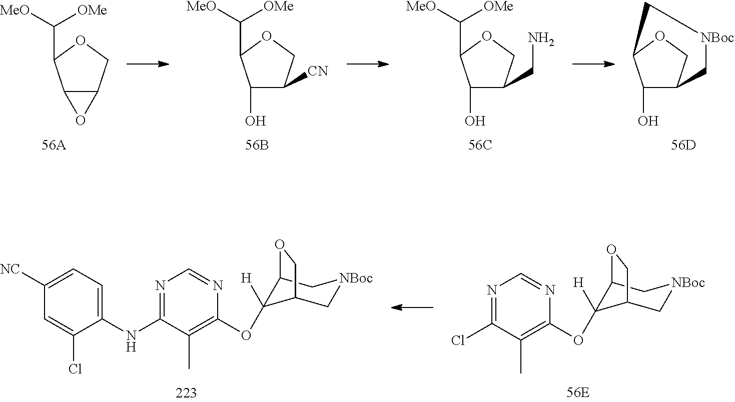

### Step A—Synthesis of Compound 568

Compound 56A (prepared according to Tetrahedron 1997, 3831, 1.00 g, 6.25 mmol) was combined with LiCN acetone solvate (prepared according to Tetrahedron Lett. 2004, 7201, 1.42 g, 15.6 mmol) in THF (12 mL). The mixture was heated at 65° C. for 4 hours, allowed to cool, concentrated in vacuo, and partitioned with EtOAc and water. The organic layer was dried (MgSO₄), filtered and concentrated in vacuo to provide a crude residue which was purified using flash column chromatography on silica to provide compound 56B as a yellow oil.

### Step B—Synthesis of Compound 56C

Compound 56B (0.533 g, 2.85 mmol) was combined with PtO₂ (0.15 g) in MeOH (5 mL) and the resulting solution was hydrogenated at 55 psi for 18 hours. The reaction mixture was then filtered through Celite to remove catalyst and the filtrate was concentrated in vacuo to provide compound 56C as a yellow oil.

### Step C—Synthesis of Compound 56D

Compound 56C (0.53 g, 2.8 mmol) was taken up in MeOH (10 mL), and 1.0N HCl (8.5 mL) was added. The reaction mixture was heated to 60° C. and allowed to stir at this temperature for 4 hours, then conc. HCl (1.0 mL) was added. After another 18 hours, the mixture was allowed to cool to room temperature, then concentrated in vacuo. The residue was taken up in EtOH (6 mL) and cooled in an ice bath. NaBH(OAc)₃ (0.95 g, 4.5 mmol) was then added, followed by 6 drops HOAc. After stirring for 10 hours at 0° C., the reaction mixture was allowed to warm to room temperature and stirred for an additional 20 hours. The reaction mixture was then concentrated in vacuo and the residue obtained was taken up in EtOH (10 mL). Et₃N (1.5 mL, 10 mmol) and Boc₂O (0.66 g, 3.0 mmol) were added and the mixture was allowed to stir for 18 hours. The reacton mixture was then concentrated in vacuo and the resulting residue was partitioned with CH₂Cl₂ and water. The organic layer was dried (MgSO₄), filtered and concentrated in vacuo, and the residue obtained was purified using flash column chromatography on silica to provide compound 56D as a yellow oil.

### Step D—Synthesis of Compound 56E

Using the method described in Example 5, compound 56D was converted to Compound 56E, a colorless oil.

### Step E—Synthesis of Compound 223

Compound 56E was treated with 4-amino-3-chlorobenzonitrile according to the method described in Example 5 to provide compound 223 as a white solid. MS: 472+474 (MH⁺).

### Example 57

### Preparation of Compound 224

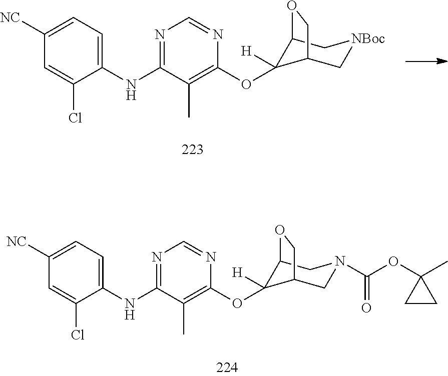

Compound 223 was treated according to the procedure of Example 8 to provide compound 224 as a white solid, MS: 470+472 (MH⁺).

### Example 58

### Preparation of Compound 225

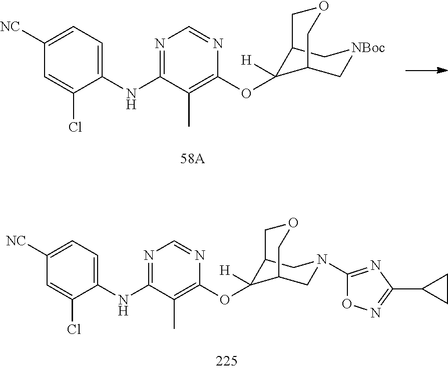

Compound 58A (prepared from Compound 66A2 analogously to Compound 70) was treated according to the procedure of Example 42 to provide compound 225 as a white solid, LC/MS: 494+496 (MH⁺).

### Example 59

### Preparation of Compounds 226 and 227

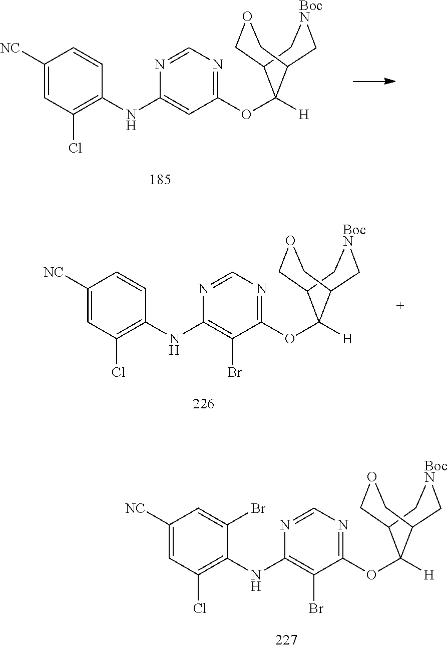

Using the method described in Example 47, compound 185 was reacted with N-bromosuccinimide to provide compound 226 as a white solid, LC/MS: 550+552+554 (MH⁺), together with Compound 227, a white solid, LC/MS: 628+630+632+634 (MH⁺).

### Example 60

### Preparation of Compounds 228 and 229

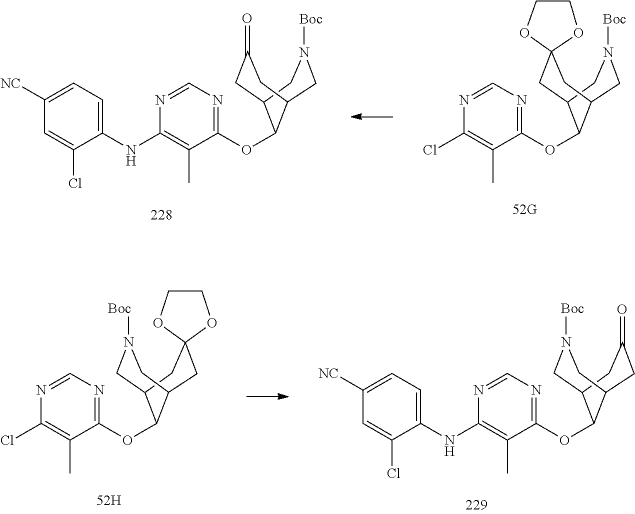

A mixture of compounds 52G and 52H was separated by PLC with 30% EtOAc/hexane. Each compound was treated with 4-amino-3-chlorobenzonitrile according to the method described in Example 5 to provide respectively compounds 228 and 229, each a white solid, LC/MS: 498+500 (MH⁺).

### Example 61

### Preparation of Compound 230

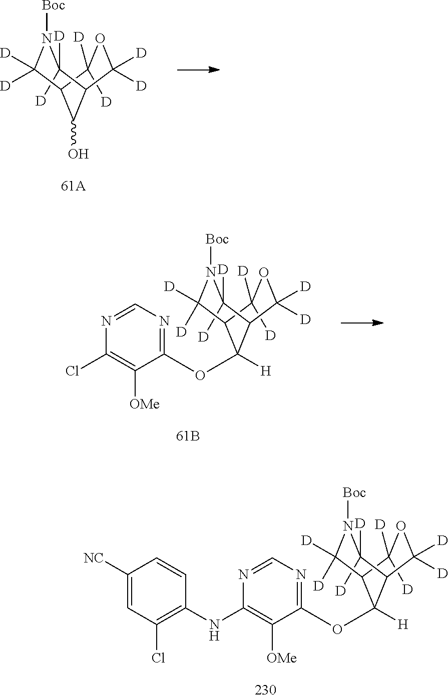

Compound 61A was prepared as described in Chem. Eur. J. 2002, 4767, employing deuteroformaldehyde in place of formaldehyde, with subsequent NaBH₄ reduction. Compound 61A was then converted to Compound 230, via intermediate 61B, using the methods described in Example 5. LC/MS: 504+506 (MH⁺).

### Example 62

### Preparation of Compound 231

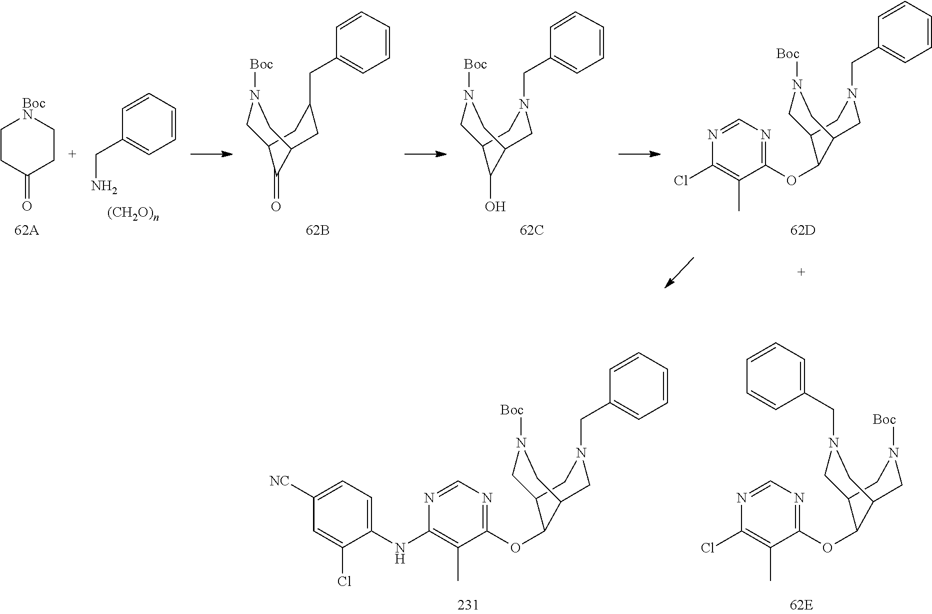

### Step A—Synthesis of Compound 628

Compound 62A was reacted using the method described in Example 52, Step A, to provide compound 62B as a colorless oil.

### Step B—Synthesis of Compound 62C

Compound 62B (1.60 g, 5.6 mmol) was treated with NaBH₄ according to the method described in Example 52, Step B, to provide compound 62C as a white solid.

### Step C—Synthesis of Compound 62D

Compound 62C was treated with 4,6-dichloro-5-methylpyrimidine using the method described in Example 52, Step D, to provide compound 62D as the major product and Compound 94E as the minor product.

### Step D—Synthesis of Compound 231

Compound 62D was treated with 4-amino-3-chlorobenzonitrile using the method described in Example 52, Step E, to provide compound 231 as a yellow solid, LC/MS: 575+577 (MH⁺).

The following compound was prepared using the method described above and substituting the appropriate reactant:

| Cpd. | | LC/ |
|---|---|---|
| No. | Structure | MS |
| 232 | 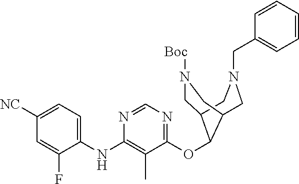 | 559 |

### Example 63

### Preparation of Compound 233

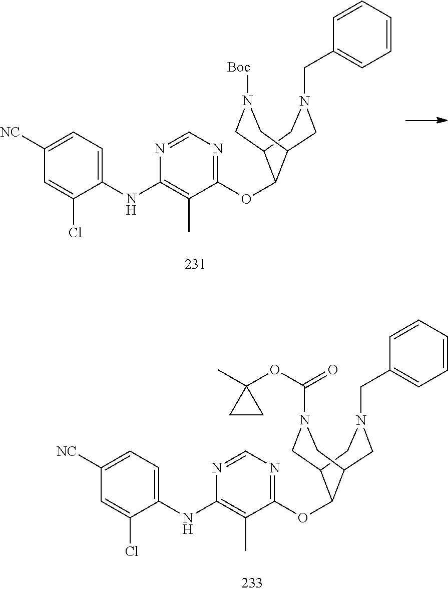

Compound 231 was reacted using the method described in Example 8 to provide compound 233 as a white solid, LC/MS: 573+575 (MH⁺).

### Example 64

### Preparation of Compound 234

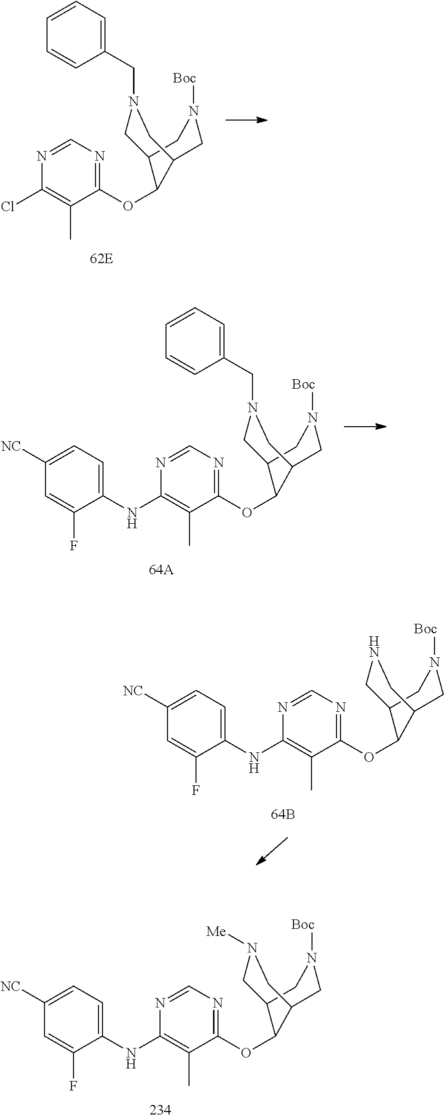

### Step A—Synthesis of Compound 64A

Compound 62E was reacted with 4-amino-3-chlorobenzonitrile using the method described in Example 62, Step D, to provide compound 64A as a yellow solid.

### Step B—Synthesis of Compound 648

A solution of compound 64A (0.38 g, 0.68 mmol) and 10% Pd/C (0.20 g) in 1:1 MeOH/EtOAc (10 mL) was hydrogenated at 1 atm for 64 hours. The catalyst was filtered and the filtrate was concentrated in vacuo. The residue obtained was purified using PLC to provide compound 64B as a white solid.

### Step C—Synthesis of Compound 234

A solution of compound 64B (0.012 g, 0.026 mmol), Et₃N (0011 mL, 0.080 mmol) and MeI (0.003 mL, 0.05 mmol) in THF (1.0 mL) was placed in a sealed tube, heated to 60° C. and allowed to remain at this temperature for 4 hours. The reaction mixture was then concentrated in vacuo and the resulting residue was purified using PLC to provide compound 234 as a white solid. LC/MS: 483 (MH⁺).

### Example 66

### Preparation of Compounds 235-607

Compounds 235-607 were made using one or more of the methods described above in Examples 5, 8 and 36.

| Cpd. | | LCMS |
|---|---|---|
| No. | Structure | (M + H) |
| 235 | 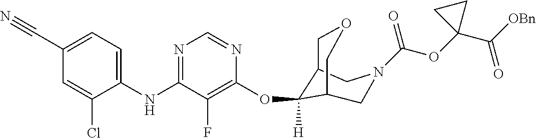 | 609 |
| 236 | 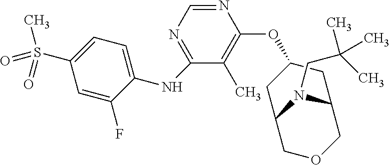 | 494 |
| 237 | 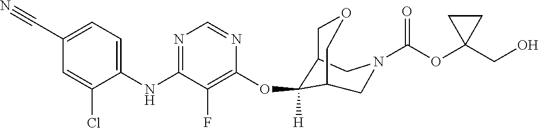 | 504 |
| 238 | 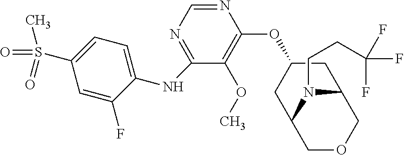 | 536 |
| 239 | 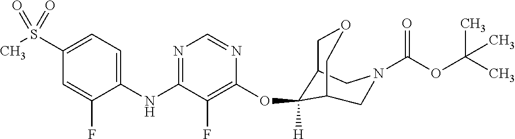 | 529 |
| 240 | 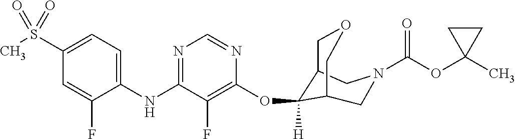 | 527 |
| 241 | 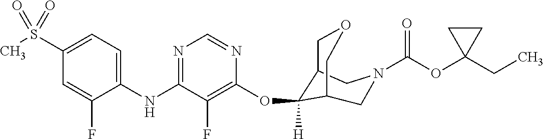 | 541 |
| 242 | 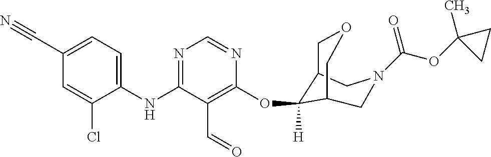 | 498 |
| 243 | 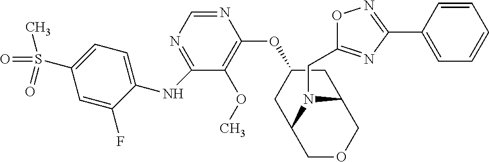 | 598 |
| 244 | 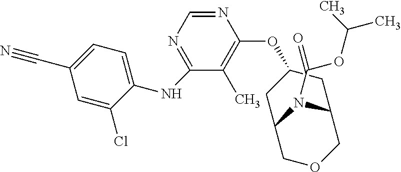 | 472 |
| 245 | 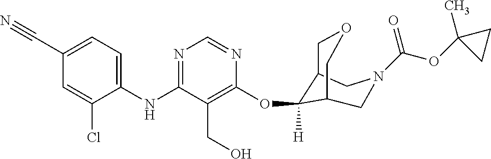 | 500 |
| 246 | 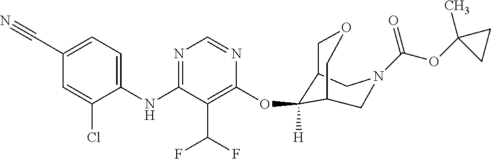 | 520 |
| 247 | 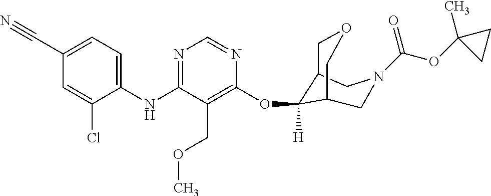 | 514 |
| 248 | 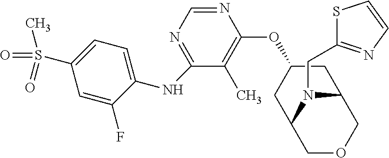 | 521 |
| 249 | 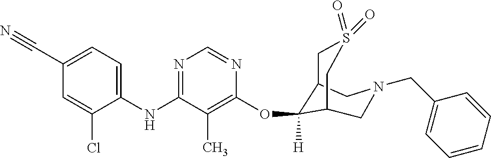 | 524 |
| 250 | 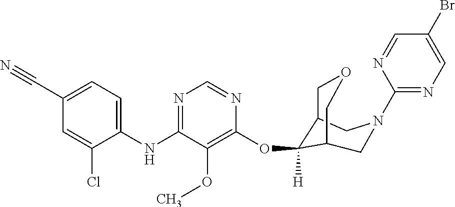 | 559 |
| 251 | 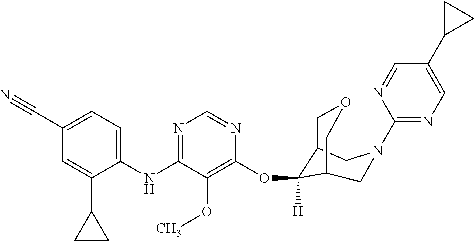 | 527 |
| 252 | 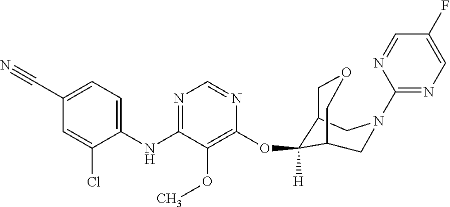 | 498 |
| 253 | 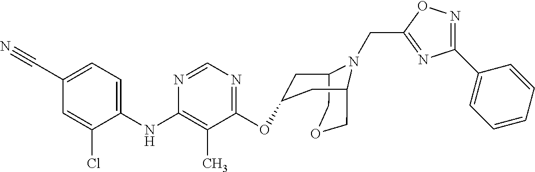 | 544 |
| 254 | 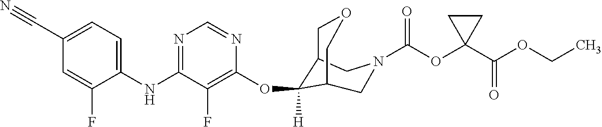 | 530 |
| 255 | 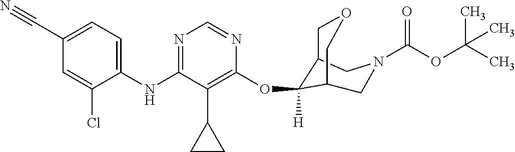 | 512 |
| 256 | 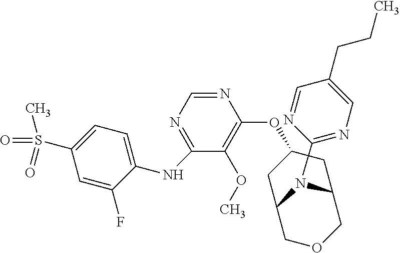 | 560 |
| 257 | 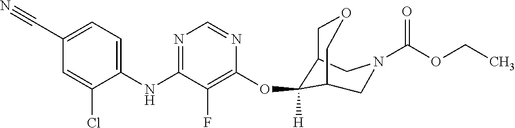 | 462 |
| 258 | 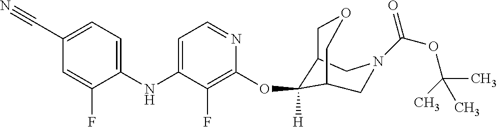 | 473 |
| 259 | 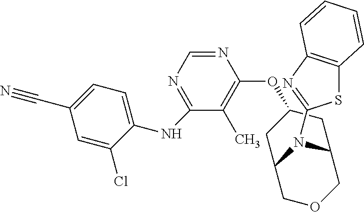 | 519 |
| 260 | 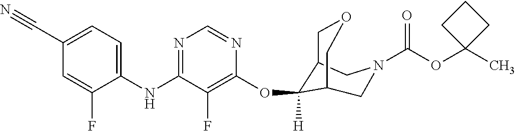 | 486 |
| 261 | 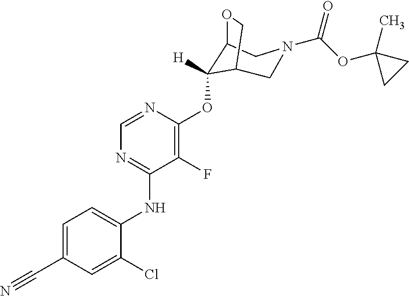 | 474 |
| 262 | 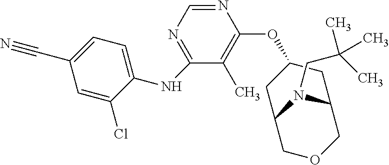 | 456 |
| 263 | 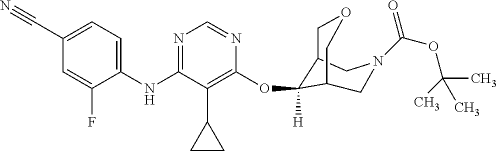 | 498 |
| 264 | 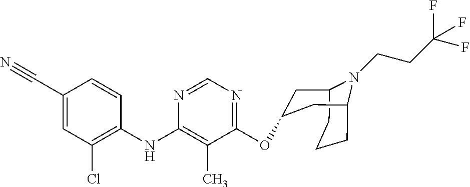 | 480 |
| 265 | 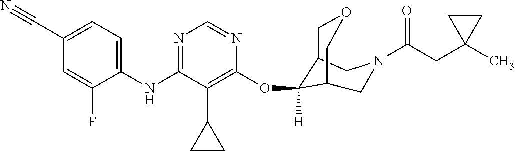 | 496 |
| 266 | 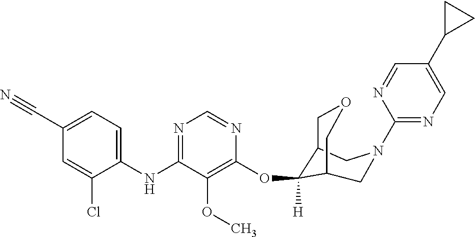 | 520 |
| 267 | 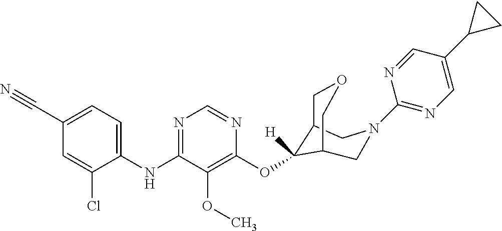 | 520 |
| 268 | 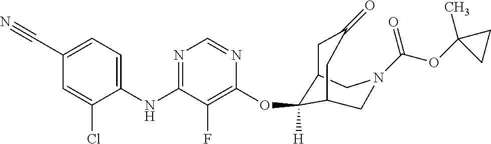 | 500 |
| 269 | 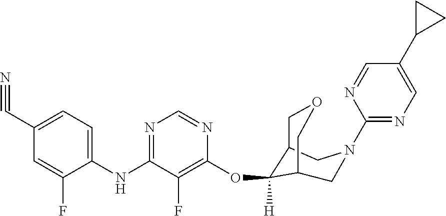 | 492 |
| 270 | 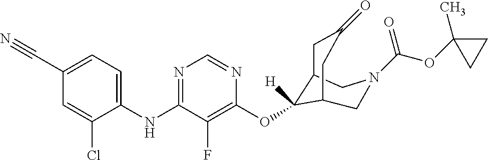 | 500 |
| 271 | 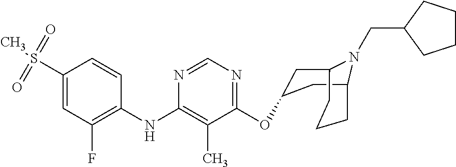 | 504 |
| 272 | 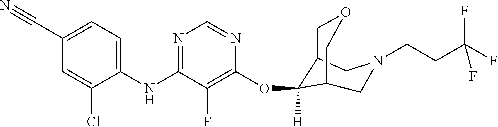 | 486 |
| 273 | 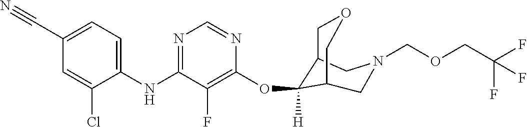 | 500 |
| 274 | 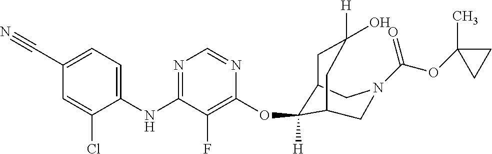 | 502 |
| 275 | 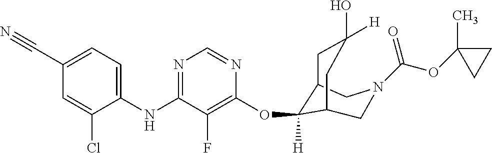 | 502 |
| 276 | 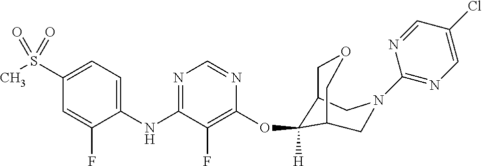 | 540 |
| 277 | 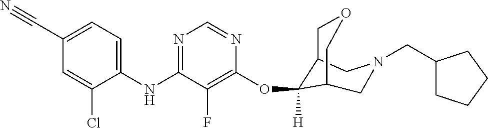 | 472 |
| 278 | 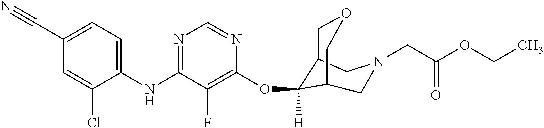 | 476 |
| 279 | 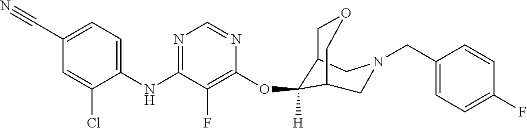 | 498 |
| 280 | 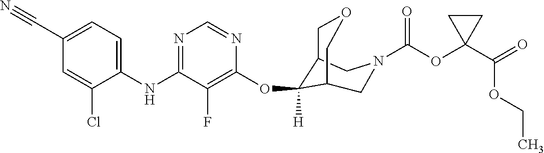 | 546 |
| 281 | 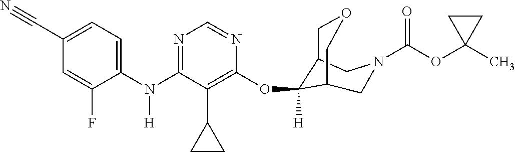 | 495 |
| 282 | 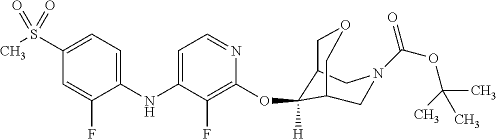 | 527 |
| 283 | 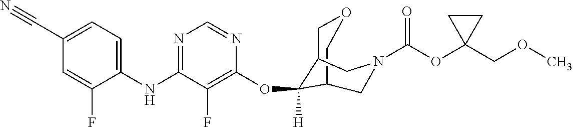 | 502 |
| 284 | 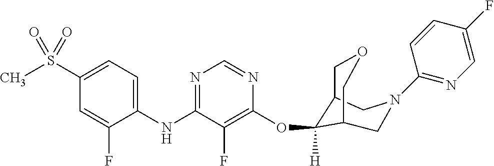 | 523 |
| 285 | 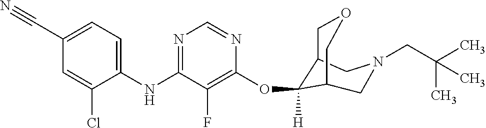 | 460 |
| 286 | 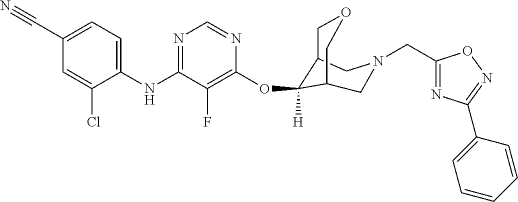 | 548 |
| 287 | 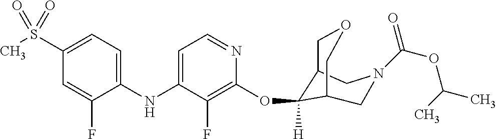 | 513 |
| 288 | 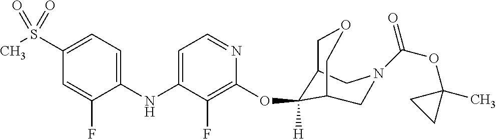 | 525 |
| 289 | 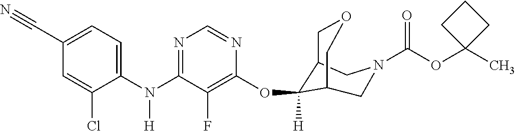 | 502 |
| 290 | 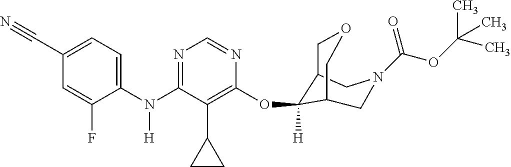 | 497 |
| 291 | 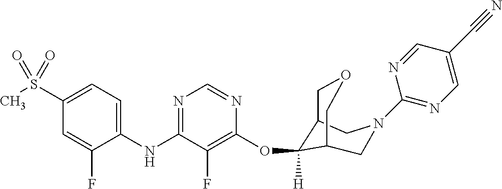 | 531 |
| 292 | 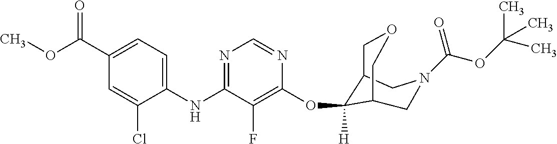 | 523 |
| 293 | 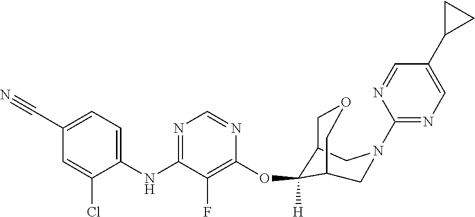 | 508 |
| 294 | 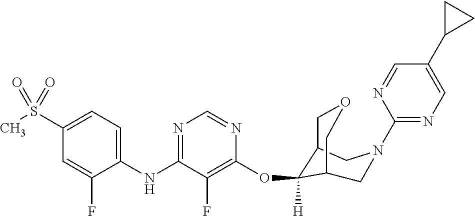 | 547 |
| 295 | 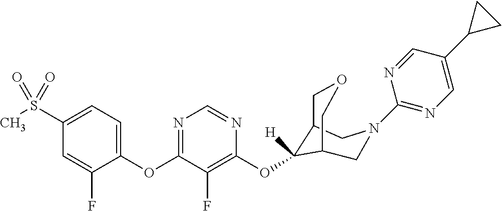 | 547 |
| 296 | 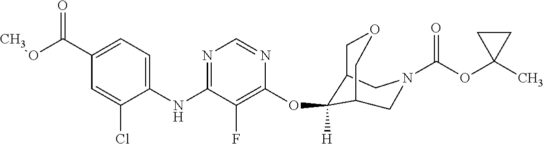 | 521 |
| 297 | 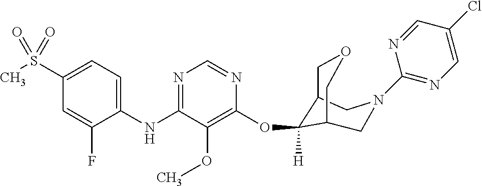 | 552 |
| 298 | 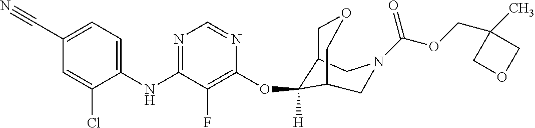 | 518 |
| 299 | 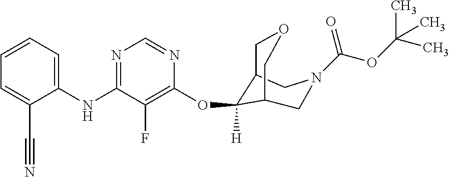 | 457 |
| 300 | 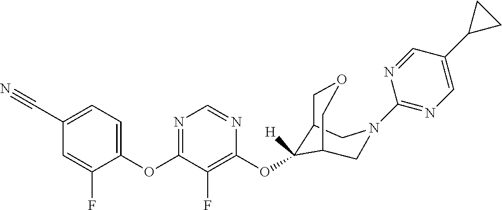 | 493 |
| 301 | 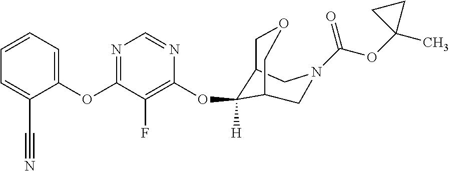 | 455 |
| 302 | 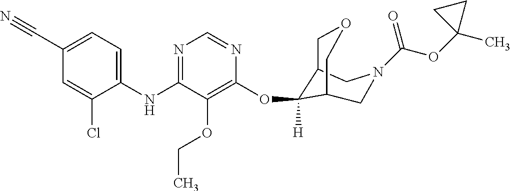 | 514 |
| 303 | 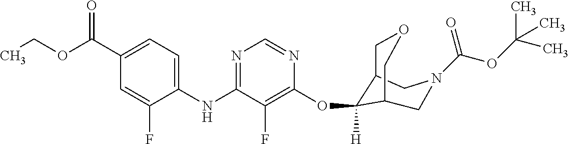 | 522 |
| 304 | 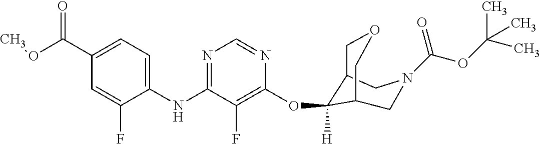 | 507 |
| 305 | 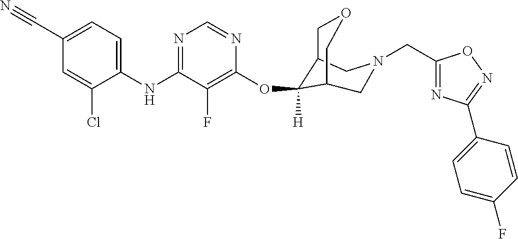 | 566 |
| 306 | 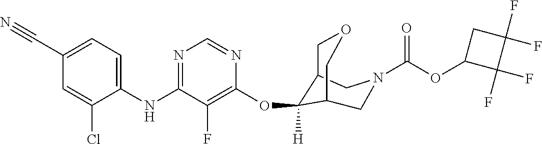 | 560 |
| 307 | 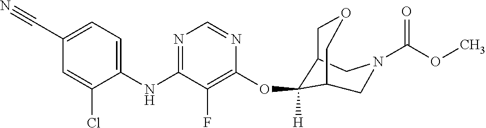 | 448 |
| 308 | 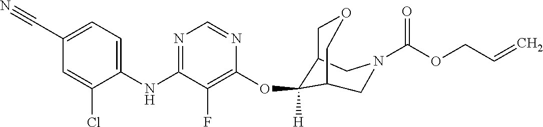 | 474 |
| 309 | 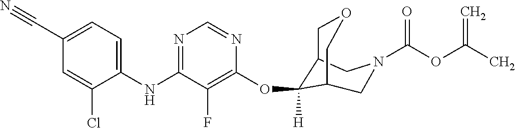 | 474 |
| 310 | 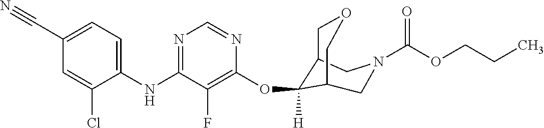 | 476 |
| 311 | 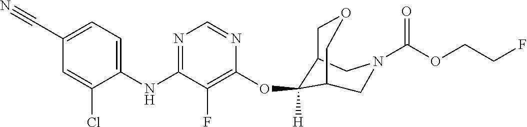 | 480 |
| 312 | 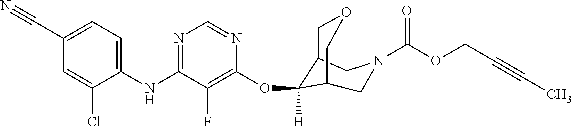 | 486 |
| 313 | 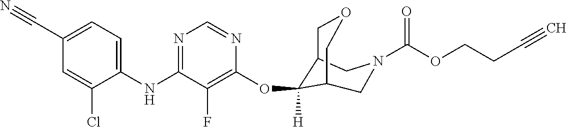 | 486 |
| 314 | 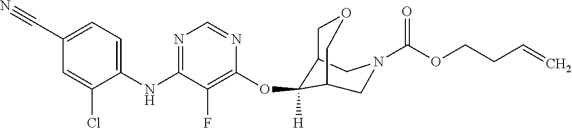 | 488 |
| 315 | 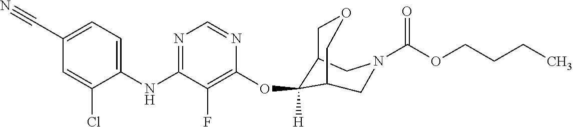 | 490 |
| 316 | 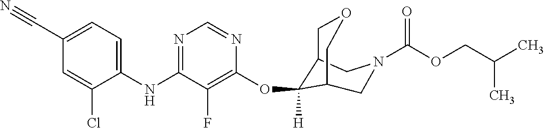 | 490 |
| 317 | 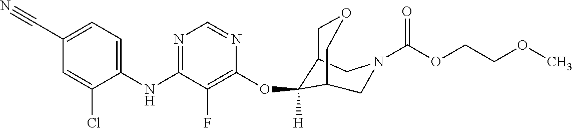 | 492 |
| 318 | 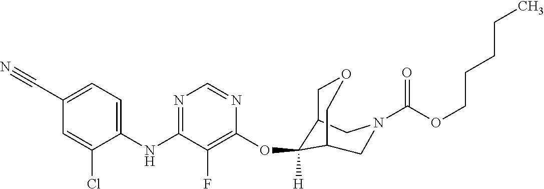 | 504 |
| 319 | 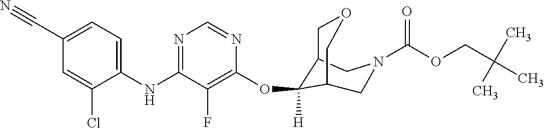 | 504 |
| 320 | 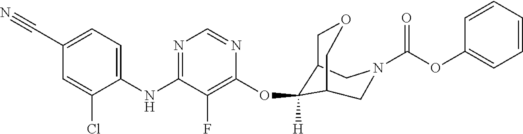 | 510 |
| 321 | 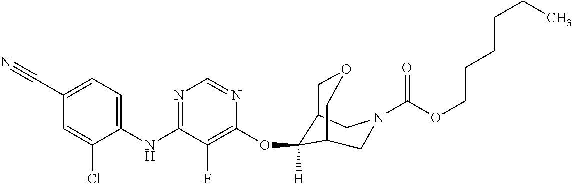 | 518 |
| 322 | 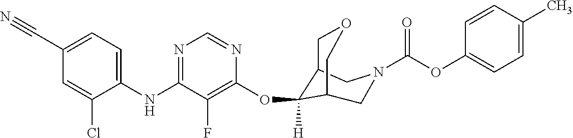 | 524 |
| 323 | 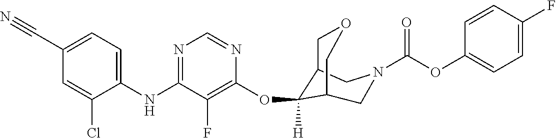 | 528 |
| 324 | 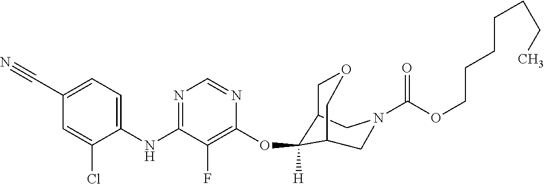 | 532 |
| 325 | 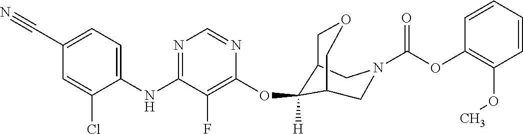 | 540 |
| 326 | 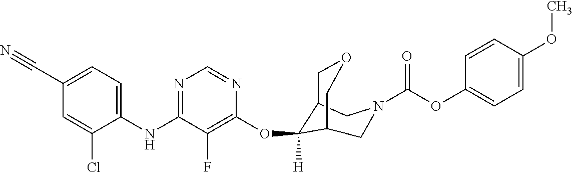 | 540 |
| 327 | 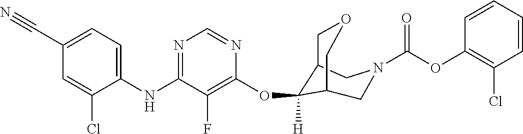 | 544 |
| 328 | 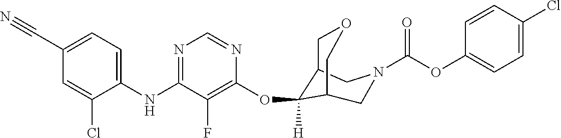 | 544 |
| 329 | 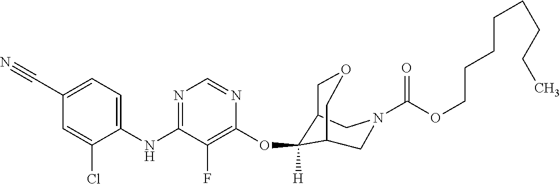 | 546 |
| 330 | 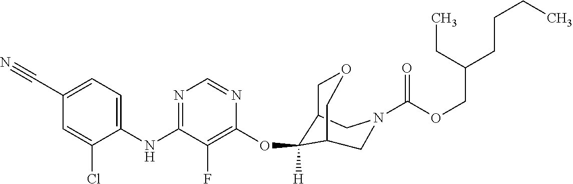 | 546 |
| 331 | 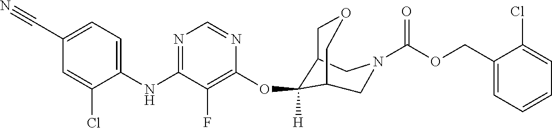 | 558 |
| 332 | 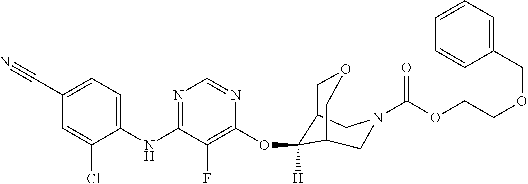 | 568 |
| 333 | 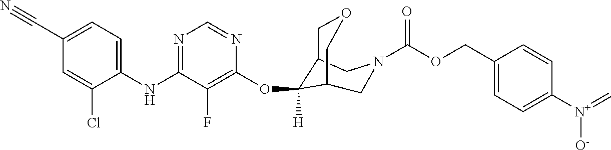 | 569 |
| 334 | 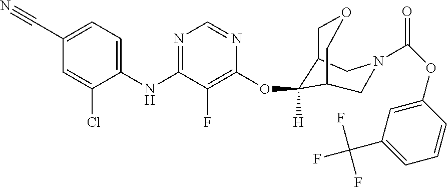 | 578 |
| 335 | 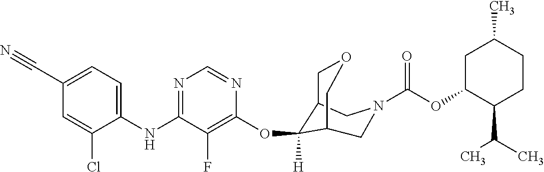 | 572 |
| 336 | 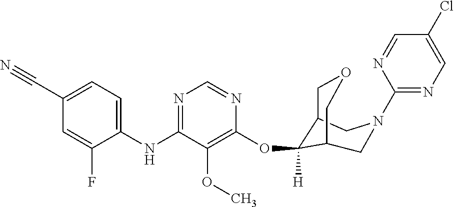 | 498 |
| 337 | 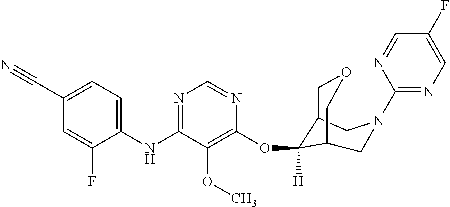 | 482 |
| 338 | 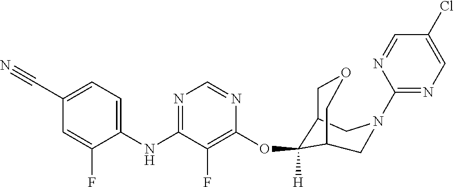 | 486 |
| 339 | 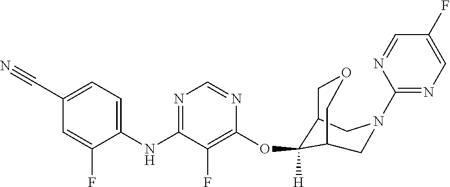 | 470 |
| 340 | 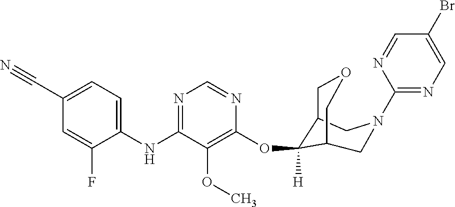 | 543 |
| 341 | 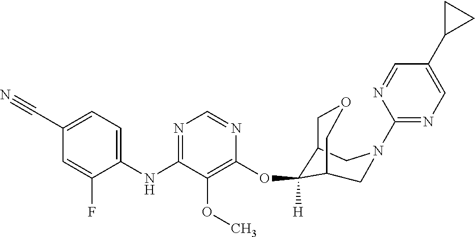 | 505 |
| 342 | 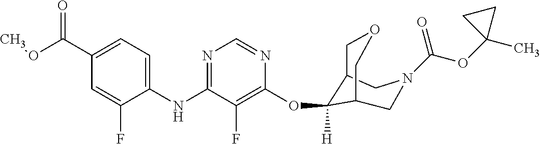 | 505 |
| 343 | 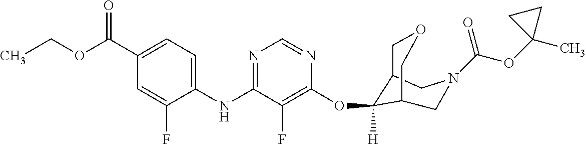 | 520 |
| 344 | 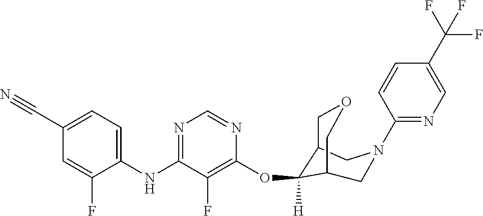 | 519 |
| 345 | 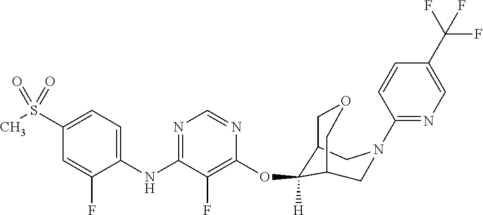 | 573 |
| 346 | 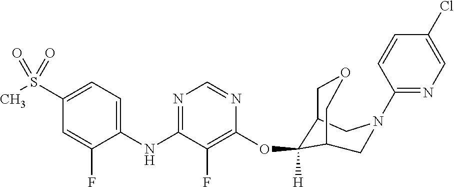 | 538 |
| 347 | 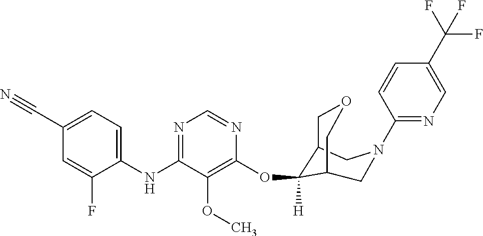 | 531 |
| 348 | 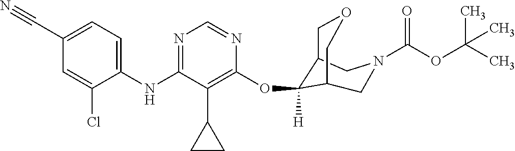 | 513 |
| 349 | 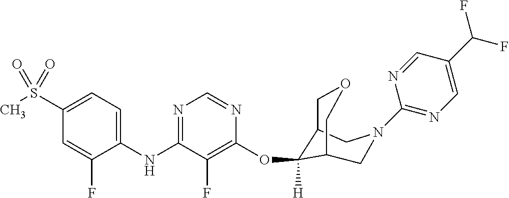 | 556 |
| 350 | 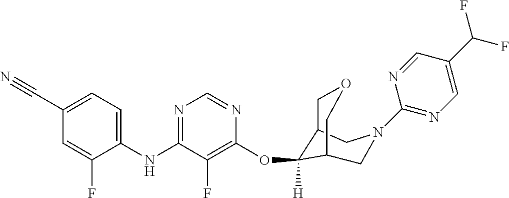 | 502 |
| 351 | 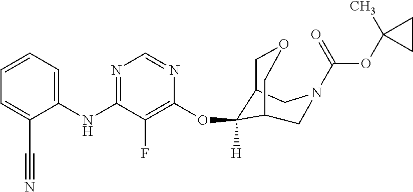 | 454 |
| 352 | 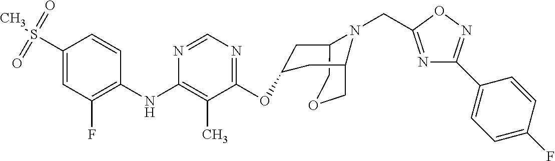 | 600 |
| 353 | 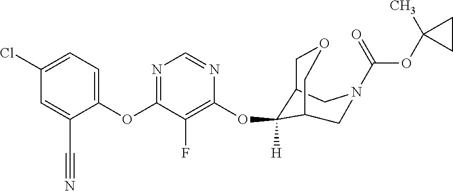 | 489 |
| 354 | 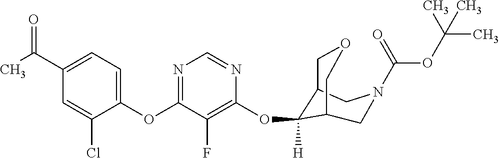 | 508 |
| 355 | 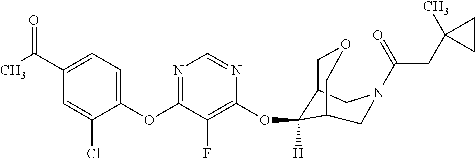 | 506 |
| 356 | 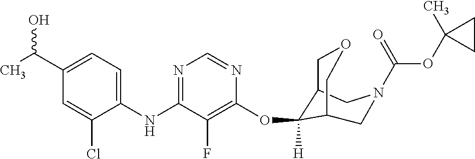 | 508 |
| 357 | 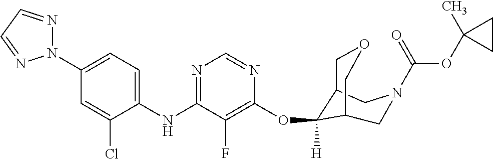 | 530 |
| 358 | 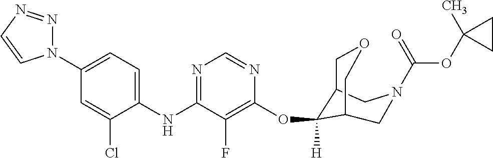 | 530 |
| 359 | 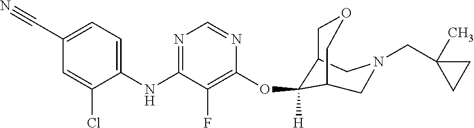 | 458 |
| 360 | 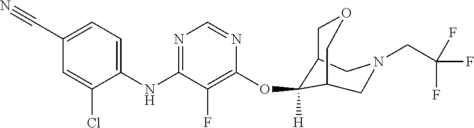 | 472 |
| 361 | 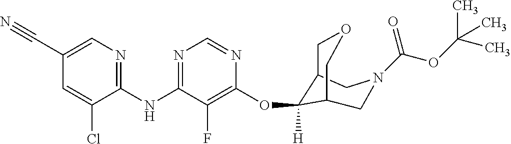 | 491 |
| 362 | 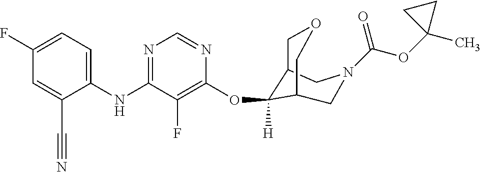 | 472 |
| 363 | 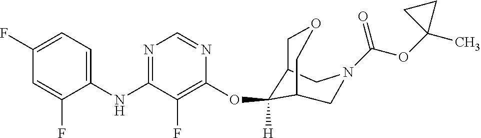 | 465 |
| 364 | 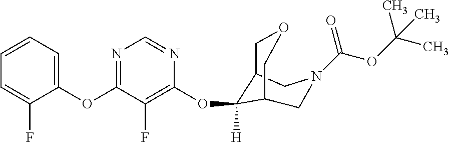 | 450 |
| 365 | 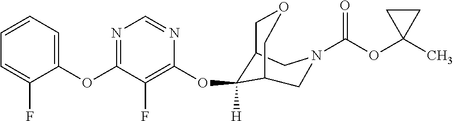 | 448 |
| 366 | 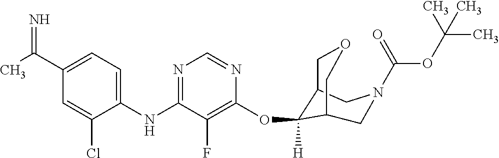 | 506 |
| 367 | 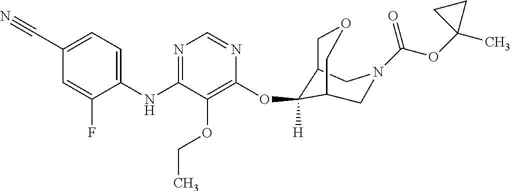 | 499 |
| 368 | 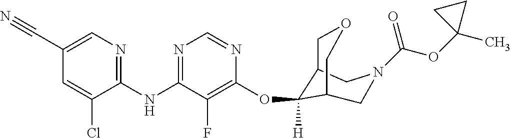 | 489 |
| 369 | 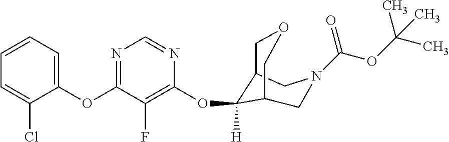 | 466 |
| 370 | 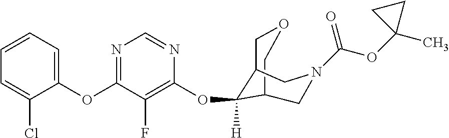 | 464 |
| 371 | 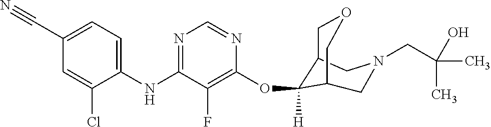 | 462 |
| 372 | 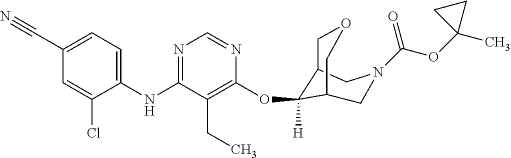 | 498 |
| 373 | 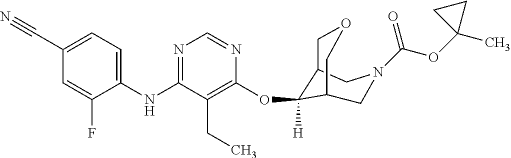 | 483 |
| 374 | 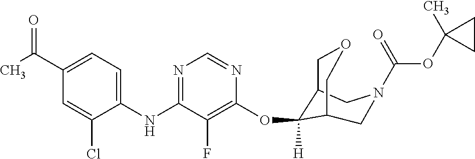 | 505 |
| 375 | 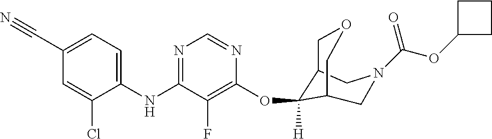 | 488 |
| 376 | 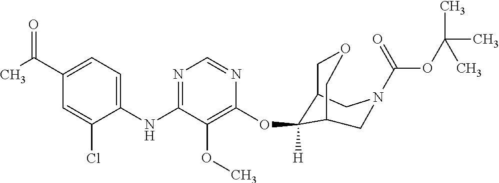 | 519 |
| 377 | 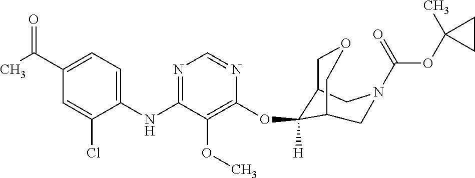 | 517 |
| 378 | 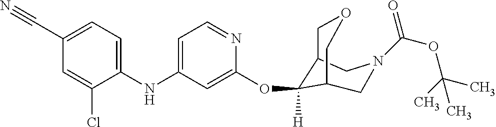 | 471 |
| 379 | 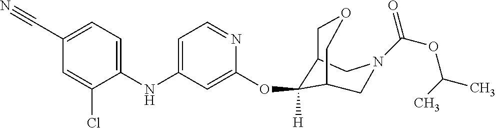 | 457 |
| 380 | 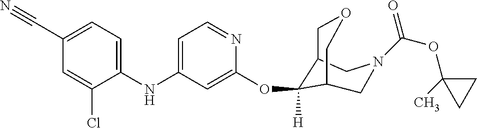 | 469 |
| 381 | 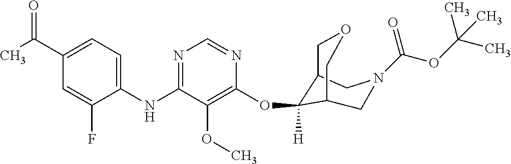 | 504 |
| 382 | 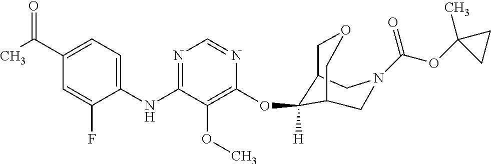 | 502 |
| 383 | 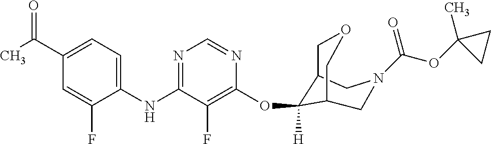 | 489 |
| 384 | 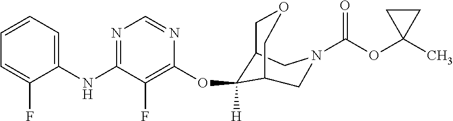 | 447 |
| 385 | 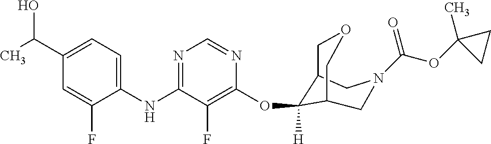 | 491 |
| 386 | 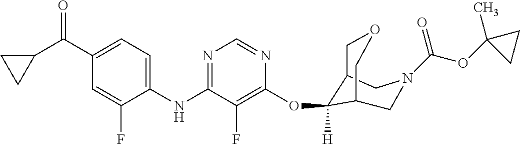 | 516 |
| 387 | 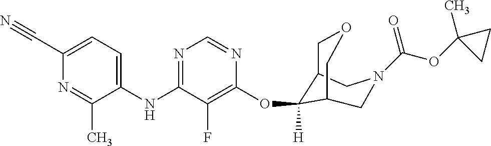 | 469 |
| 388 | 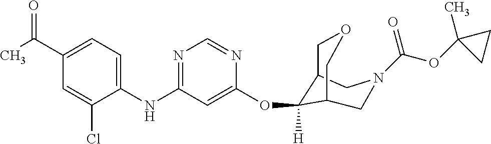 | 487 |
| 389 | 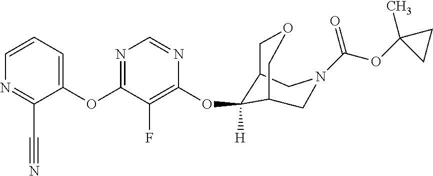 | 456 |
| 390 | 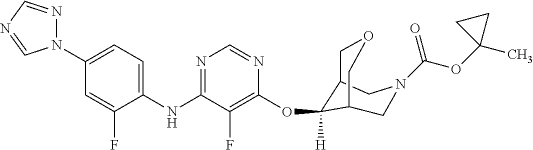 | 514 |
| 391 | 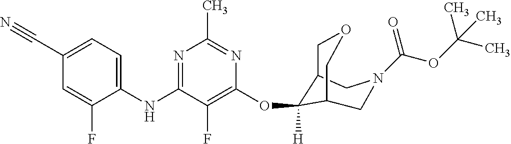 | 489 |
| 392 | 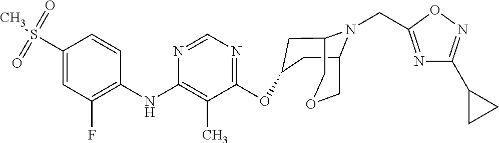 | 546 |
| 393 | 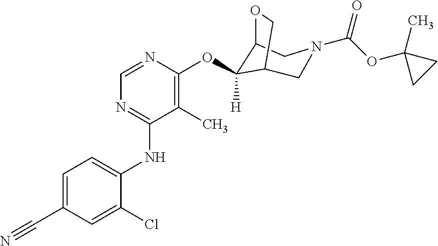 | 470 |
| 394 | 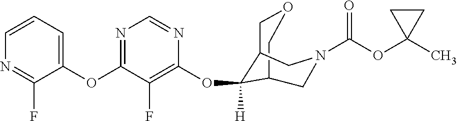 | 465 |
| 395 | 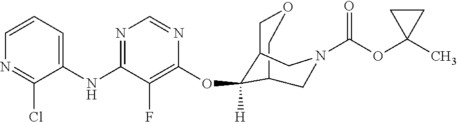 | 464 |
| 396 | 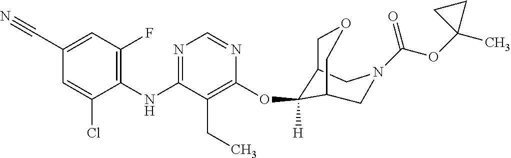 | 516 |
| 397 | 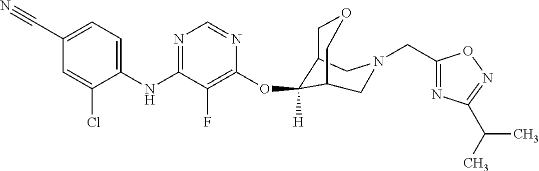 | 514 |
| 398 | 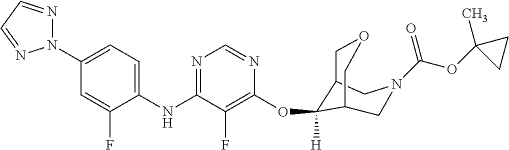 | 514 |
| 399 | 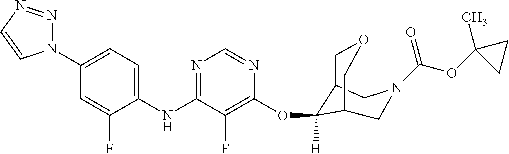 | 514 |
| 400 | 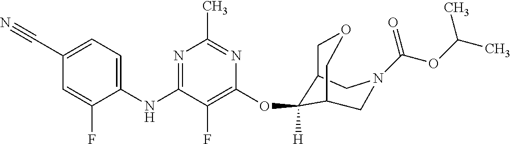 | 475 |
| 401 | 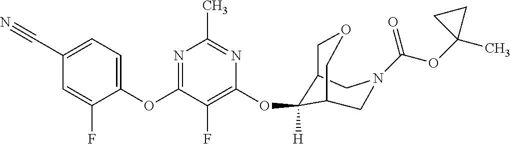 | 487 |
| 402 | 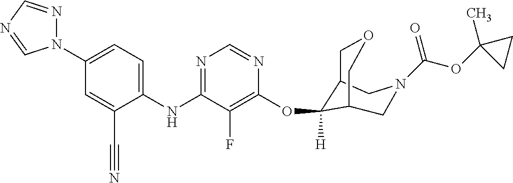 | 522 |
| 403 | 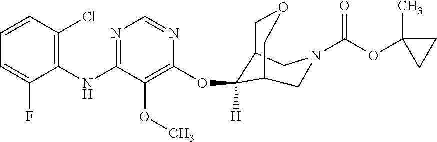 | 493 |
| 404 | 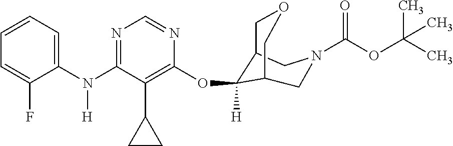 | 472 |
| 405 | 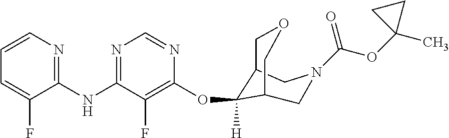 | 448 |
| 406 | 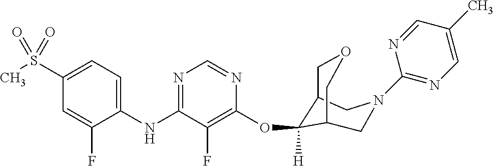 | 520 |
| 407 | 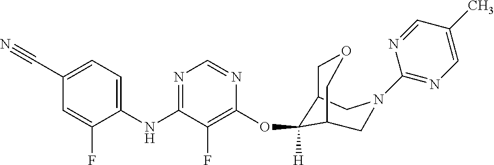 | 466 |
| 408 | 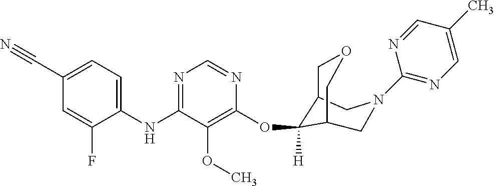 | 478 |
| 409 | 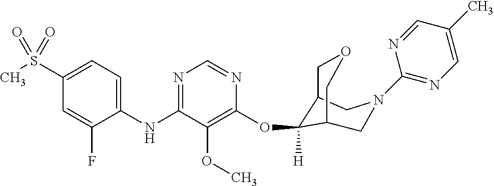 | 532 |
| 410 | 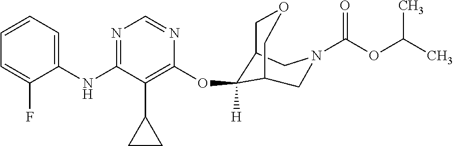 | 458 |
| 411 | 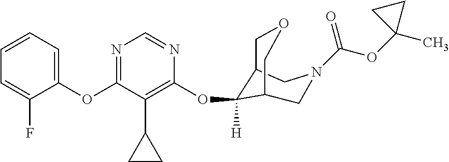 | 470 |
| 412 | 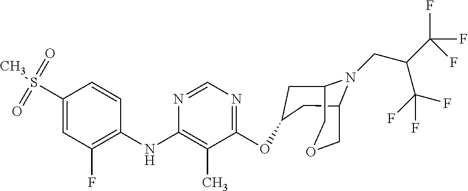 | 588 |
| 413 | 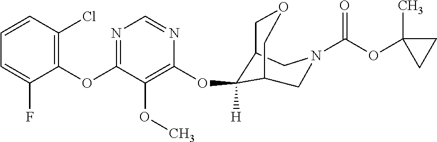 | 494 |
| 414 | 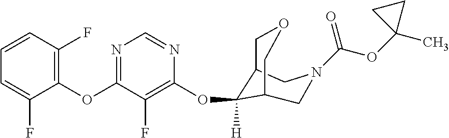 | 465 |
| 415 | 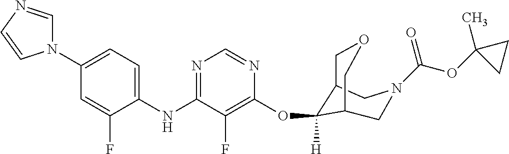 | 514 |
| 416 | 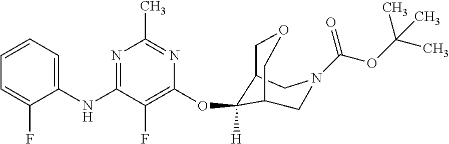 | 463 |
| 417 | 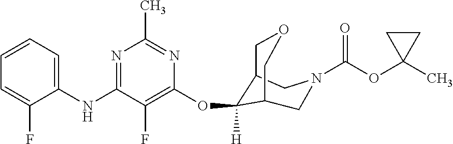 | 461 |
| 418 | 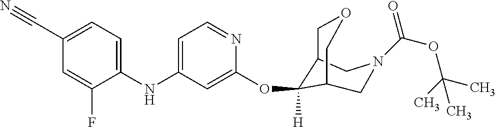 | 455 |
| 419 | 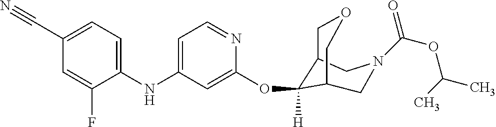 | 441 |
| 420 | 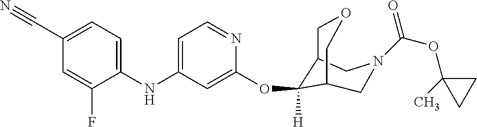 | 453 |
| 421 | 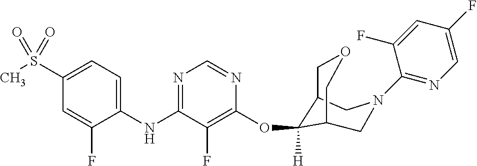 | 541 |
| 422 | 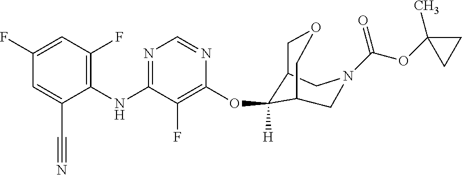 | 490 |
| 423 | 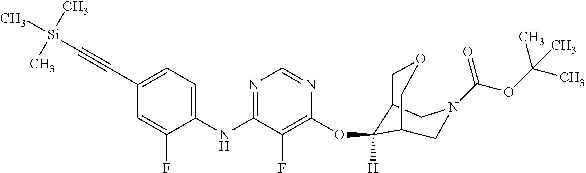 | 546 |
| 424 | 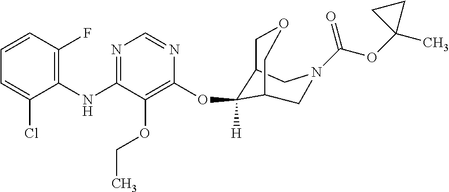 | 507 |
| 425 | 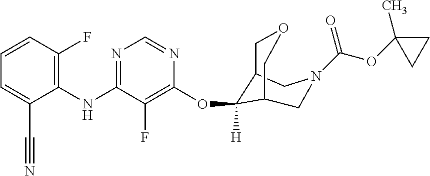 | 472 |
| 426 | 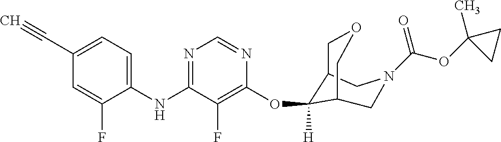 | 471 |
| 427 | 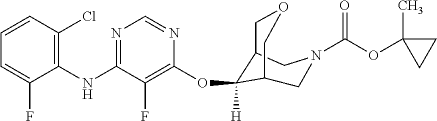 | 481 |
| 428 | 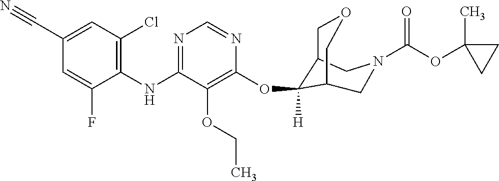 | 532 |
| 429 | 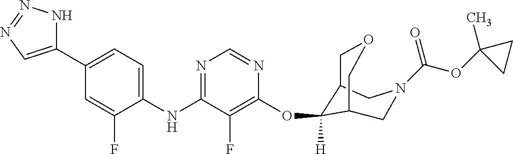 | 514 |
| 430 | 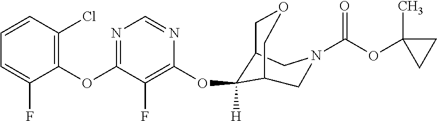 | 482 |
| 431 | 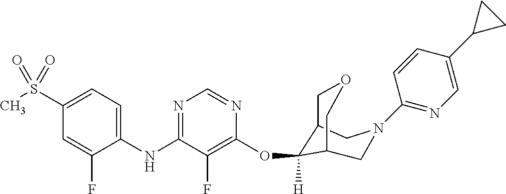 | 545 |
| 432 | 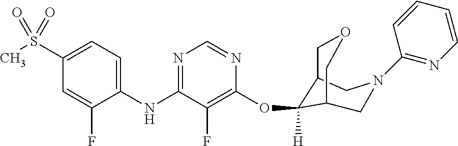 | 505 |
| 433 | 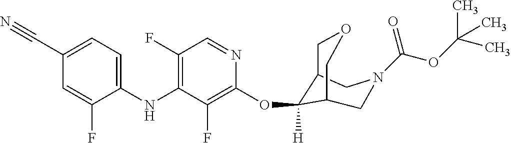 | 491 |
| 434 | 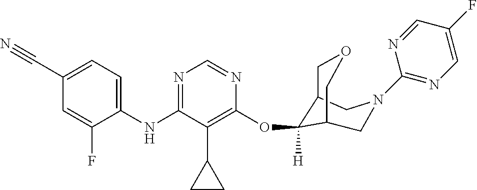 | 492 |
| 435 | 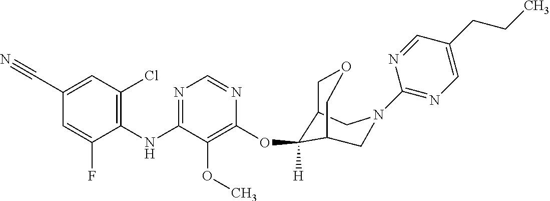 | 541 |
| 436 | 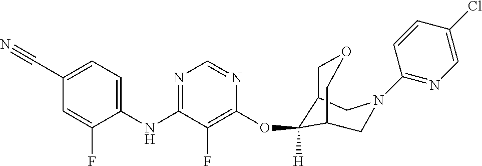 | 485 |
| 437 | 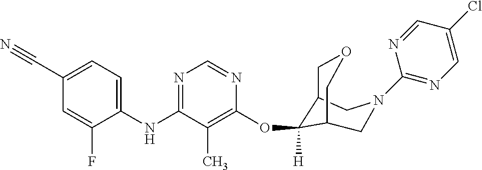 | 482 |
| 438 | 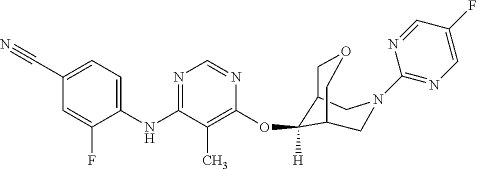 | 466 |
| 439 | 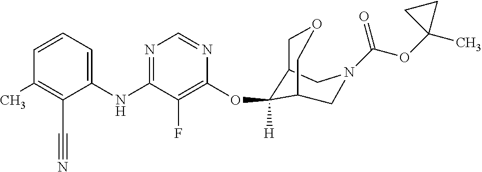 | 468 |
| 440 | 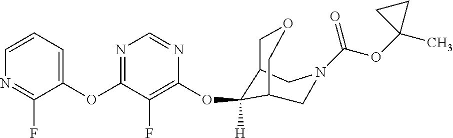 | 448 |
| 441 | 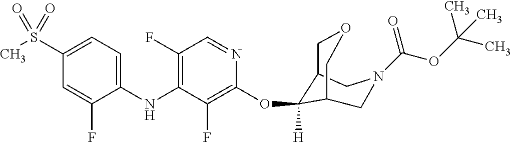 | 545 |
| 442 | 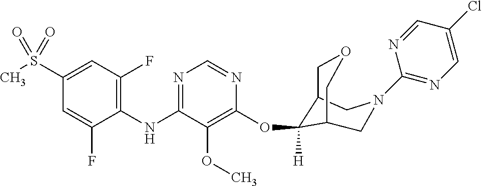 | 569 |
| 443 | 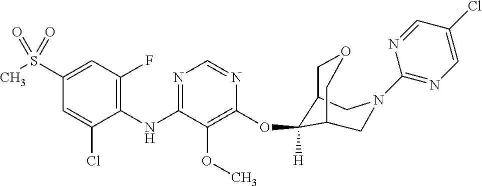 | 585 |
| 444 | 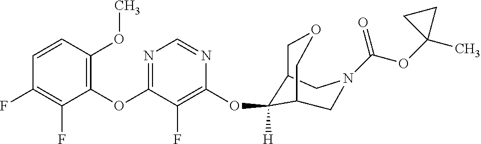 | 495 |
| 445 | 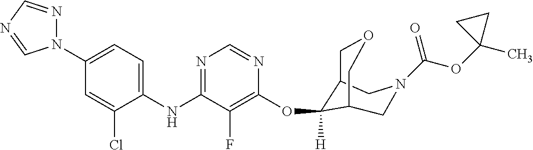 | 530 |
| 446 | 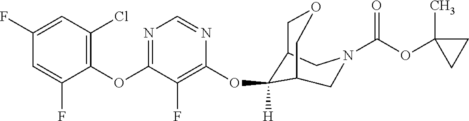 | 499 |
| 447 | 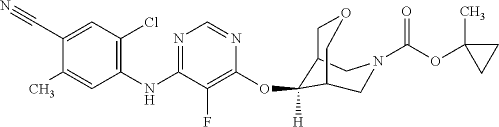 | 502 |
| 448 | 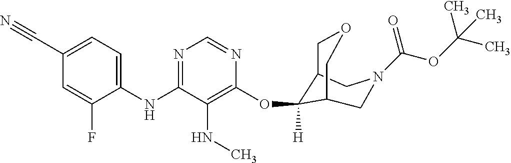 | 486 |
| 449 | 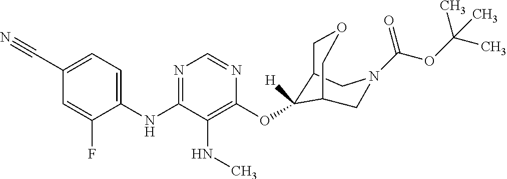 | 486 |
| 450 | 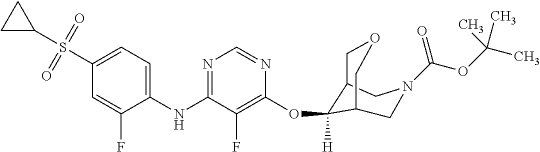 | 554 |
| 451 | 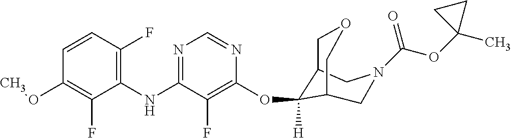 | 495 |
| 452 | 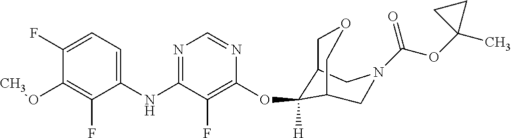 | 495 |
| 453 | 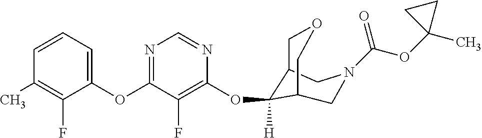 | 462 |
| 454 | 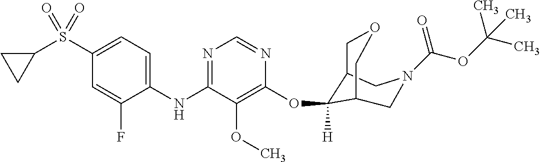 | 566 |
| 455 | 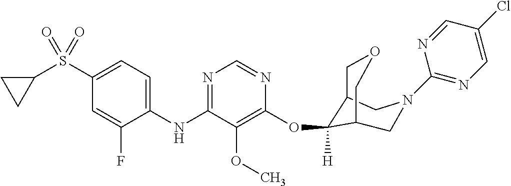 | 577 |
| 456 | 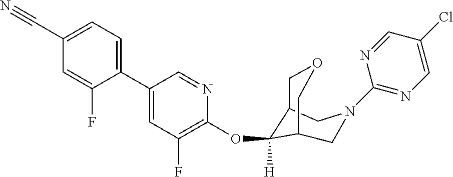 | 470 |
| 457 | 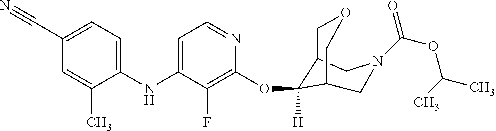 | 455 |
| 458 | 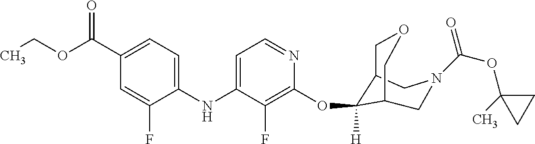 | 519 |
| 459 | 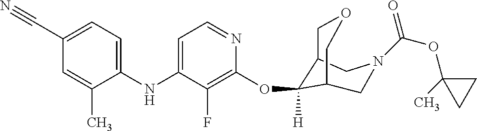 | 468 |
| 460 | 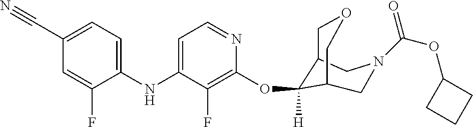 | 471 |
| 461 | 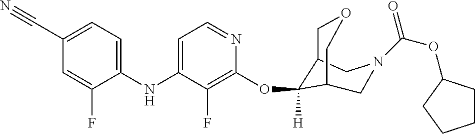 | 485 |
| 462 | 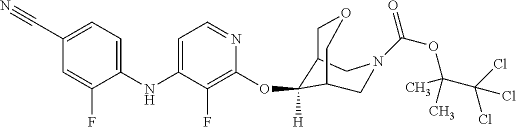 | 576 |
| 463 | 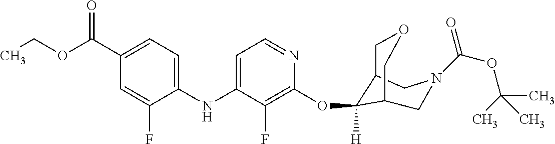 | 521 |
| 464 | 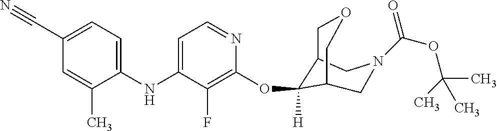 | 470 |
| 465 | 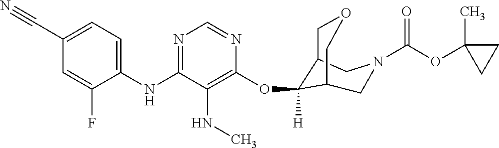 | 484 |
| 466 | 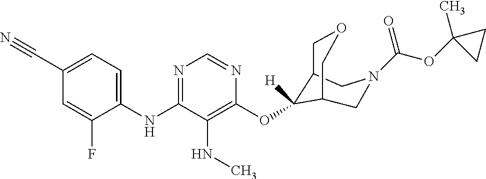 | 484 |
| 467 | 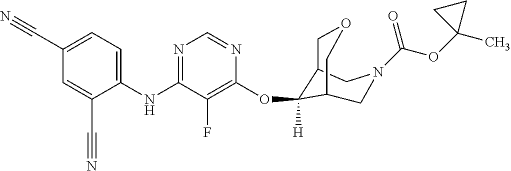 | 479 |
| 468 | 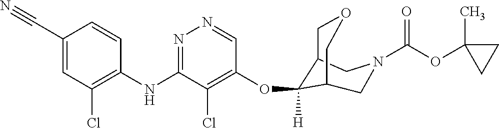 | 504 |
| 469 | 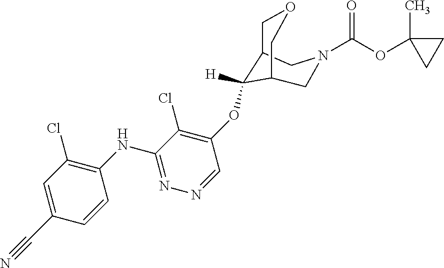 | 504 |
| 470 | 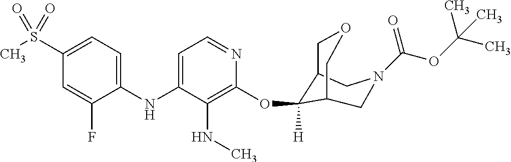 | 539 |
| 471 | 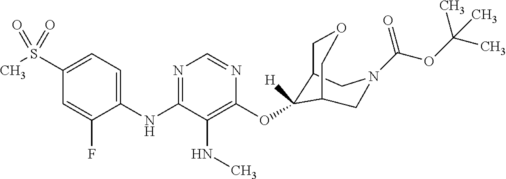 | 539 |
| 472 | 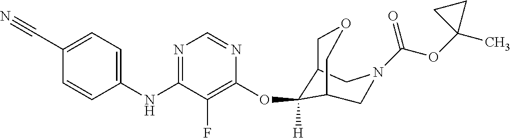 | 454 |
| 473 | 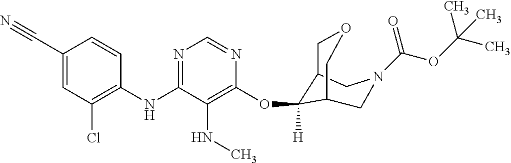 | 501 |
| 474 | 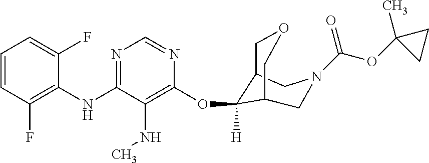 | 476 |
| 475 | 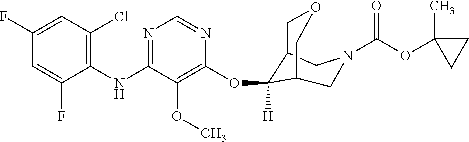 | 511 |
| 476 | 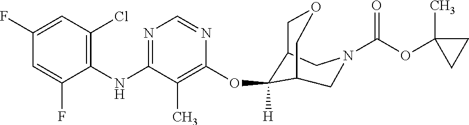 | 495 |
| 477 | 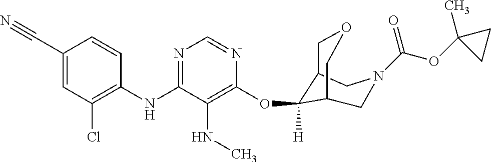 | 499 |
| 478 | 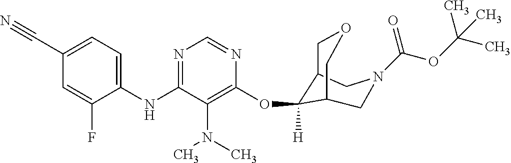 | 500 |
| 479 | 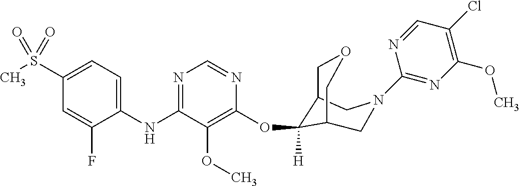 | 581 |
| 480 | 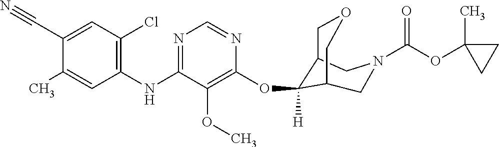 | 514 |
| 481 | 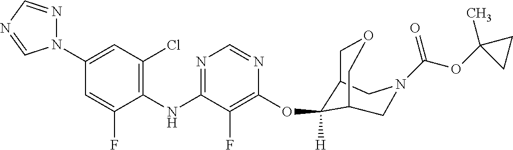 | 548 |
| 482 | 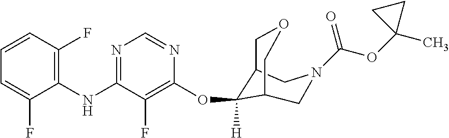 | 466 |
| 483 | 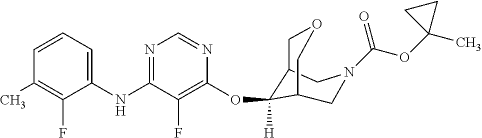 | 461 |
| 484 | 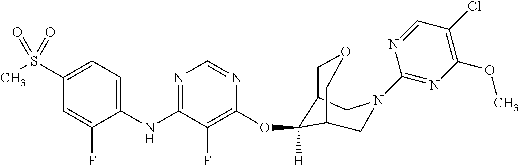 | 569 |
| 485 | 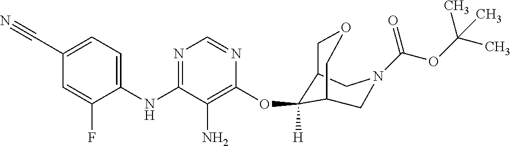 | 471 |
| 486 | 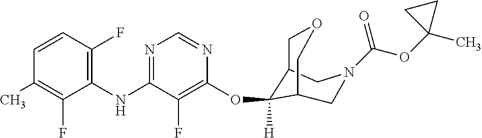 | 479 |
| 487 | 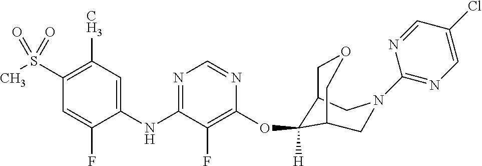 | 553 |
| 488 | 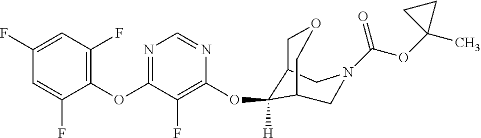 | 484 |
| 489 | 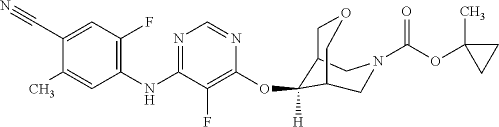 | 486 |
| 490 | 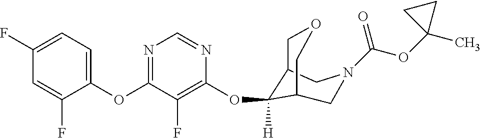 | 466 |
| 491 | 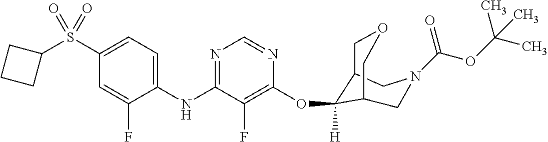 | 568 |
| 492 | 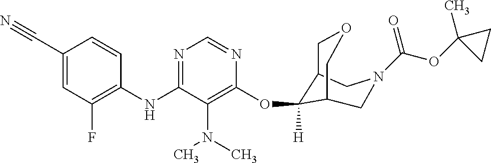 | 498 |
| 493 | 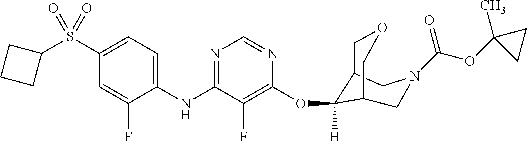 | 566 |
| 494 | 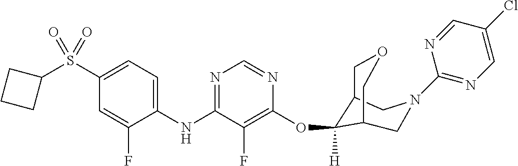 | 579 |
| 495 | 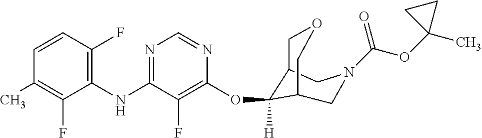 | 480 |
| 496 | 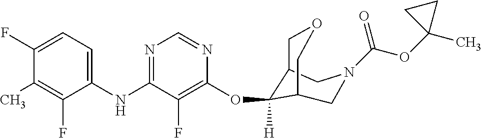 | 479 |
| 497 | 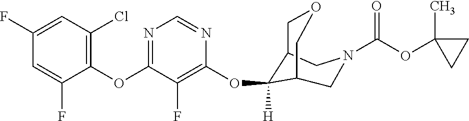 | 500 |
| 498 | 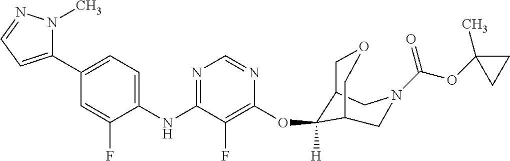 | 528 |
| 499 | 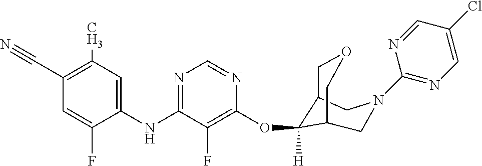 | 500 |
| 500 | 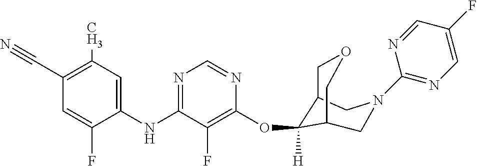 | 484 |
| 501 | 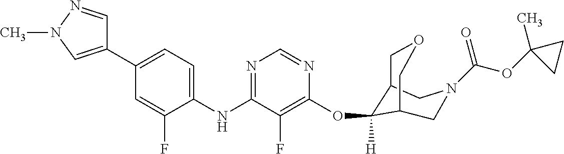 | 528 |
| 502 | 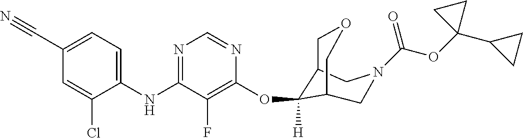 | 514 |
| 503 | 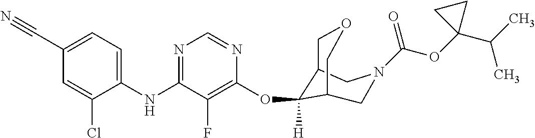 | 516 |
| 504 | 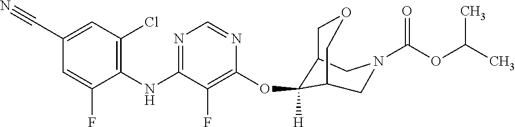 | 494 |
| 505 | 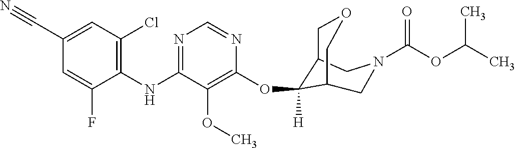 | 506 |
| 506 | 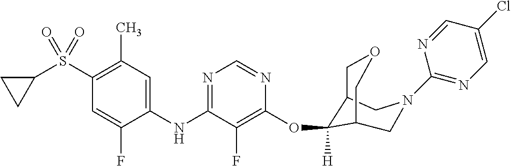 | 579 |
| 507 | 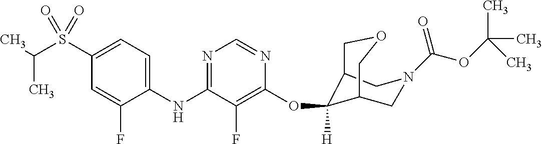 | 556 |
| 508 | 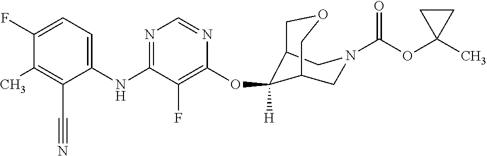 | 486 |
| 509 | 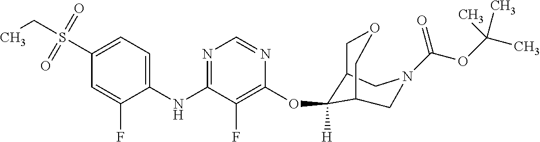 | 542 |
| 510 | 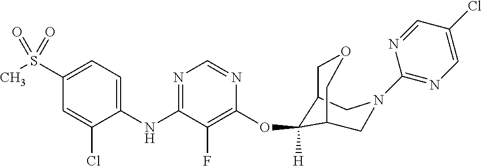 | 555 |
| 511 | 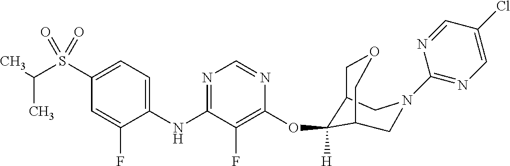 | 567 |
| 512 | 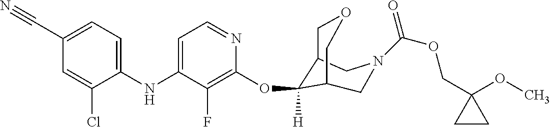 | 518 |
| 513 | 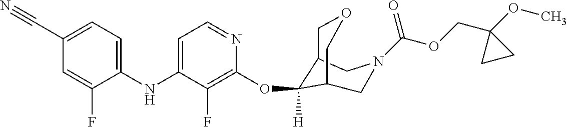 | 501 |
| 514 | 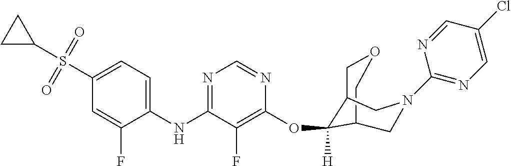 | 565 |
| 515 | 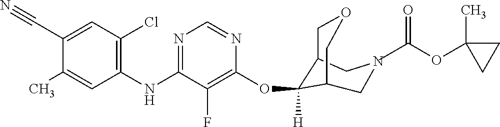 | 503 |
| 516 | 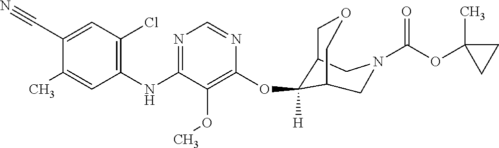 | 515 |
| 517 | 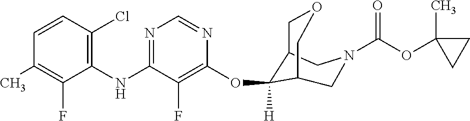 | 495 |
| 518 | 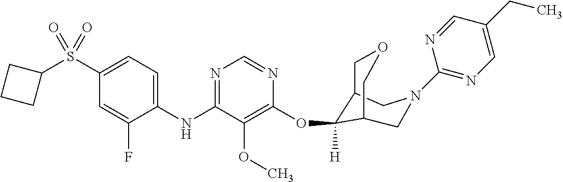 | 586 |
| 519 | 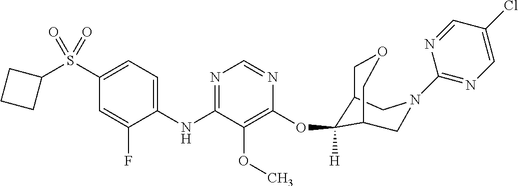 | 591 |
| 520 | 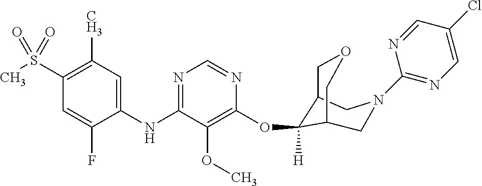 | 565 |
| 521 | 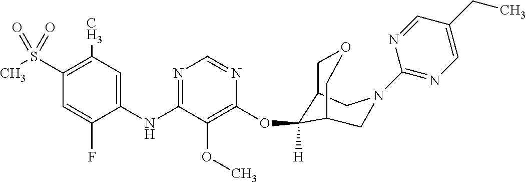 | 560 |
| 522 | 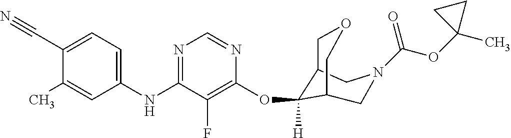 | 468 |
| 523 | 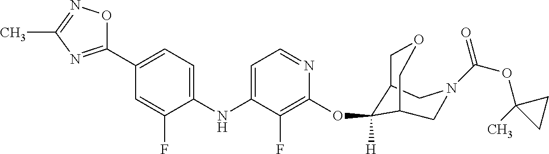 | 529 |
| 524 | 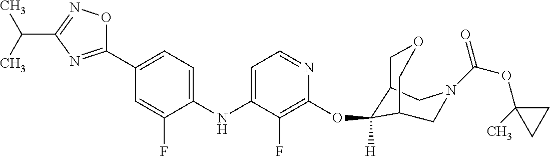 | 557 |
| 525 | 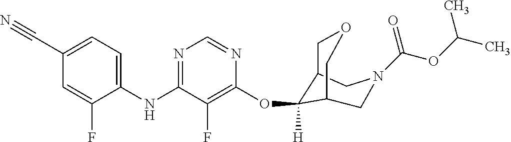 | 460 |
| 526 | 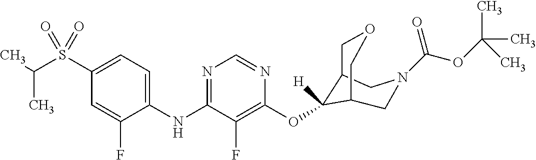 | 556 |
| 527 | 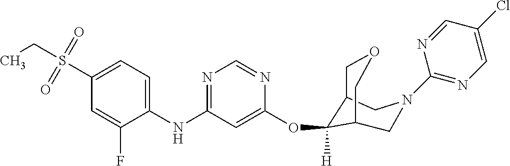 | 535 |
| 528 | 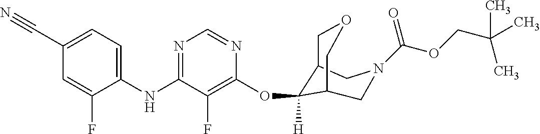 | 488 |
| 529 | 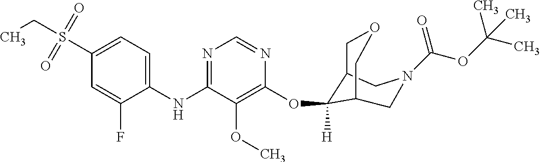 | 554 |
| 530 | 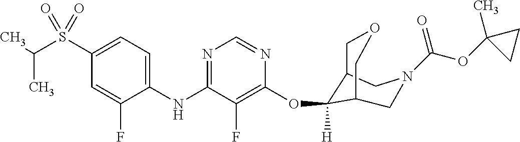 | 554 |
| 531 | 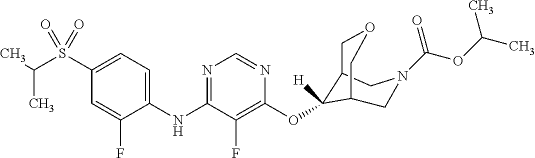 | 542 |
| 532 | 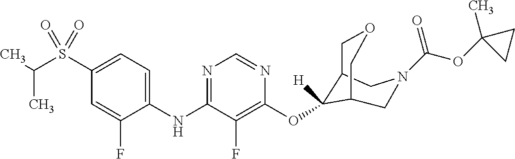 | 554 |
| 533 | 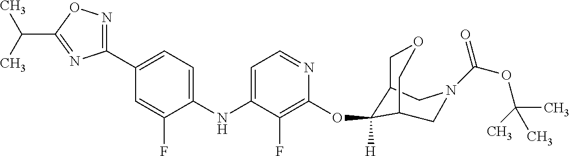 | 559 |
| 534 | 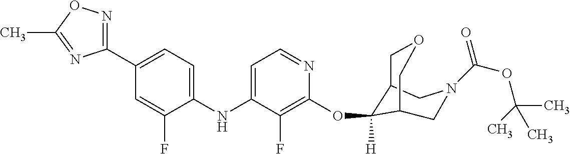 | 531 |
| 535 | 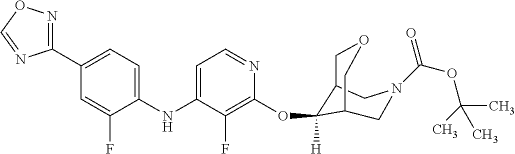 | 517 |
| 536 | 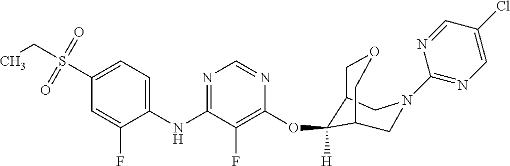 | 553 |
| 537 | 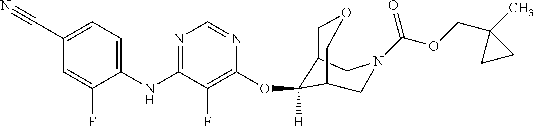 | 486 |
| 538 | 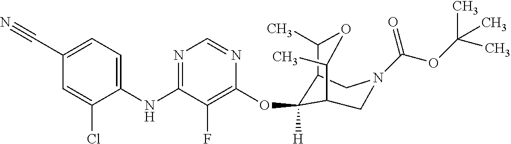 | 519 |
| 539 | 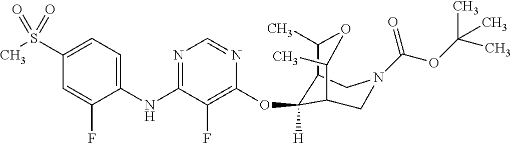 | 556 |
| 540 | 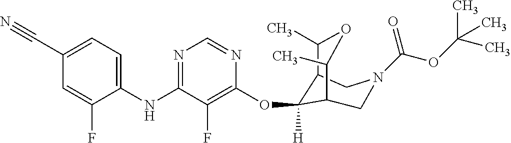 | 503 |
| 541 | 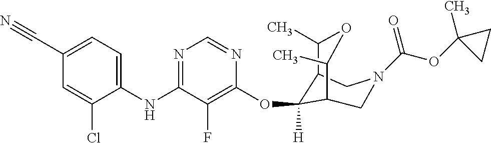 | 516 |
| 542 | 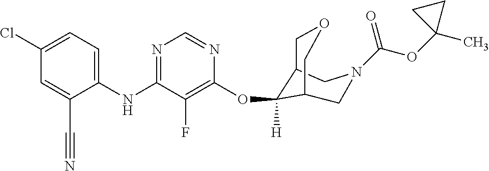 | 488 |
| 543 | 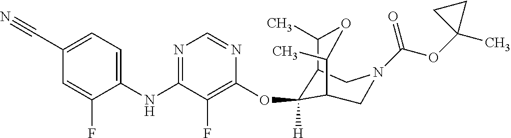 | 501 |
| 544 | 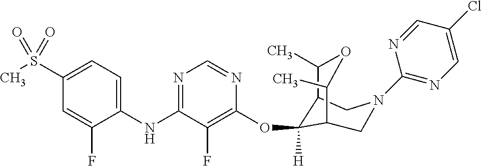 | 567 |
| 545 | 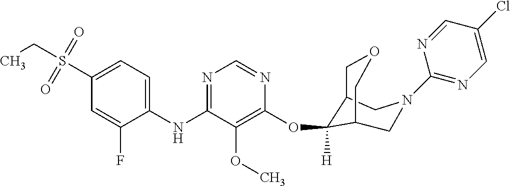 | 565 |
| 546 | 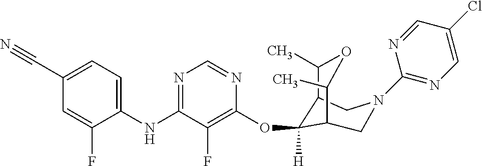 | 514 |
| 547 | 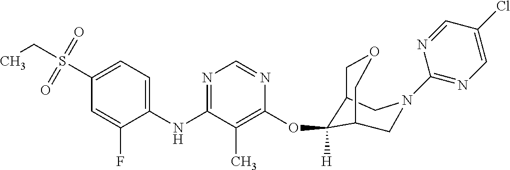 | 549 |
| 548 | 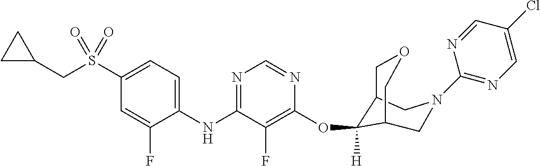 | 579 |
| 549 | 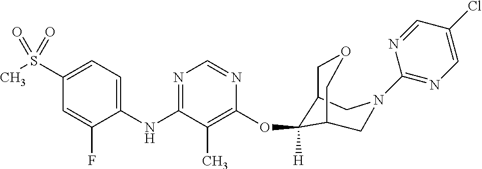 | 535 |
| 550 | 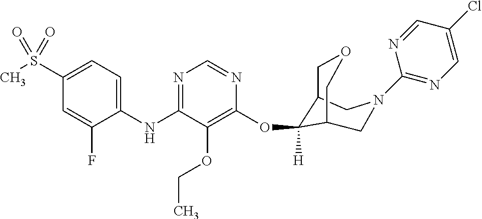 | 565 |
| 551 | 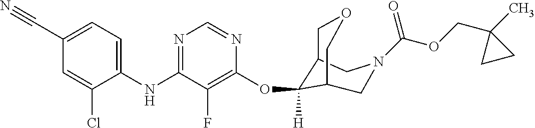 | 502 |
| 552 | 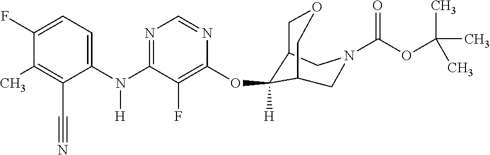 | 488 |
| 553 | 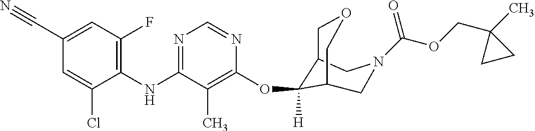 | 516 |
| 554 | 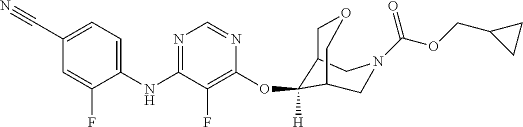 | 472 |
| 555 | 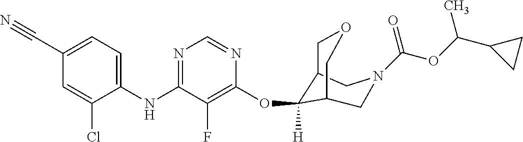 | 502 |
| 556 | 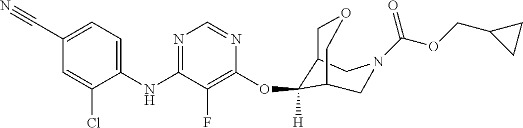 | 488 |
| 557 | 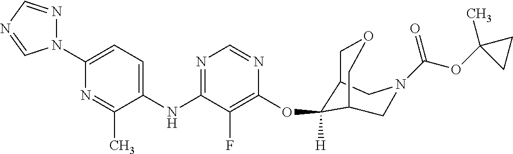 | 512 |
| 558 | 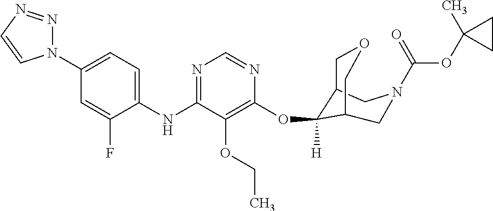 | 541 |
| 559 | 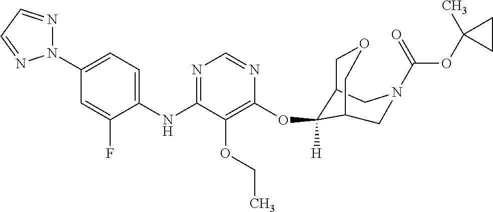 | 541 |
| 560 | 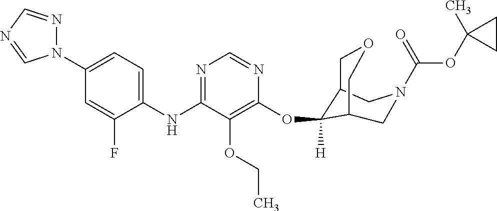 | 541 |
| 561 | 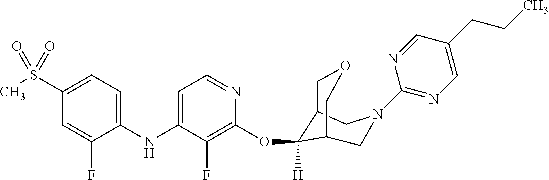 | 547 |
| 562 | 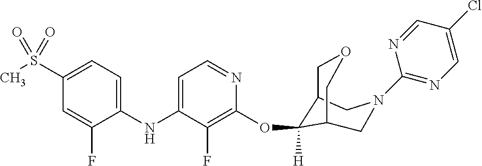 | 538 |
| 563 | 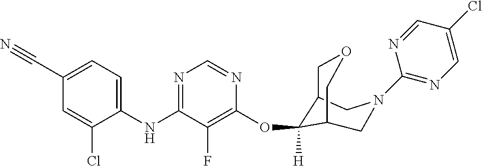 | 502 |
| 564 | 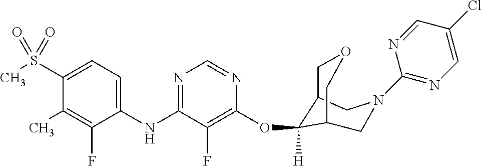 | 553 |
| 565 | 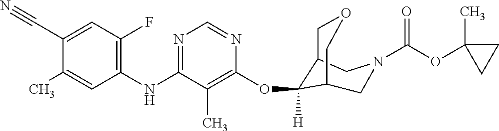 | 483 |
| 566 | 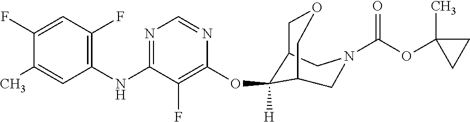 | 479 |
| 567 | 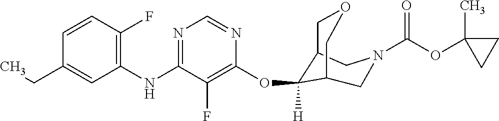 | 476 |
| 568 | 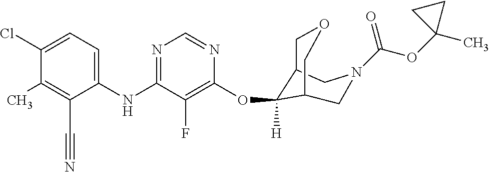 | 502 |
| 569 | 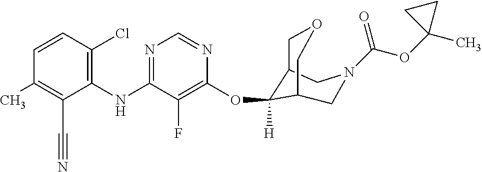 | 502 |
| 570 | 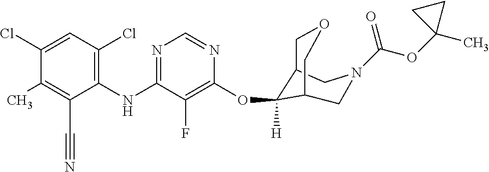 | 536 |
| 571 | 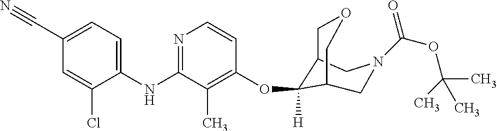 | 485 |
| 572 | 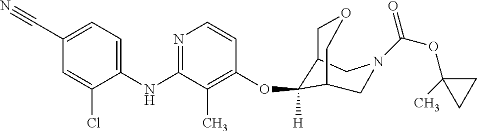 | 483 |
| 573 | 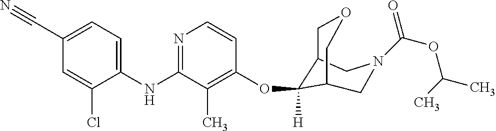 | 471 |
| 574 | 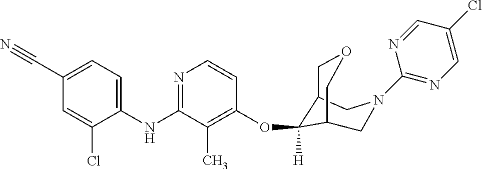 | 497 |
| 575 | 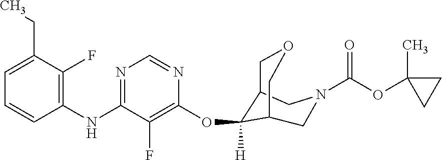 | 476 |
| 576 | 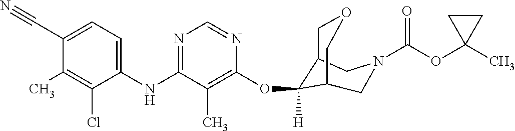 | 502 |
| 577 | 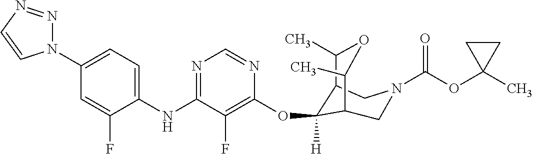 | 543 |
| 578 | 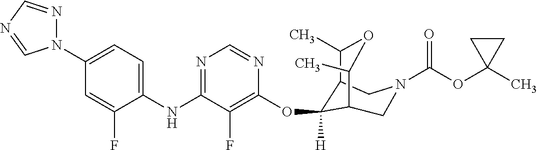 | 543 |
| 579 | 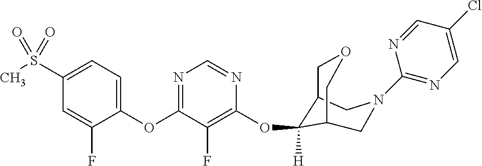 | 540 |
| 580 | 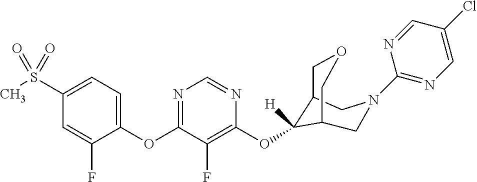 | 540 |
| 581 | 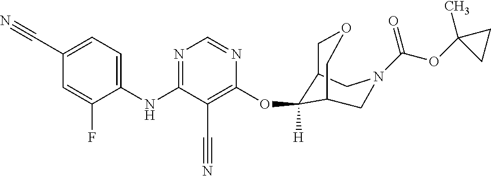 | 479 |
| 582 | 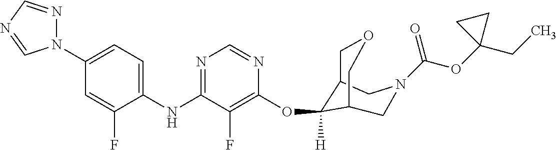 | 529 |
| 583 | 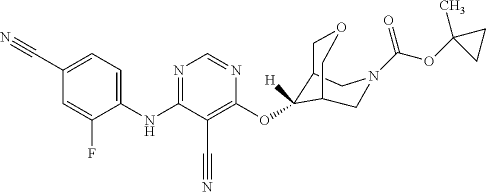 | 479 |
| 584 | 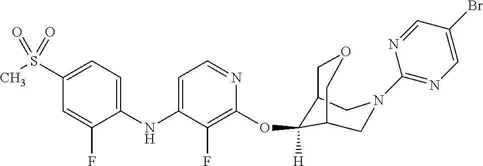 | 583 |
| 585 | 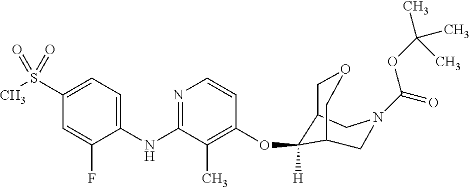 | 523 |
| 586 | 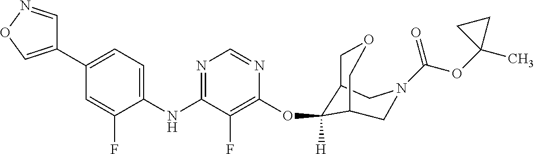 | 514 |
| 587 | 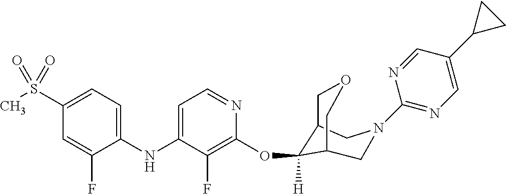 | 545 |
| 588 | 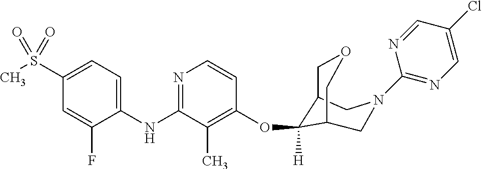 | 534 |
| 589 | 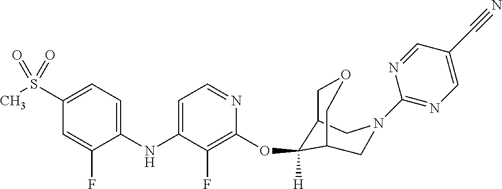 | 530 |
| 590 | 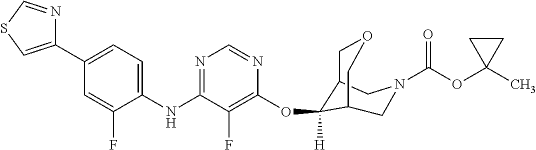 | 531 |
| 591 | 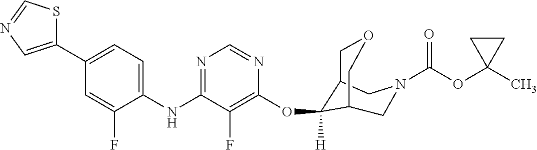 | 531 |
| 592 | 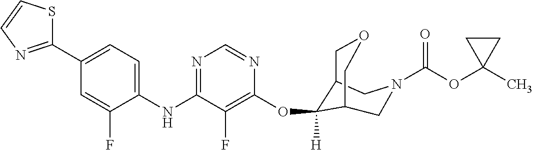 | 531 |
| 593 | 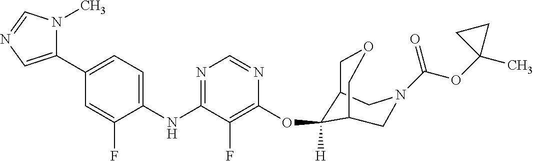 | 528 |
| 594 | 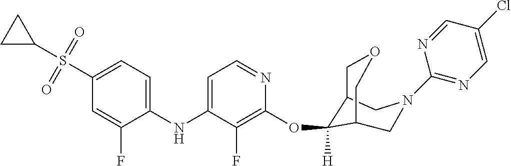 | 564 |
| 595 | 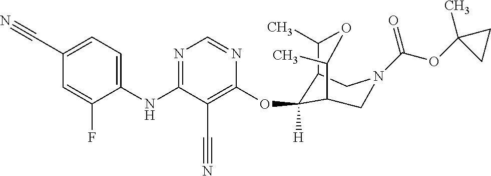 | 508 |
| 596 | 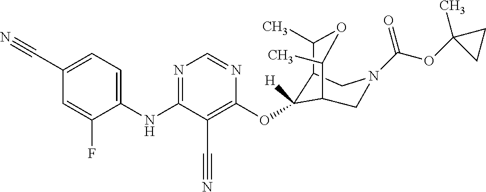 | 508 |
| 597 | 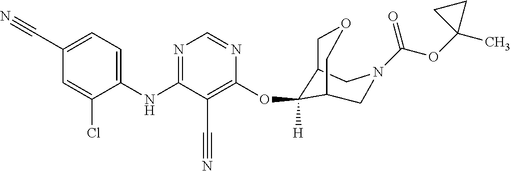 | 496 |
| 598 | 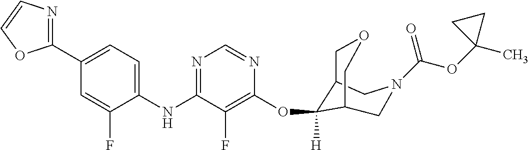 | 514 |
| 599 | 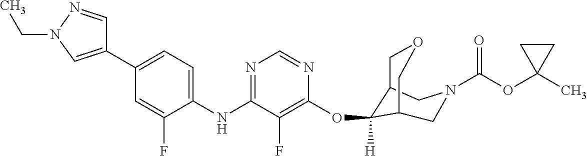 | 542 |
| 600 | 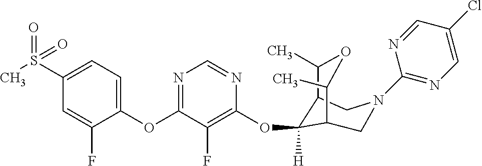 | 568 |
| 601 | 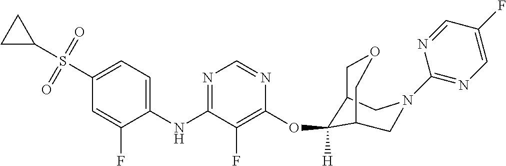 | 550 |
| 602 | 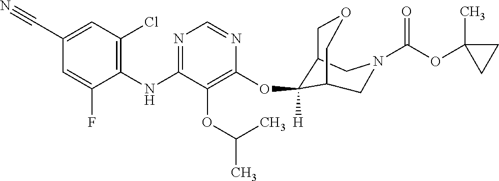 | 546 |
| 603 | 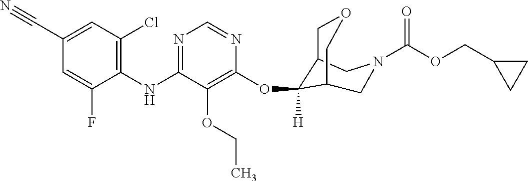 | 532 |
| 604 | 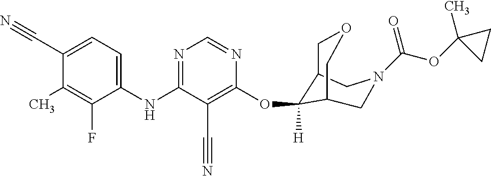 | 494 |
| 605 | 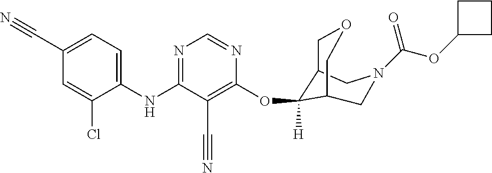 | 495 |
| 606 | 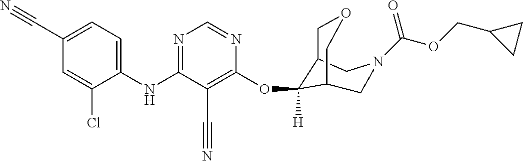 | 495 |
| 607 | 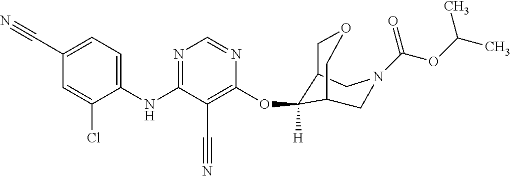 | 483 |

### Example 66

### cAMP Assay

The ability of illustrative compounds of the invention to activate GPR119 and stimulate increases in cAMP levels was determined using the LANCE™ cAMP kit (Perkin Elmer). HEK293 cells expressing human GPR119 were maintained in culture flasks at 37° C./5% CO₂ in DMEM containing 10% fetal bovine serum, 100 U/ml Pen/Strep, and 0.5 mg/ml geneticin. The media was changed to Optimem and cells were incubated for about 15 hours at 37° C./5% CO₂. The Optimem was then aspirated and the cells were removed from the flasks using room temperature Hank's balanced saline solution (HBSS). The cells were pelleted using centrifugation (1300 rpm, 7 minutes, room temperature), then resuspended in stimulation buffer (HBSS, 0.1% BSA, 5 mM HEPES, 15 μM RO-20) at 2.5×10⁶ cells/mL. Alexa Fluor 647-anti cAMP antibody (1:100) was then added to the cell suspension and incubated for 30 minutes. A representative Bridged Bicyclic Heterocycle Derivative (6 μl at 2× concentration) in stimulation buffer containing 2% DMSO were then added to white 384 well Matrix plates. Cell suspension mix (6 μl) was added to each well and incubated with the Bridged Bicyclic Heterocycle Derivative for 30 minutes. A cAMP standard curve was also created in each assay according to the kit protocol. Standard concentrations of cAMP in stimulation buffer (6 μl) were added to white 384 well plates. Subsequently, 6 μl of 1:100 anti-cAMP antibody was added to each well. Following the 30 minute incubation period, 12 μl of detection mix (included in kit) was added to all wells and incubated for 2-3 hours at room temperature. Fluorescence was detected on the plates using an Envision instrument. The level of cAMP in each well is determined by extrapolation from the cAMP standard curve.

Using this assay, EC₅₀ values for various illustrative Bridged Bicyclic Heterocycle Derivatives of the present invention were calculated and range from about 3 nM to about 200 nM.

### Example 67

### Effect of the Compounds of the Invention in Oral Glucose Tolerance Test

Male C57BI/6NCrI mice (6-8 week old) were fasted overnight and randomly dosed with either vehicle (20% hydroxypropyl-β-cyclodextrin) or a representative compound of the invention (at 3, 10 or 30 mg/kg) via oral gavage (n=8 mice/group). Glucose was administered to the animals 30 minutes post-dosing (3 g/kg p.o.). Blood glucose was measured prior to administration of test compound and glucose, and at 20 minutes after glucose administration using a hand-held glucometer (Ascensia Elite, Bayer).

Using this protocol, the effects of various Bridged Bicyclic Heterocycle Derivatives of the present invention were measured and the results indicated that the Bridged Bicyclic Heterocycle Derivatives of the present invention were effective in lowering blood glucose levels after glucose challenge at concentrations ranging from 0.1 mg/kg to 30 mg/kg.

### Example 68

### Effect of the Compounds of the Invention in an Animal Model of Diabetes

Four week old male C57BI/6NCrI mice can be used to generate a nongenetic model of type 2 diabetes mellitus as previously described (Metabolism 47(6): 663-668, 1998). Briefly, mice are made insulin resistant by high fat feeding (60% of kcal as fat) and hyperglycemia is then induced using a low dose of streptozotocin (100 mg/kg i.p.). Eight weeks after streptozotocin administration, the diabetic mice are placed into one of 4 groups (n=13/gp) receiving the following treatments: vehicle (20% hydroxypropyl-β-cyclodextrin p.o.), compound to be tested (30 mg/kg p.o.), glipizide (20 mg/kg p.o.) or exendin-4 (10 ug/kg i.p.). Mice are dosed once daily for 13 consecutive days, and blood glucose levels are measured daily using, for example, a hand held glucometer, to determine the effects of the test compound(s) on glucose levels of the diabetic animals.

### Uses of the Bridged Bicyclic Heterocycle Derivatives

The Bridged Bicyclic Heterocycle Derivatives are useful in human and veterinary medicine for treating or preventing a Condition in a patient. In accordance with the invention, the Bridged Bicyclic Heterocycle Derivatives can be administered to a patient in need of treatment or prevention of a Condition.

In one embodiment, the present invention provides for the use of a Compound of Formula (I) or a pharmaceutically acceptable salt thereof, in the manufacture of a medicament for use in treating a condition selected from the group consisting of obesity, diabetes, a diabetic complication, a metabolic disorder, a cardiovascular disease or a disorder related to the activity of a G-Protein Coupled Receptor (“GPCR”) in a patient.

### Treatment of Obesity and Obesity-Related Disorders

The Bridged Bicyclic Heterocycle Derivatives can also be useful for treating obesity or an obesity-related disorder.

Accordingly, in one embodiment, the invention provides methods for treating obesity or an obesity-related disorder in a patient, wherein the method comprises administering to the patient an effective amount of one or more Bridged Bicyclic Heterocycle Derivatives, or a pharmaceutically acceptable salt, solvate, ester, prodrug or stereoisomer thereof.

### Treatment of Diabetes

The Bridged Bicyclic Heterocycle Derivatives are useful for treating diabetes in a patient. Accordingly, in one embodiment, the present invention provides a method for treating diabetes in a patient, comprising administering to the patient an effective amount of one or more Bridged Bicyclic Heterocycle Derivatives.

Examples of diabetes treatable or preventable using the Bridged Bicyclic Heterocycle Derivatives include, but are not limited to, type I diabetes (insulin-dependent diabetes mellitus), type II diabetes (non-insulin dependent diabetes mellitus), gestational diabetes, autoimmune diabetes, insulinopathies, idiopathic type I diabetes (Type 1b), latent autoimmumne diabetes in adults, early-onset type 2 diabetes (EOD), youth-onset atypical diabetes (YOAD), maturity onset diabetes of the young (MODY), malnutrition-related diabetes, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by β-cell toxins, and diabetes induced by drug therapy (such as diabetes induced by antipsychotic agents).

In one embodiment, the diabetes is type I diabetes.

In another embodiment, the diabetes is type II diabetes.

### Treatment of a Diabetic Complication

The Bridged Bicyclic Heterocycle Derivatives are also useful for treating a diabetic complication in a patient. Accordingly, in one embodiment, the present invention provides a method for treating a diabetic complication in a patient, comprising administering to the patient an effective amount of one or more Bridged Bicyclic Heterocycle Derivatives.

Examples of diabetic complications treatable or preventable using the Bridged Bicyclic Heterocycle Derivatives include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, cataract, hypertension, syndrome of insulin resistance, coronary artery disease, a fungal infection, a bacterial infection, and cardiomyopathy.

### Treatment of a Metabolic Disorder

The Bridged Bicyclic Heterocycle Derivatives can also be useful for treating a metabolic disorder. Examples of metabolic disorders treatable include, but are not limited to, metabolic syndrome (also known as “Syndrome X”), impaired glucose tolerance, impaired fasting glucose, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

Accordingly, in one embodiment, the invention provides methods for treating a metabolic disorder in a patient, wherein the method comprises administering to the patient an effective amount of one or more Bridged Bicyclic Heterocycle Derivatives, or a pharmaceutically acceptable salt, solvate, ester, prodrug or stereoisomer thereof.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

### Methods for Treating a Cardiovascular Disease

The Bridged Bicyclic Heterocycle Derivatives are useful for treating or preventing a cardiovascular disease in a patient.

Accordingly, in one embodiment, the present invention provides a method for treating a cardiovascular disease in a patient, comprising administering to the patient an effective amount of one or more Bridged Bicyclic Heterocycle Derivatives.

Illustrative examples of cardiovascular diseases treatable or preventable using the present methods, include, but are not limited to atherosclerosis, congestive heart failure, cardiac arrhythmia, myocardial infarction, atrial fibrillation, atrial flutter, circulatory shock, left ventricular hypertrophy, ventricular tachycardia, supraventricular tachycardia, coronary artery disease, angina, infective endocarditis, non-infective endocarditis, cardiomyopathy, peripheral artery disease, Reynaud's phenomenon, deep venous thrombosis, aortic stenosis, mitral stenosis, pulmonic stenosis and tricuspid stenosis.

In one embodiment, the cardiovascular disease is atherosclerosis.

In another embodiment, the cardiovascular disease is congestive heart failure.

In another embodiment, the cardiovascular disease is coronary artery disease.

### Combination Therapy

In one embodiment, the present invention provides methods for treating a Condition in a patient, the method comprising administering to the patient one or more Bridged Bicyclic Heterocycle Derivatives, or a pharmaceutically acceptable salt, solvate, ester, prodrug or stereoisomer thereof and at least one additional therapeutic agent that is not a Bridged Bicyclic Heterocycle Derivative, wherein the amounts administered are together effective to treat or prevent a Condition.

Non-limiting examples of additional therapeutic agents useful in the present methods for treating or preventing a Condition include, anti-obesity agents, antidiabetic agents, any agent useful for treating metabolic syndrome, any agent useful for treating a cardiovascular disease, cholesterol biosynthesis inhibitors, cholesterol absorption inhibitors, bile acid sequestrants, probucol derivatives, IBAT inhibitors, nicotinic acid receptor (NAR) agonists, ACAT inhibitors, cholesteryl ester transfer proten (CETP) inhibitors, low-denisity lipoprotein (LDL) activators, fish oil, water-soluble fibers, plant sterols, plant stanols, fatty acid esters of plant stanols, or any combination of two or more of these additional therapeutic agents.

Non-limiting examples of anti-obesity agents useful in the present methods for treating a Condition include CB1 antagonists or inverse agonists such as rimonabant, neuropeptide Y antagonists, MCR4 agonists, MCH receptor antagonists, histamine H₃ receptor antagonists or inverse agonists, metabolic rate enhancers, nutrient absorption inhibitors, leptin, appetite suppressants and lipase inhibitors.

Non-limiting examples of appetite suppressant agents useful in the present methods for treating or preventing a Condition include cannabinoid receptor 1 (CB₁) antagonists or inverse agonists (e.g., rimonabant); Neuropeptide Y (NPY1, NPY2, NPY4 and NPY5) antagonists; metabotropic glutamate subtype 5 receptor (mGluR5) antagonists (e.g., 2-methyl-6-(phenylethynyl)-pyridine and 3[(2-methyl-1,4-thiazol-4-yl)ethynyl]pyridine); melanin-concentrating hormone receptor (MCH1R and MCH2R) antagonists; melanocortin receptor agonists (e.g., Melanotan-II and Mc4r agonists); serotonin uptake inhibitors (e.g., dexfenfluramine and fluoxetine); serotonin (5HT) transport inhibitors (e.g., paroxetine, fluoxetine, fenfluramine, fluvoxamine, sertaline and imipramine); norepinephrine (NE) transporter inhibitors (e.g., desipramine, talsupram and nomifensine); ghrelin antagonists; leptin or derivatives thereof; opioid antagonists (e.g., nalmefene, 3-methoxynaltrexone, naloxone and nalterxone); orexin antagonists; bombesin receptor subtype 3 (BRS3) agonists; Cholecystokinin-A (CCK-A) agonists; ciliary neurotrophic factor (CNTF) or derivatives thereof (e.g., butabindide and axokine); monoamine reuptake inhibitors (e.g., sibutramine); glucagon-like peptide 1 (GLP-1) agonists; topiramate; and phytopharm compound 57.

Non-limiting examples of metabolic rate enhancers useful in the present methods for treating or preventing a Condition include acetyl-CoA carboxylase-2 (ACC2) inhibitors; beta adrenergic receptor 3 (β3) agonists; diacylglycerol acyltransferase inhibitors (DGAT1 and DGAT2); fatty acid synthase (FAS) inhibitors (e.g., Cerulenin); phosphodiesterase (PDE) inhibitors (e.g., theophylline, pentoxifylline, zaprinast, sildenafil, aminone, milrinone, cilostamide, rolipram and cilomilast); thyroid hormone β agonists; uncoupling protein activators (UCP-1, 2 or 3) (e.g., phytanic acid, 4-[(E)-2-(5,6,7,8-tetramethyl-2-naphthalenyl)-1-propenyl]benzoic acid and retinoic acid); acyl-estrogens (e.g., oleoyl-estrone); glucocorticoid antagonists; 11-beta hydroxy steroid dehydrogenase type 1 (11β HSD-1) inhibitors; melanocortin-3 receptor (Mc3r) agonists; and stearoyl-CoA desaturase-1 (SCD-1) compounds.

Non-limiting examples of nutrient absorption inhibitors useful in the present methods for treating or preventing a Condition include lipase inhibitors (e.g., orlistat, lipstatin, tetrahydrolipstatin, teasaponin and diethylumbelliferyl phosphate); fatty acid transporter inhibitors; dicarboxylate transporter inhibitors; glucose transporter inhibitors; and phosphate transporter inhibitors.

Non-limiting examples of cholesterol biosynthesis inhibitors useful in the present methods for treating or preventing a Condition include HMG-CoA reductase inhibitors, squalene synthase inhibitors, squalene epoxidase inhibitors, and mixtures thereof.

Non-limiting examples of cholesterol absorption inhibitors useful in the present methods for treating or preventing a Condition include ezetimibe. In one embodiment, the cholesterol absorption inhibitor is ezetimibe.

HMG-CoA reductase inhibitors useful in the present methods for treating or preventing a Condition include, but are not limited to, statins such as lovastatin, pravastatin, fluvastatin, simvastatin, atorvastatin, cerivastatin, CI-981, resuvastatin, rivastatin, pitavastatin, rosuvastatin or L-659,699 ((E,E)-11-[3′R-(hydroxy-methyl)-4′-oxo-2′R-oxetanyl]-3,5,7R-trimethyl-2,4-undecadienoic acid).

Squalene synthesis inhibitors useful in the present methods for treating or preventing a Condition include, but are not limited to, squalene synthetase inhibitors; squalestatin 1; and squalene epoxidase inhibitors, such as NB-598 ((E)-N-ethyl-N-(6,6-dimethyl-2-hepten-4-ynyl)-3-[(3,3′-bithiophen-5-yl)methoxy]benzene-methanamine hydrochloride).

Bile acid sequestrants useful in the present methods for treating or preventing a Condition include, but are not limited to, cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl)alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Probucol derivatives useful in the present methods for treating or preventing a Condition include, but are not limited to, AGI-1067 and others disclosed in U.S. Pat. Nos. 6,121,319 and 6,147,250.

IBAT inhibitors useful in the present methods for treating or preventing a Condition include, but are not limited to, benzothiepines such as therapeutic compounds comprising a 2,3,4,5-tetrahydro-1-benzothiepine 1,1-dioxide structure such as are disclosed in International Publication No. WO 00/38727.

Nicotinic acid receptor agonists useful in the present methods for treating or preventing a Condition include, but are not limited to, those having a pyridine-3-carboxylate structure or a pyrazine-2-carboxylate structure, including acid forms, salts, esters, zwitterions and tautomers, where available. Other examples of nicotinic acid receptor agonists useful in the present methods include nicotinic acid, niceritrol, nicofuranose and acipimox. An example of a suitable nicotinic acid product is NIASPAN® (niacin extended-release tablets) which are available from Kos Pharmaceuticals, Inc. (Cranbury, N.J.). Further nicotinic acid receptor agonists useful in the present methods for treating or preventing a Condition include, but are not limited to, the compounds disclosed in U.S. Patent Publication Nos. 2006/0264489 and 2007/0066630, and U.S. patent application Ser. No. 11/771,538, each of which is incorporated herein by reference.

ACAT inhibitors useful in the present methods for treating or preventing a Condition include, but are not limited to, avasimibe, HL-004, lecimibide and CL-277082 (N-(2,4-difluorophenyl)-N-[[4-(2,2-dimethylpropyl)phenyl]-methyl]-N-heptylurea). See P. Chang et al., “Current, New and Future Treatments in Dyslipidaemia and Atherosclerosis”, Drugs 2000 July, 60(1); 55-93, which is incorporated by reference herein.

CETP inhibitors useful in the present methods for treating or preventing a Condition include, but are not limited to, those disclosed in International Publication No. WO 00/38721 and U.S. Pat. No. 6,147,090, which are incorporated herein by reference.

LDL-receptor activators useful in the present methods for treating or preventing a Condition include, but are not limited to, include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Natural water-soluble fibers useful in the present methods for treating or preventing a Condition include, but are not limited to, psyllium, guar, oat and pectin.

Fatty acid esters of plant stanols useful in the present methods for treating or preventing a Condition include, but are not limited to, the sitostanol ester used in BENECOL® margarine.

Non-limiting examples of antidiabetic agents useful in the present methods for treating a Condition include insulin sensitizers, α-glucosidase inhibitors, DPP-IV inhibitors, insulin secretagogues, hepatic glucose output lowering compounds, antihypertensive agents, sodium glucose uptake transporter 2 (SGLT-2) inhibitors, insulin and insulin-containing compositions, and anti-obesity agents as set forth above.

In one embodiment, the antidiabetic agent is an insulin secretagogue. In one embodiment, the insulin secretagogue is a sulfonylurea.

Non-limiting examples of sulfonylureas useful in the present methods include glipizide, tolbutamide, glyburide, glimepiride, chlorpropamide, acetohexamide, gliamilide, gliclazide, gliquidone, glibenclamide and tolazamide.

In another embodiment, the insulin secretagogue is a meglitinide.

Non-limiting examples of meglitinides useful in the present methods for treating a Condition include repaglinide, mitiglinide, and nateglinide.

In still another embodiment, the insulin secretagogue is GLP-1 or a GLP-1 mimetic.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

Other non-limiting examples of insulin secretagogues useful in the present methods include exendin, GIP and secretin.

In another embodiment, the antidiabetic agent is an insulin sensitizer.

Non-limiting examples of insulin sensitizers useful in the present methods include PPAR activators or agonists, such as troglitazone, rosiglitazone, pioglitazone and englitazone; biguanidines such as metformin and phenformin; PTP-1B inhibitors; and glucokinase activators.

In another embodiment, the antidiabetic agent is a α-Glucosidase inhibitor.

Non-limiting examples of α-Glucosidase inhibitors useful the present methods include miglitol, acarbose, and voglibose.

In another embodiment, the antidiabetic agent is an hepatic glucose output lowering agent.

Non-limiting examples of hepatic glucose output lowering agents useful in the present methods include Glucophage and Glucophage XR.

In yet another embodiment, the antidiabetic agent is insulin, including all formualtions of insulin, such as long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In another embodiment, the antidiabetic agent is a DPP-IV inhibitor.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

In a further embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of antihypertensive agents useful in the present methods for treating a Condition include β-blockers and calcium channel blockers (for example diltiazem, verapamil, nifedipine, amlopidine, and mybefradil). ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In one embodiment, the antidiabetic agent is an agent that slows or blocks the breakdown of starches and certain sugars.

Non-limiting examples of antidiabetic agents that slow or block the breakdown of starches and certain sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol, camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in International Publication No. WO 00/07617.

Other specific additional therapeutic agents useful in the present methods for treating or preventing a Condition include, but are not limited to, rimonabant, 2-methyl-6-(phenylethynyl)-pyridine, 3[(2-methyl-1,4-thiazol-4-yl)ethynyl]pyridine, Melanotan-II, dexfenfluramine, fluoxetine, paroxetine, fenfluramine, fluvoxamine, sertaline, imipramine, desipramine, talsupram, nomifensine, leptin, nalmefene, 3-methoxynaltrexone, naloxone, nalterxone, butabindide, axokine, sibutramine, topiramate, phytopharm compound 57, Cerulenin, theophylline, pentoxifylline, zaprinast, sildenafil, aminone, milrinone, cilostamide, rolipram, cilomilast, phytanic acid, 4-[(E)-2-(5,6,7,8-tetramethyl-2-naphthalenyl)-1-propenyl]benzoic acid, retinoic acid, oleoyl-estrone, orlistat, lipstatin, tetrahydrolipstatin, teasaponin and diethylumbelliferyl phosphate.

In one embodiment, the present combination therapies for treating or preventing diabetes comprise administering a Bridged Bicyclic Heterocycle Derivative, an antidiabetic agent and/or an antiobesity agent.

In another embodiment, the present combination therapies for treating or preventing diabetes comprise administering a Bridged Bicyclic Heterocycle Derivative and an antidiabetic agent.

In another embodiment, the present combination therapies for treating or preventing diabetes comprise administering a Bridged Bicyclic Heterocycle Derivative and an anti-obesity agent.

In one embodiment, the present combination therapies for treating or preventing obesity comprise administering a Bridged Bicyclic Heterocycle Derivative, an antidiabetic agent and/or an antiobesity agent.

In another embodiment, the present combination therapies for treating or preventing obesity comprise administering a Bridged Bicyclic Heterocycle Derivative and an antidiabetic agent.

In another embodiment, the present combination therapies for treating or preventing obesity comprise administering a Bridged Bicyclic Heterocycle Derivative and an anti-obesity agent.

In one embodiment, the present combination therapies for treating or preventing metabolic syndrome comprise administering a Bridged Bicyclic Heterocycle Derivative and one or more additional therapeutic agents selected from: anti-obesity agents, antidiabetic agents, any agent useful for treating metabolic syndrome, any agent useful for treating a cardiovascular disease, cholesterol biosynthesis inhibitors, sterol absorption inhibitors, bile acid sequestrants, probucol derivatives, IBAT inhibitors, nicotinic acid receptor (NAR) agonists, ACAT inhibitors, cholesteryl ester transfer proten (CETP) inhibitors, low-denisity lipoprotein (LDL) activators, fish oil, water-soluble fibers, plant sterols, plant stanols and fatty acid esters of plant stanols.

In one embodiment, the additional therapeutic agent is a cholesterol biosynthesis inhibitor. In another embodiment, the cholesterol biosynthesis inhibitor is a squalene synthetase inhibitor. In another embodiment, the cholesterol biosynthesis inhibitor is a squalene epoxidase inhibitor. In still another embodiment, the cholesterol biosynthesis inhibitor is an HMG-CoA reductase inhibitor. In another embodiment, the HMG-CoA reductase inhibitor is a statin. In yet another embodiment, the statin is lovastatin, pravastatin, simvastatin or atorvastatin.

In one embodiment, the additional therapeutic agent is a cholesterol absorption inhibitor. In another embodiment, the cholesterol absorption inhibitor is ezetimibe.

In one embodiment, the additional therapeutic agent comprises a cholesterol absorption inhibitor and a cholesterol biosynthesis inhibitor. In another embodiment, the additional therapeutic agent comprises a cholesterol absorption inhibitor and a statin. In another embodiment, the additional therapeutic agent comprises ezetimibe and a statin. In another embodiment, the additional therapeutic agent comprises ezetimibe and simvastatin.

In one embodiment, the present combination therapies for treating or preventing metabolic syndrome comprise administering a Bridged Bicyclic Heterocycle Derivative, an antidiabetic agent and/or an antiobesity agent.

In another embodiment, the present combination therapies for treating or preventing metabolic syndrome comprise administering a Bridged Bicyclic Heterocycle Derivative and an antidiabetic agent.

In another embodiment, the present combination therapies for treating or preventing metabolic syndrome comprise administering a Bridged Bicyclic Heterocycle Derivative and an anti-obesity agent.

In one embodiment, the present combination therapies for treating or preventing a cardiovascular disease comprise administering one or more Bridged Bicyclic Heterocycle Derivatives, and an additional agent useful for treating or preventing a cardiovascular disease.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Bridged Bicyclic Heterocycle Derivatives are administered during a time when the additional therapeutic agent(s) exert their prophylactic or therapeutic effect, or vice versa.

In another embodiment, the one or more Bridged Bicyclic Heterocycle Derivatives and the additional therapeutic agent(s) are administered in doses commonly employed when such agents are used as monotherapy for treating a Condition.

In another embodiment, the one or more Bridged Bicyclic Heterocycle Derivatives and the additional therapeutic agent(s) are administered in doses lower than the doses commonly employed when such agents are used as monotherapy for treating a Condition.

In still another embodiment, the one or more Bridged Bicyclic Heterocycle Derivatives and the additional therapeutic agent(s) act synergistically and are administered in doses lower than the doses commonly employed when such agents are used as monotherapy for treating a Condition.

In one embodiment, the one or more Bridged Bicyclic Heterocycle Derivatives and the additional therapeutic agent(s) are present in the same composition. In one embodiment, this composition is suitable for oral administration. In another embodiment, this composition is suitable for intravenous administration.

The one or more Bridged Bicyclic Heterocycle Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Bridged Bicyclic Heterocycle Derivatives and the additional therapeutic agent(s) may inhibit the resistance of a Condition to these agents.

In one embodiment, when the patient is treated for diabetes or a diabetic complication, the additional therapeutic agent is an antidiabetic agent which is not a Bridged Bicyclic Heterocycle Derivative. In another embodiment, the additional therapeutic agent is an agent useful for reducing any potential side effect of a Bridged Bicyclic Heterocycle Derivative. Such potential side effects include, but are not limited to, nausea, vomiting, headache, fever, lethargy, muscle aches, diarrhea, general pain, and pain at an injection site.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Bridged Bicyclic Heterocycle Derivative(s) and the other agent(s) for treating diseases or conditions listed above can be administered simultaneously or sequentially. This particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Bridged Bicyclic Heterocycle Derivatives and the additional therapeutic agent(s)can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Bridged Bicyclic Heterocycle Derivatives or a pharmaceutically acceptable salt, solvate, ester, prodrug or stereoisomer thereof, and a pharmaceutically acceptable carrier.

For preparing compositions comprising one or more Bridged Bicyclic Heterocycle Derivatives, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, a Bridged Bicyclic Heterocycle Derivative is administered orally.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, the condition and size of the patient, as well as severity of the symptoms being treated. A typical recommended daily dosage regimen for oral administration can range from about 1 mg/day to about 1000 mg/day, 1 mg/day to about 500 mg/day, 1 mg/day to about 300 mg/day, 1 mg/day to about 75 mg/day, 1 mg/day to about 50 mg/day, or 1 mg/day to about 20 mg/day, in one dose or in two to four divided doses.

When the invention comprises a combination of one or more Bridged Bicyclic Heterocycle Derivatives and an additional therapeutic agent, the two active components may be co-administered simultaneously or sequentially, or a single composition comprising one or more Bridged Bicyclic Heterocycle Derivatives and the additional therapeutic agent(s) in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of an advantageous effect of the combination.

In one embodiment, the components of a combination therapy regimen are to be administered simultaneously, they can be administered in a single composition with a pharmaceutically acceptable carrier.

In another embodiment, when the components of a combination therapy regimen are to be administered separately or sequentially, they can be administered in separate compositions, each containing a pharmaceutically acceptable carrier.

### Kits

In one aspect, the present invention provides a kit comprising an effective amount of one or more Bridged Bicyclic Heterocycle Derivatives, or a pharmaceutically acceptable salt, solvate, ester, prodrug or stereoisomer thereof, and a pharmaceutically acceptable carrier.

In another aspect the present invention provides a kit comprising an amount of one or more Bridged Bicyclic Heterocycle Derivatives, or a pharmaceutically acceptable salt, solvate, ester, prodrug or stereoisomer thereof, and an amount of one or more additional therapeutic agents listed above, wherein the combined amounts are effective for treating or preventing a Condition in a patient.

When the components of a combination therapy regimen are to be administered in more than one composition, they can be provided in a kit comprising a single package containing one or more containers, wherein one container contains one or more Bridged Bicyclic Heterocycle Derivatives in a pharmaceutically acceptable carrier, and a second, separate container comprises an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited herein, the entire disclosures of which are incorporated herein by reference.

1. A compound having the formula: 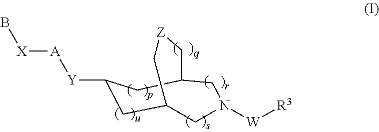
or a pharmaceutically acceptable salt, solvate, ester, prodrug or stereoisomer thereof, wherein:
A is pyridyl or pyrimidinyl, wherein said pyridyl and said pyrimidinyl can be optionally substituted with up to 2 groups, which can be the same or different, and are selected from alkyl, cycloalkyl, halo and —O-alkyl;
B is phenyl or 6-membered heteroaryl, wherein said phenyl or said 6-membered heteroaryl can be optionally substituted with up to 3 groups, which can be the same or different, and are selected from alkyl, heterocycloalkyl, heteroaryl, halo, —CN, —S(O)₂alkyl and —S(O)₂cycloalkyl, wherein said heterocycloalkyl or heteroaryl substituent groups can be unsubstituted or optionally substituted with alkyl, and wherein a ring —CH₂— group on said heterocycloalkyl substituent group can be optionally replaced with a —C(O)— group;
W is a bond, —C(O)—, —C(O)NH—, —C(O)—O—, —C(O)—S— or —S(O)₂—;
X is —O-(alkylene)ₜ- or —NH—;
Y is —O— or —NH—;
Z is a bond, —C(O)—, —C(R¹)₂—, —O—, —S(O)₂— or —N(R⁴)—
each occurrence of R¹ is independently H or —OH; wherein two R¹ groups, together with the carbon atom(s) to which they are attached, can join to form a 3- to 6-membered cycloalkyl group or a 3- to 6-membered heterocycloalkyl group;
R³ is -(alkylene)ₜ-cycloalkyl, wherein the cycloalkyl moiety of said -(alkylene)ₜ-cycloalkyl group can be optionally substituted with alkyl, —O-alkyl or -alkylene-O-alkyl;
R⁴ is H, haloalkyl, aryl, cycloalkyl, cycloalkenyl, heterocycloalkyl, heterocycloalkenyl or heteroaryl;
n is an integer ranging from 1 to 4.
p is 0 or 1;
q is 0 or 1;
r is 0 or 1;
s is 0 or 1;
t is 0 or 1; and
u is 0 or 1.

2. The compound of claim 1, wherein W is —C(O)O— or a bond.

3-4. (canceled)

5. The compound of claim 1, wherein Y is —O— and X is —O— or —NH—.

6. The compound of claim 1, wherein A is pyridyl or pyrimidinyl, wherein said pyridyl group can be optionally substituted with a 5-membered heteroaryl group, and wherein said pyrimidinyl group can be optionally substituted with alkyl, halo, cycloalkyl or —O-alkyl.

7. The compound of claim 6, wherein A is: 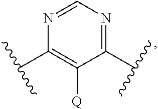
wherein Q is F, methyl, ethyl, ethoxy or methoxy.

8. The compound of claim 6, wherein B is: (i) phenyl, which is optionally substituted with up to 3 groups, which can be the same or different, and are selected from alkyl, —S(O)₂alkyl, halo and —CN, or (ii) pyridyl, which is optionally substituted with up to 2 groups, which can be the same or different, and are selected from 5-membered heteroaryl and —S(O)₂alkyl.

9. The compound of claim 1, wherein the group —B—X-A-Y— is: 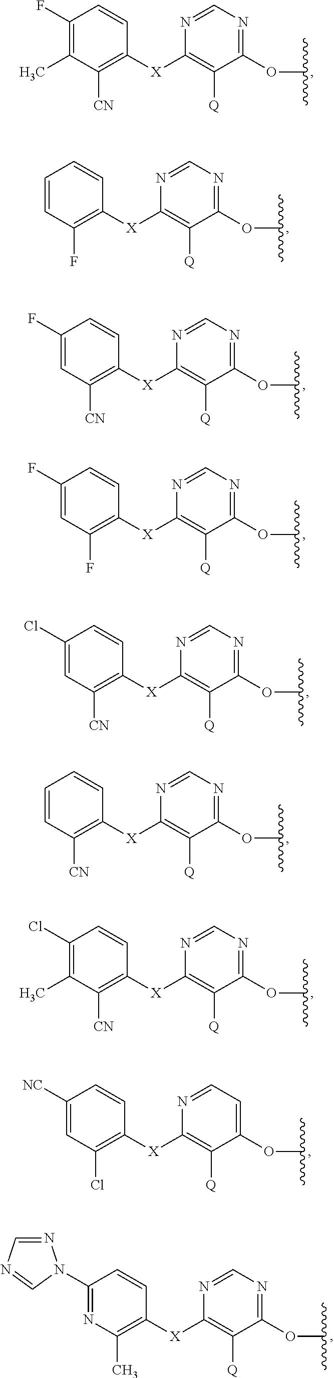 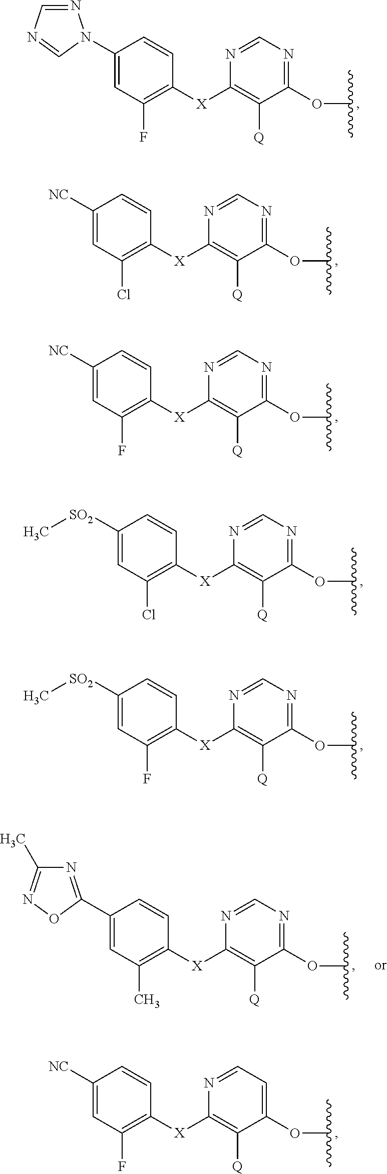
wherein X is —O— or —NH—, and Q is H, halo, alkyl, cycloalkyl or —O-alkyl.

10. The compound of claim 1, wherein the group B—X-A-Y— is:

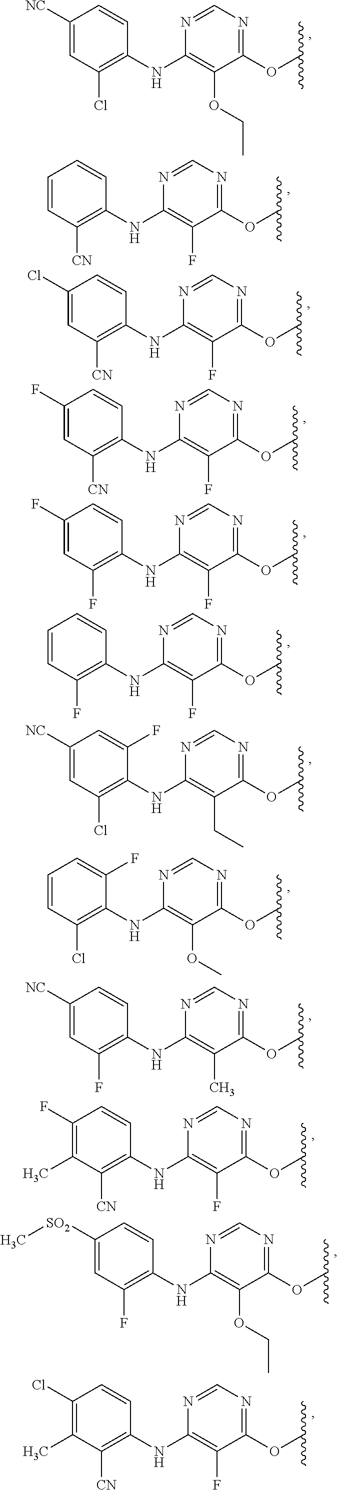

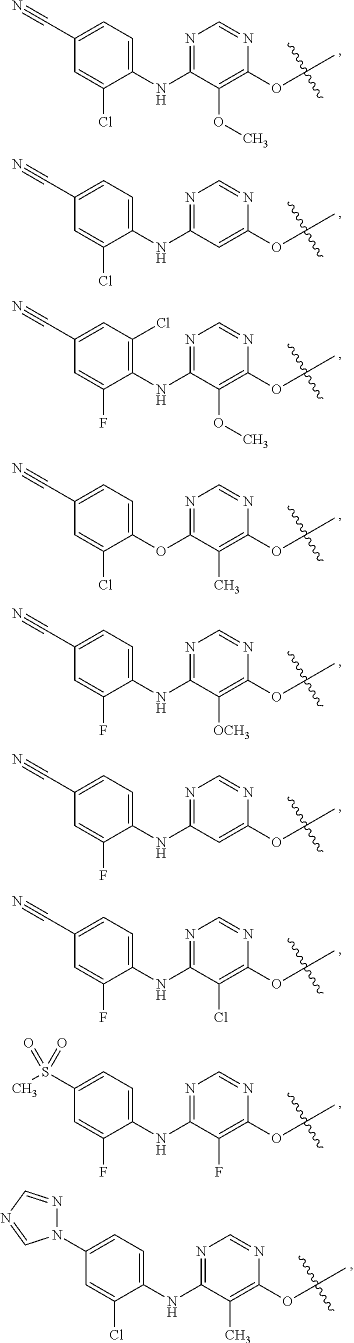

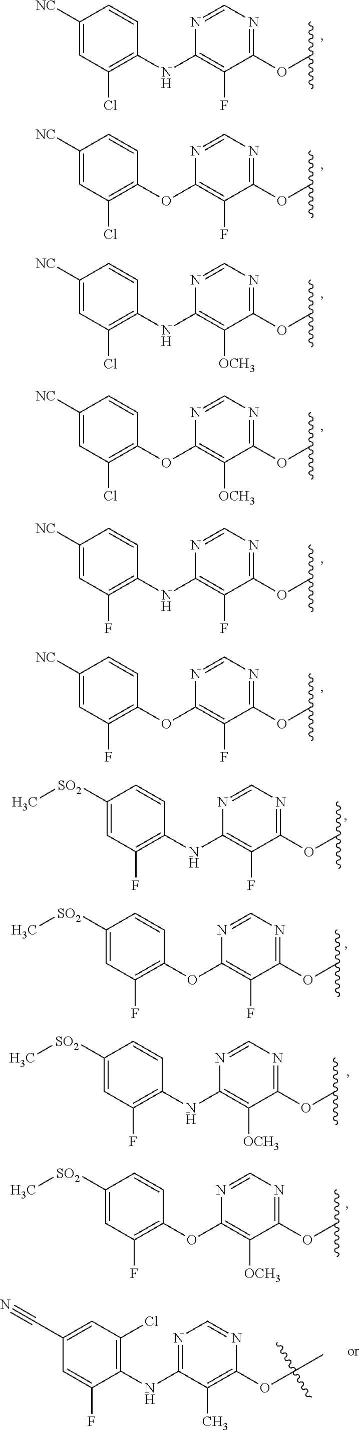

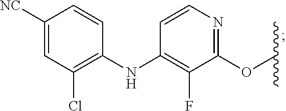

11. The compound of claim 1, wherein the group

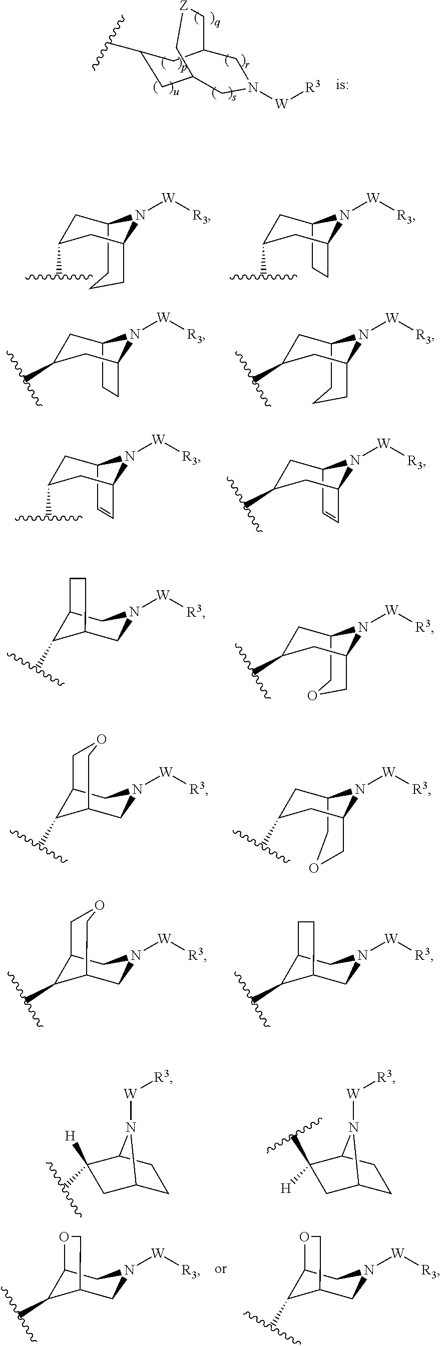

12. The compound of claim 11, wherein the group

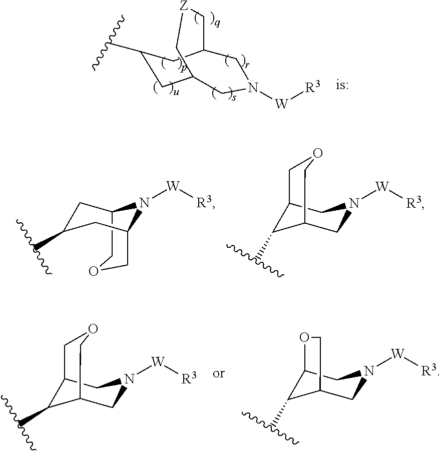

13-15. (canceled)

17. A compound having the structure: 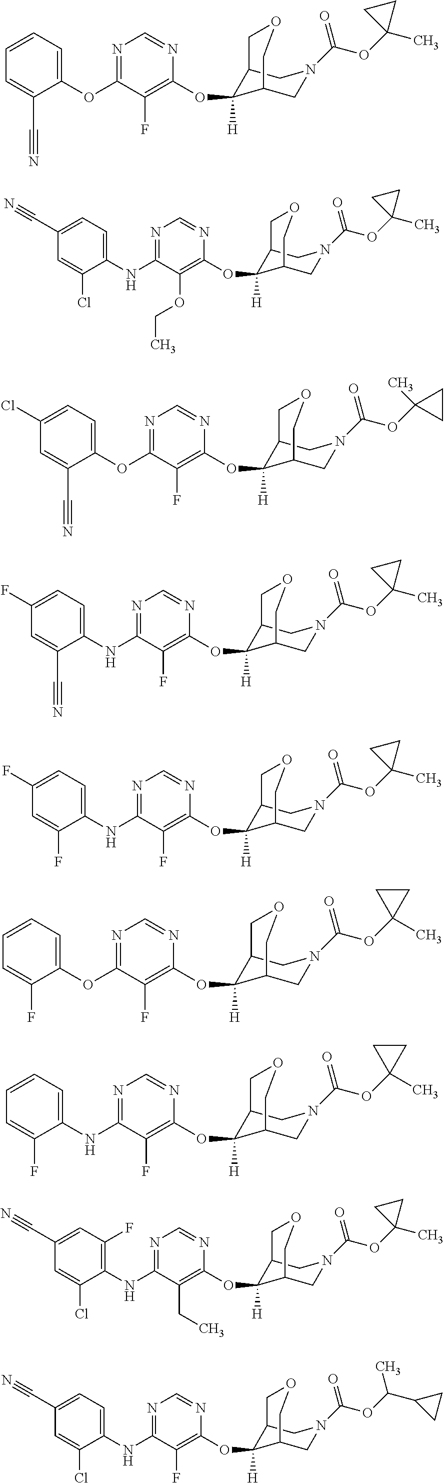 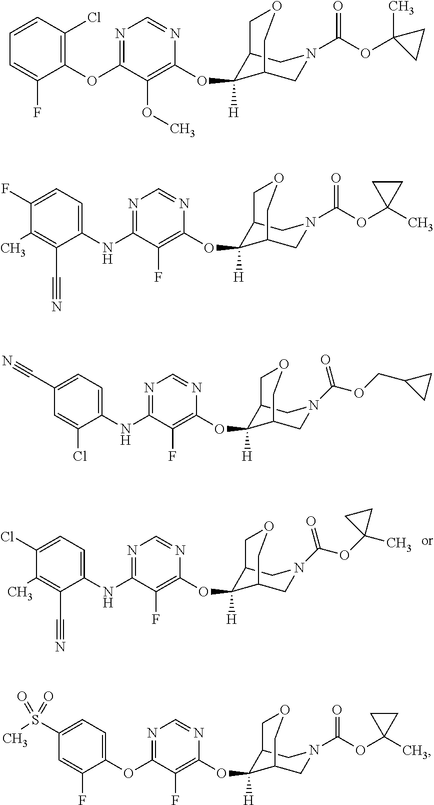
or a pharmaceutically acceptable salt, solvate, ester, prodrug or stereoisomer thereof.

18. A composition comprising an effective amount of one or more compounds of claim 1 or a pharmaceutically acceptable salt, solvate, ester, prodrug or stereoisomer thereof, and at least one pharmaceutically acceptable carrier.

19. A method for treating diabetes, obesity or metabolic syndrome in a patient, the method comprising administering to the patient an effective amount of one or more compounds of claim 1 or a pharmaceutically acceptable salt, solvate, ester, prodrug or stereoisomer thereof.

20. The method of claim 19, further comprising administering an effective amount of one or more additional therapeutic agents, wherein the additional therapeutic agents are selected from antidiabetic agents and antiobesity agents.
